(19) United States
(12) Patent Application Publication
(10) Pub. No.: US 2025/0361253 A1
(43) Pub. Date: Nov. 27, 2025

(54) STING AGONIST COMPOUNDS

(71) Applicant: Sutro Biopharma, Inc., South San Francisco, CA (US)

(72) Inventors: Krishna BAJJURI, Union City, CA (US); Andreas Maderna, Walnut Creek, CA (US); Scott Baggett, San Mateo, CA (US); Huiyong Hu, Fremont, CA (US); Adam Antoni Galan, Alameda, CA (US); Wen Liu, Foster City, CA (US); Valery Rostislavovich Polyakov, Oakley, CA (US); Xiangwei Zhu, Albany, CA (US); Ravi Prakash Singh, Burlingame, CA (US)

(21) Appl. No.: 18/871,630

(22) PCT Filed: Jun. 5, 2023
(86) PCT No.: PCT/US2023/024491

§ 371 Date: Dec. 4, 2024

Related U.S. Application Data
(60) Provisional application No. 63/365,919, filed on Jun. 6, 2022.

(51) Int. Cl. C07F 9/6558; A61K 31/381; A61K 31/4709; A61K 31/4725; A61K 31/498; A61K 31/502; A61K 31/517; A61K 31/67; A61K 31/675; C07D 215/20; C07D 333/56; C07D 333/64; C07D 409/12

(52) U.S. Cl. CPC C07F 9/65586; A61K 31/381; A61K 31/4709; A61K 31/4725; A61K 31/498; A61K 31/502; A61K 31/517; A61K 31/67; A61K 31/675; C07D 215/20; C07D 333/56; C07D 333/64; C07D 409/12

(57) ABSTRACT

The present disclosure is related to STING agonists, pharmaceutical compositions thereof, and the use of the agonists and pharmaceutical compositions to induce a STING-mediated immune response and/or to treat diseases and disorders mediated by STING, such as cellular proliferative disorders, including, but not limited to, cancer.

### CROSS REFERENCE TO RELATED APPLICATION

This application claims the benefit of U.S. Provisional Application No. 63/365,919 filed Jun. 6, 2022, the contents of which are hereby incorporated by reference in its entirety.

### FIELD

The present disclosure is related to STING agonists, pharmaceutical compositions thereof, and the use of the agonists and pharmaceutical compositions to induce a STING-mediated immune response and/or to treat diseases and disorders mediated by STING, such as cellular proliferative disorders, including, but not limited to, cancer.

### BACKGROUND

The innate and adaptive immune systems work closely together to fight foreign substances and invading pathogens. While the adaptive system is highly specific and long-lasting due to the production of memory T-cells, the innate system acts quickly as the body's first-line of defence. The innate system responds non-specifically to both pathogen-derived cytosolic DNA and host cytosolic DNA. In doing this, the innate immune system not only provides broad protection against threats such as bacteria and viruses, but also responds to signals of cellular and tissue damage.

One protein that is important for innate immunity is stimulator of interferon genes (STING), and the cGAS-STING pathway in particular helps to sense and protect against harmful cytosolic DNA. cGAS recognizes cytosolic DNA and catalyses the synthesis of cyclic dinucleotides (CDNs), including cGAMP, which in turn bind and activate STING. Once STING is bound to a CDN, STING undergoes a conformational change, translocates from the endoplasmic reticulum to the Golgi apparatus, and triggers the transcription factor TBK1 to phosphorylate transcription factors interferon regulatory factor 3 (IRF3) and nuclear factor κB (NF-κB). This induces type I interferons (IFNs) and the production of pro-inflammatory cytokines, such as IL-6, TNF-α, and IFN-γ.

Through this mechanism, various DNA viruses have been shown to activate the STING pathway, including the herpes simplex virus type I (HSV-1), Kaposi sarcoma herpes virus (KSHV), cytomegalovirus (CMV), hepatitis B (HBV), human papillomavirus (HPV), adenoviruses, and baculoviruses. Studies have also shown that STING can protect against RNA infections. For example, STING knockout mice are more susceptible to RNA viruses (Ishikawa, H., et al. Nature, 2009, 461, pages 788-792). Additionally, intracellular bacteria have been shown to produce CDNs that can activate the STING pathway and induce an immune response. For example, the bacteria strain Listeria monocytogenes elicits a STING-induced immune response.

While foreign agents can activate the STING pathway, many viruses have developed methods to suppress or inhibit the STING-promoted immune response. For example, a number of HSV-1 viral genes are capable of suppressing STING signalling pathways, including HSV-1 γ34.5, which disrupts STING trafficking from the endoplasmic reticulum to the Golgi apparatus (Christensen, M. H., et al., EMBO, 2016, 35, 568). Similarly, it has also been demonstrated that Kaposi sarcoma herpes virus (KSHV), human papillomavirus (HPV), cytomegalovirus (CMV), and hepatitis B (HBV) viral genes have developed mechanisms for evading the STING pathway. (Ahn, J. et al. Experimental and Molecular Medicine, 2019, 51, 155).

Furthermore, certain RNA viruses affect IFN production. For instance, it has been reported that the protease of Dengue Fever (DENV), a single-positive-stranded RNA virus, can inhibit type I INF production by targeting and cleaving STING. In STING-deficient primary cells, DENV replication is highly increased (Yu, C. et al. PLoS Pathog. 2012, 8, e1002780; Aguirre, S., et al. PLoS Pathog. 2012, 8, e1002934). Similarly, the protease for Zika virus (ZIKV), another single-positive-stranded RNA virus of the same Flaviviridae family, can also cleave STING and through downstream effects, reduce type I IFN production (Ding, et al. Proc. Natl. Acad. Sci. USA 2018, 115, E6310; Zheng et al. EMBO, 2018, 37:e99347).

In addition to playing a role in mounting an immune response to the invasion of foreign pathogens, the STING pathway also recognizes host-cytosolic DNA. Because the cytosol is normally free of DNA, leaked cytosolic DNA is often an indication of DNA damage events and can be indicative of tumorigenesis. Detection of host-cytosolic DNA by STING leads to the production of IFNs, immune-stimulated genes, and pro-inflammatory cytokines. It has been well-established that IFNs can inhibit tumor cell proliferation via multiple mechanisms. As described in Jiang, M. et al. Journal of Hematology & Oncology, 2020, 81, 13, STING-deficiency is correlated with cancer incidence in at least melanoma cell lines, colorectal adenocarcinoma human cell lines, and lung cancer.

A number of STING agonists have been developed and studied for oncological indications (Le Naour et al. Oncoimmunology, 2020; 9(1): 1777624), including DMXAA (or Vadimezan), a tumor-vascular disrupting agent that has been studied in clinical trials for its effect on advanced solid tumors, prostate cancer, urothelial carcinoma, and small cell lung cancer. Despite promising preclinical results, DMXAA has thus far only yielded poor results in human clinical trials. MIW815 (ADU-S100) in combination with pembrolizumab was recently studied in a Phase 2 clinical trial for patients with head and neck cancer, but was terminated due to a lack of substantial anti-tumor activity (NCT03937141). A Phase 1 trial to study the effect of MIW815 as a single agent and in combination with ipilimumab in patients with advanced/metastatic solid tumors or lymphomas (NCT02675439) was also terminated for showing a lack of substantial anti-tumor activity.

Other STING agonists for various cancer treatments include BMS-986301, E7766, GSK3745417, MK-1454, MK-2118, BI 1387446 and SB11285. Dimeric STING agonists have been described in PCT Applications WO 2021/113679 assigned to Mersana Therapeutics, Inc. and U.S. Pat. No. 10,793,557 assigned to Merck Sharp and Dohme. Additional STING agonists are also described in US Application No. US2021/0009608 assigned to Merck.

Given the importance of the STING pathway in inducing an immune response both in response to foreign pathogens and damaged DNA associated with cellular proliferative disorders, there is a medical need to develop STING agonists. It is therefore an object of the present disclosure to provide novel compounds and compositions that can induce a STING-mediated immune response and/or provide treatment of diseases and disorders mediated by STING, such as cellular proliferative disorders.

### SUMMARY

In one aspect, the STING agonist is a compound of the Formula I, Formula II, or Formula III:

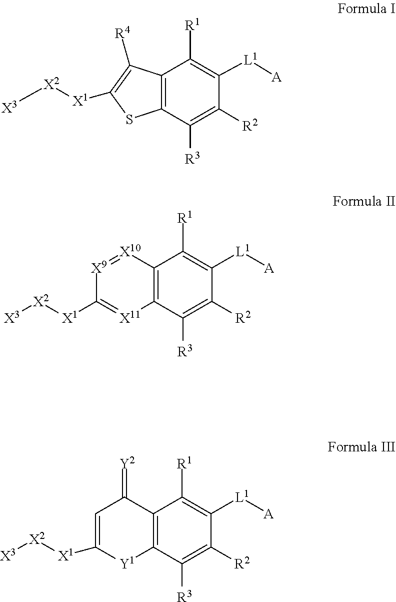

or a pharmaceutically acceptable salt, solvate, or stereoisomer thereof;
wherein:
L¹ is selected from C₁-C₆alkylene,

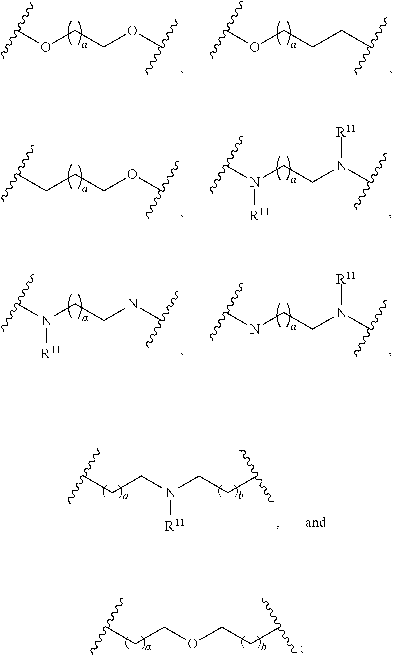

wherein any substitutable carbon in L¹ is optionally substituted with one or more substituents selected from halogen, alkoxy, C₁₋₆alkyl, C₁₋₆alkylamino, C₃₋₁₀cycloalkyl, hydroxy, cyano, —NR^{9a}R^{9b}, and —COOR¹⁰;
A is selected from

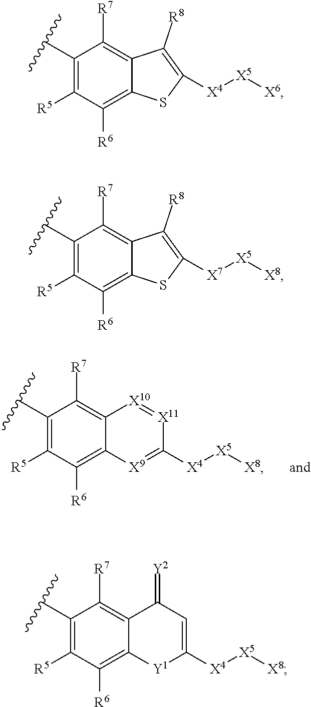

R¹, R², R³, R⁴, R⁵, R⁶, R⁷ and R⁸ are independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, amino-C₁₋₆alkyl, C₁₋₆alkylamino-C₁₋₆alkyl, di-C₁₋₆alkylamino-C₁₋₆alkyl, and —COOR¹⁰;
X¹ and X⁴ are independently selected from —C(O)—, —C(R^{12a}R^{12b})—, —CH═CH—, and —C(R^{12a}R^{12b})C(R^{13a}R^{13b})—;
X² and X⁵ are independently selected from —(C(R^{12a}R^{12b}))₁₋₆— and —(C(R^{12a}R^{12b})C(R^{13a}R^{13b}))₁₋₃;
X³ is selected from —COOR¹⁰, —CONR^{9a}R^{9b}, —C(O)NR^{9a}S(O)₂R¹⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁶ is selected from —C(O)NR^{9a}S(O)₂R¹⁷, —C(O)NR^{9a}R¹⁸, —S(O)₂OH, —S(O)OH, —P(O)(OR¹⁵)₂, —OP(O)(OR¹⁵)₂, and —P(O)(NR^{16a}R^{16b})(OR¹⁵);
X⁷ is —CH═CH—, C₂alkynyl, or —C(R^{12a}R^{12c})C(R^{13a}R^{13c})—;
X⁸ is selected from —COOR¹⁰, —CONR^{9a}R^{9b}, —C(O)NR^{9a}S(O)₂R¹⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁹, X¹⁰, and X¹¹ are independently selected from CR¹⁹ and N;
Y¹ is independently selected from CH₂, O, NH, and S;
Y² is independently selected from O, NH, and S;
R^{9a} and R^{9b} are independently selected from hydrogen, C₁₋₆alkyl, C₃₋₁₀cycloalkyl, aryl, aryl-C₁₋₆alkyl, amino-C₁₋₆alkyl, C₁₋₆alkylamino-C₁₋₆alkyl, di-C₁₋₆alkylamino-C₁₋₆alkyl, and heteroaryl;
R¹⁰ is independently selected from hydrogen, C₁₋₆alkyl, C₃₋₁₀cycloalkyl, aryl, aryl-C₁₋₆alkyl, and heteroaryl;
R¹¹ is independently selected from hydrogen and C₁₋₆alkyl;
R^{12a}, R^{12b}, R^{13a}, and R^{13b} are independently selected from hydrogen and C₁₋₆alkyl; or R^{12b} and R^{13b} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R^{12c} and R^{13c} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R¹⁴ is selected from C₁₋₆alkyl, C₃₋₁₀cycloalkyl, halo-C₁₋₆alkyl, amino-C₁₋₆alkyl, C₁-6alkyl-amino-C₁₋₆alkyl, di-C₁₋₆alkylamino-C₁₋₆alkyl, aryl, aryl-C₁₋₆alkyl, and heteroaryl;
R¹⁵, R^{16a}, and R^{16b} are independently selected from hydrogen, C₁₋₆alkyl, and C₃₋₁₀cycloalkyl;
R¹⁷ is amino-C₁₋₆alkyl, C₁₋₆alkyl-amino-C₁₋₆alkyl, di-C₁₋₆alkylamino-C₁₋₆alkyl, C₃₋₁₀cycloalkyl-amino-C₁₋₆alkyl, aryl, aryl-C₁₋₆alkyl, or heteroaryl;
R¹⁸ is amino-C₁₋₆alkyl, C₁₋₆alkylamino-C₁₋₆alkyl, di-C₁₋₆alkylamino-C₁₋₆alkyl, C₃₋₁₀cycloalkyl-amino-C₁₋₆alkyl, C₁₋₆alkyl, C₃₋₁₀cycloalkyl, aryl, aryl-C₁₋₆alkyl, or heteroaryl; and
R¹⁹ is independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, and —COOR¹⁰; and
each of a and b is an integer independently selected from 0, 1, 2, 3, 4, and 5.

In one embodiment, the STING agonist is a compound of Formula Ia:

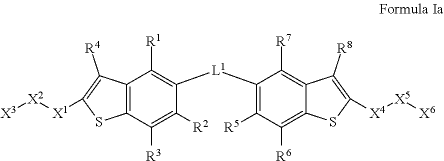

or a pharmaceutically acceptable salt, solvate, or stereoisomer thereof;
wherein L¹, X¹, X², X³, X⁴, X⁵, X⁶, R¹, R², R³, R⁴, R¹, R⁶, R⁷, R¹, R^{9a}, R^{9b}, R¹⁰, R¹¹, R^{12a}, R^{12b}, R^{13a}, R^{13b}, R¹⁴, R, R^{16a}, R^{16b}, a, and b are as defined herein.

In one embodiment, the STING agonist is a compound of Formula Ib:

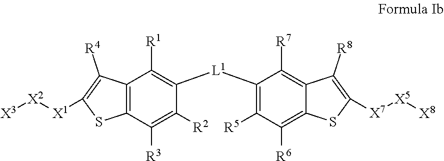

or a pharmaceutically acceptable salt, solvate, or stereoisomer thereof;
wherein L¹, X¹, X², X³, X⁵, X⁷, X⁸, R¹, R², R³, R⁴, R⁵, R⁶, R⁷, R⁸, R^{9a}, R^{9b}, R¹⁰, R¹¹, R^{12a}, R^{12b}, R^{12c}, R^{13a}R^{13b}, R¹³, R¹⁴, R¹⁵, R^{16a}, R^{16b}, a, and b are as defined herein.

In one embodiment, the STING agonist is a compound of Formula Ic:

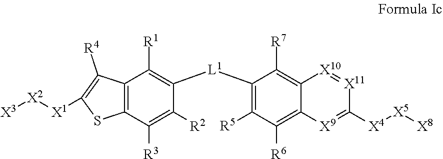

or a pharmaceutically acceptable salt, solvate, or stereoisomer thereof;
wherein L¹, X¹, X², X³, X⁴, X⁵, X⁸, X⁹, X¹⁰, X¹¹, R¹, R², R³, R⁴, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, R¹⁹, a, and b are as defined herein.

In one embodiment, the STING agonist is a compound of Formula Id:

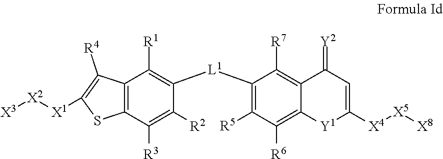

or a pharmaceutically acceptable salt, solvate, or stereoisomer thereof;
wherein L¹, X¹, X², X³, X⁴, X⁵, X⁸, Y¹, Y², R¹, R², R³, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{6b}, R¹⁹, a, and b are as defined herein.

In one embodiment, the STING agonist is a compound of Formula IIa:

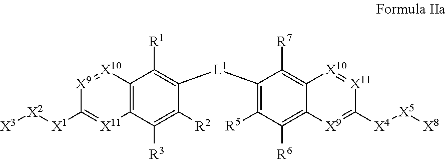

or a pharmaceutically acceptable salt, solvate, or stereoisomer thereof;
wherein L¹, X¹, X², X³, X⁴, X⁵, X⁸, X⁹, X¹⁰, X¹¹, R¹, R², R³, R⁵, R⁶, R⁷, R⁸, R^{9a}, R^{9b}, R¹⁰, R¹¹, R^{12a}, R¹²⁶, R^{13a}, R^{13b}, R¹⁴, R¹⁵, R¹⁶, R^{16b}, R¹⁹, a, and b are as defined herein.

In one embodiment, the STING agonist is a compound of Formula IIb:

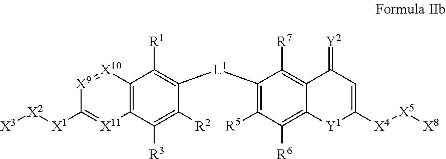

or a pharmaceutically acceptable salt, solvate, or stereoisomer thereof;
wherein L¹, X¹, X², X³, X⁴, X⁵, X⁸, Y¹, Y², R¹, R², R³, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, a, and b are as defined herein.

In one embodiment, the STING agonist is a compound of Formula IIIa:

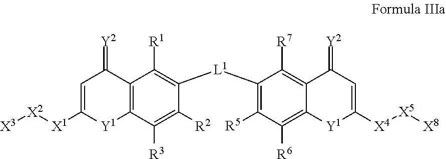

or a pharmaceutically acceptable salt, solvate, or stereoisomer thereof;
wherein L¹, X¹, X², X³, X⁴, X⁵, X⁸, Y¹, Y², R¹, R², R³, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, a, and b are as defined herein.

The present disclosure provides at least the following embodiments:
a) A compound of Formula I, Formula II, Formula III, Formula Ia, Formula Ib, Formula Ic, Formula Id, Formula IIa, Formula IIb, or Formula IIIa or a pharmaceutically acceptable salt, solvate, or stereoisomer thereof;
b) A compound selected from Compound 1-Compound 31 or a pharmaceutically acceptable salt, solvate, or stereoisomer thereof;
c) A compound selected from Compound 32-Compound 62 or a pharmaceutically acceptable salt, solvate, or stereoisomer thereof;
d) A pharmaceutical composition comprising a compound of Formula I, Formula II, Formula III, Formula Ia, Formula Ib, Formula Ic, Formula Id, Formula Ila, Formula IIb, or Formula IIIa or a pharmaceutically acceptable salt, solvate, or stereoisomer thereof in a pharmaceutically acceptable excipient, diluent, or carrier;
e) A pharmaceutical composition comprising a compound of (b) or (c) in a pharmaceutically acceptable excipient, diluent, or carrier;
f) A method of treating a disease or disorder mediated by STING in a subject in need thereof comprising administering a therapeutically effective amount of a compound of (a)-(c) or a pharmaceutical composition of (d)-(e);
g) The method of (f) wherein the disease or disorder is a cellular proliferative disorder, including, but not limited to cancer;
h) The method of (g) wherein the cancer is selected from acute myeloid leukemia; breast cancer; colorectal cancer; glioma; head and neck squamous cell carcinoma; lung cancer, including non-small cell lung cancer; head and neck cancer; lymphoma, including a malignant lymphoma; melanoma; nasopharyngeal carcinoma; ovary cancer; pancreatic cancer; prostate cancer; urothelial cancer; and tongue squamous cell carcinoma;
i) A method to induce an immune response in a subject in need thereof comprising administering a therapeutically effective amount of a compound of (a)-(c) or a pharmaceutical composition of (d)-(e);
j) A method to induce STING-dependent type I interferon production in a subject in need thereof comprising administering a therapeutically effective amount of a compound of (a)-(c) or a pharmaceutical composition of (d)-(e);
k) A method to induce STING-dependent cytokine production in a subject in need thereof comprising administering a therapeutically effective amount of a compound of a compound of (a)-(c) or a pharmaceutical composition of (d)-(e);
l) The method of any one of embodiments (f)-(k) wherein the compound of (a)-(c) or the pharmaceutical composition of (d)-(e) is administered in combination with an immune modulator, including but not limited to a checkpoint inhibitor, for example, a PD-1 inhibitor or a CTLA-4 inhibitor;
m) Use of a therapeutically effective amount of a compound of (a)-(c) or a pharmaceutical composition of (d)-(e) for the treatment of a disease or disorder mediated by STING in a subject in need thereof;
n) Use of a therapeutically effective amount of a compound of (a)-(c) or a pharmaceutical composition of (d)-(e) to induce an immune response in a subject in need thereof;
o) Use of a therapeutically effective amount of a compound of (a)-(c) or a pharmaceutical composition of (d)-(e) to induce STING-dependent type I interferon production in a subject in need thereof;
p) Use of a therapeutically effective amount of a compound of (a)-(c) or a pharmaceutical composition of (d)-(e) to induce STING-dependent cytokine production in a subject in need thereof;
q) Use of a therapeutically effective amount of a compound of (a)-(c) or a pharmaceutical composition of (d)-(e) in the manufacture of a medicament for the treatment of a disease or disorder mediated by STING in a subject in need thereof;
r) Use of a therapeutically effective amount of a compound of (a)-(c) or a pharmaceutical composition of (d)-(e) in the manufacture of a medicament to induce an immune response in a subject in need thereof;
s) Use of a therapeutically effective amount of a compound of (a)-(c) or a pharmaceutical composition of (d)-(e) in the manufacture of a medicament to induce STING-dependent type I interferon production in a subject in need thereof;
t) Use of a therapeutically effective amount of a compound of (a)-(c) or a pharmaceutical composition of (d)-(e) in the manufacture of a medicament to induce STING-dependent cytokine production in a subject in need thereof; and
u) A kit comprising a therapeutically effective amount of a compound of (a)-(c) or a pharmaceutical composition of (d)-(e) and instructions for use of the compound.

### DETAILED DESCRIPTION

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes a saturated straight or branched hydrocarbon having one to six carbon atoms, i.e., C₁ to C₆ alkyl, or lower alkyl. The term includes both substituted and unsubstituted moieties. In some or any embodiments, the alkyl is unsubstituted. In some or any embodiments, the alkyl is substituted. In certain embodiments, the alkyl group is a fluorinated alkyl group. Non-limiting examples of moieties with which the alkyl group can be substituted are selected from the group consisting of halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. In certain embodiments, the alkyl group is selected from the group consisting of methyl, CF₃, CC13, CFCl₂, CF₂Cl, ethyl, CH₂CF₃, CF₂CF₃, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl.

The term “alkylene,” as used herein, unless otherwise specified, refers to a divalent alkyl group, as defined herein. In some or any embodiments, alkylene is unsubstituted.

The term “aryl” indicates an aromatic group containing only carbon in the aromatic ring or rings. In one embodiment, the aryl group contains 1 to 3 separate or fused rings and is 6 to about 14 or 18 ring atoms, without heteroatoms as ring members. When indicated, such aryl groups may be further substituted with carbon or non-carbon atoms or groups. Such substitution may include fusion to a 4 to 7 or a 5 to 7-membered saturated or partially unsaturated cyclic group that optionally contains 1, 2 or 3 heteroatoms independently selected from N, O, B, P, Si and/or S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described above.

The terms “alkoxy” and “alkoxyl,” refer to the group —OR″ where R″ is alkyl or cycloalkyl. Alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

The term “amino” refers to the radical —NH₂.

The term “aminoalkyl” refers to an alkyl group, as defined herein, which is substituted with one or more amino groups. In some or any embodiments, the aminoalkyl is an alkyl group substituted with one —NH₂ group (e.g., R′(NH₂) wherein R′ is alkyl, as defined herein). In some or any embodiments, the aminoalkyl is an alkyl group substituted with two —NH₂ groups. In some embodiments, “aminoalkyl” is amino-C₁₋₆alkyl.

The term “alkylamino,” refers to the group —NHR′ wherein R′ is alkyl, as defined herein. In some or any embodiments, the alkylamino is C₁₋₆alkylamino.

The term “dialkylamino,” refers to the group —NR′R′ wherein R′ is alkyl, as defined herein. In some or any embodiments, the dialkylamino is di-C₁₋₆alkylamino.

The term “alkylaminoalkyl,” refers to an alkyl group, as defined herein, which is substituted with one or more alkylamino groups as defined herein. In some embodiments, an “alkylaminoalkyl” is C₁₋₆alkyl-amino-C₁₋₆alkyl. In some embodiments, each alkyl in alkylaminoalkyl is independently selected.

The term “dialkylaminoalkyl” refers to an alkyl group, as defined herein, which is substituted with one or more dialkylamino groups as defined herein. In some embodiments, “dialkylaminoalkyl” is di-C₁₋₆alkylamino-C₁₋₆alkyl. In some embodiments, each alkyl in dialkylaminoalkyl is independently selected.

The term “arylalkyl” refers to an alkyl group, as defined herein, substituted with an aryl group as defined herein. In one embodiment, “arylalkyl” is benzyl.

The term “haloalkyl” refers to an alkyl group, as defined herein, substituted with one or more halo groups. Haloalkyl groups include, but are not limited to, —CF₃, —CH₂F, —CHF₂, and —CH₂F₃.

The term “hydroxyalkyl” refers to an alkyl group, as defined herein, substituted with one or more hydroxy groups.

The terms “halogen” and “halo,” as used herein, and unless otherwise specified, are synonymous and refer to chloro, bromo, fluoro or iodo.

The term “hydroxy” refers to an —OH group.

The term “cyano” refers to an —CN group.

The term “cycloalkyl”, as used herein, unless otherwise specified, refers to a saturated cyclic hydrocarbon. In certain embodiments, the cycloalkyl group may be a saturated, and/or bridged, and/or non-bridged, and/or a fused bicyclic group. In certain embodiments, the cycloalkyl group includes three to ten carbon atoms, i.e., C₃ to C₁₀ cycloalkyl. In some embodiments, the cycloalkyl has from 3 to 10 (C₃₋₁₀) or from 3 to 6 (C₃₋₆) carbon atoms. In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl. In some or any embodiments, cycloalkyl is substituted with 1, 2, or three groups independently selected from halogen (fluoro, chloro, bromo or iodo), alkyl, haloalkyl, hydroxyl, amino, alkylamino, and alkoxy.

The term “heteroaryl” refers to a monovalent monocyclic aromatic group and/or multicyclic aromatic group, wherein at least one aromatic ring contains one or more heteroatoms independently selected from O, S, and N in the ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, imidazolyl, triazolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is heteroaryl substituted as defined for aryl.

The term “protecting group” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “substantially free of” or “substantially in the absence of” with respect to a composition refers to a composition that includes at least 85 or 90% by weight, in certain embodiments 95%, 98%, 99% or 100% by weight, of the compound. In certain embodiments, in the methods and compounds provided herein, the compounds are substantially free of enantiomers.

Similarly, the term “isolated” with respect to a composition refers to a composition that includes at least 85, 90%, 95%, 98%, 99% to 100% by weight, of the compound, the remainder comprising other chemical species or enantiomers.

“Solvate” refers to a compound provided herein or a salt thereof, that further includes a stoichiometric or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen”, the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “aryl,” “alkoxy,” “aminoalkyl,” “alkylamino,” “dialkylamino,” “alkylaminoalkyl,” “dialkylaminoalkyl,” “arylalkyl,” “haloalkyl,” “cycloalkyl,” and “heteroaryl” groups optionally comprise deuterium at one or more positions where hydrogen atoms are present, and wherein the deuterium composition of the atom or atoms is other than the natural isotopic composition.

Also as used herein, “alkyl,” “alkylene,” “aryl,” “alkoxy,” “aminoalkyl,” “alkylamino,” “dialkylamino,” “alkylaminoalkyl,” “dialkylaminoalkyl,” “arylalkyl,” “haloalkyl,” “cycloalkyl,” and “heteroaryl” groups optionally comprise carbon-13 at an amount other than the natural isotopic composition.

The term “optionally substituted,” unless otherwise specified, means that a group is unsubstituted or substituted by one or more (e.g., 1, 2, 3, 4, or 5) of the substituents listed for that group, in which the substituents may be the same or different. In certain embodiments, an optionally substituted group is unsubstituted. In certain embodiments, an optionally substituted group has one substituent. In certain embodiments, an optionally substituted group has two substituents. In certain embodiments, an optionally substituted group has three substituents. In certain embodiment, an optionally substituted group has four substituents. In certain embodiments, an optionally substituted group has 1 to 2, 1 to 3, 1 to 4, or 1 to 5 substituents. When multiple substituents are present, each substituent is independently chosen unless indicated otherwise. For example, each (C₁-C₄ alkyl) substituent on the group —N(C₁-C₄ alkyl)(C₁-C₄ alkyl) can be selected independently from the other, so as to generate groups such as —N(CH₃)(CH₂CH₃), etc.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As one of ordinary skill in the art will recognize, combinations of substituents envisioned by this description are those combinations that result in the formation of stable or chemically feasible compounds.

As used herein, the phrase “stable or chemically feasible” refers to compounds that are not substantially altered when subjected to conditions to allow for their production, detection, and their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Unless a specific isotope of an element is indicated in a formula, the disclosure includes all isotopologues of the compounds disclosed herein, such as, for example, deuterated derivatives of the compounds (where H can be 2H, i.e., D). Isotopologues can have isotopic replacements at any or at all locations in a structure, or can have atoms present in natural abundance at any or all locations in a structure.

The disclosure also includes any or all of the stereochemical forms, including any enantiomeric or diastereomeric forms of the compounds described herein, and cis/trans or E/Z isomers. Unless stereochemistry is explicitly indicated in a chemical structure or name, the structure or name is intended to embrace all possible stereoisomers of a compound depicted. In addition, where a specific stereochemical form is depicted, it is understood that all other stereochemical forms are also described and embraced by the disclosure, as well as the general non-stereospecific form and mixtures of the disclosed compounds in any ratio, including mixtures of two or more stereochemical forms of a disclosed in any ratio, such that racemic, non-racemic, enantioenriched and scalemic mixtures of a compound are embraced. Compounds with alkenyl functionality can exist as either the E-isomer, the Z-isomer, or a mixture of the E-isomer and Z-isomer.

Compositions comprising a disclosed compound also are intended, such as a composition of substantially pure compound, including a specific stereochemical form thereof. Compositions comprising a mixture of disclosed compounds in any ratio also are embraced by the disclosure, including compositions comprising mixtures of two or more stereochemical forms of a disclosed compound in any ratio, such that racemic, non-racemic, enantioenriched and scalemic mixtures of a compound are embraced by the disclosure. If stereochemistry is explicitly indicated for one portion or portions of a molecule, but not for another portion or portions of a molecule, the structure is intended to embrace all possible stereoisomers for the portion or portions where stereochemistry is not explicitly indicated.

The disclosure also embraces any and all tautomeric forms of the compounds described herein.

As used herein, EC₅₀ refers to a dosage, concentration, or amount of a particular test compound that elicits a dose-dependent response at 50% of maximal expression of a particular response that is induced, provoked or potentiated by the particular test compound.

As used herein, the IC₅₀ refers to an amount, concentration, or dosage of a particular test compound that achieves a 50% inhibition of a maximal response in an assay that measures such response.

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In another embodiment, the subject is a farm animal (e.g., a horse, a cow, a pig, etc.) or a pet (e.g., a dog or a cat). In certain embodiments, the subject is a human.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent which is known to be useful for, or has been or is currently being used for the treatment or prevention of a disorder or one or more symptoms thereof.

As used herein, the term “therapeutically effective amount” or “effective amount” refers to an amount of a compound or composition that when administered to a subject is effective to treat a disease or disorder. In some embodiments, a therapeutically effective amount or effective amount refers to an amount of a compound for composition that when administered to a subject is effective to prevent or ameliorate a disease or the progression of the disease, or result in amelioration of symptoms. A “therapeutically effective amount” can vary depending on, inter alia, the compound, the disease and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscemible by the subject. In yet another embodiment, “treating” or “treatment” includes modulating the disease or disorder, either physically (e.g., stabilization of a discemible symptom) or physiologically (e.g., stabilization of a physical parameter) or both. In yet another embodiment, “treating” or “treatment” includes delaying or preventing the onset of the disease or disorder, or delaying or preventing recurrence of the disease or disorder. In yet another embodiment, “treating” or “treatment” includes the reduction or elimination of either the disease or disorder, or to retard the progression of the disease or disorder or of one or more symptoms of the disease or disorder, or to reduce the severity of the disease or disorder or of one or more symptoms of the disease or disorder.

The term “cancer” is used throughout the specification to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant hematogenous, ascitic and solid tumors.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell proliferation, immune cell differentiation, and cytokine expression.

As used herein, the term “inhibits growth” (e.g., referring to cells, such as tumor cells) is intended to include any measurable decrease in cell growth (e.g., tumor cell growth) when contacted with a compound described herein, as compared to the growth of the same cells not in contact with the compound described herein. In some embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to apoptosis, necrosis, and/or effector function-mediated activity.

### STING Agonists

In one aspect, the STING agonist is a compound of the Formula I, Formula II, or Formula III:

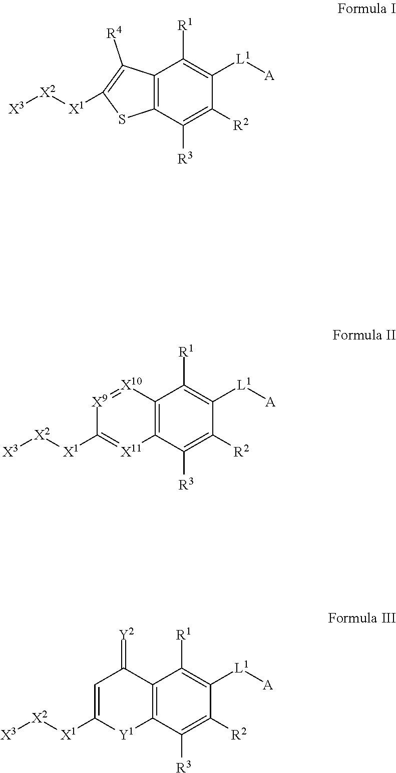

or a pharmaceutically acceptable salt, solvate, or stereoisomer thereof;
wherein:
L¹ is selected from C₁-C₆alkylene,

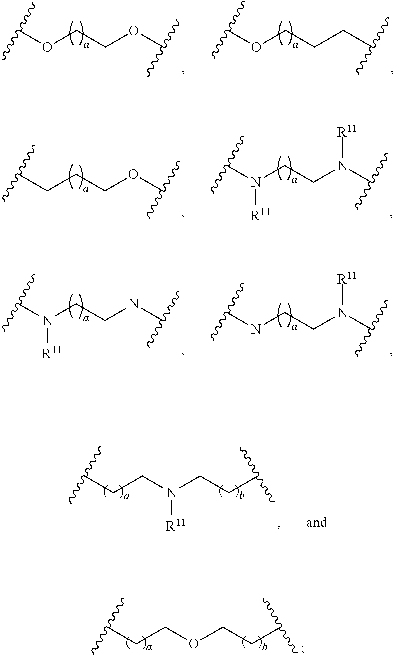

wherein any substitutable carbon in L is optionally substituted with one or more substituents selected from halogen, alkoxy, C₁₋₆alkyl, C₁₋₆alkylamino, C₃₋₁₀cycloalkyl, hydroxy, cyano, —NR^{9a}R^{9b}, and —COOR¹⁰;

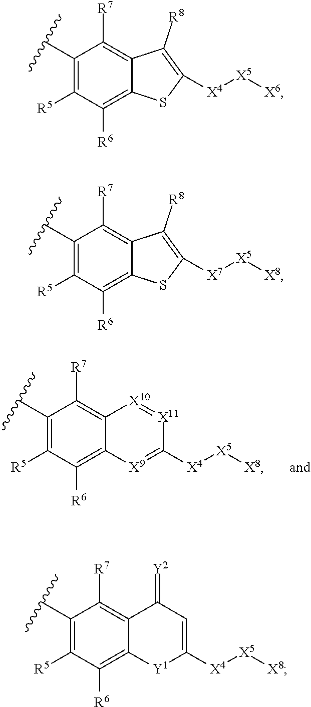

A is selected from
R¹, R², R³, R⁴, R⁵, R⁶, R⁷ and R⁸ are independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, amino-C₁₋₆alkyl, C₁₋₆alkylamino-C₁₋₆alkyl, di-C₁₋₆alkylamino-C₁₋₆alkyl, and —COOR¹⁰;
X¹ and X⁴ are independently selected from —C(O)—, —C(R^{12a}R^{12b})—, —CH═CH—, and —C(R^{12a}R^{12b})C(R^{13a}R^{13b})—;
X² and X⁵ are independently selected from —(C(R^{12a}R^{12b}))₁₋₆— and —(C(R^{12a}R^{12b})C(R^{13a}R^{13b}))₁₋₃—;
X³ is selected from —COOR¹⁰, —CONR^{9a}R^{9b}, —C(O)NR^{9a}S(O)₂R¹⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁶ is selected from —C(O)NR^{9a}S(O)₂R¹⁷, —C(O)NR^{9a}R¹⁸, —S(O)₂OH, —S(O)OH, —P(O)(OR¹⁵)₂, —OP(O)(OR¹⁵)₂, and —P(O)(NR^{16a}R^{16b})(OR¹⁵);
X⁷ is —CH═CH—, C₂alkynyl, or —C(R^{12a}R^{12c})C(R^{13a}R^{13c})—;
X⁸ is selected from —COOR¹⁰, —CONR^{9a}R^{9b}, —C(O)NR^{9a}S(O)₂R¹⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁰);
X⁹, X¹⁰, and X¹¹ are independently selected from CR¹⁹ and N;
Y¹ is independently selected from CH₂, O, NH, and S;
Y² is independently selected from O, NH, and S;
R^{9a} and R^{9b} are independently selected from hydrogen, C₁₋₆alkyl, C₃₋₁₀cycloalkyl, aryl, aryl-C₁₋₆alkyl, amino-C₁₋₆alkyl, C₁₋₆alkylamino-C₁₋₆alkyl, di-C₁₋₆alkylamino-C₁₋₆alkyl, and heteroaryl;
R¹⁰ is independently selected from hydrogen, C₁₋₆alkyl, C₃₋₁₀cycloalkyl, aryl, aryl-C₁₋₆alkyl, and heteroaryl;
R¹¹ is independently selected from hydrogen and C₁₋₆alkyl;
R^{12a}, R^{12b}, R^{13a}, and R^{13b} are independently selected from hydrogen and C₁₋₆alkyl; or R^{12b} and R^{13b} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R^{12c} and R^{13c} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R¹⁴ is selected from C₁₋₆alkyl, C₃₋₁₀cycloalkyl, halo-C₁₋₆alkyl, amino-C₁₋₆alkyl, C₁₋₆alkyl-amino-C₁₋₆alkyl, di-C₁₋₆alkylamino-C₁₋₆alkyl, aryl, aryl-C₁₋₆alkyl, and heteroaryl;
R¹⁵, R^{16a}, and R^{16b} are independently selected from hydrogen, C₁₋₆alkyl, and C₃₋₁₀cycloalkyl;
R¹⁷ is amino-C₁₋₆alkyl, C₁₋₆alkyl-amino-C₁₋₆alkyl, di-C₁₋₆alkylamino-C₁₋₆alkyl, C₃₋₁₀ cycloalkyl-amino-C₁₋₆alkyl, aryl, aryl-C₁₋₆alkyl, or heteroaryl;
R¹⁸ is amino-C₁₋₆alkyl, C₁₋₆alkylamino-C₁₋₆alkyl, di-C₁₋₆alkylamino-C₁₋₆alkyl, C₃₋₁₀cycloalkyl-amino-C₁₋₆alkyl, C₁₋₆alkyl, C₃₋₁₀cycloalkyl, aryl, aryl-C₁₋₆alkyl, or heteroaryl; and
R¹⁹ is independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, and —COOR¹⁰; and each of a and b is an integer independently selected from 0, 1, 2, 3, 4, and 5.

In certain embodiments, the compound of Formula I is a structure selected from:

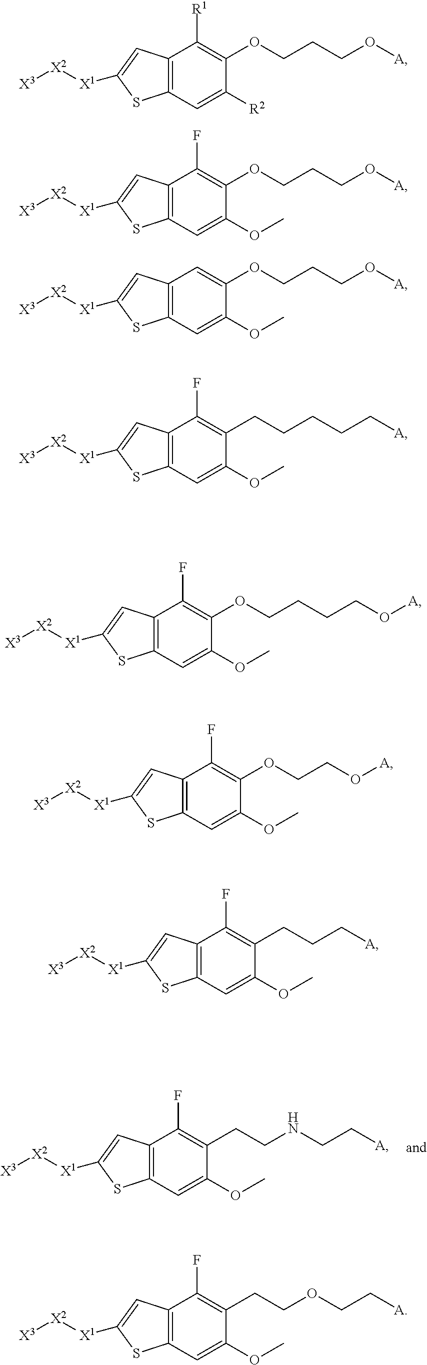

In certain embodiments, the compound of Formula I is a structure selected from:

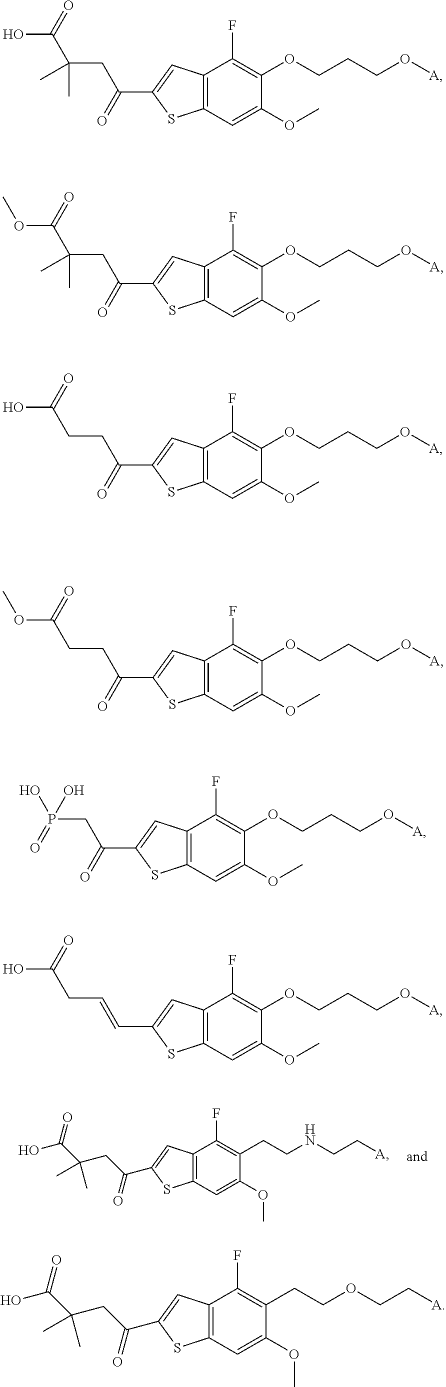

In certain embodiments, the compound of Formula I is a structure selected from:

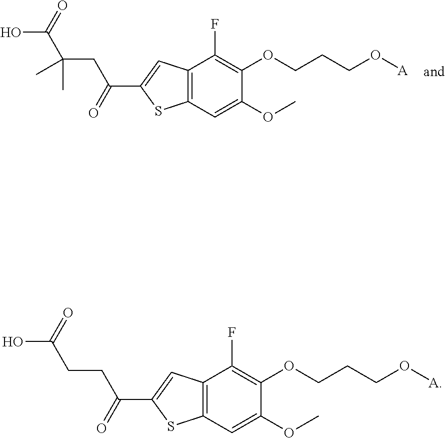

In certain embodiments, the compound of Formula II is a structure selected from:

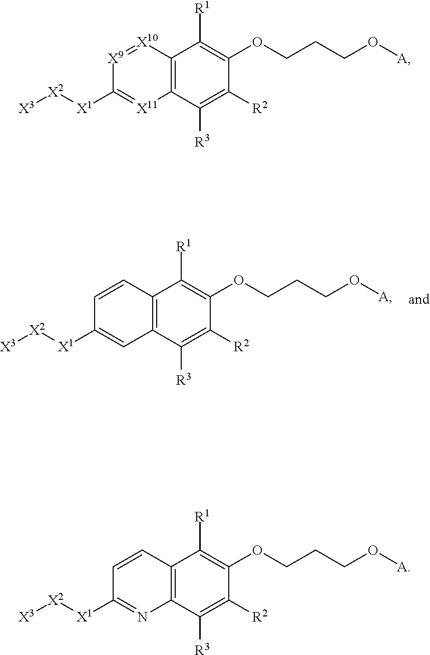

In certain embodiments, the compound of Formula III is a structure selected from:

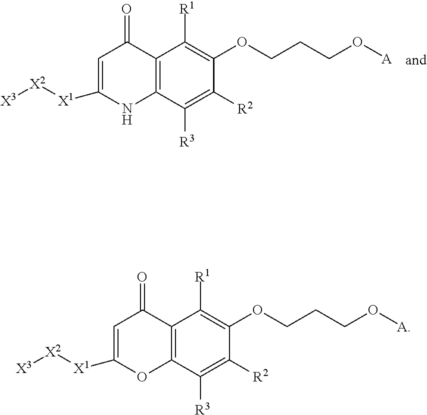

In one embodiment, A is

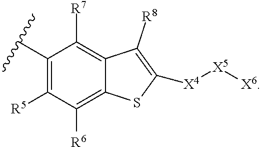

In certain embodiments, A is selected from:

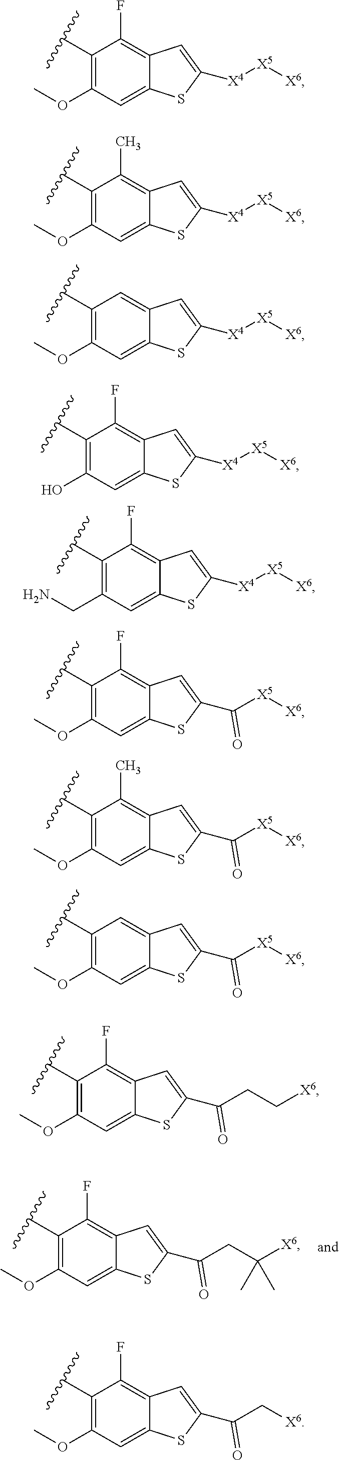

In one embodiment, A is

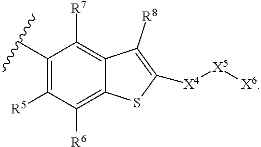

In certain embodiments, A is selected from:

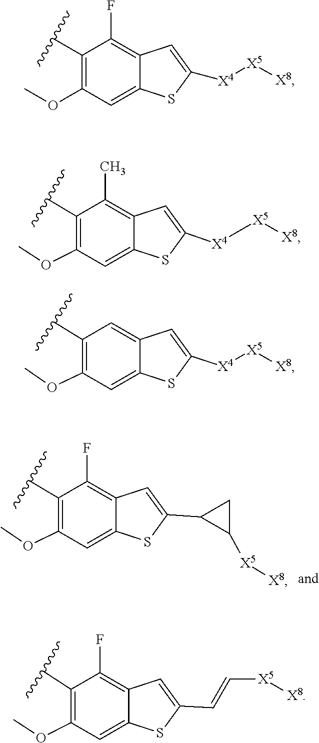

In one embodiment, A is

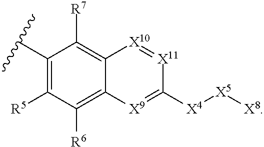

In certain embodiments, A is selected from:

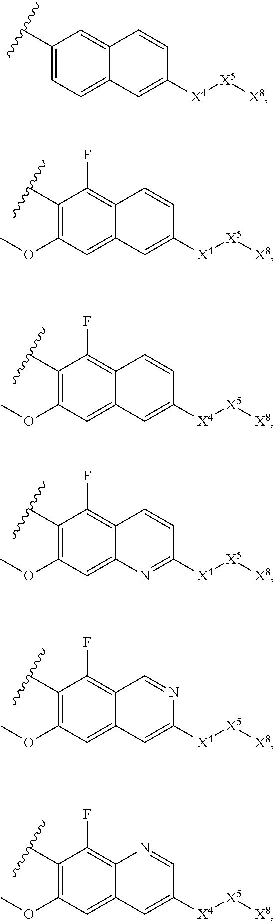

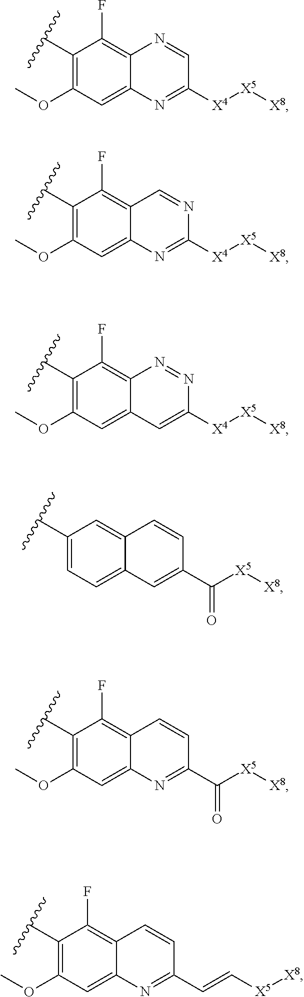

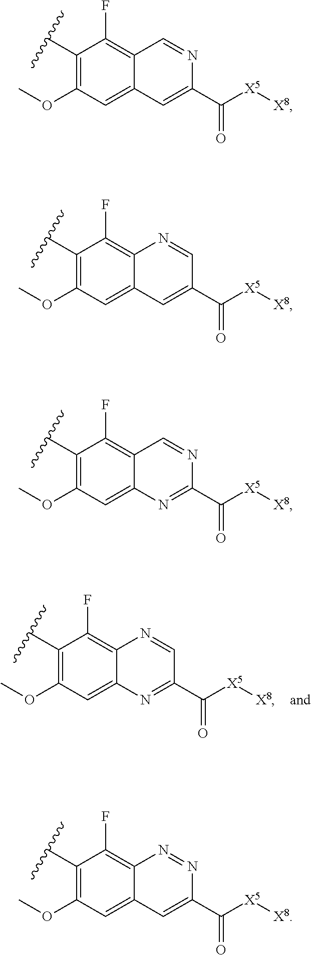

In one embodiment, A is

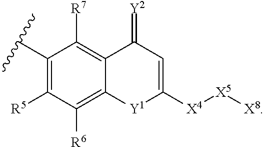

In certain embodiments, A is selected from:

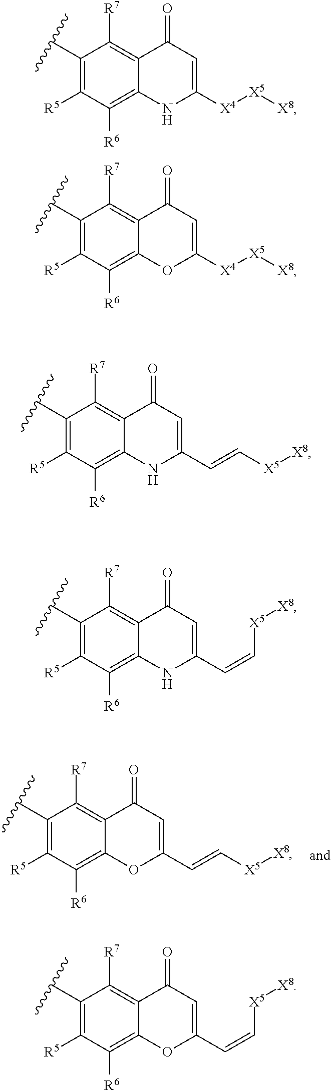

Non-limiting examples of A include:

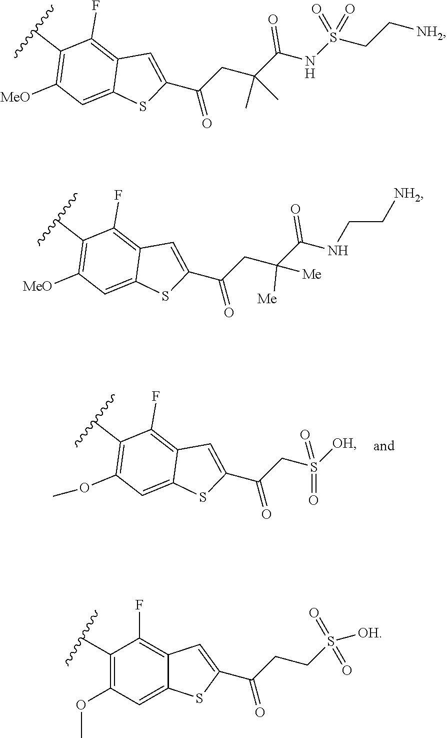 [SMILES: *c1c(OC)cc2sc(C(=O)CC(C)(C)C(=O)NS(=O)(=O)CCN)cc2c1F ; *c1c(OC)cc2sc(C(=O)CC(C)(C)C(=O)NCCN)cc2c1F ; *c1c(OC)cc2sc(C(=O)CS(=O)(=O)O)cc2c1F ; *c1c(OC)cc2sc(C(=O)CCS(=O)(=O)O)cc2c1F]

Additional non-limiting examples of A include:

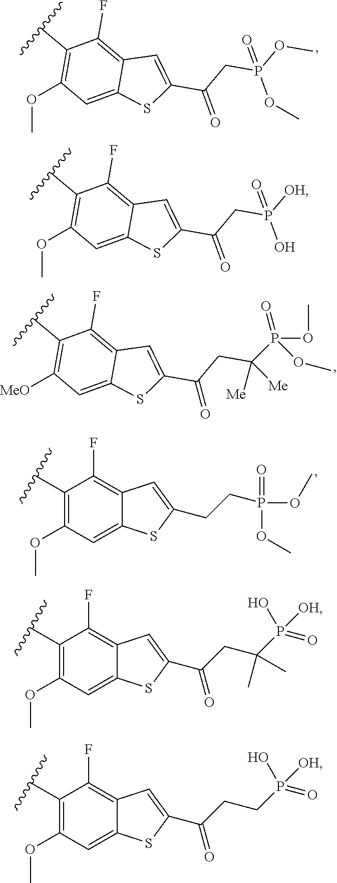

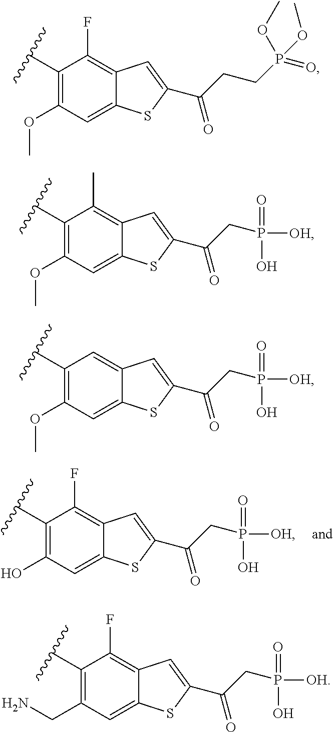

Additional non-limiting examples of A include:

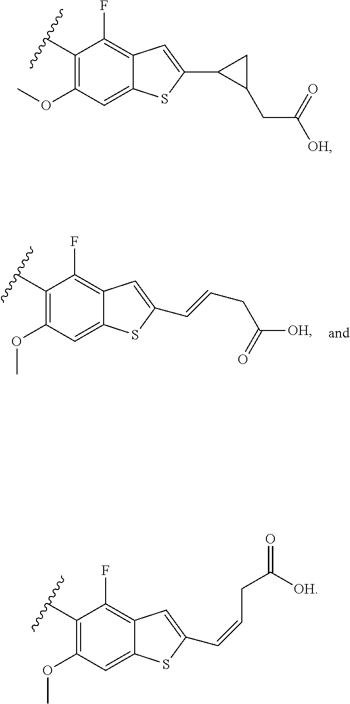 [SMILES: *c1c(OC)cc2sc(C3CC3CC(=O)O)cc2c1F ; *c1c(OC)cc2sc(/C=C/CC(=O)O)cc2c1F ; *c1c(OC)cc2sc(/C=C\CC(=O)O)cc2c1F]

Additional non-limiting examples of A include:

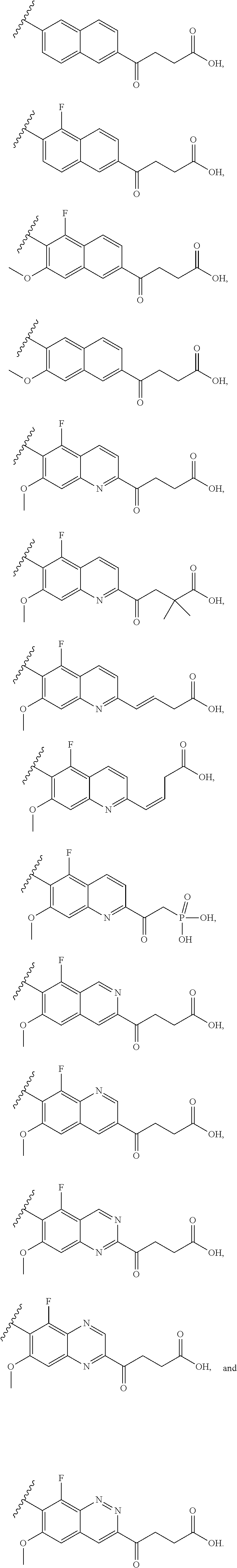

Additional non-limiting examples of A include:

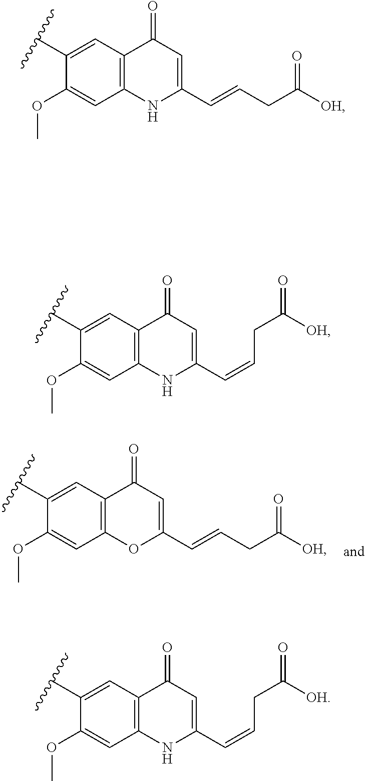 [SMILES: *c1cc2c(=O)cc(/C=C/CC(=O)O)[nH]c2cc1OC ; *c1cc2c(=O)cc(/C=C\CC(=O)O)[nH]c2cc1OC ; *c1cc2c(=O)cc(/C=C/CC(=O)O)oc2cc1OC ; *c1cc2c(=O)cc(/C=C\CC(=O)O)[nH]c2cc1OC]

In one embodiment, the STING agonist is a compound of Formula Ia:

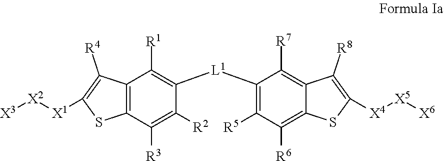

or a pharmaceutically acceptable salt, solvate, or stereoisomer thereof;
wherein L¹, X¹, X², X³, X⁴, X⁵, X⁶, R¹, R², R³, R⁴, R¹, R⁶, R⁷, R¹, R^{9a}, R^{9b}, R¹⁰, R¹¹, R^{12a}, R^{12b}, R^{13a}, R^{13b}, R¹⁴, R¹⁵, R^{16a}, R^{16b}, a, and b are as defined herein.

In one embodiment, the compound of Formula Ia is selected from the following:

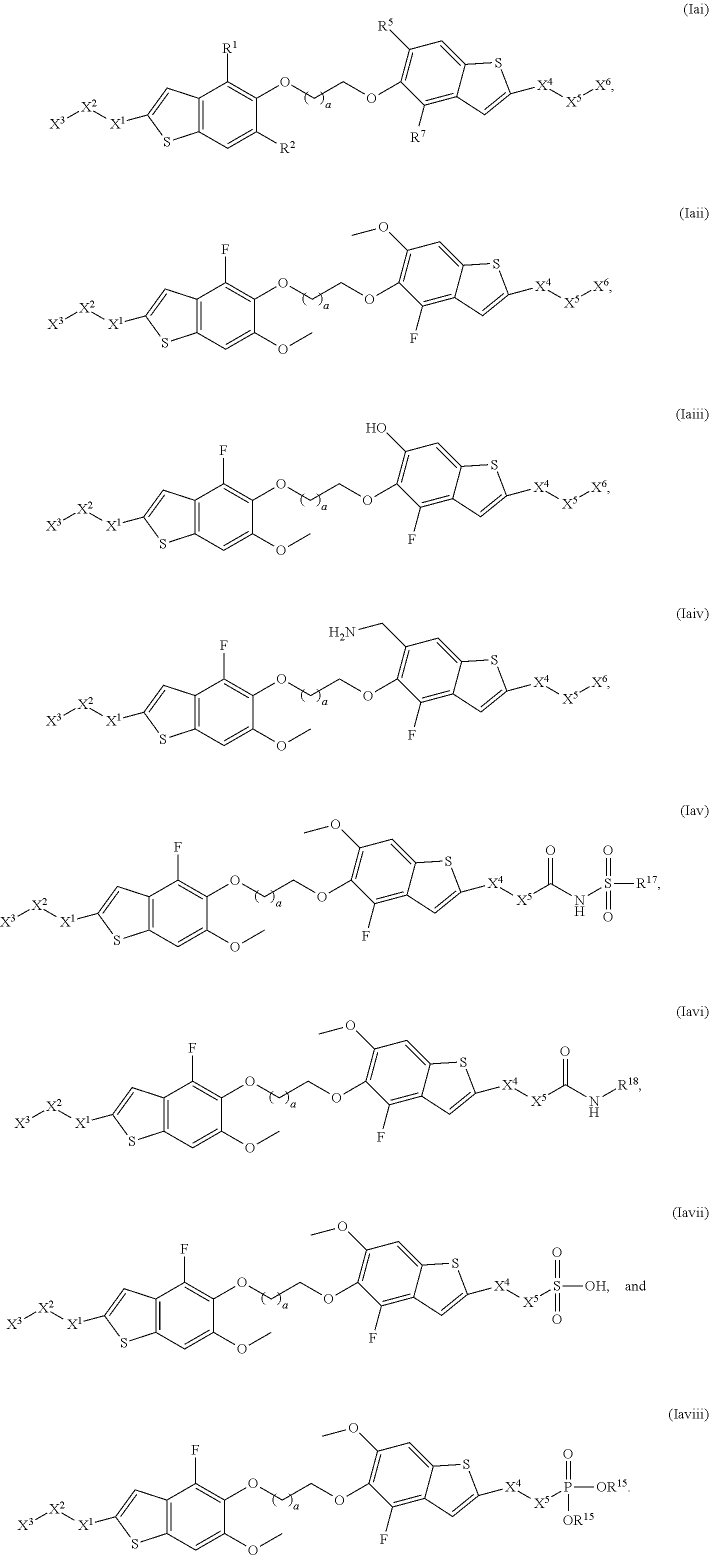

Non-limiting examples of Formula Ia include, but are not limited to, the following:

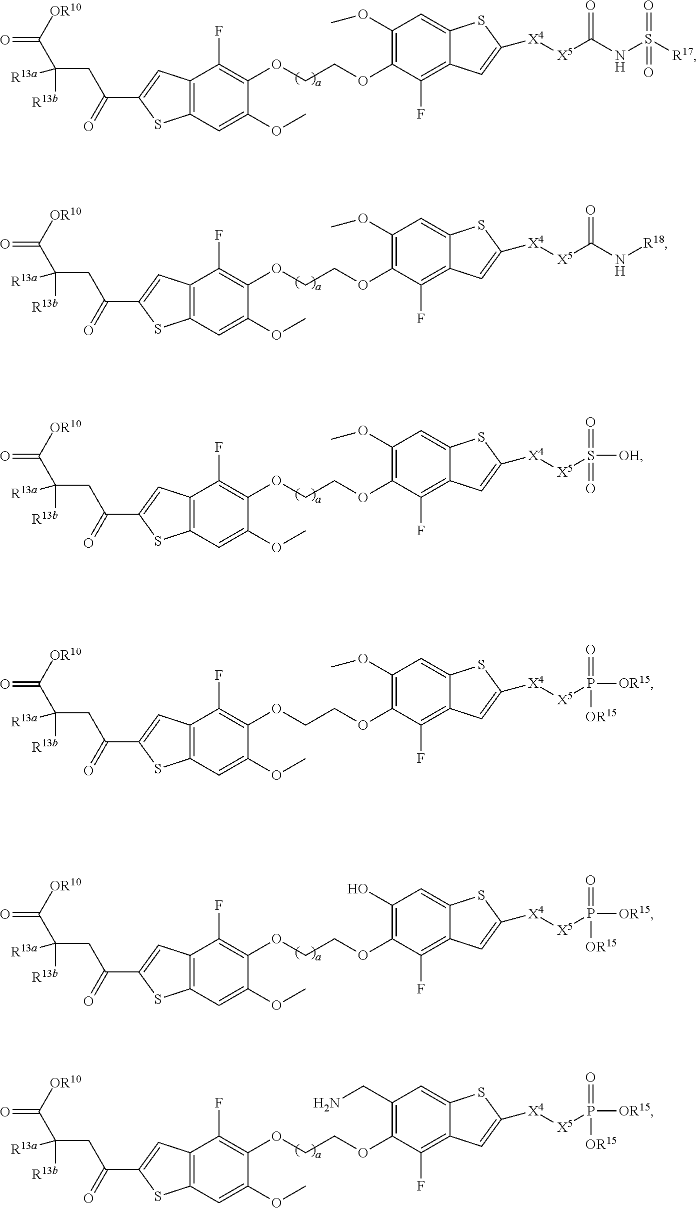

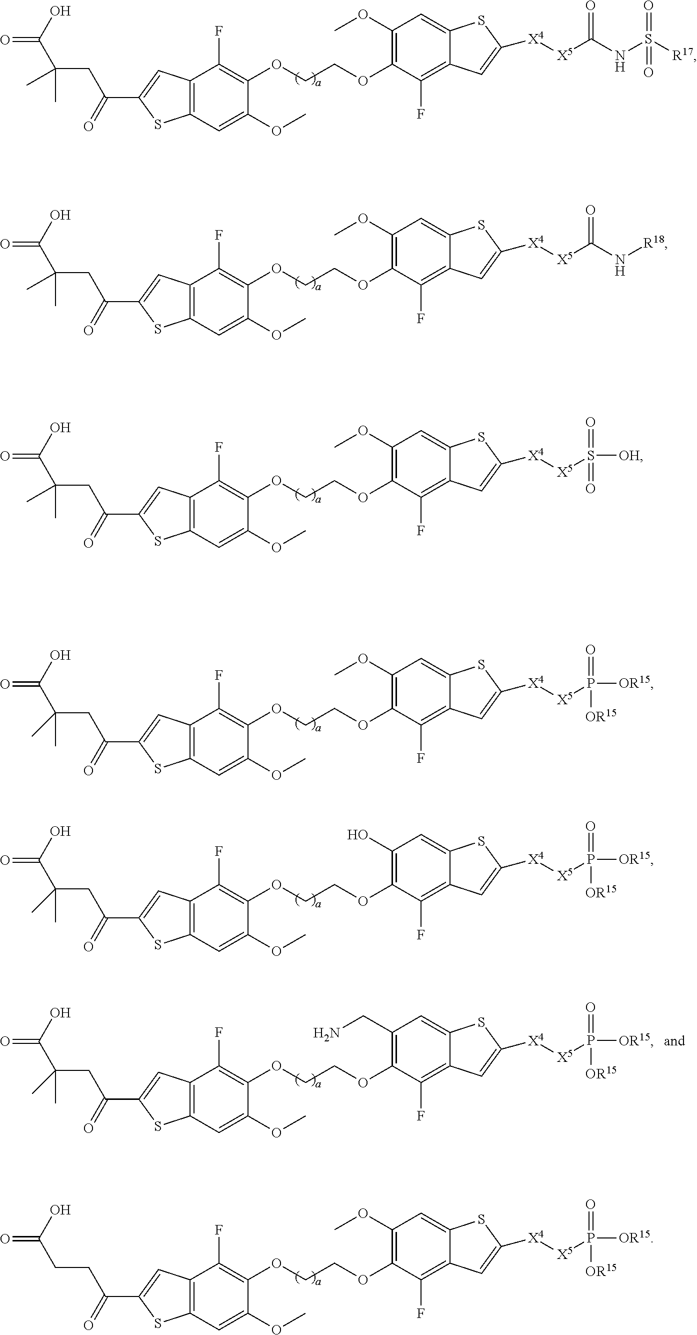

In one embodiment, the STING agonist is a compound of Formula Ib:

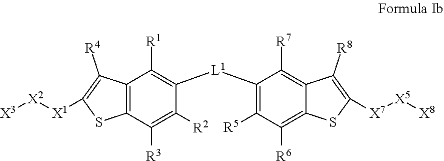

or a pharmaceutically acceptable salt, solvate, or stereoisomer thereof;
wherein L¹, X¹, X², X³, X⁵, X⁷, X⁸, R¹, R², R³, R⁴, R⁵, R⁶, R⁷, R⁸, R^{9a}, R^{9b}, R¹⁰, R¹¹, R^{12a}, R^{12b}, R^{12c}, R^{13a}, R^{13b}, R^{13c}, R¹⁴, R¹⁵, R^{16a}, R^{16b}, a, and b are as defined herein.

In one embodiment, the compound of Formula Ib is selected from the following:

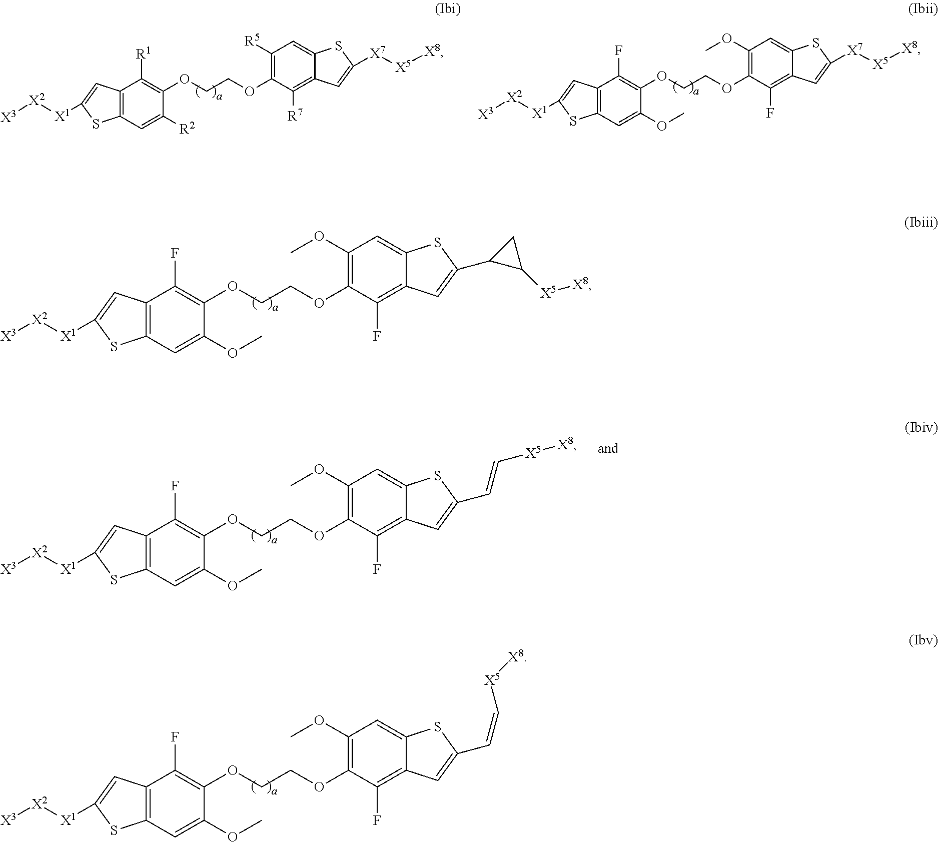

Non-limiting examples of Formula Ib include, but are not limited to, the following:

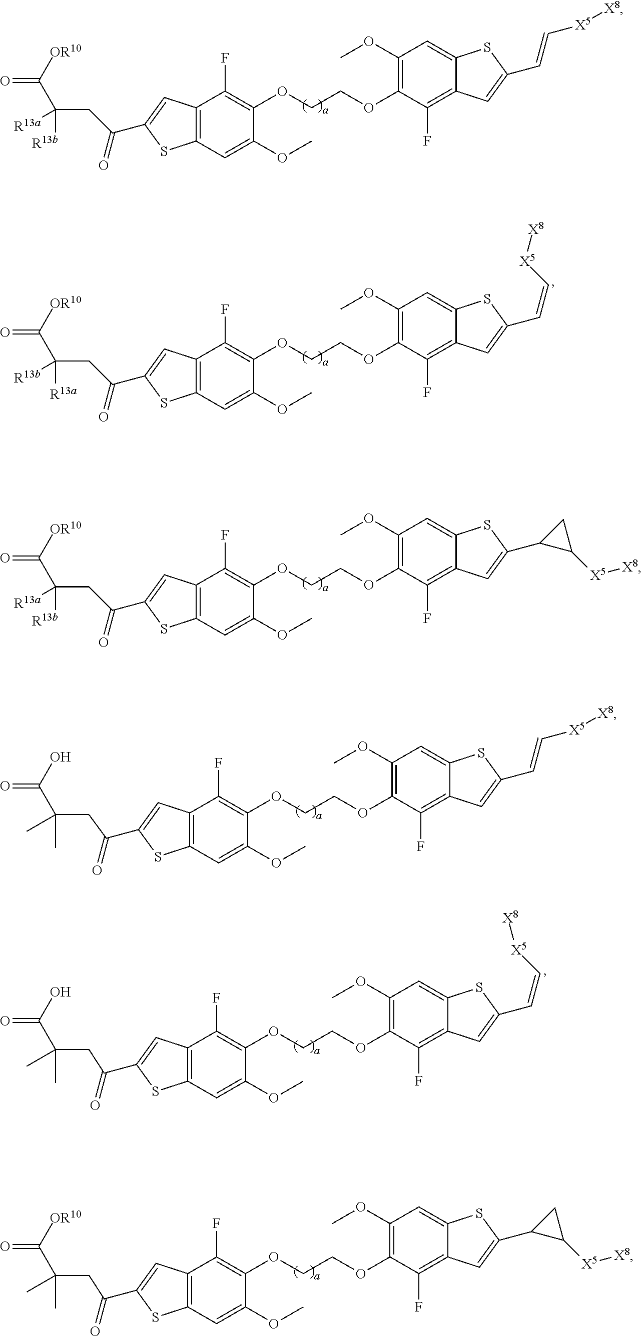

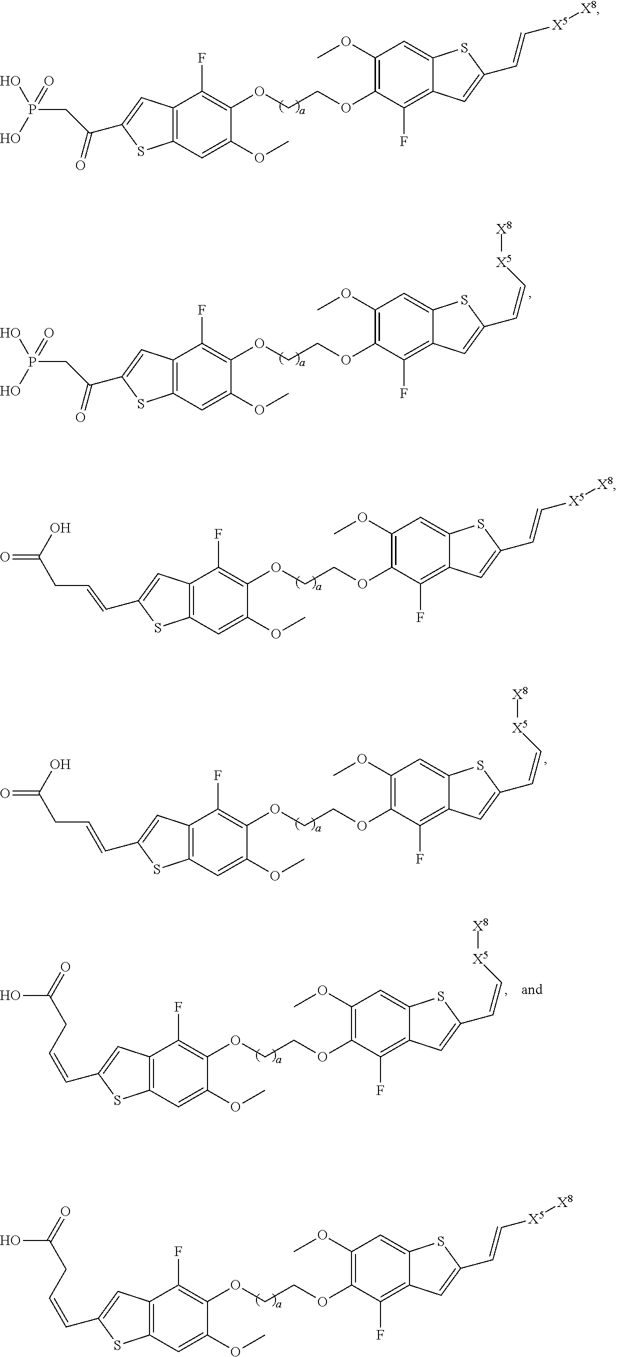

In one embodiment, the STING agonist is a compound of Formula Ic:

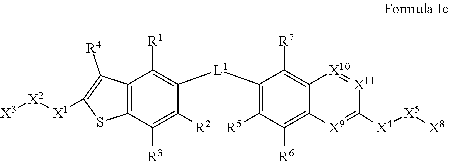

or a pharmaceutically acceptable salt, solvate, or stereoisomer thereof;
wherein L¹, X¹, X², X³, X⁴, X⁵, X⁸, X⁹, X¹⁰, X¹¹, R¹, R², R³, R⁴, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, R¹⁹, a, and b are as defined herein.

In one embodiment, the compound of Formula Ic is selected from the following:

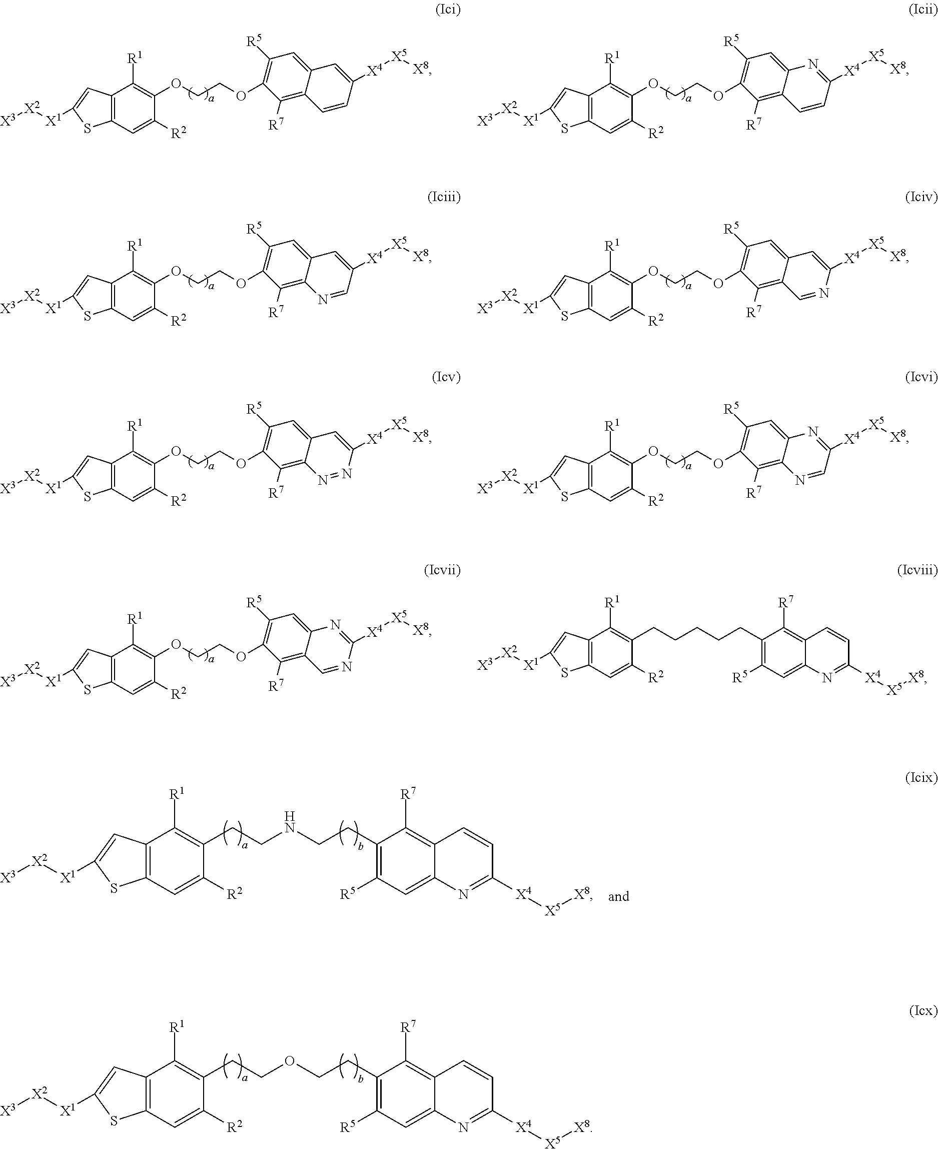

Non-limiting examples of Formula Ic include, but are not limited to, the following:

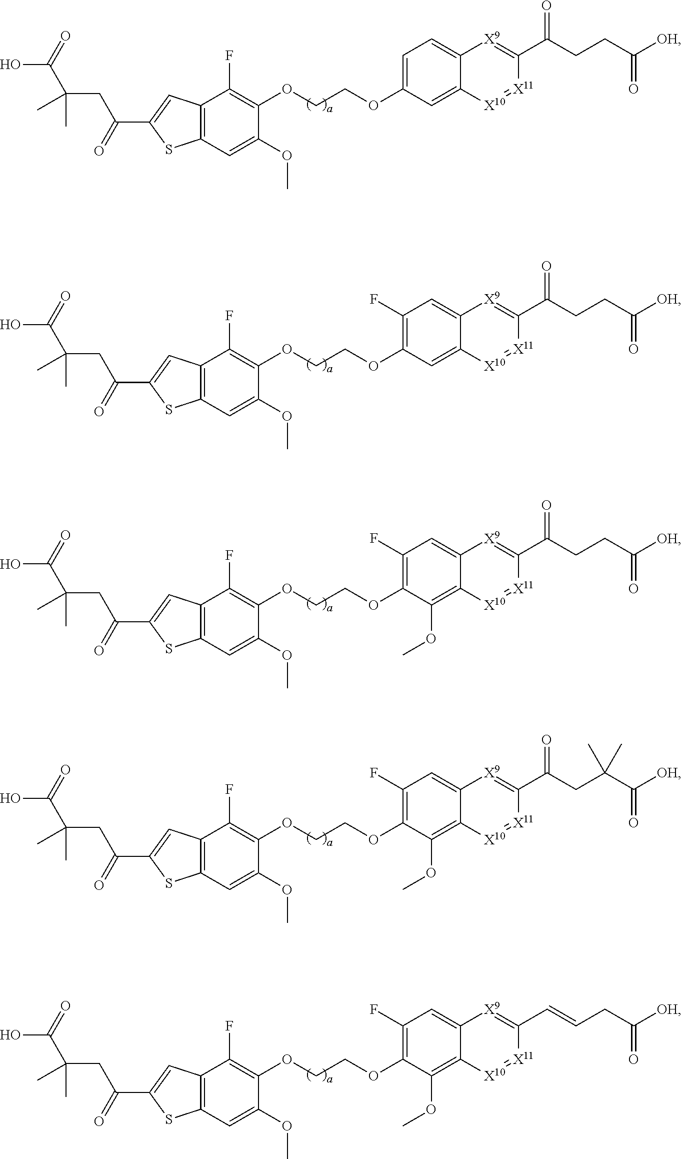

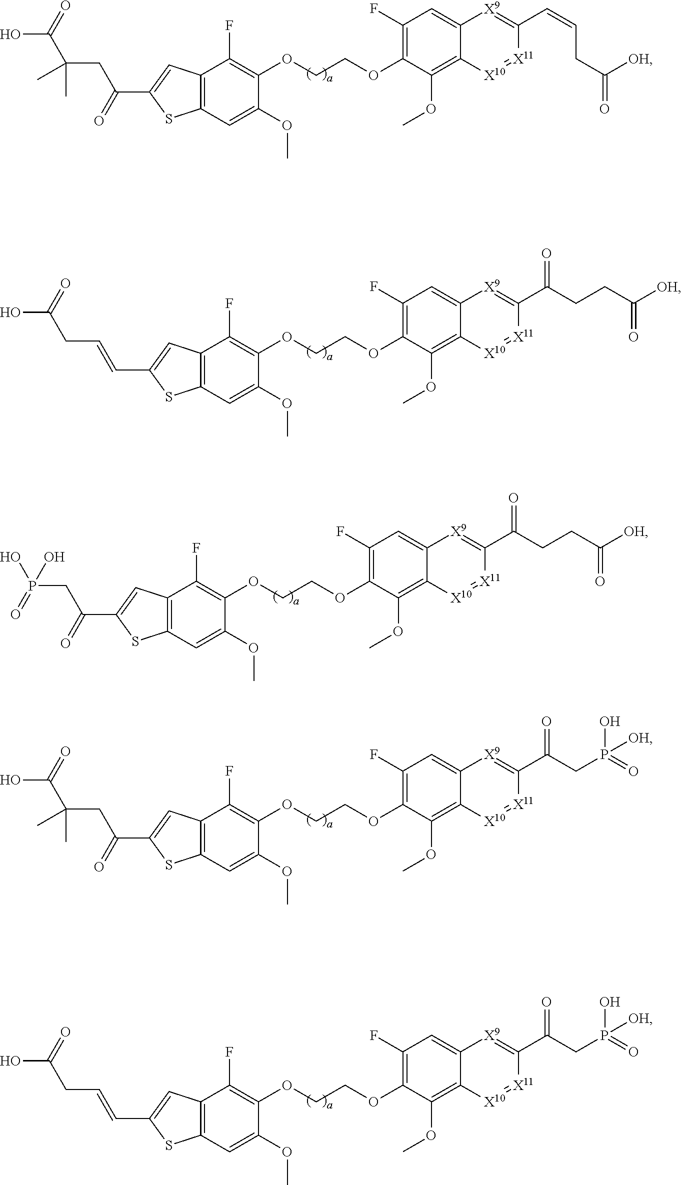

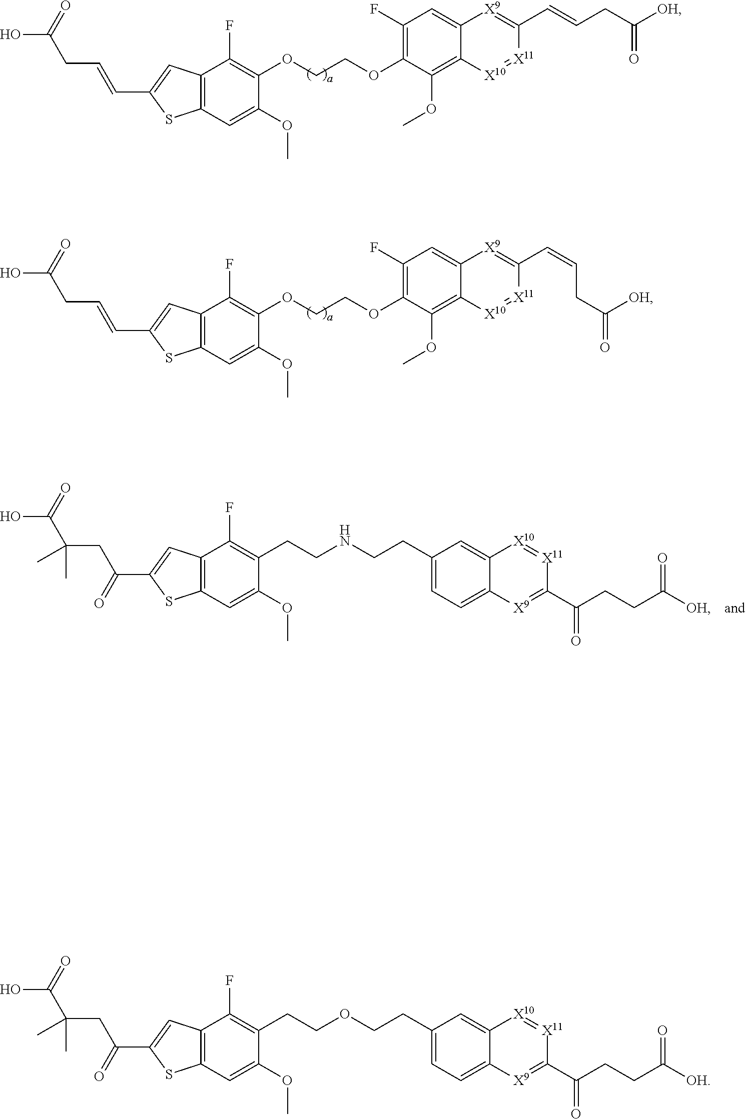

In one embodiment, the STING agonist is a compound of Formula Id:

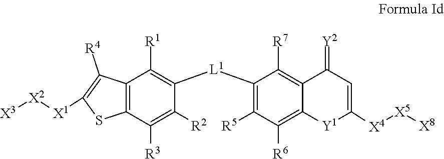

or a pharmaceutically acceptable salt, solvate, or stereoisomer thereof;
wherein L¹, X¹, X², X³, X⁴, X⁵, X⁸, Y¹, Y², R¹, R², R³, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, R¹⁹, a, and b are as defined herein.

In one embodiment, the compound of Formula Id is selected from the following:

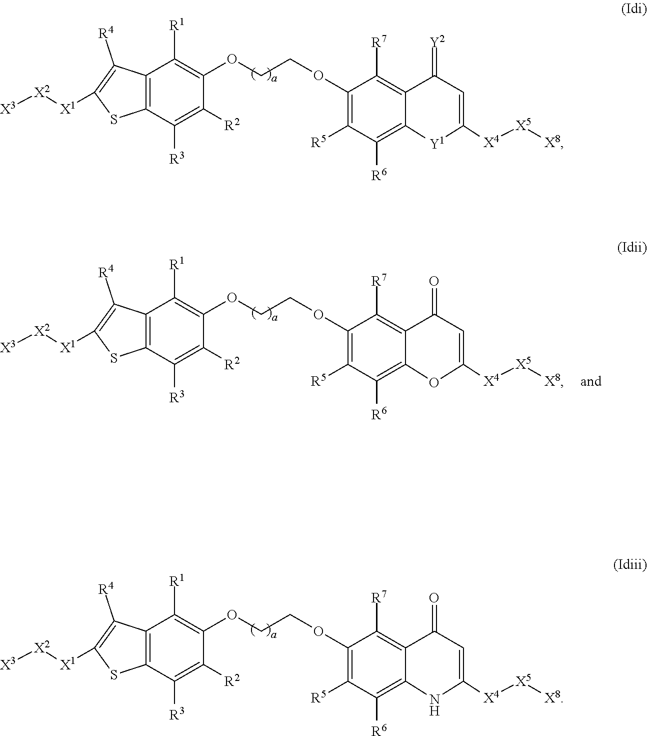

Non-limiting examples of Formula Id include, but are not limited to, the following:

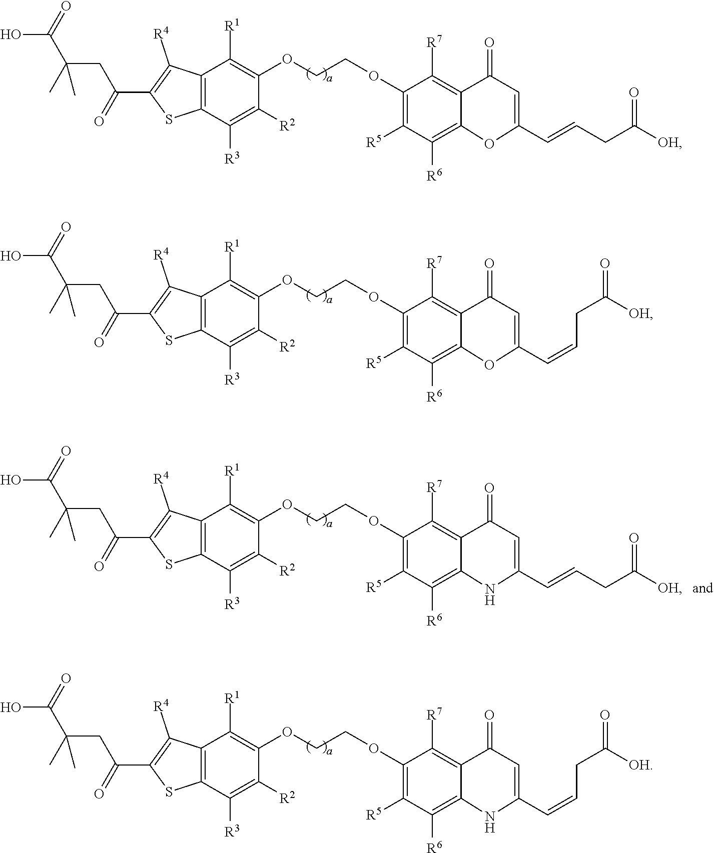

In one embodiment, the STING agonist is a compound of Formula IIa:

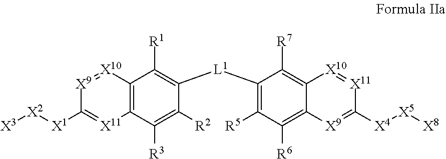

or a pharmaceutically acceptable salt, solvate, or stereoisomer thereof;
wherein L¹, X¹, X², X³, X⁴, X⁵, X⁸, X⁹, X¹⁰, X¹¹, R¹, R², R³, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, R¹⁹, a, and b are as defined herein.

In one embodiment, the compound of Formula IIa is selected from the following:

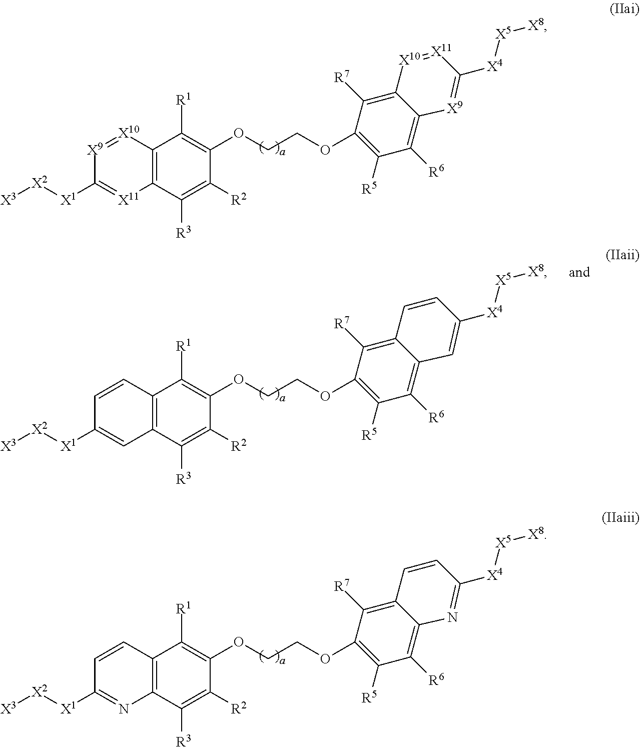

Non-limiting examples of Formula IIa include, but are not limited to, the following:

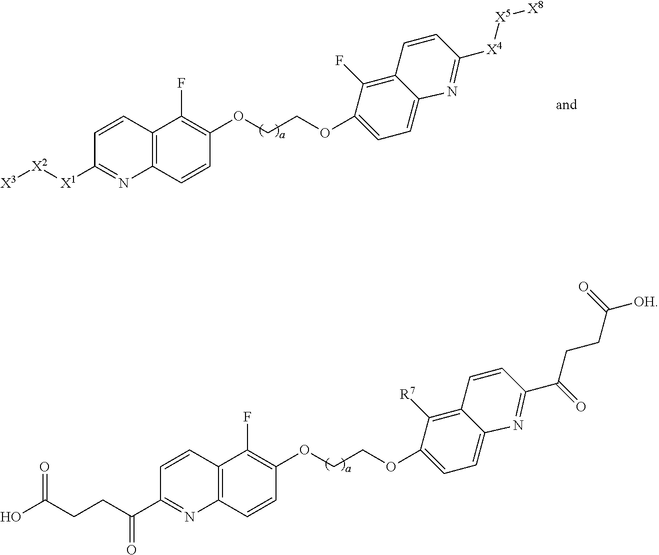

In one embodiment, the STING agonist is a compound of Formula IIb:

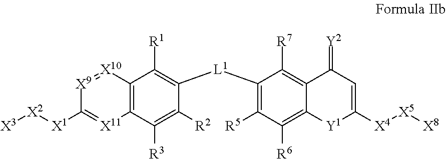

or a pharmaceutically acceptable salt, solvate, or stereoisomer thereof;
wherein L¹, X¹, X², X³, X⁴, X⁵, X⁸, Y¹, Y², R¹, R², R³, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, a, and b are as defined herein.

In one embodiment, the compound of Formula IIb is selected from the following:

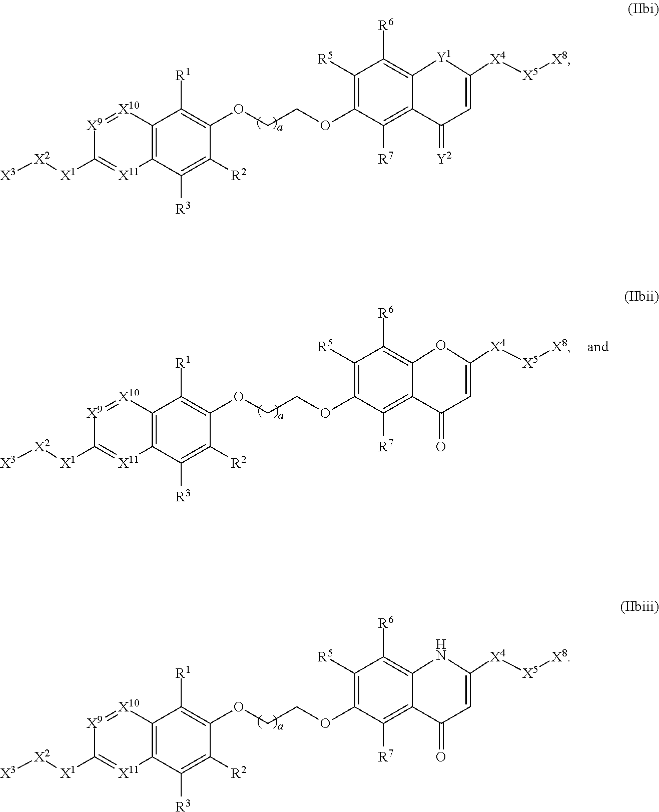

Non-limiting examples of Formula IIb include, but are not limited to, the following:

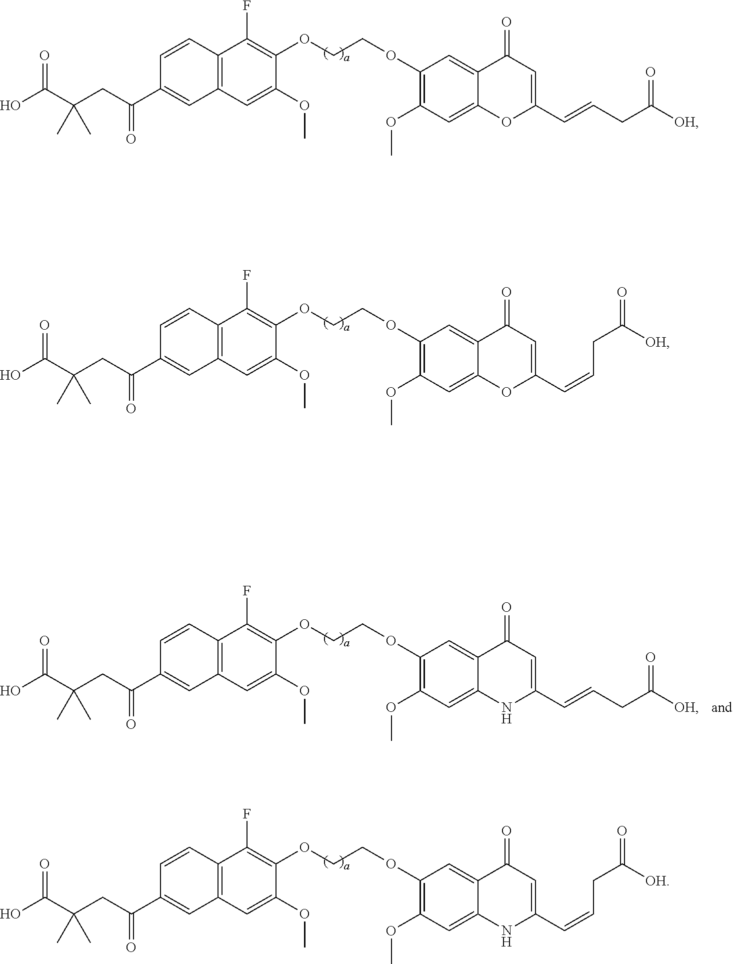

In one embodiment, the STING agonist is a compound of Formula IIIa:

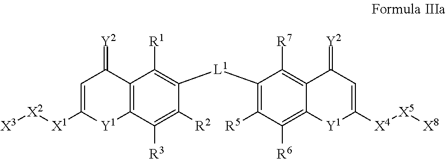

or a pharmaceutically acceptable salt, solvate, or stereoisomer thereof;
wherein L¹, X¹, X², X³, X⁴, X⁵, X⁸, Y¹, Y², R¹, R², R³, R⁵, R⁶, R⁷, R⁸, R^{9a}, R^{9b}, R¹⁰, R¹¹, R^{12a}, R^{12b}, R^{13a}, R^{13b}, R¹⁴, R¹⁵, R¹⁶, R^{16b}, a, and b are as defined herein.

In one embodiment, the compound of Formula IIIa is:

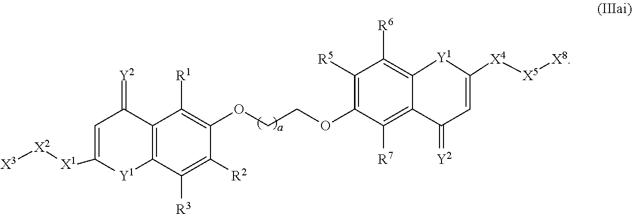

Non-limiting examples of Formula IIIa include, but are not limited to, the following:

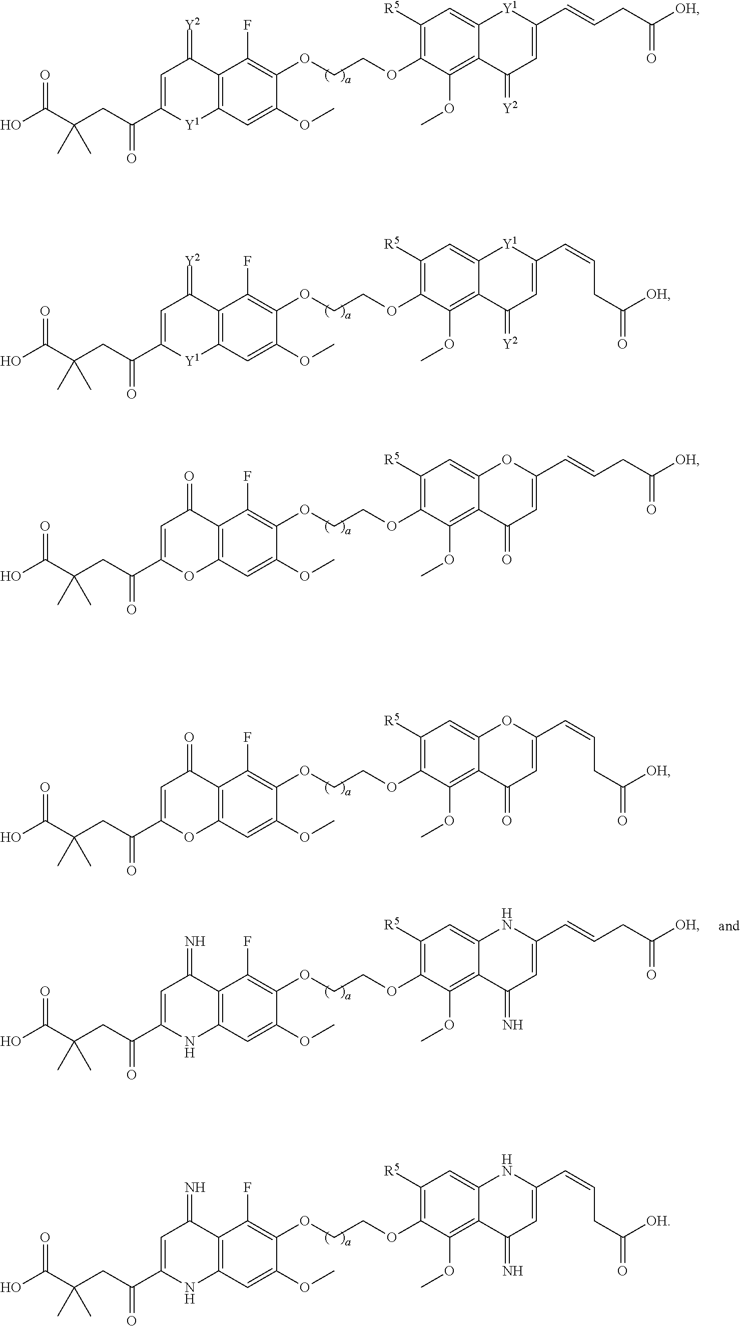

In one embodiment, including any of the foregoing, L¹ is C₁₋₆alkylene. In one embodiment, including any of the foregoing, L¹ is C₃alkylene. In one embodiment, including any of the foregoing, L¹ is C₄alkylene. In one embodiment, including any of the foregoing, L¹ is C₅alkylene. In one embodiment, including any of the foregoing, L¹ is C₆alkylene.

In one embodiment, including any of the foregoing, L¹ is

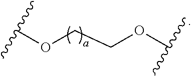

In certain embodiments, including any of the foregoing, L¹ is

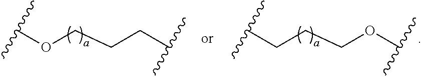

In certain embodiments, including any of the foregoing, L¹ is

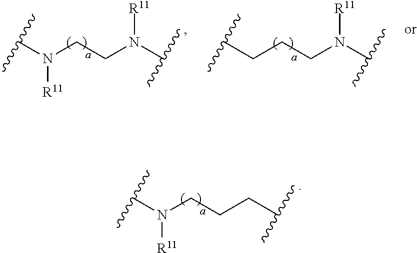

In certain embodiments, including any of the foregoing, L¹ is

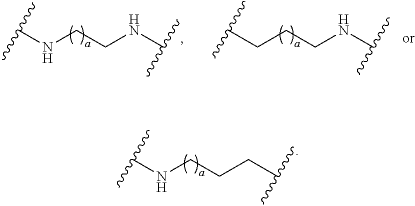

In certain embodiments, including any of the foregoing, L¹ is

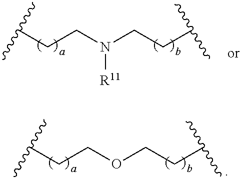

In one embodiment, including any of the foregoing, a is 0. In one embodiment, including any of the foregoing, a is 1. In one embodiment, including any of the foregoing, a is 2. In one embodiment, including any of the foregoing, a is 3. In one embodiment, including any of the foregoing, a is 4. In one embodiment, including any of the foregoing, a is 5.

In one embodiment, including any of the foregoing, b is 0. In one embodiment, including any of the foregoing, b is 1. In one embodiment, including any of the foregoing, b is 2. In one embodiment, including any of the foregoing, b is 3. In one embodiment, including any of the foregoing, b is 4. In one embodiment, including any of the foregoing, b is 5.

In one embodiment, including any of the foregoing, both a and b are 0. In one embodiment, including any of the foregoing, both a and b are 1. In one embodiment, including any of the foregoing, both a and b are 2. In one embodiment, including any of the foregoing, both a and b are 3. In one embodiment, including any of the foregoing, both a and b are 4. In one embodiment, including any of the foregoing, both a and b are 5.

In one embodiment, including any of the foregoing, one of a or b is 0. In one embodiment, including any of the foregoing, one of a or b is 1. In one embodiment, including any of the foregoing, one of a or b is 2. In one embodiment, including any of the foregoing, one of a or b is 3. In one embodiment, including any of the foregoing, one of a or b is 4. In one embodiment, including any of the foregoing, one of a or b is 5.

In certain embodiments, including any of the foregoing, R¹¹ is independently selected from hydrogen and methyl. In one embodiment, including any of the foregoing, R¹¹ is hydrogen. In one embodiment, including any of the foregoing, R¹¹ is methyl.

In certain embodiments, L¹ is selected from

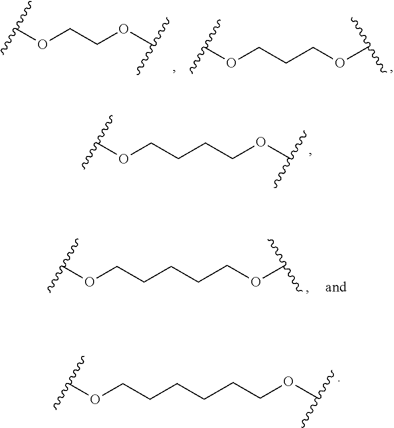 [SMILES: *OCCO* ; *OCCCO* ; *OCCCCO* ; *OCCCCCO* ; *OCCCCCCO*]

In one embodiment, L¹ is

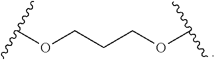 [SMILES: *OCCCO*]

In certain embodiments, including any of the foregoing, L¹ is

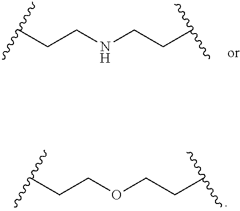 [SMILES: *CCNCC* ; *CCOCC*]

In certain embodiments, including any of the foregoing, R¹ is selected from hydrogen and halogen. In certain embodiments, including any of the foregoing, R⁷ is selected from hydrogen, halogen, and C₁₋₆alkyl. In one embodiment, including any of the foregoing, both R¹ and R⁷ are halogen. In one embodiment, including any of the foregoing, both R¹ and R⁷ are C₁₋₆alkyl. In one embodiment, including any of the foregoing, both R¹ and R⁷ are hydrogen. In one embodiment, including any of the foregoing, R¹ is halogen and R⁷ are hydrogen. In one embodiment, including any of the foregoing, R¹ is hydrogen and R⁷ are halogen. In one embodiment, including any of the foregoing, R¹ and R⁷ are both fluorine. In one embodiment, R¹ is hydrogen and R⁷ is fluorine. In one embodiment, R¹ is fluorine and R⁷ is hydrogen. In one embodiment, including any of the foregoing, R¹ and R⁷ are both methyl.

In certain embodiments, including any of the foregoing, R² is selected from alkoxy and hydrogen. In certain embodiments, including any of the foregoing, R⁵ is selected from alkoxy, hydroxy, amino-C₁₋₆alkyl, and hydrogen. In certain embodiments, including any of the foregoing, R⁵ is selected from alkoxy and hydrogen. In certain embodiments, including any of the foregoing, R⁵ is selected from hydroxyl and amino-C₁₋₆alkyl. In one embodiment, including any of the foregoing, both R² and R⁵ are alkoxy. In one embodiment, including any of the foregoing, R² is hydrogen and R⁵ is alkoxy. In one embodiment, including any of the foregoing, R² is alkoxy and R⁵ is hydrogen. In one embodiment, including any of the foregoing, both R² and R⁵ are methoxy. In one embodiment, including any of the foregoing, R² is hydrogen and R⁵ is methoxy. In one embodiment, including any of the foregoing, R² is methoxy and R⁵ is hydrogen. In certain embodiments, including any of the foregoing, R⁵ is hydroxy and R² is alkoxy. In certain embodiments, including any of the foregoing, R⁵ is amino-C₁₋₆alkyl and R² is alkoxy. In certain embodiments, including any of the foregoing, R⁵ is hydroxy or amino-C₁₋₆alkyl and R² is methoxy. In certain embodiments, including any of the foregoing, R⁵ is —CH₂NH₂ and R² is alkoxy.

In certain embodiments, including any of the foregoing, R¹ and R⁷ are selected from hydrogen, halogen, and C₁₋₆alkyl and R² and R⁵ are both alkoxy. In one embodiment, including any of the foregoing, R¹ and R⁷ are both halogen and R² and R⁵ are both alkoxy. In one embodiment, including any of the foregoing, R¹ and R⁷ are both C₁₋₆alkyl and R² and R⁵ are both alkoxy. In one embodiment, including any of the foregoing, R¹ and R⁷ are both fluorine and R² and R⁵ are both methoxy. In one embodiment, including any of the foregoing, R¹ and R⁷ are both methyl and R² and R⁵ are both alkoxy.

In certain embodiments, including any of the foregoing, R¹ and R⁷ are both fluorine, R² is alkoxy, and R⁵ is hydroxyl or amino-C₁₋₆alkyl. In certain embodiments, including any of the foregoing, R¹ and R⁷ are both halogen, R² is alkoxy, and R⁵ is hydroxy. In certain embodiments, including any of the foregoing, R¹ and R⁷ are both halogen, R² is alkoxy, and R⁵ is amino-C₁₋₆alkyl.

In one embodiment, including any of the foregoing, R³, R⁴, R⁶, and R⁸ are hydrogen.

In certain embodiments, including any of the foregoing, R¹ and R⁷ are selected from hydrogen, halogen, and C₁₋₆alkyl; R² and R⁵ are selected from hydrogen, alkoxy, hydroxyl, and amino-C₁₋₆alkyl; and, R³, R⁴, R⁶, and R⁸ are hydrogen. In certain embodiments, including any of the foregoing, R¹ and R⁷ are selected from hydrogen, halogen, and C₁₋₆alkyl; R² and R⁵ are selected from hydrogen and alkoxy; and, R³, R⁴, R⁶, and R⁸ are hydrogen. In certain embodiments, including any of the foregoing, R¹ and R⁷ are selected from hydrogen, halogen, and C₁₋₆alkyl; R² and R⁵ are selected from hydrogen and alkoxy; R³, R⁴, R⁶, and R⁸ are hydrogen; and L¹ is

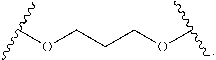 [SMILES: *OCCCO*]

In certain embodiments, including any of the foregoing, R¹ and R⁷ are halogen; R² and R⁵ are alkoxy; R³, R⁴, R⁶, and R⁸ are hydrogen; and L¹ is

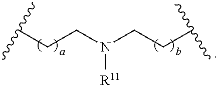

In certain embodiments, including any of the foregoing, R¹ and R⁷ are halogen; R² and R⁵ are alkoxy; R³, R⁴, R⁶, and R⁸ are hydrogen; and L¹ is

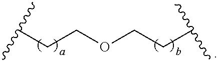

In one embodiment, including any of the foregoing, X¹ is —C(O)—. In one embodiment, including any of the foregoing, X¹ is —CH═CH—. In one embodiment, including any of the foregoing, X² is —(C(R^{12a}R^{12b}))₁₋₆—. In one embodiment, including any of the foregoing, X² is —(CH₂)₁₋₆—. In one embodiment, including any of the foregoing, X² is —(CH₂)— or —(CH₂)₂—. In one embodiment, including any of the foregoing, X² is —(C(R^{12a}R^{12b})C(R^{13a}R^{13b}))₁₋₃—. In one embodiment, including any of the foregoing, X² is —(CH₂C(CH₃)₂)₁₋₃—. In one embodiment, including any of the foregoing, X² is —(CH₂C(CH₃)₂)—. In one embodiment, including any of the foregoing, X³ is —COOR¹⁰. In one embodiment, including any of the foregoing, X³ is —COOH. In one embodiment, including any of the foregoing, X³ is —C(O)OC₁₋₆alkyl. In one embodiment, including any of the foregoing, X³ is —C(O)OCH₃. In one embodiment, including any of the foregoing, X³ is —P(O)(OR¹⁵)₂. In one embodiment, including any of the foregoing, X³ is —P(O)(OH)₂.

In one embodiment, including any of the foregoing,

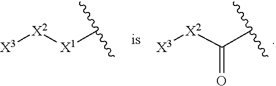

In one embodiment, including any of the foregoing,

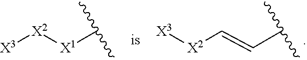

In one embodiment, including any of the foregoing,

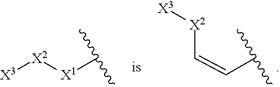

In certain embodiments, including any of the foregoing,

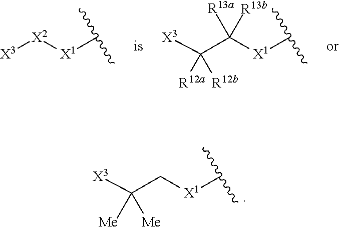

In one embodiment, including any of the foregoing,

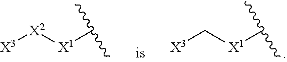

In certain embodiments, including any of the foregoing,

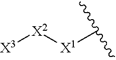

is selected from

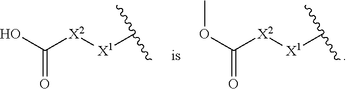

In one embodiment, including any of the foregoing,

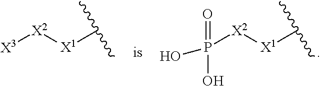

In one embodiment, including any of the foregoing,

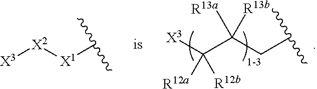

In one embodiment, including any of the foregoing,

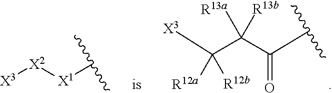

In one embodiment, including any of the foregoing,

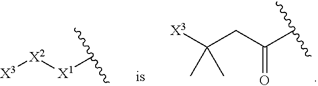

In one embodiment, including any of the foregoing,

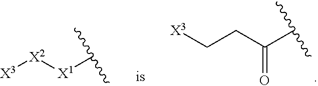

In one embodiment, including any of the foregoing,

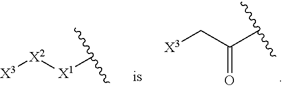

In one embodiment, including any of the foregoing,

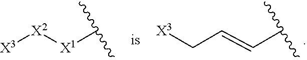

In one embodiment, including any of the foregoing,

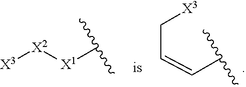

In certain embodiments, including any of the foregoing,

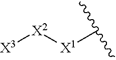

is selected from

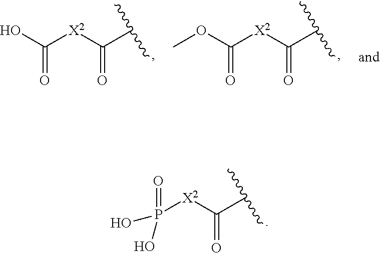

In certain embodiments, including any of the foregoing,

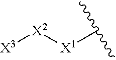

is selected from

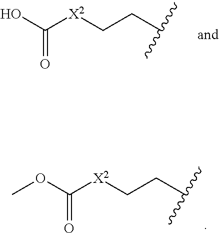

In certain embodiments, including any of the foregoing,

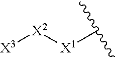

is selected from

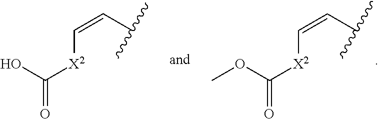

In one embodiment, including any of the foregoing,

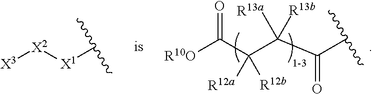

In one embodiment, including any of the foregoing,

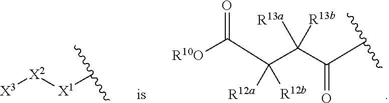

In one embodiment, including any of the foregoing,

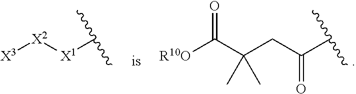

In one embodiment, including any of the foregoing,

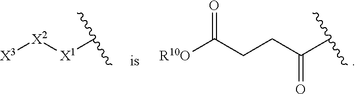

Non-limiting examples of

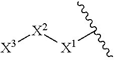

include

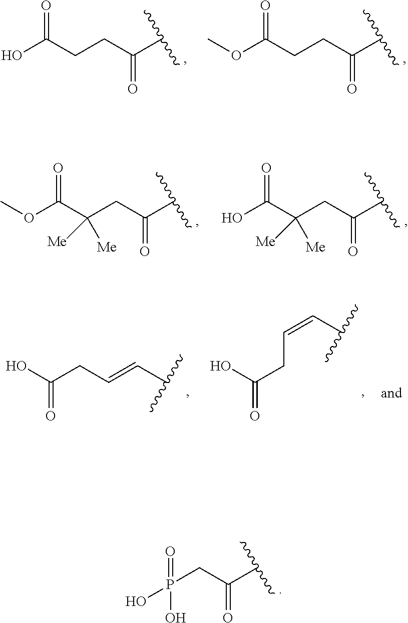

In certain embodiments, including any of the foregoing,

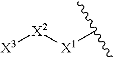

is selected from

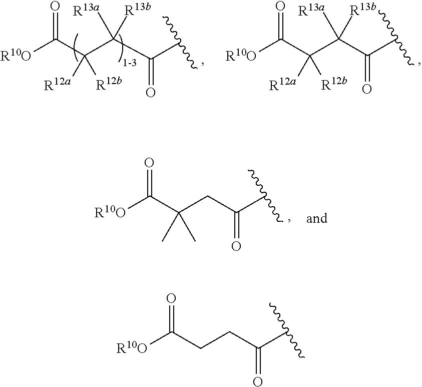

and L¹ is selected from

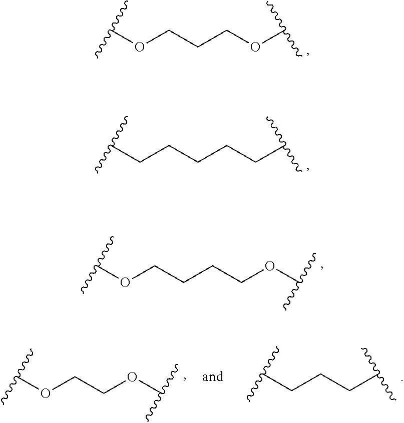 [SMILES: *OCCCO* ; *CCCCC* ; *OCCCCO* ; *OCCO* ; *CCC*]

In the foregoing embodiments,

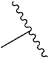

indicates a point of attachment to the rest of the compound.

In one embodiment, including any of the foregoing, X⁴ is —C(O)—. In one embodiment, including any of the foregoing, X⁴ is —CH₂—. In one embodiment, including any of the foregoing, X⁴ is —CH═CH—.

In one embodiment, including any of the foregoing, X⁵ is —(C(R^{12a}R^{12b}))₁₋₆—. In one embodiment, including any of the foregoing, X⁵ is —(CH₂)₁₋₆—. In one embodiment, including any of the foregoing, X⁵ is —(CH₂)—. In one embodiment, including any of the foregoing, X⁵ is —(CH₂)₂—. In one embodiment, including any of the foregoing, X⁵ is —(C(R^{12a}R^{12b})C(R^{13a}R^{13b}))₁₋₃—. In one embodiment, including any of the foregoing, X⁵ is —(CH₂C(CH₃)₂)₁₋₃—. In one embodiment, including any of the foregoing, X⁵ is —(CH₂C(CH₃)₂)—.

In one embodiment, including any of the foregoing, X⁶ is —C(O)NR^{9a}S(O)₂R¹⁷. In one embodiment, including any of the foregoing, X⁶ is —C(O)NR⁹'S(O)₂R¹⁷. In one embodiment, including any of the foregoing, X⁶ is —C(O)NHS(O)₂R¹⁷. In one embodiment, including any of the foregoing, X⁶ is —C(O)NHS(O)₂-amino-C₁₋₆alkyl. In one embodiment, including any of the foregoing, X⁶ is —C(O)NHS(O)₂(CH₂)₂NH₂. In one embodiment, including any of the foregoing, X⁶ is —S(O)₂OH. In one embodiment, including any of the foregoing, X⁶ is —P(O)(OR¹⁵)₂. In one embodiment, including any of the foregoing, X⁶ is —P(O)(OH)₂. In one embodiment, including any of the foregoing, X⁶ is —P(O)(OC₁₋₆alkyl)₂. In one embodiment, including any of the foregoing, X⁶ is —P(O)(OCH₃)₂. In one embodiment, including any of the foregoing, X⁶ is —C(O)NR^{9a}R¹⁸. In one embodiment, including any of the foregoing, X⁶ is —C(O)NHR¹⁸. In one embodiment, including any of the foregoing, X⁶ is —C(O)NH-amino-C₁₋₆alkyl. In one embodiment, including any of the foregoing, X⁶ is —C(O)NH(CH₂)₂NH₂.

In one embodiment, including any of the foregoing, X⁷ is —C(R^{12a}R^{12c})C(R^{13a}R^{13c})—. In one embodiment, including any of the foregoing, X⁷ is

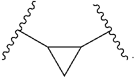 [SMILES: *C1CC1*]

In one embodiment, including any of the foregoing, X⁷ is

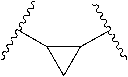 [SMILES: *C1CC1*]

wherein the cycloalkyl is optionally substituted with a substituent selected from halogen, alkoxy, C₁₋₆alkyl, C₃₋₁₀cycloalkyl, hydroxy, cyano, —NR^{9a}R^{9b}, and —COOR¹⁰. In one embodiment, including any of the foregoing, X⁷ is —CH═CH—.

In one embodiment, including any of the foregoing, X⁸ is —COOR¹⁰. In one embodiment, including any of the foregoing, X⁸ is —COOH. In one embodiment, including any of the foregoing, X⁸ is —C(O)OC₁₋₆alkyl. In one embodiment, including any of the foregoing, X⁸ is

—C(O)OCH₃. In one embodiment, including any of the foregoing, X⁸ is —P(O)(OR¹⁵)₂. In one embodiment, including any of the foregoing, X⁸ is —P(O)(OH)₂.

In one embodiment, including any of the foregoing, R^{9a} is hydrogen. In one embodiment, including any of the foregoing, R^{9a} is C₁₋₆alkyl, including, but not limited to methyl.

In one embodiment, including any of the foregoing, R¹⁰ is hydrogen. In one embodiment, including any of the foregoing, R¹⁰ is C₁₋₆alkyl. In one embodiment, including any of the foregoing, R¹⁰ is C₃₋₁₀cycloalkyl. In one embodiment, including any of the foregoing, R¹⁰ is selected from aryl, aryl-C₁₋₆alkyl, and heteroaryl.

In certain embodiments, including any of the foregoing, R¹⁴ is amino-C₁₋₆alkyl, C₁-6alkyl-amino-C₁₋₆alkyl, di-C₁₋₆alkylamino-C₁₋₆alkyl, or C₃₋₁₀cycloalkyl-amino-C₁₋₆alkyl. In one embodiment, including any of the foregoing, R¹⁴ is amino-C₁₋₆alkyl. In one embodiment, including any of the foregoing, R¹⁴ is halo-C₁₋₆alkyl. In one embodiment, including any of the foregoing, R¹⁴ is C₁₋₆alkyl.

In certain embodiments, including any of the foregoing, R¹⁷ is amino-C₁₋₆alkyl, C₁₋₆alkyl-amino-C₁₋₆alkyl, di-C₁₋₆alkylamino-C₁₋₆alkyl, or C₃₋₁₀cycloalkyl-amino-C₁₋₆alkyl. In one embodiment, including any of the foregoing, R¹⁷ is amino-C₁₋₆alkyl. In certain embodiments, including any of the foregoing, R¹⁸ is amino-C₁₋₆alkyl, C₁₋₆alkyl-amino-C₁₋₆alkyl, di-C₁₋₆alkylamino-C₁₋₆alkyl, or C₃₋₁₀cycloalkyl-amino-C₁₋₆alkyl. In one embodiment, including any of the foregoing, R¹⁸ is amino-C₁₋₆alkyl.

In certain embodiments, including any of the foregoing, R¹⁵ is independently selected from hydrogen and C₁₋₆alkyl. In one embodiment, including any of the foregoing, R¹⁵ is hydrogen. In one embodiment, including any of the foregoing, R⁵ is C₁₋₆alkyl. In one embodiment, including any of the foregoing, R¹⁵ is methyl.

In certain embodiments, including any of the foregoing,

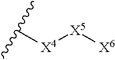

is selected from

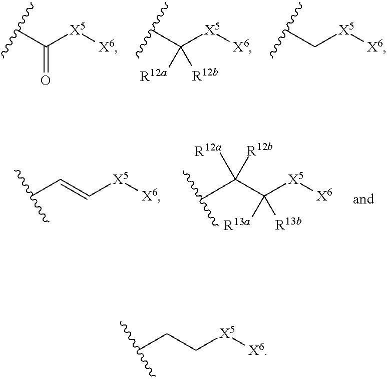

In one embodiment, including any of the foregoing,

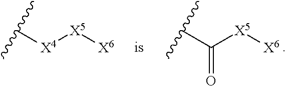

In one embodiment, including any of the foregoing,

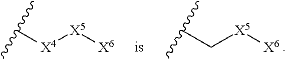

In certain embodiments, including any of the foregoing,

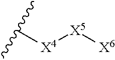

is selected from

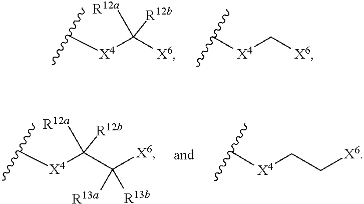

In certain embodiments, including any of the foregoing

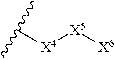

selected from

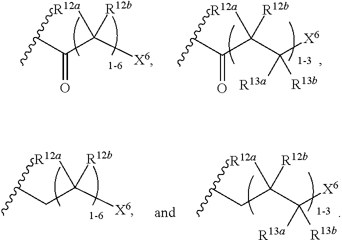

In certain embodiments, including any of the foregoing,

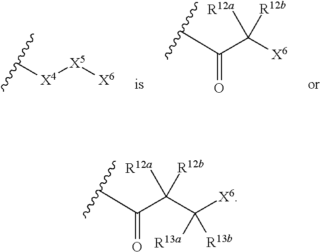

In one embodiment, including any of the foregoing,

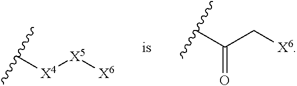

In certain embodiments, including any of the foregoing,

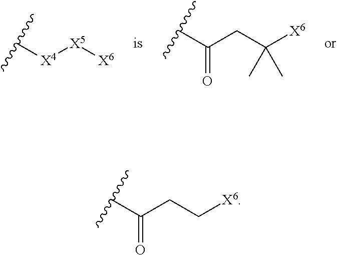

In certain embodiments, including any of the foregoing,

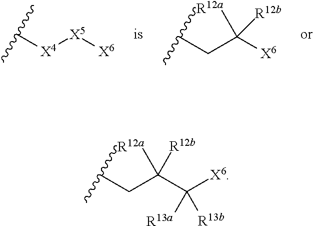

In one embodiment, including any of the foregoing,

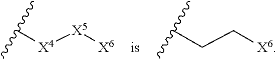

In certain embodiments, including any of the foregoing,

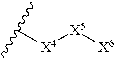

is selected from

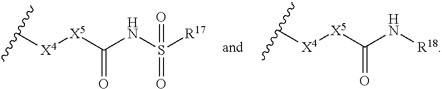

In certain embodiments, including any of the foregoing,

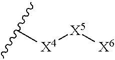

is selected from

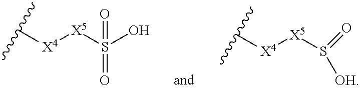

In certain embodiments, including any of the foregoing,

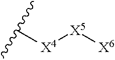

is selected from

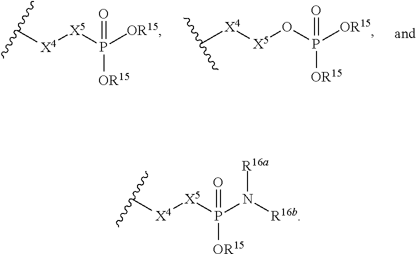

In certain embodiments, including any of the foregoing,

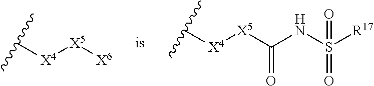

wherein R¹⁷ is amino-C₁₋₆alkyl, C₁₋₆alkyl-amino-C₁₋₆alkyl, di-C₁₋₆alkylamino-C₁₋₆alkyl, or C₃₋₁₀cycloalkyl-amino-C₁₋₆alkyl. In one embodiment, including any of the foregoing,

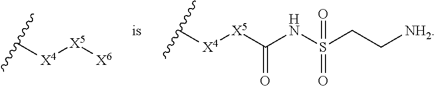

In certain embodiments, including any of the foregoing,

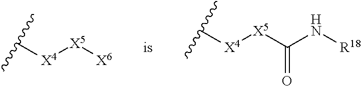

wherein R¹⁸ is amino-C₁₋₆alkyl, C₁₋₆alkyl-amino-C₁₋₆alkyl, di-C₁₋₆alkylamino-C₁₋₆alkyl, or C₃₋₁₀cycloalkyl-amino-C₁₋₆alkyl. In one embodiment, including any of the foregoing,

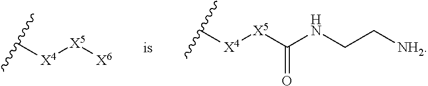

In certain embodiments, including any of the foregoing,

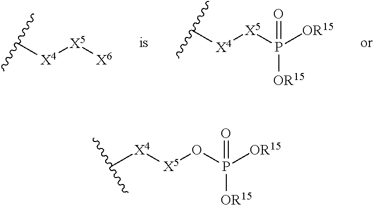

and R¹⁵ is independently selected from hydrogen and C₁₋₆alkyl. In one embodiment, including any of the foregoing, R¹⁵ is methyl.

In certain embodiments, including any of the foregoing,

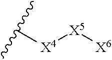

is selected from

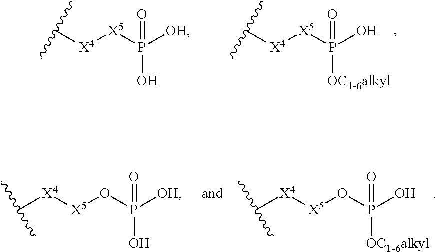

In one embodiment, including any of the foregoing,

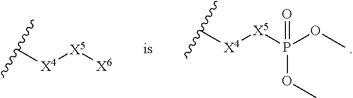

In certain embodiments, including any of the foregoing,

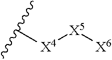

is selected from

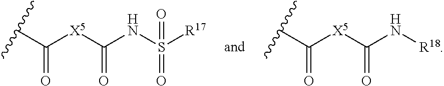

In certain embodiments, including any of the foregoing,

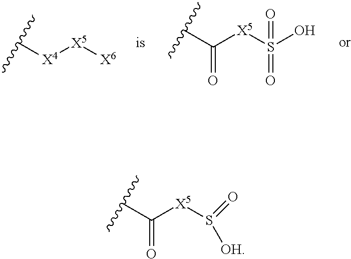

In one embodiment, including any of the foregoing,

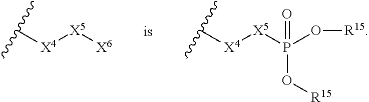

In certain embodiments, including any of the foregoing,

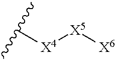

is selected from

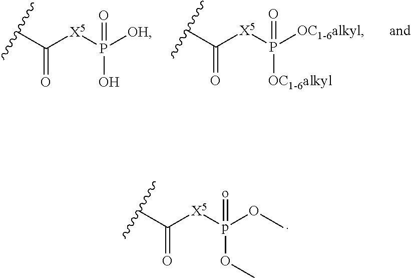

In certain embodiments, including any of the foregoing,

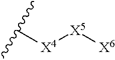

is selected from

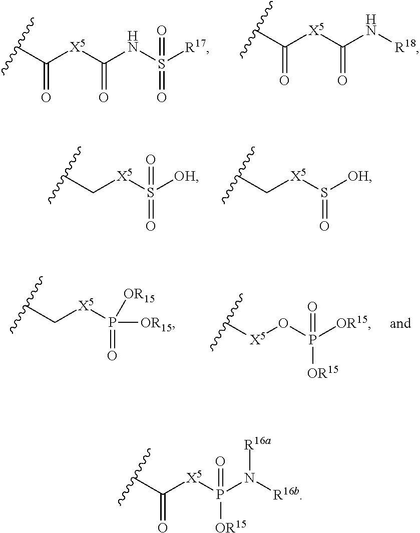

In certain embodiments, including any of the foregoing,

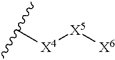

is selected from

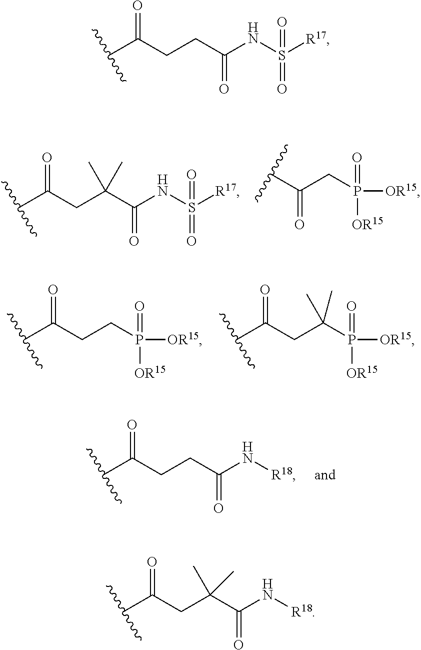

Non-limiting examples of

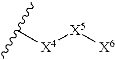

include:

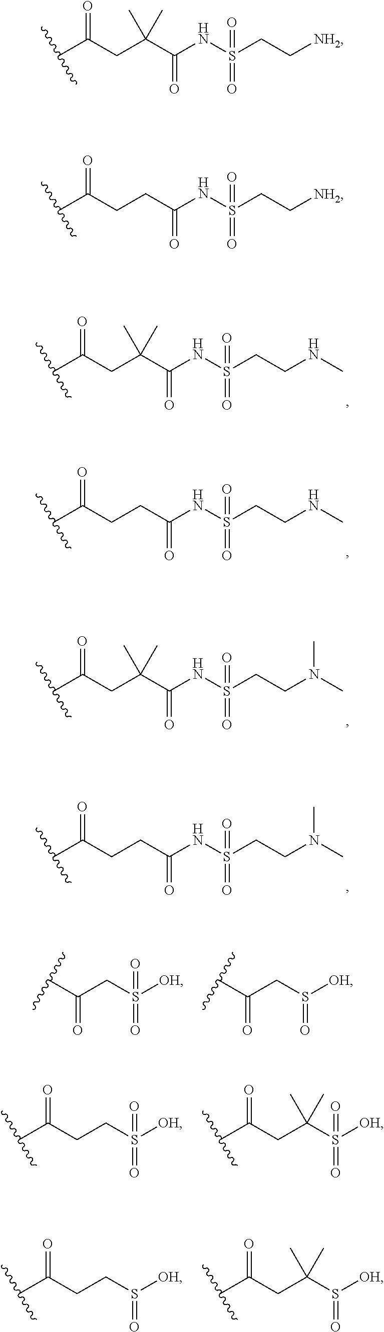

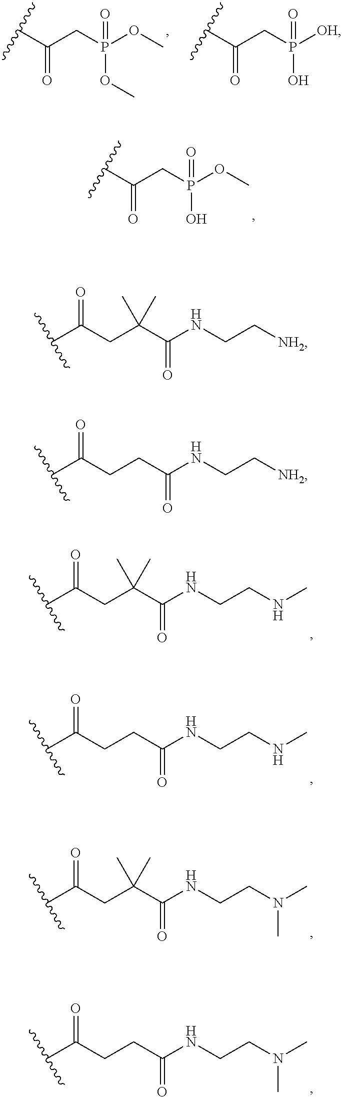

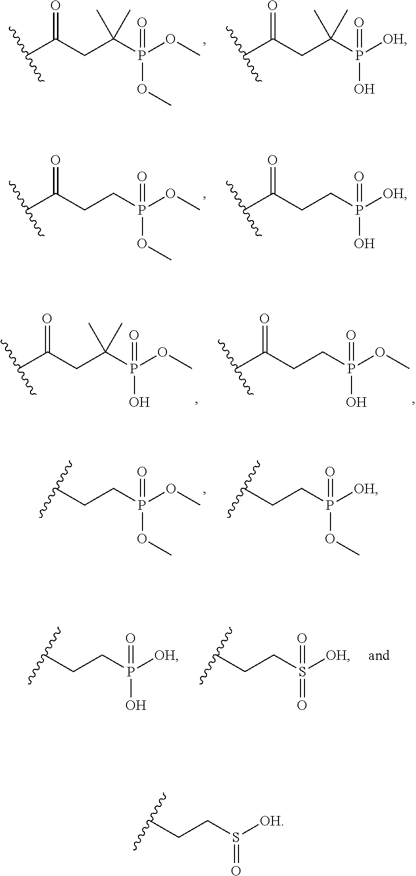

In certain embodiments, including any of the foregoing,

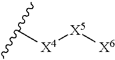

is selected from

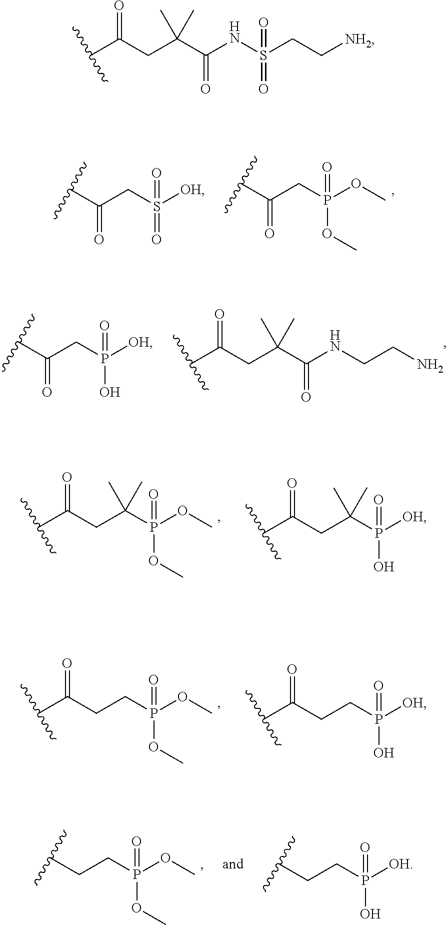

In certain embodiments, including any of the foregoing,

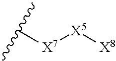

is selected from

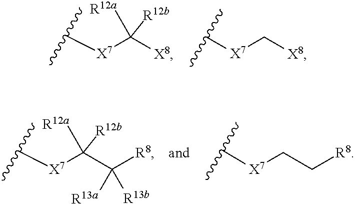

In certain embodiments, including any of the foregoing,

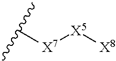

is selected from

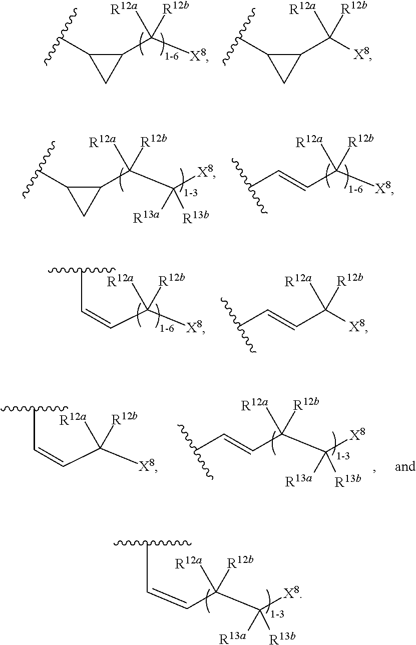

In certain embodiments, including any of the foregoing,

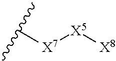

is selected from

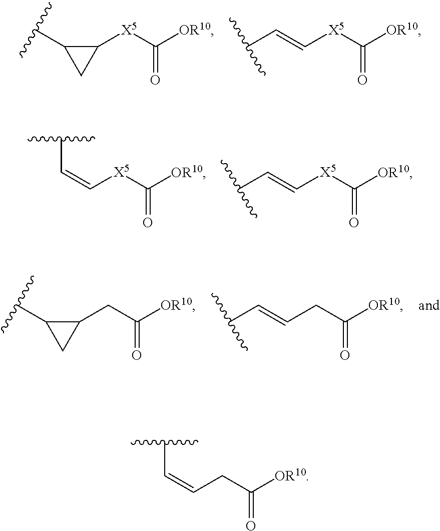

In certain embodiments, including any of the foregoing,

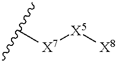

is selected from

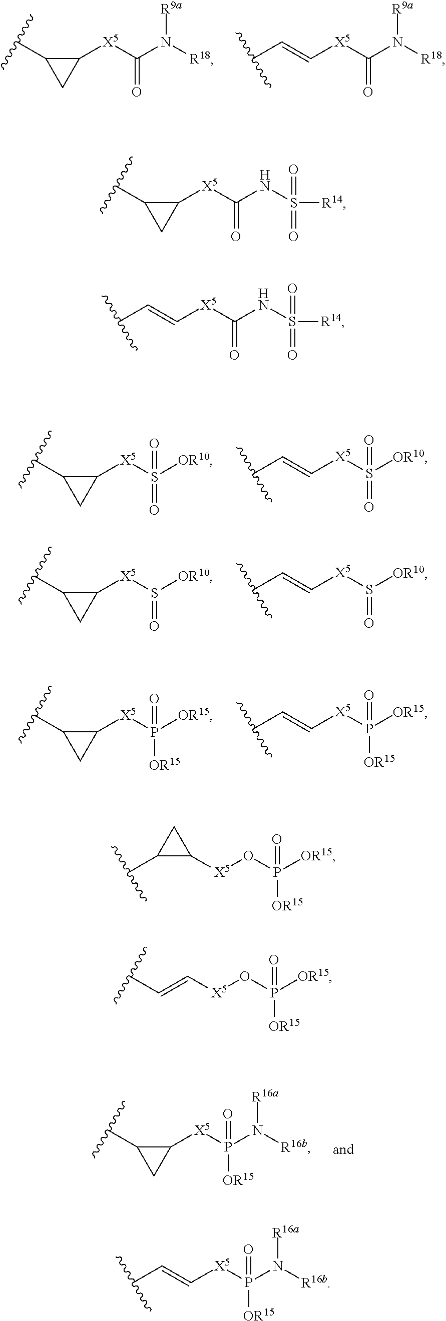

In certain embodiments, including any of the foregoing,

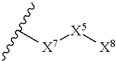

selected from

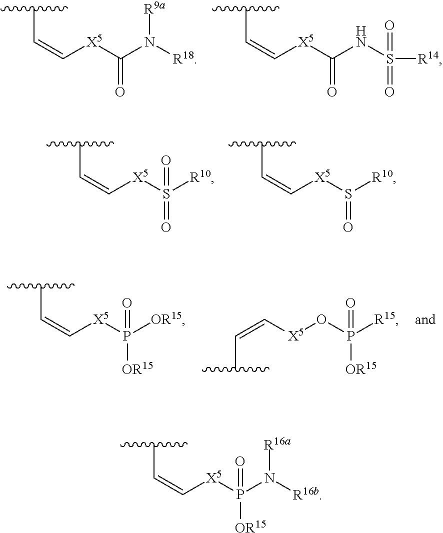

Non-limiting examples of

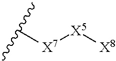

include

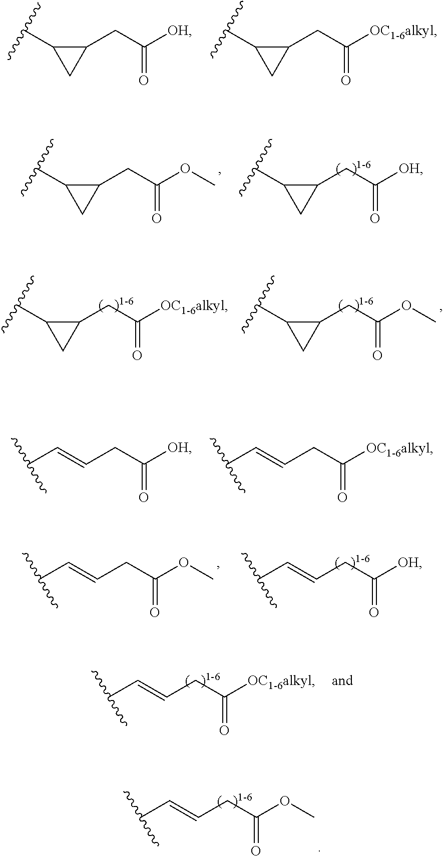

Non-limiting examples of

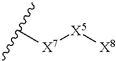

include

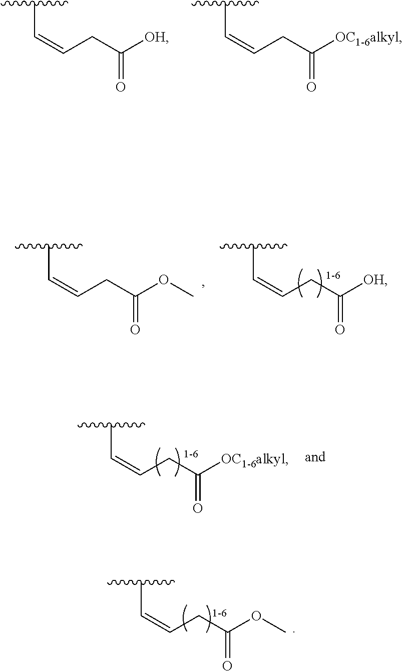

In certain embodiments, including any of the foregoing,

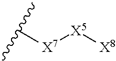

is selected from

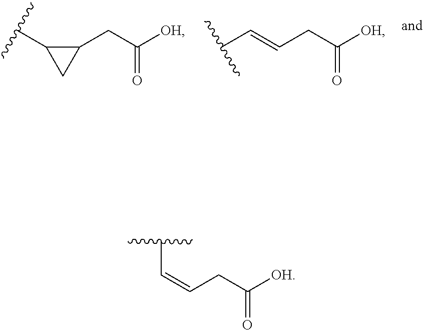 [SMILES: *C1CC1CC(=O)O ; */C=C/CC(=O)O ; */C=C\CC(=O)O]

In certain embodiments, including any of the foregoing,

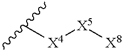

is selected from

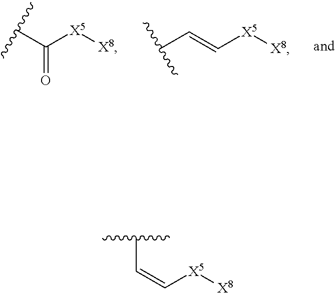

In certain embodiments, including any of the foregoing,

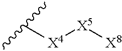

is selected from

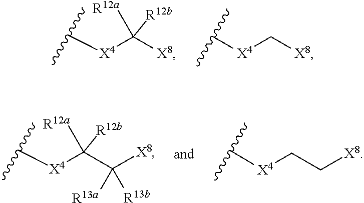

In certain embodiments, including any of the foregoing,

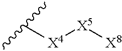

is selected from

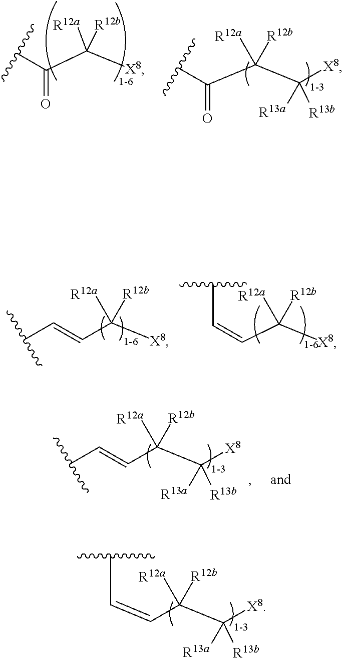

In certain embodiments, including any of the foregoing,

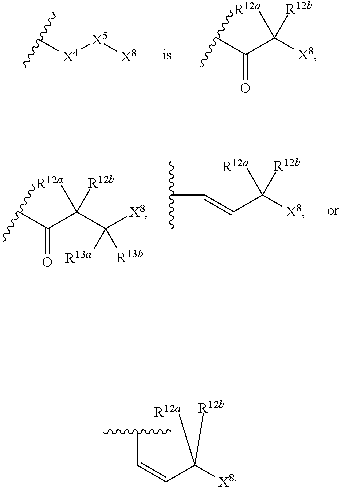

In certain embodiments, including any of the foregoing,

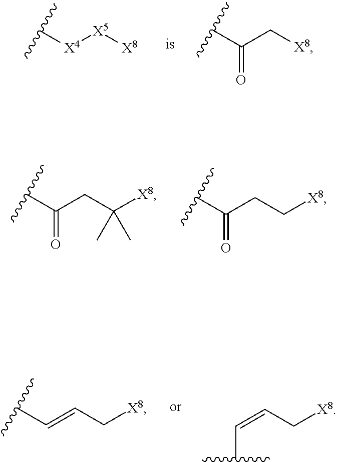

Non-limiting examples of

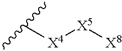

include

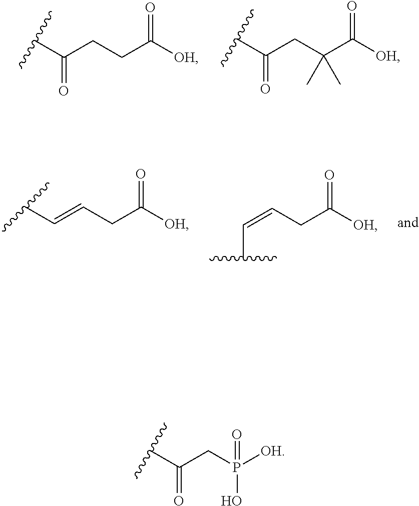

In certain embodiments, including any of the foregoing,

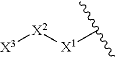

is selected from

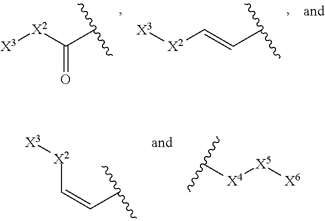

is selected from

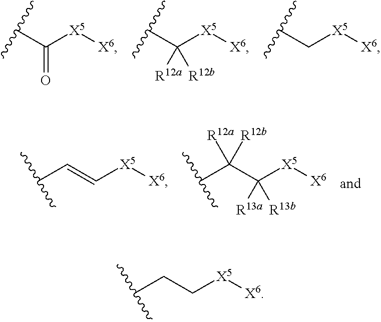

In certain embodiments, including any of the foregoing,

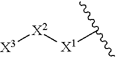

is selected from

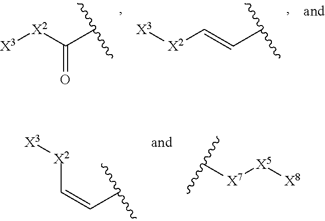

is selected from

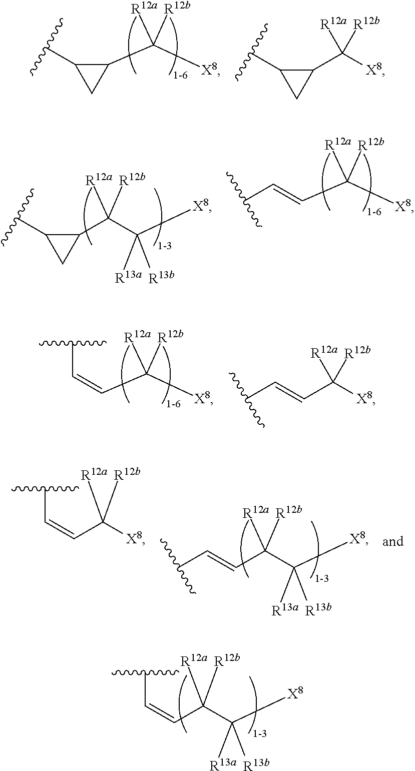

In certain embodiments, including any of the foregoing,

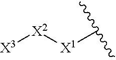

is selected from

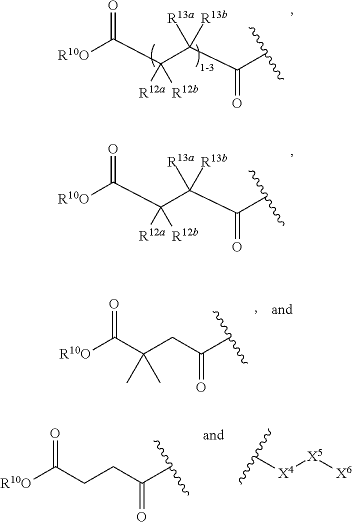

is selected from

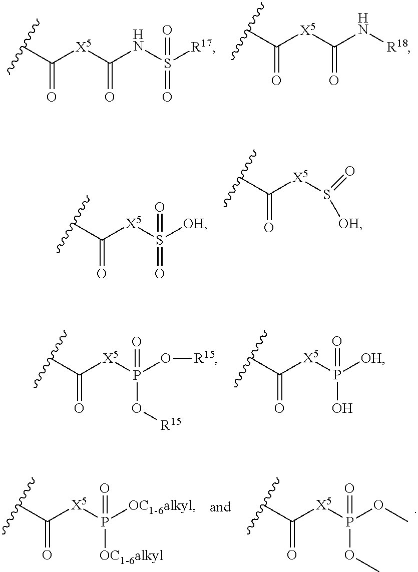

In certain embodiments, including any of the foregoing,

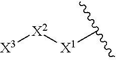

is selected from

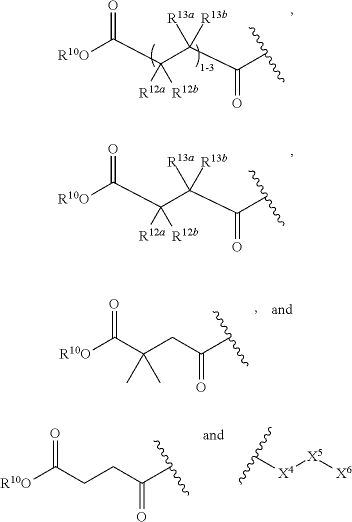

is selected from

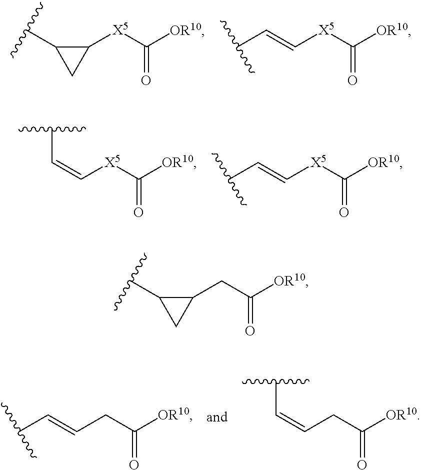

In certain embodiments, including any of the foregoing,

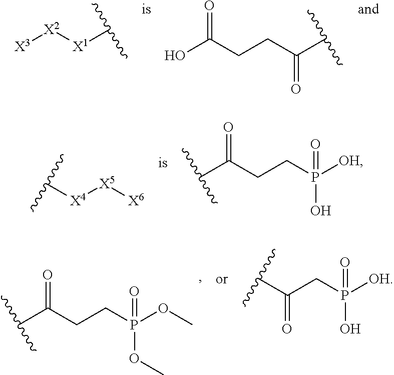

In certain embodiments, including any of the foregoing,

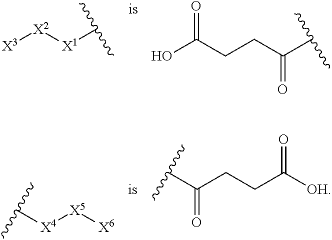

In certain embodiments, including any of the foregoing,

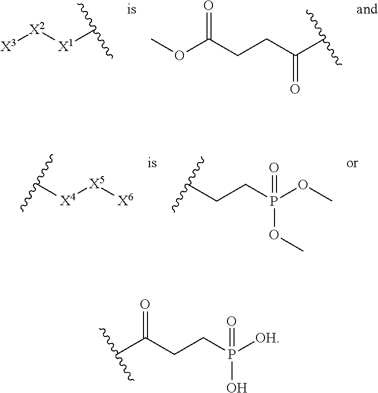

In certain embodiments, including any of the foregoing,

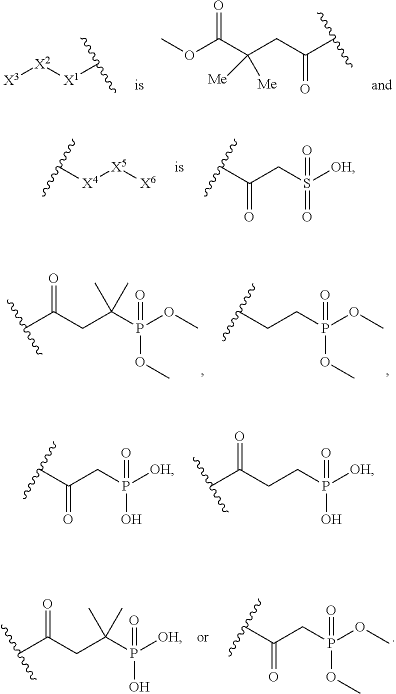

In certain embodiments, including any of the foregoing,

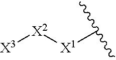

is

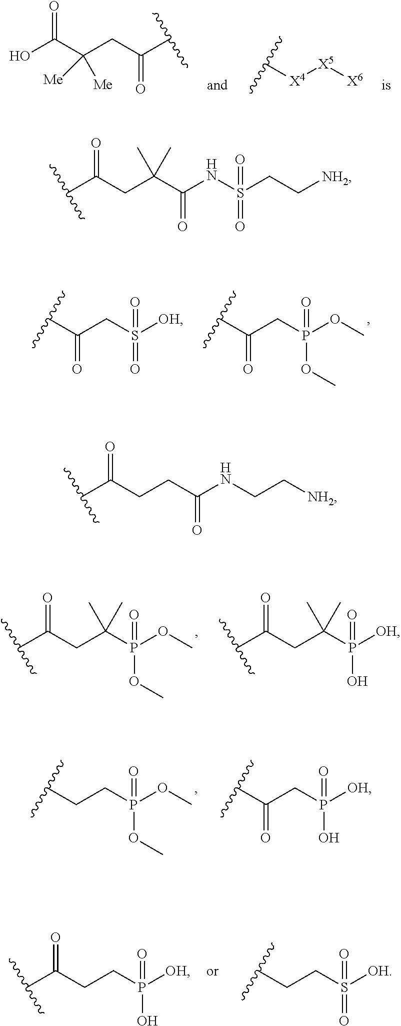

In certain embodiments, including any of the foregoing,

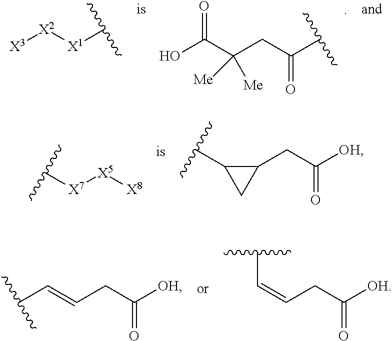

In certain embodiments, including any of the foregoing,

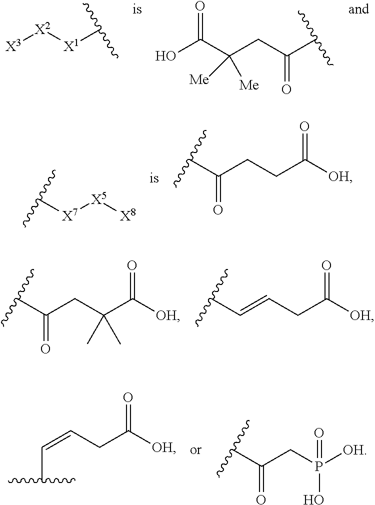

In certain embodiments, including any of the foregoing,

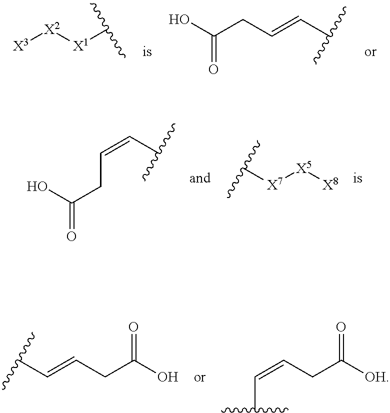

In certain embodiments, including any of the foregoing,

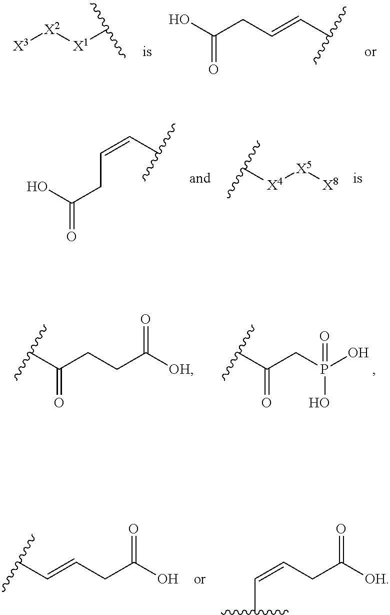

In certain embodiments, including any of the foregoing,

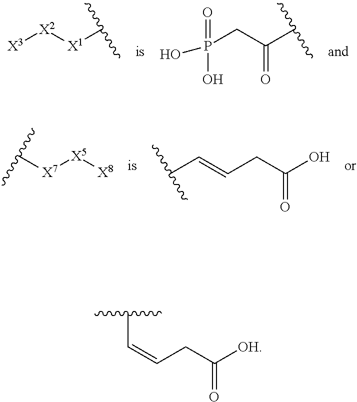

In certain embodiments, including any of the foregoing,

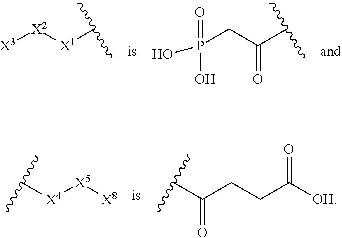

In one embodiment, including any of the foregoing, R^{12a} and R^{12b} are each hydrogen. In one embodiment, including any of the foregoing, R^{12a}, R^{12b}, R^{13a}, and are R^{13b} are each hydrogen. In one embodiment, including any of the foregoing, R^{12a} and R^{12b} are each hydrogen and R^{13a} and are R^{13b} are each C₁₋₆alkyl. In one embodiment, including any of the foregoing, R^{12a} and R^{12b} are each hydrogen and R^{13a} and R^{13b} are each methyl.

In certain embodiments, including any of the foregoing, R^{12a} and R^{13b} are each hydrogen and R^{12a} and R^{12a} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰. In certain embodiments, including any of the foregoing, R^{12a} and R^{13b} are each hydrogen and R^{12c} and R^{12a} are joined together to form a cyclopropyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰. In one embodiment, including any of the foregoing, R^{12a} and R^{13b} are each hydrogen and R^{12a} and R^{12a} are joined together to form an unsubstituted cyclopropyl.

In certain embodiments, including any of the foregoing, X⁹, X¹⁰, and X¹¹ are independently selected from CR¹⁹ and N and R¹⁹ is hydrogen. In certain embodiments, including any of the foregoing, X⁹, X¹⁰, and X¹¹ are independently selected from CR¹⁹ and N and R¹⁹ is independently selected from hydrogen, halogen, C₁₋₆alkyl, and alkoxy. In certain embodiments, including any of the foregoing, X⁹, X¹⁰, and X¹¹ are each CR¹⁹ and R¹⁹ is hydrogen. In certain embodiments, including any of the foregoing, X⁹, X¹⁰, and X¹¹ are each CR¹⁹ and R¹⁹ is selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, and —COOR¹⁰. In one embodiment, including any of the foregoing, X⁹ is N and X¹⁰ and X¹¹ are CR¹⁹. In one embodiment, including any of the foregoing, X¹⁰ is N and X⁹ and X¹¹ are CR¹⁹. In one embodiment, including any of the foregoing, X¹ is N and X¹⁰ and X⁹ are CR¹⁹. In one embodiment, including any of the foregoing, X⁹ is N, X¹⁰ is N, and X¹¹ is CR¹⁹. In one embodiment, including any of the foregoing, X⁹ is N, X¹¹ is N, and X¹⁰ is CR¹⁹. In one embodiment, including any of the foregoing, X¹⁰ is N, X¹¹ is N, and X⁹ is CR¹⁹.

In certain embodiments, a STING agonist of the present disclosure is a compound or a pharmaceutically acceptable salt, solvate, or stereoisomer thereof selected from:

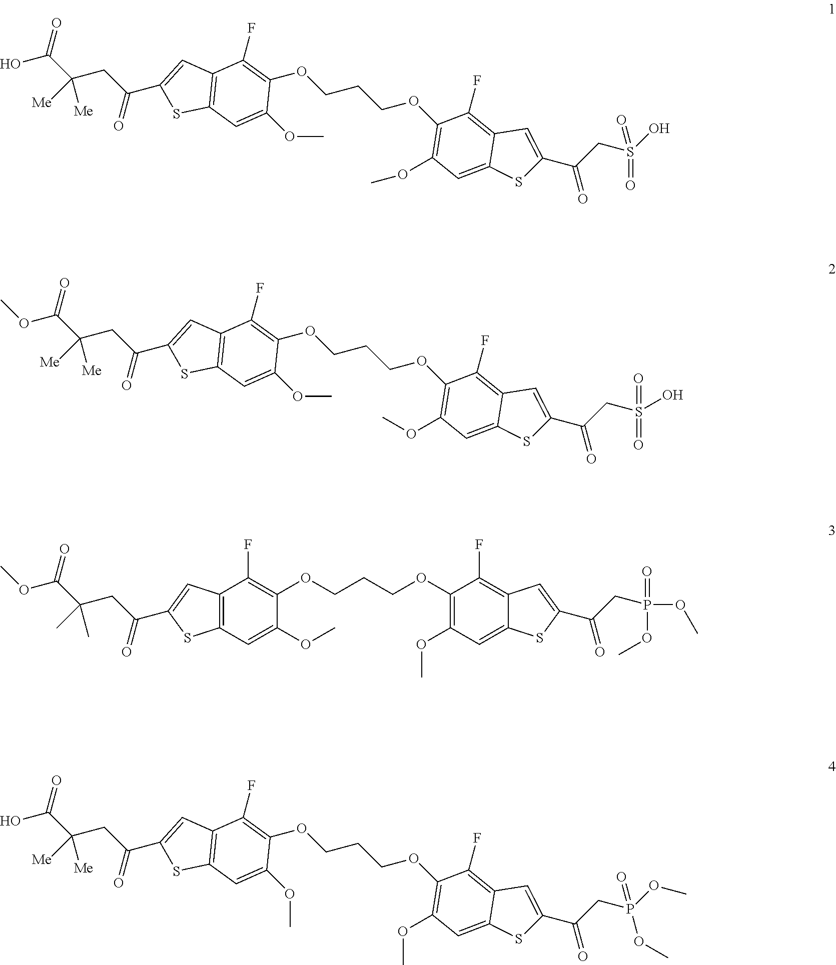

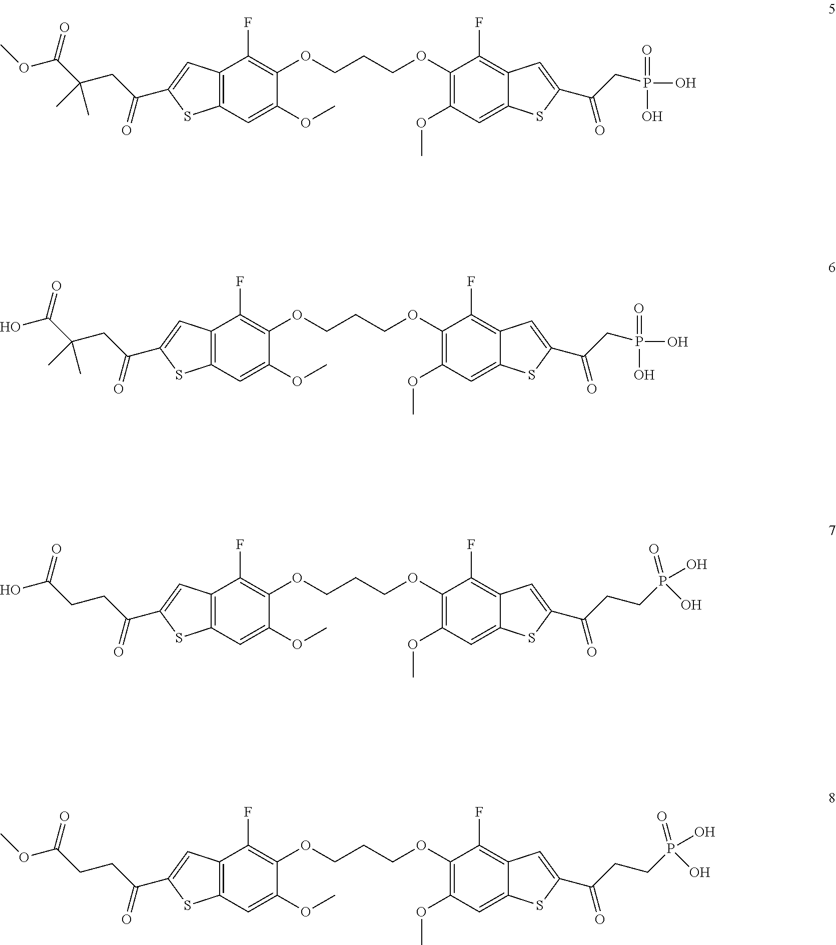 [SMILES: COC(=O)C(C)(C)CC(=O)c1cc2c(F)c(OCCCOc3c(OC)cc4sc(C(=O)CP(=O)(O)O)cc4c3F)c(OC)cc2s1 ; COc1cc2sc(C(=O)CC(C)(C)C(=O)O)cc2c(F)c1OCCCOc1c(OC)cc2sc(C(=O)CP(=O)(O)O)cc2c1F ; COc1cc2sc(C(=O)CCC(=O)O)cc2c(F)c1OCCCOc1c(OC)cc2sc(C(=O)CCP(=O)(O)O)cc2c1F ; COC(=O)CCC(=O)c1cc2c(F)c(OCCCOc3c(OC)cc4sc(C(=O)CCP(=O)(O)O)cc4c3F)c(OC)cc2s1]

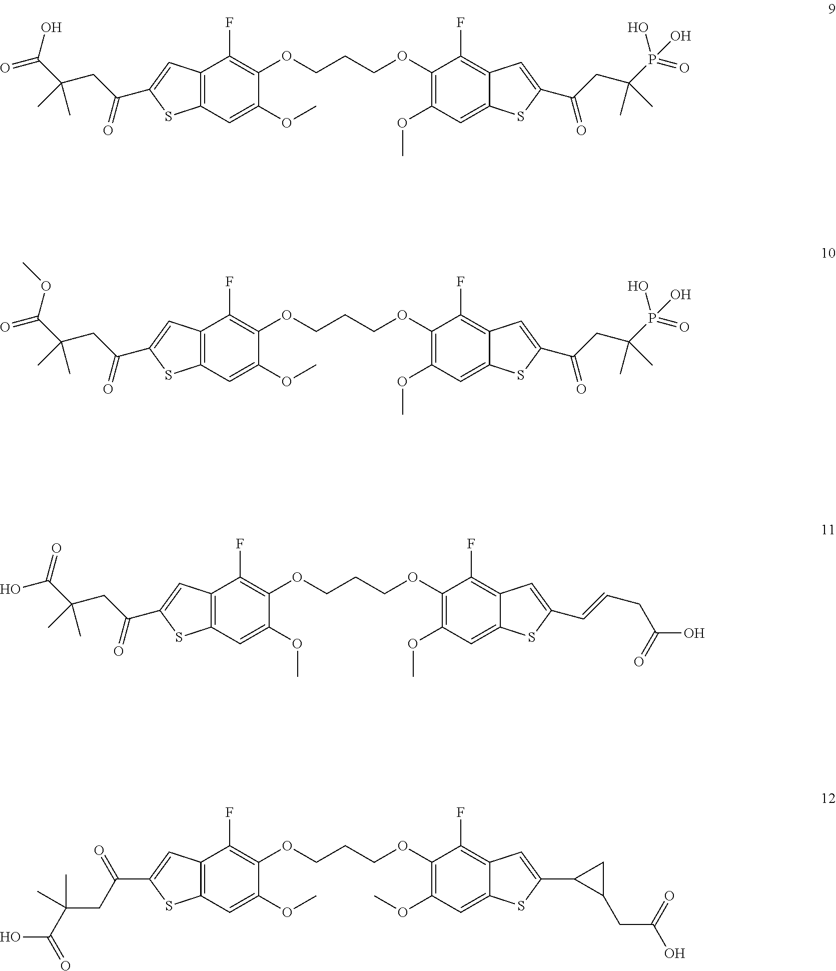 [SMILES: COc1cc2sc(C(=O)CC(C)(C)C(=O)O)cc2c(F)c1OCCCOc1c(OC)cc2sc(C(=O)CC(C)(C)P(=O)(O)O)cc2c1F ; COC(=O)C(C)(C)CC(=O)c1cc2c(F)c(OCCCOc3c(OC)cc4sc(C(=O)CC(C)(C)P(=O)(O)O)cc4c3F)c(OC)cc2s1 ; COc1cc2sc(/C=C/CC(=O)O)cc2c(F)c1OCCCOc1c(OC)cc2sc(C(=O)CC(C)(C)C(=O)O)cc2c1F ; COc1cc2sc(C(=O)CC(C)(C)C(=O)O)cc2c(F)c1OCCCOc1c(OC)cc2sc(C3CC3CC(=O)O)cc2c1F]

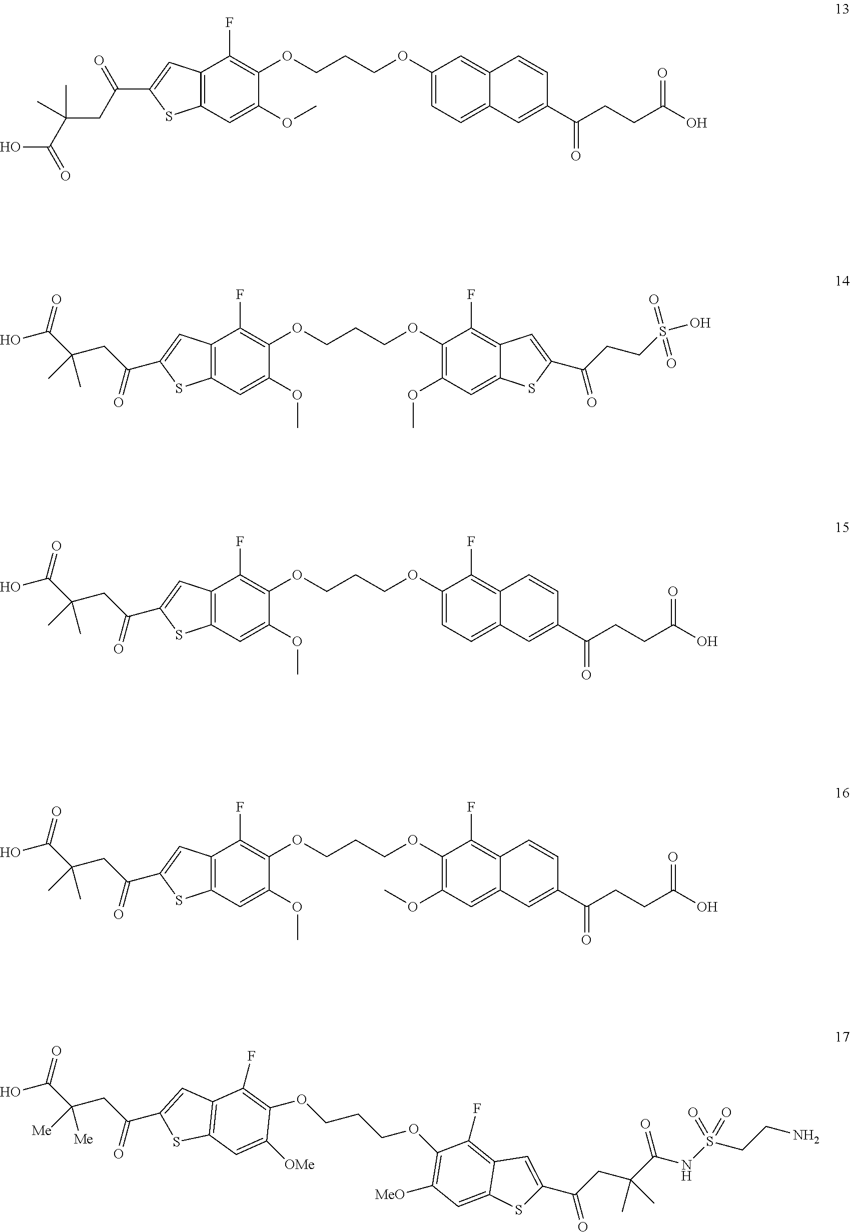 [SMILES: COc1cc2sc(C(=O)CC(C)(C)C(=O)O)cc2c(F)c1OCCCOc1ccc2cc(C(=O)CCC(=O)O)ccc2c1 ; COc1cc2sc(C(=O)CCS(=O)(=O)O)cc2c(F)c1OCCCOc1c(OC)cc2sc(C(=O)CC(C)(C)C(=O)O)cc2c1F ; COc1cc2sc(C(=O)CC(C)(C)C(=O)O)cc2c(F)c1OCCCOc1ccc2cc(C(=O)CCC(=O)O)ccc2c1F ; COc1cc2cc(C(=O)CCC(=O)O)ccc2c(F)c1OCCCOc1c(OC)cc2sc(C(=O)CC(C)(C)C(=O)O)cc2c1F ; COc1cc2sc(C(=O)CC(C)(C)C(=O)O)cc2c(F)c1OCCCOc1c(OC)cc2sc(C(=O)CC(C)(C)C(=O)NS(=O)(=O)CCN)cc2c1F]

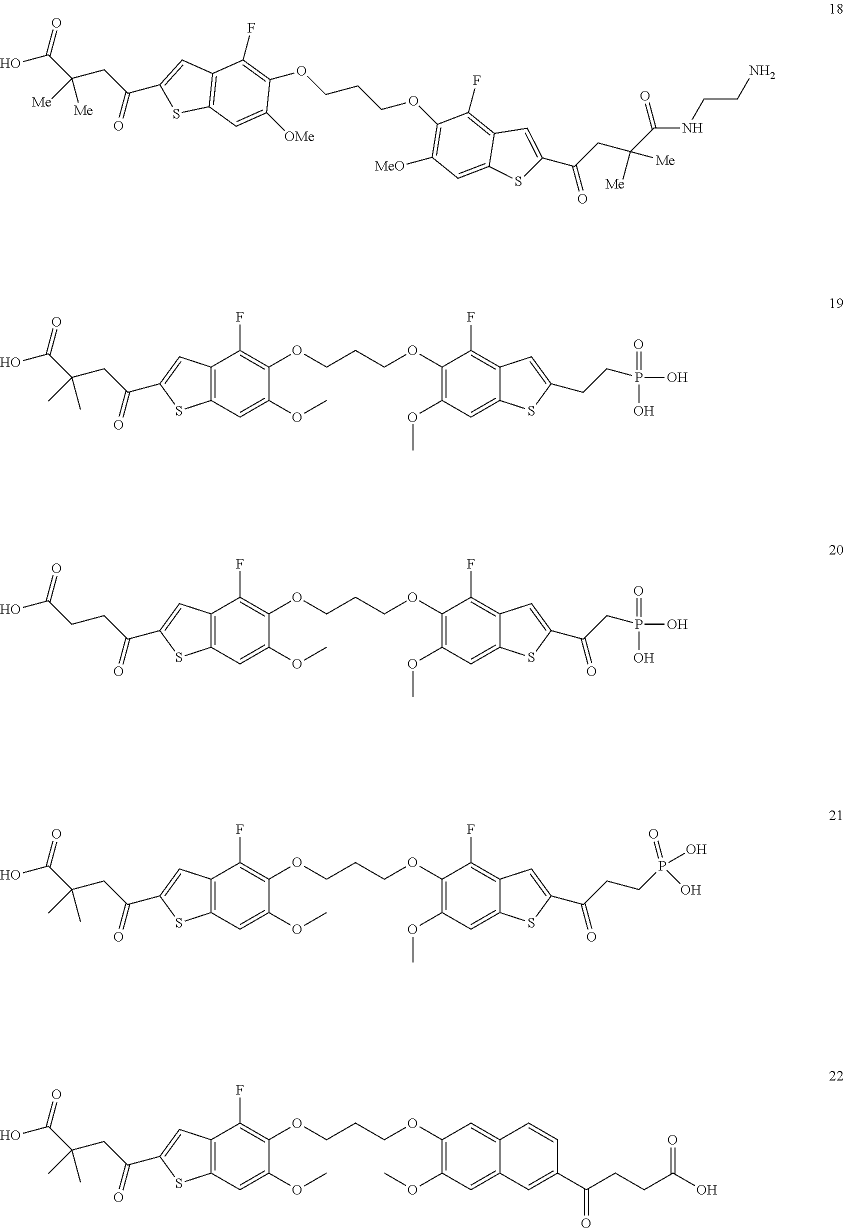 [SMILES: COc1cc2sc(C(=O)CC(C)(C)C(=O)O)cc2c(F)c1OCCCOc1c(OC)cc2sc(C(=O)CC(C)(C)C(=O)NCCN)cc2c1F ; COc1cc2sc(CCP(=O)(O)O)cc2c(F)c1OCCCOc1c(OC)cc2sc(C(=O)CC(C)(C)C(=O)O)cc2c1F ; COc1cc2sc(C(=O)CCC(=O)O)cc2c(F)c1OCCCOc1c(OC)cc2sc(C(=O)CP(=O)(O)O)cc2c1F ; COc1cc2sc(C(=O)CCP(=O)(O)O)cc2c(F)c1OCCCOc1c(OC)cc2sc(C(=O)CC(C)(C)C(=O)O)cc2c1F ; COc1cc2cc(C(=O)CCC(=O)O)ccc2cc1OCCCOc1c(OC)cc2sc(C(=O)CC(C)(C)C(=O)O)cc2c1F]

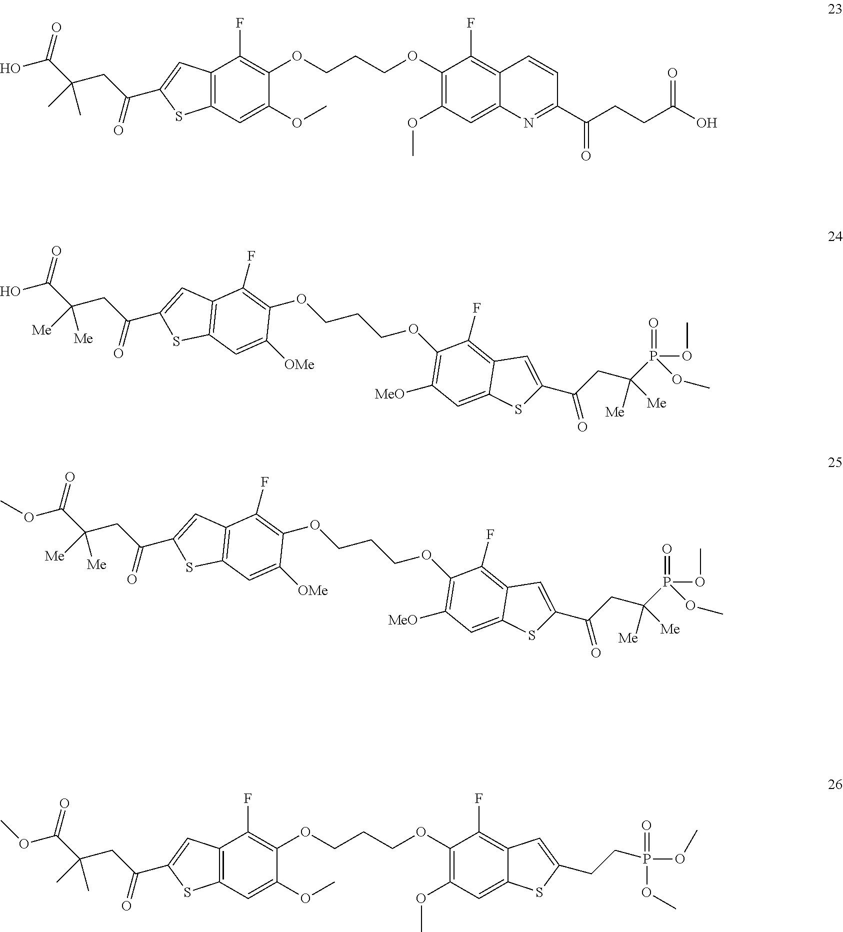

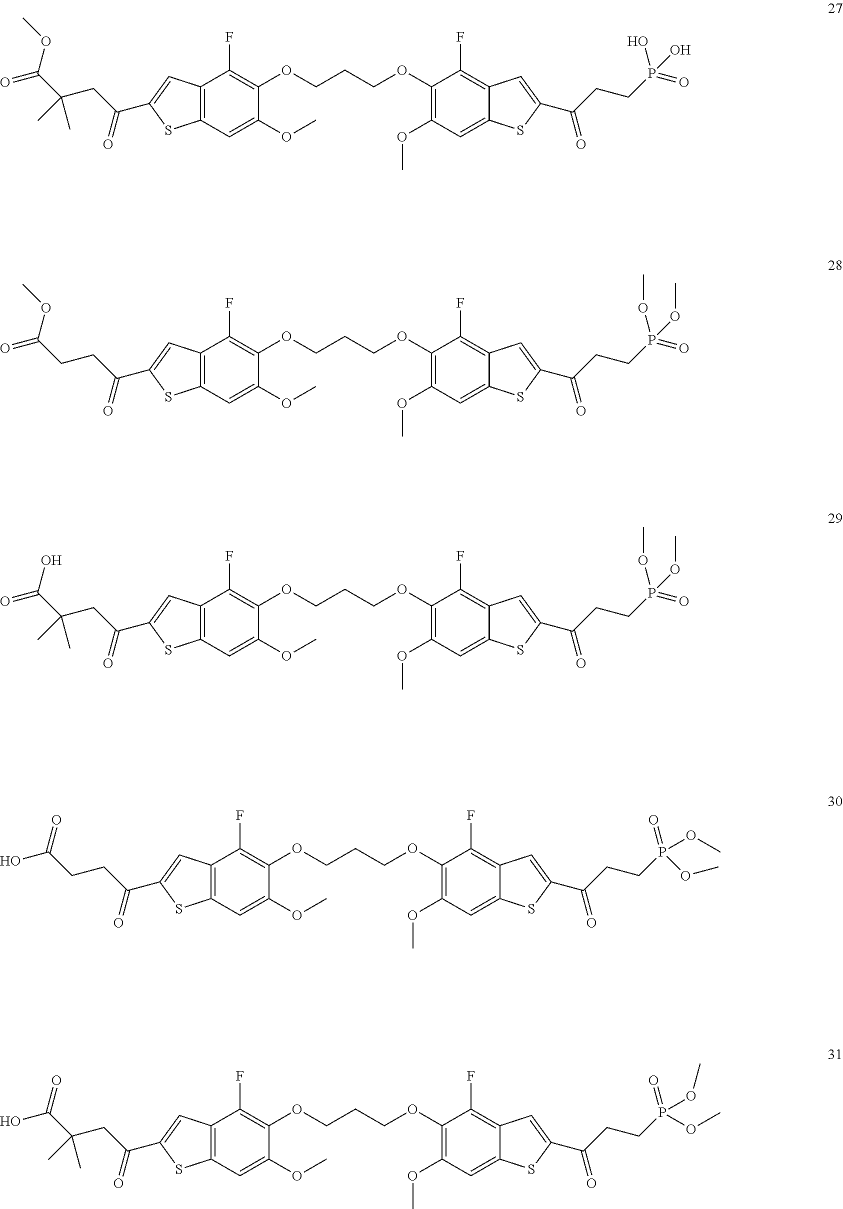 [SMILES: COC(=O)C(C)(C)CC(=O)c1cc2c(F)c(OCCCOc3c(OC)cc4sc(C(=O)CCP(=O)(O)O)cc4c3F)c(OC)cc2s1 ; COC(=O)CCC(=O)c1cc2c(F)c(OCCCOc3c(OC)cc4sc(C(=O)CCP(=O)(OC)OC)cc4c3F)c(OC)cc2s1 ; COc1cc2sc(C(=O)CCP(=O)(OC)OC)cc2c(F)c1OCCCOc1c(OC)cc2sc(C(=O)CC(C)(C)C(=O)O)cc2c1F ; COc1cc2sc(C(=O)CCC(=O)O)cc2c(F)c1OCCCOc1c(OC)cc2sc(C(=O)CCP(=O)(OC)OC)cc2c1F ; COc1cc2sc(C(=O)CCP(=O)(OC)OC)cc2c(F)c1OCCCOc1c(OC)cc2sc(C(=O)CC(C)(C)C(=O)O)cc2c1F]

In certain embodiments, a STING agonist of the present disclosure is a compound or a pharmaceutically acceptable salt, solvate, or stereoisomer thereof selected from:

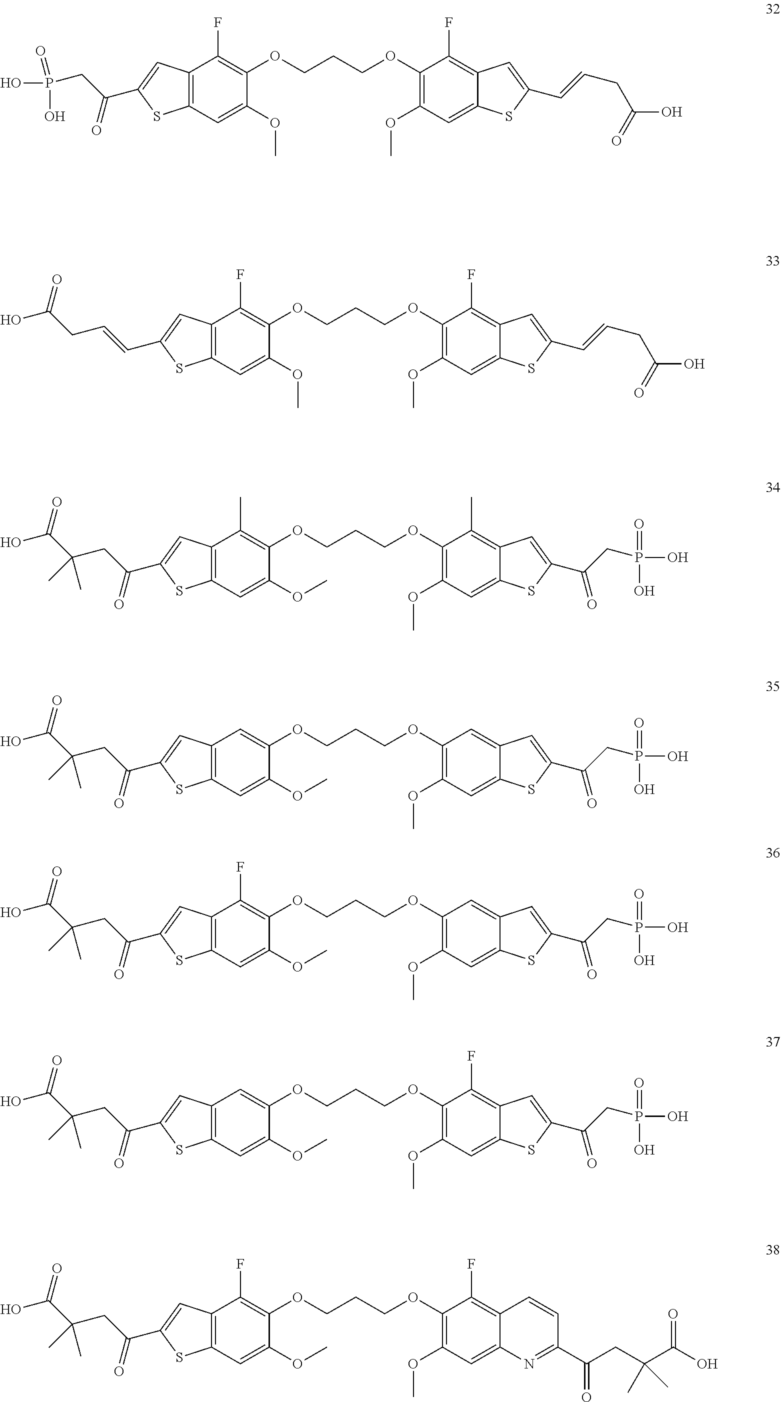

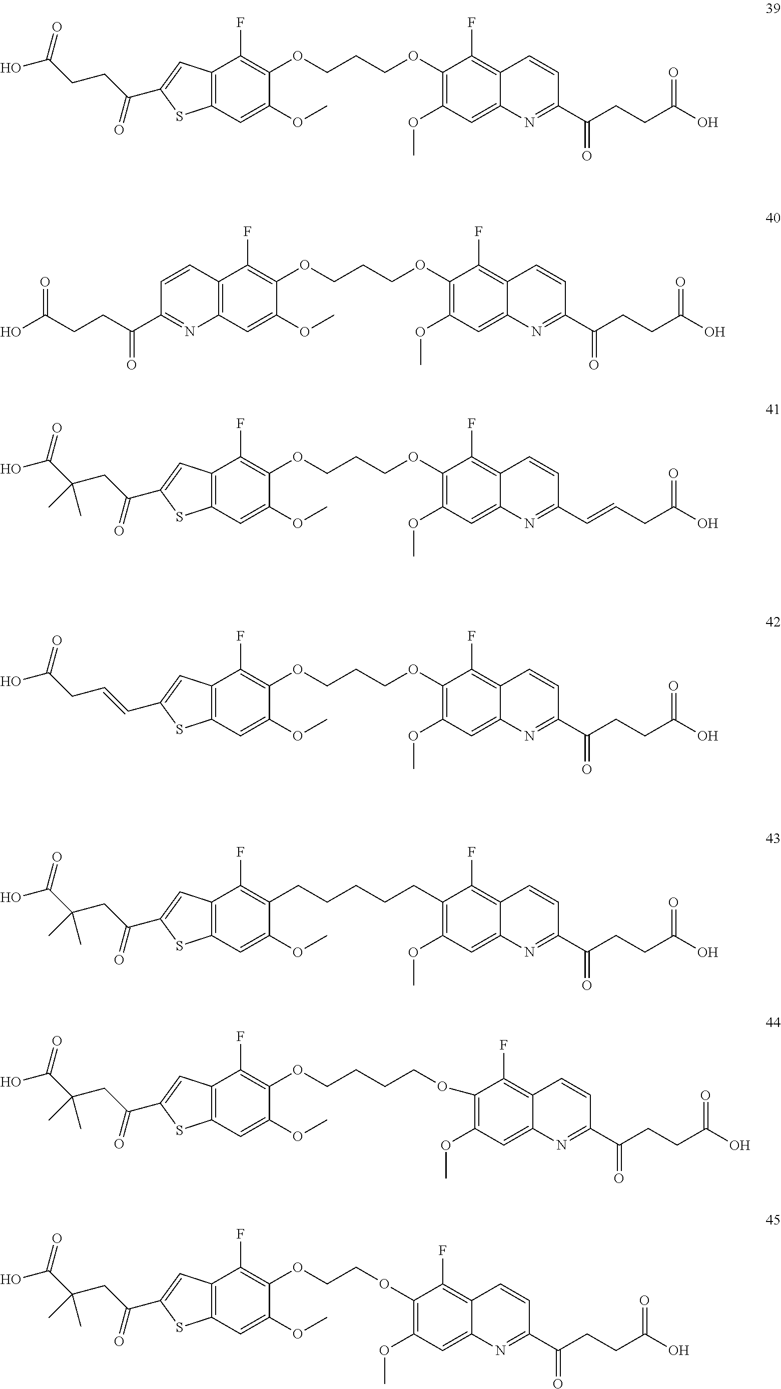

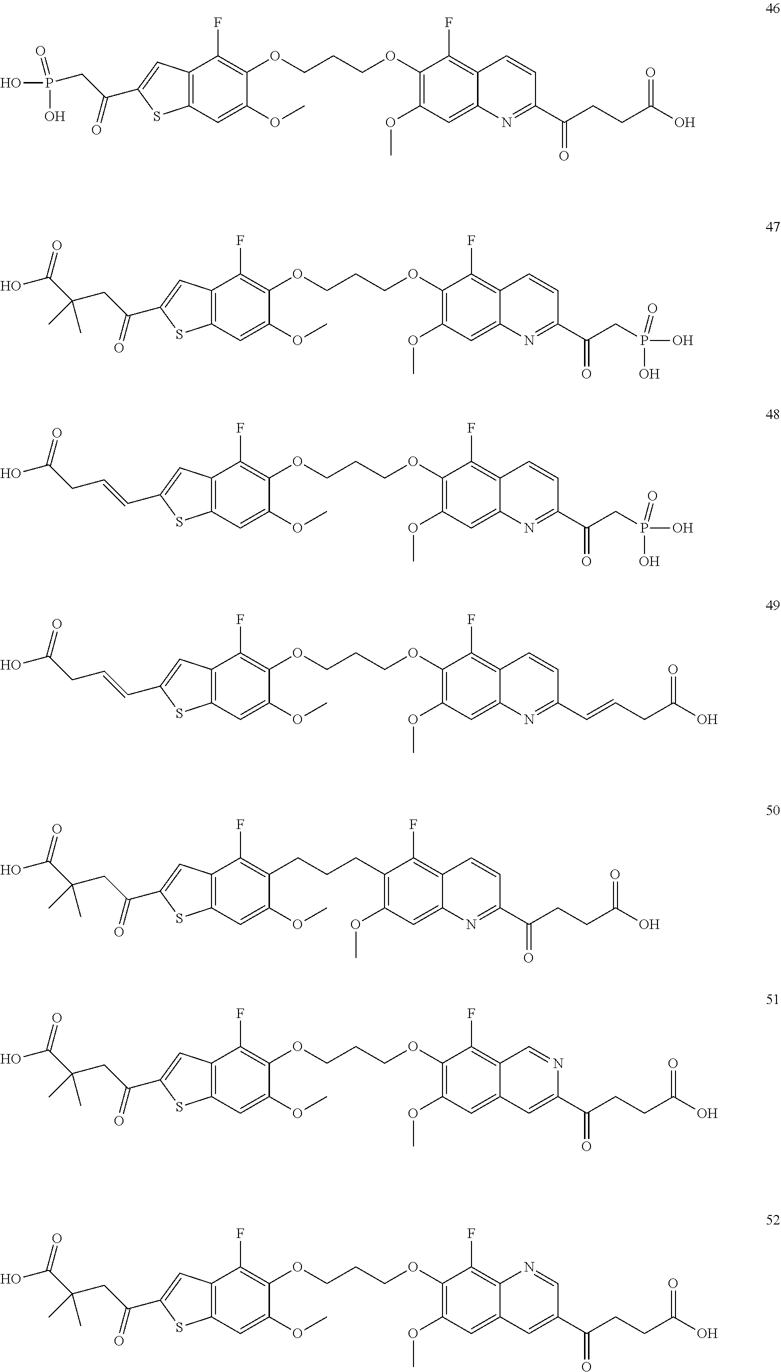

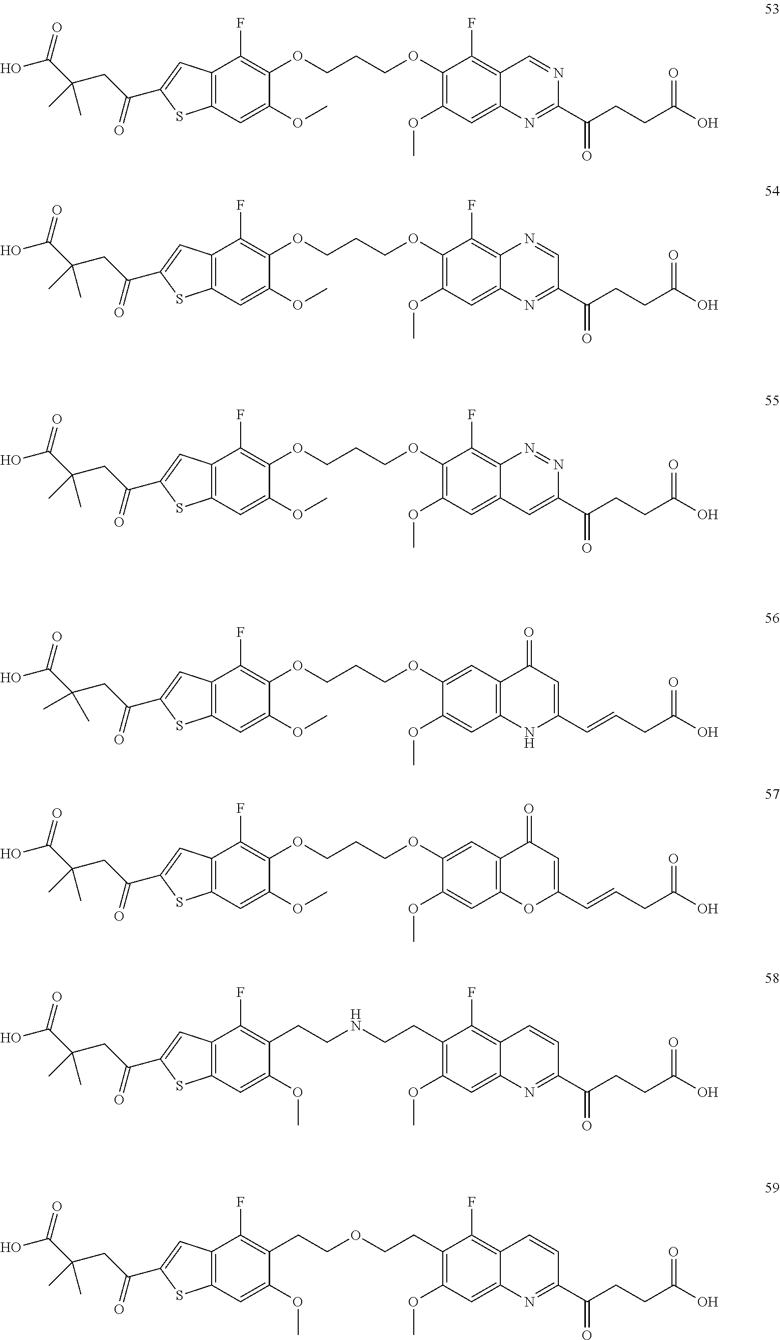

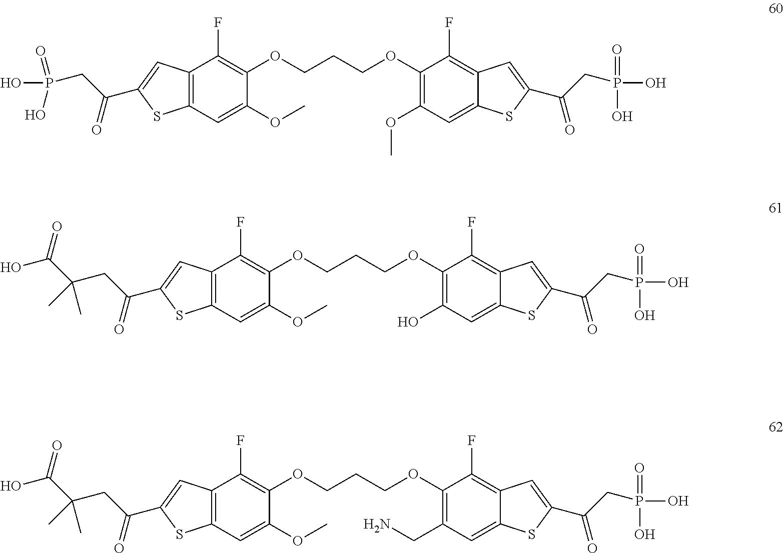 [SMILES: COc1cc2sc(C(=O)CP(=O)(O)O)cc2c(F)c1OCCCOc1c(OC)cc2sc(C(=O)CP(=O)(O)O)cc2c1F ; COc1cc2sc(C(=O)CC(C)(C)C(=O)O)cc2c(F)c1OCCCOc1c(O)cc2sc(C(=O)CP(=O)(O)O)cc2c1F ; COc1cc2sc(C(=O)CC(C)(C)C(=O)O)cc2c(F)c1OCCCOc1c(CN)cc2sc(C(=O)CP(=O)(O)O)cc2c1F]

### Uses of the Compounds and Compositions

In one aspect, an effective amount of a compound or composition described herein is used to treat a medical disorder or disease mediated by STING in a subject in need thereof. In one embodiment, the medical disorder or disease is a cellular proliferative disorder, including, but not limited to cancer. In another aspect, an effective amount of a compound or composition described herein is used to induce an immune response in a subject need thereof. In yet another aspect, an effective amount of a compound or composition described herein is used to induce STING-dependent type I interferon production in a subject in need thereof. In yet another aspect, an effective amount of a compound or composition described herein is used to induce STING-dependent cytokine production in a subject in need thereof.

In one embodiment, an effective amount of a compound or composition described herein is used to treat abnormal cellular proliferation, including, but not limited to, cancer. In certain embodiments, In certain embodiments, the term “cancer” includes, but is not limited to, the following cancers: epidermoid oral: buccal cavity, lip, tongue, mouth, pharynx, squamous cell carcinoma of the head and neck (HNSCC); cardiac: sarcoma (angiosarcoma, fibrosarcoma, rhabdomyosarcoma, liposarcoma), myxoma, rhabdomyoma, fibroma, lipoma, and teratoma; lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, vipoma), small bowel or small intestines (adenocarcinoma, lymphoma, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, hepatocellular adenoma, hemangioma, biliary passages; bone: osteogenic sarcoma (osteosarcoma), fibrosarcoma, malignant fibrous histiocytoma, Ewing's sarcoma, malignant lymphoma (reticulum cell sarcoma), malignant giant cell tumor osteochronfroma (osteocartilaginous exostoses), benign chondroma, chondroblastoma, chondromyxofibroma, osteoid osteoma and giant cell tumors; nervous system: skull (osteoma, hemangioma, granuloma, xanthoma, osteitis deformans), meninges (meningioma, meningiosarcoma, gliomatosis), brain (astrocytoma, medulloblastoma, glioma, germinoma (pinealoma), glioblastoma multiform, oligodendroglioma, schwannoma, retinoblastoma, congenital tumors), spinal cord neurofibroma, meningioma, glioma, sarcoma); gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); hematologic: blood (myeloid leukemia (acute and chronic), acute lymphoblastic leukemia, chronic lymphocytic leukemia, myeloproliferative diseases, multiple myeloma (MM), myelodysplastic syndrome), Hodgkin's disease, non-Hodgkin's lymphoma (malignant lymphoma) hairy cell; lymphoid disorders (e.g., mantle cell lymphoma, Waldenström's macroglobulinemia, Marginal zone lymphoma, and Follicular lymphoma); skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis; thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; adrenal glands: neuroblastoma; and metatstaic melanoma.

In certain embodiments, the cancer is selected from acute myeloid leukemia, breast cancer, colorectal cancer, glioma, head and neck squamous cell carcinoma, lung cancer, including non-small cell lung cancer, head and neck cancer, lymphoma, including a malignant lymphoma, melanoma, nasopharyngeal carcinoma, ovary cancer, pancreatic cancer, prostate cancer, urothelial cancer, and tongue squamous cell carcinoma.

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas. Additional examples of solid tumors include, but are not limited to, squamous cell carcinoma, colon cancer, breast cancer, prostate cancer, lung cancer, liver cancer, pancreatic cancer, and melanoma. In one embodiment, the solid tumor is an advanced solid tumor.

Non-limiting examples of cancers that can be treated using the compounds described herein include, but are not limited to, acoustic neuroma, an adenocarcinoma, adrenal gland cancer, anal cancer, an angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma, glioma, astrocytoma, oligodendroglioma; medulloblastoma), bronchus cancer, carcinoid tumor, cervical cancer, choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), eye cancer (e.g., intraocular melanoma, retinoblastoma), familiar hypereosinophilia, gallbladder cancer, gastric cancer (e.g., stomach adenocarcinoma), gastrointestinal stromal tumor (GIST), head and neck cancer (e.g., head and neck squamous cell carcinoma), oral cancer (e.g., oral squamous cell carcinoma (OSCC)), throat cancer (e.g., laryngeal cancer, pharyngeal cancer, nasopharyngeal cancer, oropharyngeal cancer), a hematopoietic cancer, heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), Wilms' tumor, urethral cancer, vaginal cancer and vulvar cancer (e.g., Paget's disease of the vulva).

In one embodiment, the cancer is a sarcoma, including, but not limited to, Ewing's sarcoma, Kaposi's sarcoma, liposarcoma, myosarcomas, peripheral neuroepithelioma, synovial sarcoma, gliomas, astrocytomas (including anaplastic astrocytoma, diffuse astrocytoma and low-grade astrocytoma), oligodendrogliomas, gliobastomas, neuroblastomas, ganglioneuromas, gangliogliomas, medulloblastomas, pineal cell tumors, meningiomas, meningeal sarcomas, neurofibromas, and Schwannomas.

In one embodiment, the cancer is a hematopoietic cancer, including, but not limited to, leukemia, such as acute lymphocytic leukemia (ALL), also known as acute lymphoblastic leukemia or acute lymphoid leukemia (e.g., B-cell ALL, T-cell ALL), acute myelocytic leukemia (AML) (e.g., B-cell AMIL, T-cell AML), acute granulocytic leukemia, chronic myelocytic leukemia (CML) (e.g., B-cell CML, T-cell CML), a chronic lymphocytic leukemia (CLL) (e.g., B-cell CLL, T-cell CLL), and hairy cell leukemia (HCL). In one embodiment, the hematopoietic cancer is a lymphoma, such as Hodgkin lymphoma (HL) (e.g., B-cell HL, T-cell HL), non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma), and a mixture of one or more leukemia/lymphoma as described above. Additional leukemias and lymphomas include T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, splenic diffuse red pulp small B-cell lymphoma; lymphoplasmacytic lymphoma;

In certain embodiments, the cancer that is treated using the disclosed compounds is selected from adenosarcoma, adrenal cancer, adrenocortical carcinoma, bile duct cancer, bone cancer, bone marrow cancer, brain stem glioma, breast cancer, (including, but not limited to triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, or inflammatory breast cancer (IBC), mesothelioma metastatic breast cancer), colorectal cancer, cutaneous lymphoma, cutaneous melanoma, ductal carcinoma in situ (DCIS), endometrial cancer, epithelioid sarcoma, esophageal cancer, extrahepatic eye cancer, fallopian tube cancer, fibrosarcoma, gastric cancer, gastrointestinal cancer, gastrointestinal carcinoid cancer, gastrointestinal stromal tumors (GIST), germ cell tumor glioblastoma multiforme (GBM), glioma, hairy cell leukemia, hemangioendothelioma, hypopharyngeal cancer, infiltrating ductal carcinoma (IDC), infiltrating lobular carcinoma (ILC), intestinal cancer, intrahepatic bile duct cancer, invasive/infiltrating breast cancer, Islet cell cancer, jaw cancer, kidney cancer, laryngeal cancer, leiomyosarcoma, leptomeningeal metastases, lip cancer, liposarcoma, liver cancer, lobular carcinoma in situ, lung cancer, lymph node cancer, lymphoma, male breast cancer, medullary carcinoma, medulloblastoma, melanoma, meningioma, Merkel cell carcinoma, mesenchymal chondrosarcoma, mesenchymous, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, plasma cell myeloma, solitary plasmacytoma of bone, and extraosseous plasmacytoma.

In other embodiments, the cell-proliferation disorder is selected from benign papillomatosis, benign neoplastic diseases and gestational trophoblastic diseases. In certain embodiments, the benign neoplastic disease is selected from skin papilloma (warts) and genital papilloma. In certain embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In an alternative aspect, an effective amount of a compound or composition described herein is used to treat a medical disorder or disease mediated by STING in a subject in need thereof wherein the disorder or disease is a viral infection, for example, a double stranded DNA virus. In certain embodiments, the virus is from the family Herpesviridae, including but not limited to herpes simplex virus-1 (HSV-1), herpes simplex virus-2 (HSV-2), varicella zoster virus (VZV), epstein-Barr virus (EBV), cytomegalovirus (CMV), and Kaposi's sarcoma-associated herpesvirus (KSHV). In one embodiment, the virus is an adenovirus. In certain embodiments, the virus is from the family Papillomaviridae, including but not limited to human papillomavirus (HPV). In an alternative aspect, the viral infection is an RNA viral infection, for example, a virus from the Flaviviridae family, including Flaviviruses (for example, Yellow fever virus, West Nile virus, Dengue virus, and Zika virus) and Hepacivirus (for example, hepatitis B and hepatitis C).

The compounds described herein can be administered at any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg. In certain embodiments, the dose is selected from the group consisting of 100 mg/kg, 200 mg/kg, 300 mg/kg, 450 mg/kg, 600 mg/kg, 800 mg/kg, and 1000 mg/kg. In certain embodiments, the dose is about 25 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

The dose can be administered on a schedule deemed suitable by the person of skill in the art. In certain embodiments, the dose is administered once per day. In certain embodiments, the dose is administered twice per day. In certain embodiments, the dose is administered three times per day. In certain embodiments, the dose is administered four times per day. In certain embodiments, the dose is administered in divided doses. In certain embodiments, the dose is administered in two divided doses per day. In certain embodiments, the dose is administered in three divided doses per day. In certain embodiments, the dose is administered in four divided doses per day.

Dosing can continue for any length of time deemed suitable by the person of skill in the art. In certain embodiments, the dose is administered daily for fourteen days. In certain embodiments, the dose is administered daily for thirteen days. In certain embodiments, the dose is administered daily for twelve days. In certain embodiments, the dose is administered daily for eleven days. In certain embodiments, the dose is administered daily for ten days. In certain embodiments, the dose is administered daily for nine days. In certain embodiments, the dose is administered daily for eight days. In certain embodiments, the dose is administered daily for seven days. In certain embodiments, the dose is administered daily for six days. In certain embodiments, the dose is administered daily for five days. In certain embodiments, the dose is administered daily for four days. In certain embodiments, the dose is administered daily for three days. In certain embodiments, the dose is administered daily for two days. In certain embodiments, the dose is administered for one day.

In the dosing schedule, the doses can be administered on consecutive days or cyclicly, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the dose is administered weekly. In certain embodiments, the dose is administered twice per week. In certain embodiments, the dose is administered three times per week.

In certain embodiments, the dose(s) are administered for a period of time with a first interval between dose(s), and then the dose(s) are re-administered for a period of time following the first interval between dose(s), wherein this dosing regimen can be repeated (i.e., cyclicly or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The compound can be administered by any route of administration deemed suitable by the practitioner of skill. In certain embodiments, the dose is administered orally. Formulations and techniques for administration are described in detail below.

### Pharmaceutical Compositions

The compounds described herein can be formulated into pharmaceutical compositions that further comprise a pharmaceutically acceptable carrier, diluent, excipient, or vehicle. In one embodiment, this disclosure provides a pharmaceutical composition comprising a compound described above, and a pharmaceutically acceptable carrier, diluent, excipient, or vehicle. In one embodiment, provided herein are pharmaceutical compositions comprising effective amounts of compound of this disclosure or a pharmaceutically acceptable salts thereof and one or more pharmaceutically acceptable carriers, diluents, excipients, or vehicles.

According to another embodiment, the description provides a composition comprising a compound herein or a pharmaceutically acceptable salt thereof and a pharmaceutically acceptable carrier, or vehicle. Pharmaceutical compositions of this description comprise a therapeutically effective amount of a compound of Formula Formula I, Formula II, Formula III, Formula Ia, Formula Ib, Formula Ic, Formula Id, Formula IIa, Formula IIb, or Formula IIIa or a pharmaceutically acceptable salt, stereoisomer, or salt thereof.

It also will be appreciated that certain compounds of this disclosure can exist in free form for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, a compound as otherwise described herein, or a metabolite or residue thereof.

As used herein, the term “pharmaceutically acceptable salt” or “salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences 1977, 66, 1-19, incorporated herein by reference. Pharmaceutically acceptable salts of the compounds of this description include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋4 alkyl)₄ salts. This description also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds. The pharmaceutically acceptable carriers should be biocompatible, for example, non-toxic, non-inflammatory, non-immunogenic, or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, com oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Liquid dosage forms for oral administration include, but are not limited to, pharmaceutically acceptable emulsions, microemulsions, solutions, suspensions, syrups, and elixirs. In addition to the active compounds herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polyethylene glycols and the like.

The compounds described herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, for example, in a certain part of the intestinal tract, optionally, in a delayed manner. Examples of embedding compositions that can be used include polymeric substances and waxes.

The compounds of the description are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds of this disclosure that may be combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

### Combinations

The compounds or compositions described herein can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. Additional therapeutically active agents include, but are not limited to, small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the additional therapeutically agent is a cancer agent (e.g., a biotherapeutic or chemo therapeutic cancer agent).

In one embodiment, the additional active agent(s) may be one or more agents selected from the group consisting of STING agonist compounds, anti-viral compounds, antigens, adjuvants, anti-cancer agents, CTLA-4, LAG-3 and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including, but not limited to anti-cancer vaccines.

In one embodiment, the additional therapeutically active agent is an anti-inflammatory agent. In one aspect of this embodiment, the additional therapeutically active agent is an immune modulator, including but not limited to a checkpoint inhibitor, for example, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and Medlmmune), PF-06801591 (Pfizer), MED10680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (Tecentriq), durvalumab (AstraZeneca and Medlmmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and Medlmmune), AGEN1884 and AGEN2041 (Agenus). LAG-3 checkpoint inhibitors, include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro).

In an alternative embodiment, the PD-1 antagonist as a second therapeutic agent is a monoclonal antibody (mAb), or antigen binding fragment thereof, which specifically binds to PD-1 or PD-L1, and preferably specifically binds to human PD-1 or human PD-L1. The mAb may be a human antibody, a humanized antibody, or a chimeric antibody and may include a human constant region. In some embodiments, the human constant region is selected from the group consisting of IgG1, IgG2, IgG3, and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv, and Fv fragments.

In one embodiment, the additional therapeutically active agent is a cytotoxic agent or a chemotherapy agent. Examples of cytotoxic agents that may be used in combination with the compounds or pharmaceutically acceptable salts described herein, include, but are not limited to, arsenic trioxide (sold under the tradename TRISENOX®), asparaginase (also known as L-asparaginase, and Erwinia L-asparaginase, sold under the tradenames ELSPAR® and KIDROLASE®).

Chemotherapeutic agents that may be used in combination with the compounds or pharmaceutically acceptable salts described herein include abiraterone acetate, altretamine, anhydrovinblastine, auristatin, bexarotene, bicalutamide, BMS 184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, and vinflunine. Such chemotherapeutic agents may be provided as a pharmaceutically acceptable salt, where appropriate. In one embodiment, the additional therapeutically active agent is a vascular endothelial growth factor (VEGF) receptor inhibitors including, but are not limited to, bevacizumab (AVASTIN), axitinib, brivanib alaninate ((S)—((R)-1-(4-(4-fluoro-2-methyl-TH-indol-5-yloxy)-5-methylpyrrolo[2,1-f][1,2,4]triazin-6-yloxy) propan-2-yl)2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide), pasireotide, and sunitinib (SUTENT), sorafenib (NEXAVAR).

In one embodiment, the additional therapeutically active agent is a topoisomerase II inhibitor, including, but are not limited to, etoposide (also known as VP-16 and etoposide phosphate, sold under the tradenames TOPOSAR, VEPESID, and ETOPOPHOS), and teniposide (also known as VM-26, sold under the tradename VUMON).

In one embodiment, the additional therapeutically active agent is an alkylating agent, including, but are not limited to, 5-azacytidine (VIDAZA), decitabine (DECOGEN), temozolomide (TEMCAD, TEMODAR, and TEMODAL), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (BCNU), bendamustine (TREANDA), busulfan (BUSULFEX® and MYLERAN®), carboplatin (PARAPLATIN®), lomustine (also known as CCNU, sold under the tradename CEENU®), cisplatin (also known as CDDP, sold under the tradenames PLATINOL® and PLATINOL®-AQ), chlorambucil (LEUKERAN®), cyclophosphamide (CYTOXAN® and NEOSAR®), dacarbazine (also known as DTIC, DIC and imidazole carboxamide, sold under the tradename DTIC-DOME®), altretamine (also known as hexamethylmelamine (HMM) sold under the tradename HEXALEN®), ifosfamide (IFEXR), procarbazine (MATULANE®), mechlorethamine (also known as nitrogen mustard, mustine and mechloroethamine hydrochloride, sold under the tradename MUSTARGEN®), streptozocin (ZANOSAR®), thiotepa (also known as thiophosphoamide, TESPA and TSPA, and sold under the tradename THIOPLEX®. Such alkylating agents may be provided as a pharmaceutically acceptable salt, where appropriate.

Examples of anti-tumor antibiotics include, but are not limited to, doxorubicin (sold under the tradenames ADRIAMYCIN® and RUBEX®), bleomycin (sold under the tradename LENOXANE®), daunorubicin (also known as dauorubicin hydrochloride, daunomycin, and rubidomycin hydrochloride, sold under the tradename CERUBIDINE®), daunorubicin liposomal (daunorubicin citrate liposome, sold under the tradename DAUNOXOME®), mitoxantrone (also known as DHAD, sold under the tradename NOVANTRONE®), epirubicin (sold under the tradename ELLENCE™), idarubicin (sold under the tradenames IDAMYCIN®, IDAMYCIN PFS®), and mitomycin C (sold under the tradename MUTAMYCIN®). Such anti-tumor antibiotics may be provided as a pharmaceutically acceptable salt, where appropriate.

In one embodiment, the additional therapeutically active agent is an anti-metabolite including, but are not limited to, claribine (2-chlorodeoxyadenosine, LEUSTATIN®), 5-fluorouracil (ADRUCIL®), 6-thioguanine (PURINETHOL®), pemetrexed (ALIMTA®), cytarabine (also known as arabinosylcytosine (Ara-C), sold under the tradename CYTOSAR-U®), cytarabine liposomal (also known as Liposomal Ara-C, sold under the tradename DEPOCYT™), decitabine (DACOGENO), hydroxyurea and (HYDREA®, DROXIA™ and MYLOCEL™), fludarabine (FLUDARA®), floxuridine FUDR®), cladribine (also known as 2-chlorodeoxyadenosine (2-CdA) sold under the tradename LEUSTATIN™), methotrexate (also known as amethopterin, methotrexate sodium (MTX), sold under the tradenames RHEUMATREX® and TREXALL™), and pentostatin (NIPENT®). Such anti-metabolites may be provided as a pharmaceutically acceptable salt, where appropriate.

In one embodiment, the additional therapeutically active agent is a retinoid including, but are not limited to, alitretinoin (PANRETIN®), tretinoin (all-trans retinoic acid, also known as ATRA, sold under the tradename VESANOID®), isotretinoin (13-c/s-retinoic acid, sold under the tradenames ACCUTANE®, AMNESTEEM®, CLARAVIS®, CLARUS®, DECUTAN®, ISOTANE®, IZOTECHR, ORATANE®, ISOTRET®, and SOTRET®), and bexarotene (TARGRETIN®).

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

In certain embodiments, a compound or a pharmaceutically acceptable salt described herein is administered in conjunction with one or more additional therapeutic agents including vaccines intended to stimulate an immune response. Antigens and adjuvants that may be used in combination with the compounds or pharmaceutically acceptable salts described herein include B7 costimulatory molecule, interleukin-2, interferon-y, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryllipid A, lipoteichoic acid, imiquimod, resiquimod, and in addition retinoic acid-inducible gene I (RIG-I) agonists such as poly I:C, used separately or in combination with the described compositions are also potential adjuvants. Such antigens and anjuvants as second therapeutically active agents may be provided as a pharmaceutically acceptable salt, where appropriate.

Anti-viral compounds that may be used in combination with the compounds or pharmaceutically acceptable salts described herein include hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors. Such anti-viral compounds may be provided as a pharmaceutically acceptable salt, where appropriate.

### Articles of Manufacture and Kits

Also provided are articles of manufacture comprising any of the compounds or pharmaceutical compositions described herein. The articles of manufacture include suitable containers or packaging materials for the compounds or pharmaceutical compositions. Examples of a suitable container include, but are not limited to, a bottle, a vial, a syringe, an intravenous bag, or a tube.

Also provided are kits comprising any of the compounds or pharmaceutical compositions described herein. The kits can contain the compounds or pharmaceutical compositions in suitable containers or packaging materials, including, but not limited to, a bottle, a vial, a syringe, an intravenous bag, or a tube. The kits can comprise the compounds or pharmaceutical compositions for administration to an individual in single-dose form or in multiple-dose form. The kits can further comprise instructions or a label for administering the compounds or pharmaceutical compositions to an individual according to any of the methods disclosed herein. The kits can further comprise equipment for administering the compounds or pharmaceutical compositions to an individual, including, but not limited to, needles, syringes, tubing, or intravenous bags. The kits can further comprise instructions for producing any of the compounds or pharmaceutical compositions disclosed herein.

Also provided are articles of manufacture comprising any of the compounds, vaccines, or pharmaceutical compositions described herein. The articles of manufacture include suitable containers or packaging materials for the compounds or pharmaceutical compositions. The articles of manufacture include suitable containers or packaging materials for the compounds, oncolytic viruses, or pharmaceutical compositions. Examples of a suitable container include, but are not limited to, a bottle, a vial, a syringe, an intravenous bag or a tube.

The disclosure will be more fully understood by reference to the following examples. They should not, however, be construed as limiting the scope of this disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Compounds provided herein can be prepared or synthesized according to any technique deemed suitable by the person of skill in the art. Exemplary synthetic schemes are described below.

### Preparation of Compounds

Proton nuclear magnetic resonance (NMR) spectra were obtained on Bruker Ascend™ 500 MHz spectrometer. NMR spectra are reported as follows: chemical shift δ (ppm), multiplicity, coupling constant J (Hz), and integration. The abbreviations s=singlet, d=doublet, t=triplet, q=quartet, m=multiplet and br=broad are used throughout. Mass spectral data were measured using the following systems: Agilent Technologies 1290 series, Binary Pump, Diode Array Detector. Data was acquired using agilent software and purity characterized by UV wavelength 220 nm, evaporative light scattering detection (ELSD) and electrospray positive ion (ESI) (column: Agilent Poroshell 120 EC-C18, 2.7 μm, 4.6×50 mm). Solvents used: acetonitrile/water, containing 0.100 formic acid; flow rate 1 mL/min.

Abbreviations used in the examples include:

| Abbreviation | Name |
|---|---|
| CH₃CN | acetonitrile |
| Boc | t-butoxycarbonyl |
| CDCl₃ | deuterated chloroform |
| DCM | dichloromethane |
| DMF | N,N-dimethylformamide |
| DMSO | dimethylsulfoxide |
| DMSO-d₆ | deuterated dimethylsulfoxide |
| Et₃N | triethylamine |
| EtOAc | ethyl acetate |
| EtOH | ethanol |
| g | grams |
| H, hr , or hrs | hours |
| H₂O | water |
| HATU | 1-[bis(dimethylamino)methylene]-1H-1,2,3-triazolo[4,5-b]pyridinium 3- |
| | oxide hexafluorophosphate |
| HCl | hydrogen chloride |
| HOAc | acetic acid |
| Hz | Hertz |
| HPLC | high pressure liquid chromatography |
| K₂CO₃ | potassium carbonate |
| kg | kilograms |
| LCMS | Liquid chromatography-Mass spectrometry |
| LiOH | lithium hydroxide |
| MHz | megahertz |
| MeOH | methanol |
| μL | microliters |
| mg | milligrams |
| mL | milliliters |
| mM | millimolar |
| μM | micromolar |
| mmol | millimoles |
| min | minutes |
| MS | mass spectrometry |
| MTBE | methyl tert-butyl ether |
| N₂ | nitrogen |
| Na₂SO₄ | sodium sulfate |
| NaOH | sodium hydroxide |
| NH₄Cl | ammonium chloride |
| NMR | nuclear magnetic resonance |
| Pd/C | palladium on carbon |
| Pd(OAc)₂ | palladium(II) acetate |
| RB | round-bottom |
| rt | room temperature |
| sat. | saturated |
| THF | tetrahydrofuran |
| TLC | thin layer chromatography |

For all of the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

As used herein, the symbols and conventions used in these processes, schemes and examples, regardless of whether a particular abbreviation is specifically defined, are consistent with those used in the contemporary scientific literature, for example, the Journal of Biological Chemistry and/or the Journal of the American Chemical Society.

For all of the following examples, standard work-up and purification methods known to those skilled in the art can be utilized. Unless otherwise indicated, all temperatures are expressed in ° C. (degrees Celsius). All methods are conducted at room temperature (“rt” or “r.t.” or “RT”), unless otherwise noted.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Example 1: Synthesis of Select STING Agonists

Common intermediates of the STING agonist examples described below were prepared according to the following schemes. The intermediates were then used to make the STING agonist compounds of this disclosure.

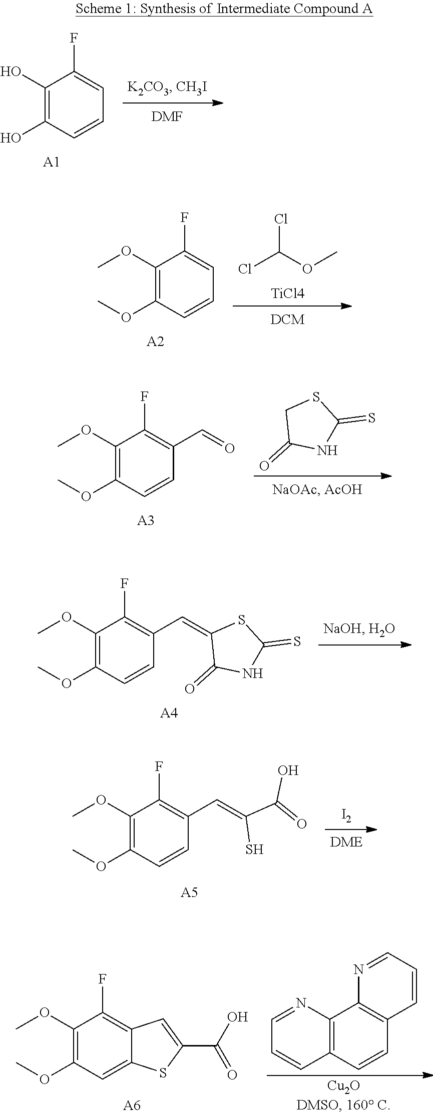

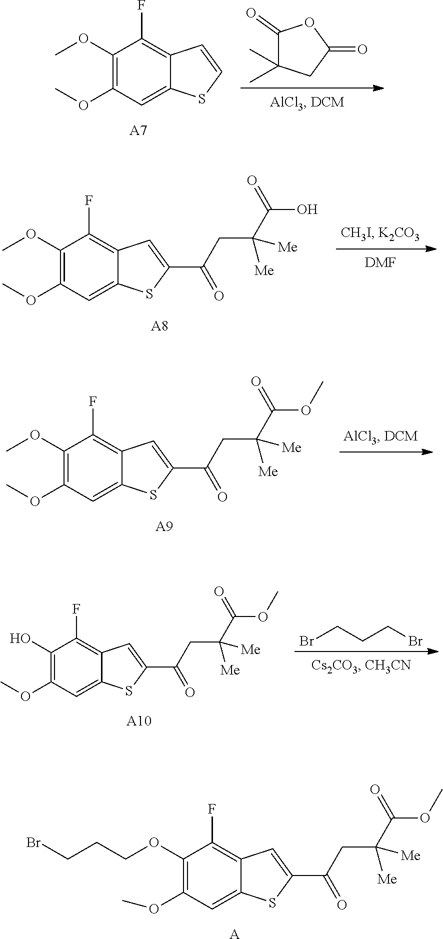

1-Fluoro-2,3-dimethoxybenzene (A2): To a stirring solution of compound A1 (30 g, 234 mmol) and K₂CO₃ (80.9 g, 585.4 mmol) in DMF (300 mL) was added CH₃I (83.1 g, 585.4 mmol) at 0° C. Then the reaction mixture was stirred overnight at room temperature. The reaction mixture was diluted with water (3 L) and extracted with EtOAc (3×1 L). The combined organic layers were washed with brine, dried over Na₂SO₄, filtered, and concentrated under reduced pressure to give the compound A2 (35 g, 95% yield) as yellow oil. ¹H NMR (400 MHz, CDCl3) δ 6.95 (td, J=8.4, 6.0 Hz, 1H), 6.75-6.66 (m, 2H), 3.92 (d, J=1.1 Hz, 3H), 3.87 (s, 3H).

2-Fluoro-3,4-dimethoxybenzaldehyde (A3): To a stirred solution of compound A2 (35 g, 224.1 mmol) in DCM (200 mL) was added dropwise TiCl₄ (63.7 g, 336.1 mmol) in DCM (100 mL) at −20° C. under N₂ atmosphere. Then dichloromethyl methyl ether (28.3 g, 246.5 mmol) in DCM (100 mL) was added to the reaction. After which, the reaction was stirred at RT for 16 h. The reaction mixture was added dropwise to water (400 mL), and extracted with (DCM/EtOAc=5/1, v/v). The combined organic layers were washed with brine, dried over Na₂SO₄, filtered and concentrated under reduced pressure. The crude material was purified by silica gel flash chromatography to afford the compound A3 (20 g, 48% yield) as a light-yellow solid. ¹H NMR (400 MHz, DMSO-d₆) δ 10.04 (s, 1H), 7.59 (dd, J=8.8, 7.7 Hz, 1H), 7.09 (dd, J=8.9, 1.5 Hz, 1H), 3.93 (s, 3H), 3.83 (s, 3H).

(E)-5-(2-Fluoro-3,4-dimethoxybenzylidene)-2-thioxothiazolidin-4-one (A4): To a solution of compound A3 (18.2 g, 98.8 mmol), 2-thioxothiazolidin-4-one (13.1 g, 98.8 mmol) and NaOAc (24.3 g, 296.4 mmol) in Acetic acid (200 mL) was stirred overnight at 100° C. The reaction mixture was cooled to RT and added slowly to water (600 mL). The precipitate was formed, filtered and washed with water (15 mL×3) to afford the compound A4 (28 g, 94% yield) as a bright yellow solid which was used directly without further purification. LCMS (ESI)=m/z 300 (M+H); ¹H NMR (400 MHz, DMSO-d₆) δ 7.53 (s, 1H), 7.20 (t, J=8.4 Hz, 1H), 7.09 (dd, J=9.0, 1.5 Hz, 1H), 3.91 (s, 3H), 3.82 (s, 3H).

(Z)-3-(2-Fluoro-3,4-dimethoxyphenyl)-2-mercaptoacrylic acid (A5): To a NaOH solution (29.1 g, 0.73 mol in 200 mL H₂O and 200 mL EtOH) was added compound A4 (54.5 g, 0.18 mmol), the reaction mixture was stirred overnight at RT, the reaction was then diluted with water (500 mL), EtOAc (250 mL). The aqueous phase was collected and acidified to pH˜3 by adding 1N HCl, extracted with EtOAC (200 mL×3), the combined organic layer was concentrated to afford the compound A5 (18.2, yield: 38.7%) as a yellow solid which was used directly to the next step. ¹H NMR (400 MHz, DMSO-d₆) δ 13.08 (s, 1H), 7.71 (s, 1H), 7.67-7.56 (m, 1H), 7.04 (dd, J=9.0, 1.7 Hz, 1H), 3.88 (s, 3H), 3.80 (s, 3H).

4-Fluoro-5,6-dimethoxybenzo[b]thiophene-2-carboxylic acid (A6): The solution of compound A5 (18.2 g, 0.07 mmol) and I₂ (53.5 g, 0.21 mmol) in DME (540 mL) was stirred overnight at 80° C. The reaction mixture was cooled to RT and quenched with aq Na₂S₂O₃ (300 mL) then 2.0 eq of NaOH was added. The mixture was extracted with EtOAc (600 mL). The aqueous phase was collected and adjusted to pH˜3, extracted with EtOAc. The combined organic phases were concentrated under reduced pressure to give the compound A6 (17.0 g, 94% yield) as a bright yellow solid. ¹H NMR (400 MHz, Chloroform-d) δ 8.12 (s, 1H), 7.09 (s, 1H), 3.99 (s, 3H), 3.97 (s, 3H).

4-Fluoro-5,6-dimethoxybenzo[b]thiophene (A7): The solution of compound A6 (10.0 g, 39 mmol), 1,10-phenanthroline (2.79 g, 19 mmol) and Cu₂O (1.76 g, 9.76 mmol) in DMSO (200 mL) was stirred at 160° C. for 8 h. The reaction mixture was cooled to RT, then added 1N HCl (40 mL), filtered through Celite. The filtrate was extracted with EtOAc. The combined organic phases were concentrated under reduced pressure, and the crude residue was purified by silica gel chromatography to afford the desired product (4.3 g, 51% yield). ¹H NMR (400 MHz, CDCl3) δ 7.34-7.30 (m, 1H), 7.27 (s, 1H), 7.14-7.11 (m, 1H), 3.98 (d, J=0.9 Hz, 3H), 3.94 (s, 3H).

4-(4-Fluoro-5,6-dimethoxybenzo[b]thiophen-2-yl)-2,2-dimethyl-4-oxobutanoic acid (A8): 3,3-dimethyldihydrofuran-2,5-dione (2.8 g, 22.6 mmol), AlCl₃ (1.9 g, 14.7 mmol) in DCM (15 mL) was stirred at 0° C. for 10 min, then compound A7 (2.4 g, 11.3 mmol) in DCM (5 mL) was added slowly to the suspension. The reaction mixture was stirred overnight at RT. After which, the reaction was carefully quenched with water (10 mL), and then extracted with CHCl₃/IPA (3/1, v/v, 15×3 mL). The combined organic phases were concentrated, and the residue was purified by silica gel chromatography to give the desired product (1.5 g, 39% yield) as a yellow solid. ¹H NMR (400 MHz, DMSO-d6) δ 8.29 (s, 1H), 7.56 (s, 1H), 3.92 (s, 3H), 3.85 (s, 3H), 3.57 (s, 2H), 1.23 (s, 6H).

Methyl 4-(4-fluoro-5,6-dimethoxybenzo[b]thiophen-2-yl)-2,2-dimethyl-4-oxobutanoate (A9): The solution of compound A8 (1.5 g, 4.4 mmol), K₂CO₃ (1.83 g, 13.2 mmol) and CH₃I (936.8 mg, 8.25 mmol) in DMF (10 mL) was stirred at RT for 12 h. The reaction mixture was quenched with water and extracted with EtOAc (15 mL×3). The combined organic phases were concentrated under reduced pressure and purified by silica gel chromatography to give the desired product A9 (840 mg, 54% yield) as a yellow solid. ¹H NMR (400 MHz, DMSO-d6) δ 8.30 (s, 1H), 7.57 (s, 1H), 3.92 (s, 3H), 3.86 (s, 3H), 3.57 (s, 3H), 3.43 (s, 2H), 1.24 (s, 6H).

Methyl 4-(4-fluoro-5-hydroxy-6-methoxybenzo[b]thiophen-2-yl)-2,2-dimethyl-4-oxobutanoate (A10): The solution of compound A9 (860 mg, 2.42 mmol) and AlCl₃ (1.9 g, 14.52 mmol) in DMF (20 mL) was stirred at RT for 12 h. The reaction mixture was quenched with ice cold water (20 mL) and then extracted with CHCl₃/IPA (3/1, v/v, 15 mL×3). The combined organic phase was concentrated, and the residue was purified by silica gel chromatography to give the desired product A10 (560 mg, 67.9% yield) as a yellow solid. ¹H NMR (400 MHz, DMSO-d₆) δ 9.52 (d, J=0.9 Hz, 1H), 8.23 (s, 1H), 7.46 (s, 1H), 3.91 (s, 3H), 3.56 (s, 3H), 3.42 (s, 2H), 1.23 (s, 6H).

Methyl 4-(5-(3-bromopropoxy)-4-fluoro-6-methoxybenzo[b]thiophen-2-yl)-2,2-dimethyl-4-oxobutanoate (A): The mixture of compound A10 (220 mg, 0.64 mmol), 1,3-dibromopropane (6.5 g, 32.3 mmol) and Cs₂CO₃ (1.0 g, 3.23 mmol) in CH₃CN (6 mL) was stirred at 65° C. for 4 h after which, the reaction was cooled to RT, quenched with water and extracted with EtOAc (15 mL×3). The combined organic phases were concentrated under reduced pressure, and the residue was purified by silica gel chromatography to afford the desired product A (260 mg, 88% yield) as a yellow solid. ¹H NMR (400 MHz, DMSO-d6) δ 8.31 (s, 1H), 7.59 (s, 1H), 4.15 (t, J=5.9 Hz, 2H), 3.92 (s, 3H), 3.73 (t, J=6.5 Hz, 2H), 3.57 (s, 3H), 3.43 (s, 2H), 2.21 (p, J=6.2 Hz, 2H), 1.24 (s, 6H).

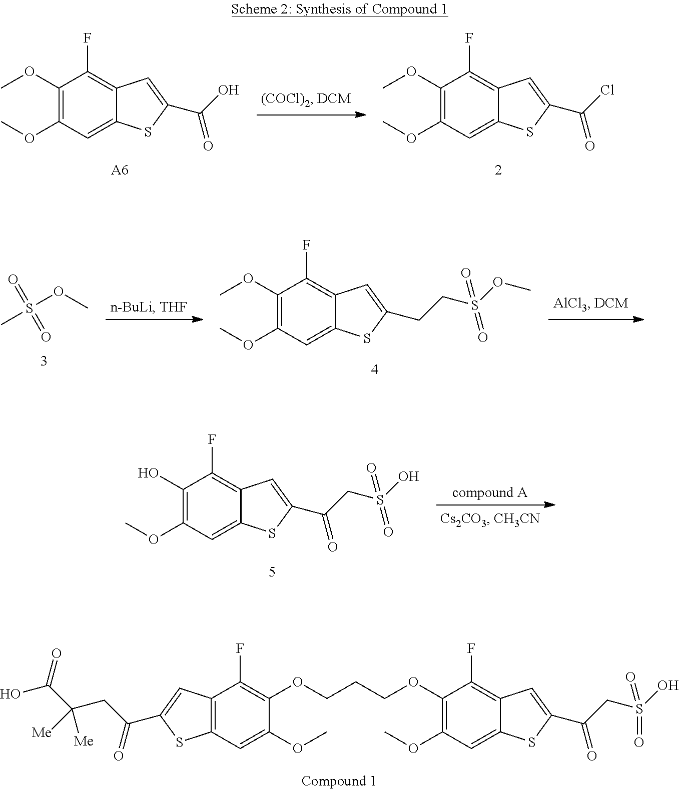

4-Fluoro-5,6-dimethoxybenzo[b]thiophene-2-carbonyl chloride (2): To a stirring solution of 4-fluoro-5,6-dimethoxybenzo[b]thiophene-2-carboxylic acid A6 (700 mg, 2.73 mmol) in DCM (7 mL) was added oxalyl chloride (1.39 g, 10.9 mmol), DMF (0.05 mL) at 0° C. The reaction mixture was stirred at RT for 3 h, after which, the reaction was concentrated under reduced pressure and carried over compound 2 directly to the next step.

Methyl 2-(4-fluoro-5, 6-dimethoxybenzo[b]thiophen-2-yl)ethane-1-sulfonate (4): To a solution of methyl methanesulfonate (613 mg, 5.57 mmol in THF (7 mL) was added n-BuLi (2.2 mL, 5.57 mmol) at −78° C. After stirring the reaction at −78° C. for 30 min then the solution of 4-fluoro-5,6-dimethoxybenzo[b]thiophene-2-carbonyl chloride (2) in THF (8 mL) was added. The reaction was stirred overnight at RT. TLC showed SM disappeared, the reaction was quenched with aqueous NH₄Cl solution and extracted with EtOAc, the combined organic layers were dried over Na₂SO₄, and concentrated under reduced pressure to give the crude product which was purified by silica gel chromatography to give compound 4 (347 mg) as a solid. ¹H NMR (400 MHz, DMSO-d₆) δ 8.60 (s, 1H), 7.63 (s, 1H), 5.40 (s, 2H), 3.98 (d, J=1.5 Hz, 3H), 3.94 (s, 3H), 3.87 (s, 3H)

2-(4-Fluoro-5-hydroxy-6-methoxybenzo[b]thiophen-2-yl)-2-oxoethane-1-sulfonic acid (5): To a solution of methyl 2-(4-fluoro-5,6-dimethoxybenzo[b]thiophen-2-yl)ethane-1-sulfonate (4) (344 mg, 0.99 mmol) in DCM (5 mL) was added AlCl₃ (921.7 mg, 6.9 mmol) at 0° C. The reaction mixture was stirred overnight at RT. TLC showed SM disappeared. The reaction was slowly added to ice cold water and extracted with DCM. The combined organic layers were dried over Na₂SO₄ and concentrated under reduced pressure to give the crude product which was purified by Reverse phase column to give the compound 5 (285 mg) as a yellow solid. ¹H NMR (400 MHz, DMSO-d₆) δ 8.24 (s, 1H), 7.44 (s, 1H), 4.17 (s, 2H), 3.91 (s, 3H).

4-(4-Fluoro-5-(3-((4-fluoro-6-methoxy-2-(2-sulfoacetyl)benzo[b]thiophen-5-yl)oxy)propoxy)-6-methoxybenzo[b]thiophen-2-yl)-2,2-dimethyl-4-oxobutanoic acid (Compound 1): To a solution of 2-(4-fluoro-5-hydroxy-6-methoxybenzo[b]thiophen-2-yl)-2-oxoethane-1-sulfonic acid 5 (140 mg, 0.44 mmol) in DMF (2 mL) was added methyl 4-(5-(3-bromopropoxy)-4-fluoro-6-methoxybenzo[b]thiophen-2-yl)-2,2-dimethyl-4-oxobutanoate (A) (241.9 mg, 0.52 mmol) and Cs₂CO₃ (996.8 mg, 3.06 mmol) at RT. The mixture was stirred overnight at 65° C. The reaction was cooled to RT and purified by Preparative HPLC to give the desired Compound 1 (11 mg) as a white solid. ¹H NMR (400 MHz, DMSO-d₆) δ 8.27 (d, J=9.7 Hz, 2H), 7.54 (d, J=6.2 Hz, 2H), 4.28 (t, J=6.1 Hz, 4H), 4.19 (s, 2H), 3.88 (d, J=3.1 Hz, 6H), 3.37 (s, 2H), 2.10 (p, J=6.1 Hz, 2H), 1.24 (s, 6H).

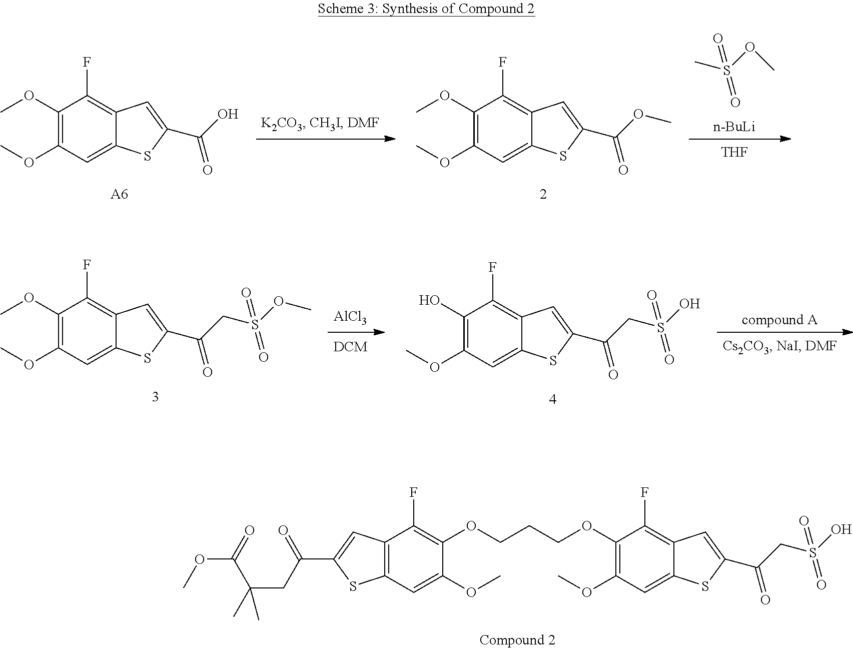

Methyl 4-fluoro-5,6-dimethoxybenzo[b]thiophene-2-carboxylate (2): To a solution of 4-fluoro-5,6-dimethoxybenzo[b]thiophene-2-carboxylic acid A6 (21 g, 0.08 mol) and K₂CO₃ (14.5 g, 0.24 mol) in DMF (180 mL) was added CH₃I (41.8 g, 0.14 mol) at 0° C. The reaction mixture was stirred overnight at RT. The mixture was then diluted with water and extracted with EtOAc, organic layers were dried over Na₂SO₄, and concentrated under reduced pressure. The crude product was purified by silica gel chromatography to afford the compound 2 (10 g) as a solid. ¹H NMR (400 MHz, Chloroform-d) δ 8.04 (s, 1H), 7.07 (s, 1H), 3.98 (d, J=1.1 Hz, 3H), 3.96 (s, 3H), 3.93 (s, 3H).

Methyl 2-(4-fluoro-5,6-dimethoxybenzo[b]thiophen-2-yl)-2-oxoethane-1-sulfonate (3): To a solution of methyl methanesulfonate (12.22 g, 0.037 mol) in THF (200 mL) was added n-BuLi (44.4 ml, 0.11 mol) at −78° C. and the reaction mixture was stirred for 30 min. After which, the solution of methyl 4-fluoro-5,6-dimethoxybenzo[b]thiophene-2-carboxylate (2) in THF (150 ml) was added and the reaction was stirred overnight at RT. The reaction was quenched with aq NH₄Cl solution and extracted with EtOAc, organic layers were dried over Na₂SO₄, and concentrated under reduced pressure. The crude product was purified by silica gel chromatography to give the compound 3 (8 g) as a solid. ¹H NMR (400 MHz, DMSO-d₆) δ 8.60 (s, 1H), 7.63 (s, 1H), 5.40 (s, 2H), 3.98 (d, J=1.5 Hz, 3H), 3.94 (s, 3H), 3.87 (s, 3H).

2-(4-Fluoro-5-hydroxy-6-methoxybenzo[b]thiophen-2-yl)-2-oxoethane-1-sulfonic acid (4): To a solution of methyl 2-(4-fluoro-5,6-dimethoxybenzo[b]thiophen-2-yl)-2-oxoethane-1-sulfonate 3 (8 g, 0.02 mol) in DCM (80 mL) was added AlCl₃ (21.4 g, 0.16 mol) at 0° C. The reaction mixture was stirred overnight at RT. After which the reaction mixture was added to ice-cold water and extracted with THF. The organic layers were dried over Na₂SO₄ and concentrated under reduced vacuum. The crude product was purified by silica gel chromatography to afford the compound 4 (4.9 g) as a yellow solid. ¹H NMR (400 MHz, DMSO-d₆) δ 8.24 (s, 1H), 7.44 (s, 1H), 4.17 (s, 2H), 3.91 (s, 3H).

2-(4-Fluoro-5-(3-((4-fluoro-6-methoxy-2-(4-methoxy-3,3-dimethyl-4-oxobutanoyl)benzo[b]thiophen-5-yl)oxy)propoxy)-6-methoxybenzo[b]thiophen-2-yl)-2-oxoethane-1-sulfonic acid (Compound 2): To a solution of 2-(4-fluoro-5-hydroxy-6-methoxybenzo[b]thiophen-2-yl)-2-oxoethane-1-sulfonic acid (4) (4.38 g, 0.013 mol) in DMF (50 mL) was added sequentially methyl 4-(5-(3-bromopropoxy)-4-fluoro-6-methoxybenzo[b]thiophen-2-yl)-2,2-dimethylpent-4-enoate (A) (7.57 g, 0.016 mol), Cs₂CO₃ (13.37 g, 0.04 mol) and NaI (4.1 g, 0.027 mol) at RT, the mixture was stirred overnight at RT then diluted with water and extracted with EtOAc, the organic layers were dried over Na₂SO₄, and concentrated under reduced pressure. The crude product was purified by preparative reverse phase HPLC to afford the desired product Compound 2 (880 mg) as a white solid. ¹H NMR (400 MHz, DMSO-d₆) δ 8.29 (s, 2H), 7.55 (d, J=9.0 Hz, 2H), 4.29 (t, J=6.1 Hz, 4H), 4.21 (s, 2H), 3.89 (d, J=3.0 Hz, 6H), 3.59 (s, 3H), 3.45 (s, 2H), 2.11 (p, J=6.1 Hz, 2H), 1.26 (s, 6H).

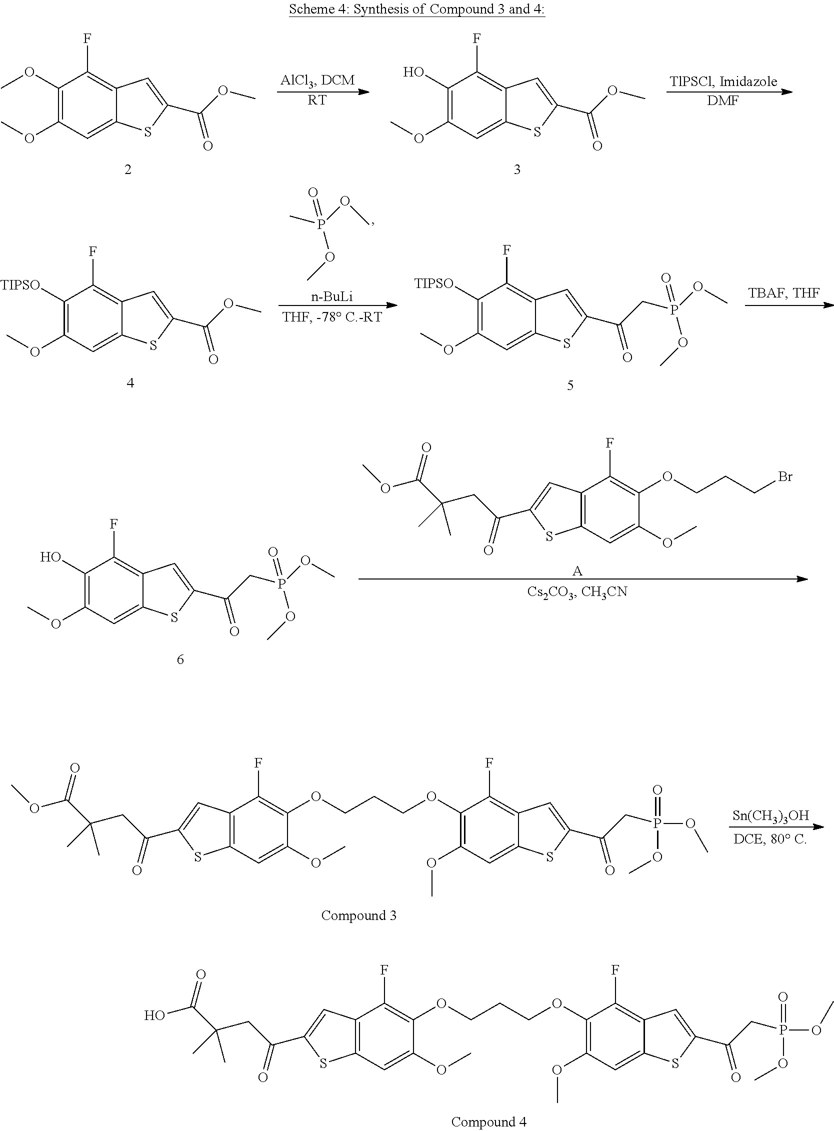

Methyl 4-fluoro-5-hydroxy-6-methoxybenzo[b]thiophene-2-carboxylate (3): To a suspension of AlCl₃ (10.8 g, 81.4 mmol) in DCM (180 mL) was added methyl 4-fluoro-5,6-dimethoxybenzo[b]thiophene-2-carboxylate 2 (4.4 g, 16.3 mmol) at 0° C. The reaction mixture was warmed up to ambient temperature and stirred for 16 h under nitrogen atmosphere. TLC showed the SM was consumed then the mixture was diluted with water (100 mL) and acidified to pH˜2 by adding 1M HCl, extracted with EtOAc (200 mL×3). The combined organic layers were washed with brine, dried over Na₂SO₄ and concentrated under reduced pressure. The residue was purified by silica gel chromatography to afford the desired product 3 (2 g, 47.9%) as a light yellow solid. LCMS (ESI)=255.1 [M−1]; ¹H NMR (400 MHz, DMSO-d₆) δ 9.58-9.52 (m, 1H), 7.94 (s, 1H), 7.48 (s, 1H), 3.91 (s, 3H), 3.86 (s, 3H).

Methyl 4-fluoro-6-methoxy-5-((triisopropylsilyl) oxy) benzo[b]thiophene-2-carboxylate (4): To a solution of methyl 4-fluoro-5-hydroxy-6-methoxybenzo[b]thiophene-2-carboxylate 3 (2 g, 7.8 mmol) in DMF (40 mL) was added 1H-imidazole (1.3 g, 18.7 mmol) and Triisopropylsilyl chloride (3 g, 15.61 mmol). The mixture was stirred overnight at room temperature under nitrogen atmosphere then the reaction mixture was diluted with water (60 mL) and extracted with EtOAc (60 mL×3). The combined organic layers were washed with brine, dried over Na₂SO₄ and concentrated under reduced pressure. The residue was purified by silica gel chromatography to give the desired product 4 (2.6 g, 81%) as a light yellow solid. ¹H NMR (400 MHz, CDCl₃) δ 8.26 (s, 1H), 7.52 (s, 1H), 4.18 (s, 3H), 4.15 (s, 3H), 1.59-1.48 (m, 4H), 1.35 (d, J=7.4 Hz, 18H).

Dimethyl (2-(4-fluoro-6-methoxy-5-((triisopropylsilyl) oxy) benzo[b]thiophen-2-yl)-2-oxoethyl) phosphonate (5): To a solution of dimethyl methylphosphonate (1.54 g, 12.46 mmol) in THF (30 mL) was added n-BuLi (5 mL, 2.5 M in THF, 12.46 mmol) at −78° C. then the reaction was stirred for 45 min. After which, compound 4 (2.57 g, 6.23 mmol) in THF (20 mL) was added to the above solution, the reaction mixture was stirred overnight at RT under nitrogen atmosphere. After which, the reaction was quenched with saturated aq NH₄Cl (40 mL) at −40° C. and extracted with EtOAc (60 mL×3). The combined organic layers were washed with brine, dried over Na₂SO₄ and concentrated in vacuum. The residue was purified by silica gel chromatography to afford the desired product 5 (2.87 g, 91.4%) as a light yellow solid. LCMS (ESI): m/z 505.2 (M+H).

Dimethyl (2-(4-fluoro-5-hydroxy-6-methoxybenzo[b]thiophen-2-yl)-2-oxoethyl) phosphonate (6): To a solution of Dimethyl (2-(4-fluoro-6-methoxy-5-((triisopropylsilyl)oxy)benzo[b]thiophen-2-yl)-2-oxoethyl)phosphonate 5 (2.87 g, 5.69 mmol) in THF (12 mL) was added dropwise tetrabutylammonium fluoride (6.8 mL, 1M in THF, 6.82 mmol). The reaction mixture was stirred at RT under nitrogen atmosphere for 2 hours, then the mixture was quenched with water (30 mL) and acidified to pH˜3 by adding 1M HCl, extracted with EtOAc (80 mL×3). The combined organic layers were dried over Na₂SO₄ and concentrated under reduced pressure. The residue was purified by silica gel chromatography to afford the desired product 6 (1.87 g, 94.4%) as a yellow solid. LCMS (ESI)=m/z 349.1 (M+H).

Methyl 4-(5-(3-((2-(2-(dimethoxyphosphoryl) acetyl)-4-fluoro-6-methoxybenzo[b]thiophen-5-yl) oxy) propoxy)-4-fluoro-6-methoxybenzo[b]thiophen-2-yl)-2, 2-dimethyl-4-oxobutanoate (Compound 3): To a solution of dimethyl (2-(4-fluoro-5-hydroxy-6-methoxybenzo[b]thiophen-2-yl)-2-oxoethyl) phosphonate 6 (1.8 g, 5.17 mmol) and methyl 4-(5-(3-bromopropoxy)-4-fluoro-6-methoxybenzo[b]thiophen-2-yl)-2, 2-dimethyl-4-oxobutanoate (A) (2.4 g, 5.17 mmol) in CH₃CN (40 mL) was added Cs₂CO₃ (5.1 g, 15.50 mmol), the mixture was stirred at 65° C. under nitrogen atmosphere for 2 hours. After which, the reaction was filtered and concentrated under reduced pressure. The residue was purified by silica gel chromatography to give the desired product Compound 3 (2.6 g, 69.1%) as a light yellow solid. ¹H NMR (400 MHz, DMSO-d₆) δ 8.43 (s, 1H), 8.26 (s, 1H), 7.55 (d, J=3.4 Hz, 2H), 4.28 (td, J=6.3, 3.3 Hz, 4H), 4.00 (s, 1H), 3.94 (s, 1H), 3.87 (d, J=1.8 Hz, 6H), 3.70 (s, 3H), 3.67 (s, 3H), 3.57 (s, 3H), 3.42 (s, 2H), 2.09 (p, J=6.2 Hz, 2H), 1.24 (s, 6H).

4-(5-(3-((2-(2-(Dimethoxyphosphoryl)acetyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid (Compound 4): To a solution of Methyl 4-(5-(3-((2-(2-(dimethoxyphosphoryl) acetyl)-4-fluoro-6-methoxybenzo[b]thiophen-5-yl) oxy) propoxy)-4-fluoro-6-methoxybenzo[b]thiophen-2-yl)-2, 2-dimethyl-4-oxobutanoate Compound 3 (20 mg, 0.03 mmol) in DCE (1 mL) was added Sn(CH₃)₃OH (20 mg, 0.11 mmol) at RT. Then the mixture was heated to 80° C. and stirred overnight under nitrogen atmosphere. The reaction was cooled to RT, concentrated under reduced pressure and the residue was purified by preparative HPLC to afford the desired product Compound 4 (8 mg, 41%) as a white solid. ¹H NMR (400 MHz, DMSO-d₆) δ 8.26 (s, 2H), 7.54 (s, 1H), 7.50 (s, 1H), 4.31-4.22 (m, 4H), 3.87 (d, J=5.6 Hz, 6H), 3.57 (s, 3H), 3.46 (s, 2H), 3.42 (s, 3H), 3.33 (m, 2H) 2.08 (m, 2H), 1.23 (s, 6H).

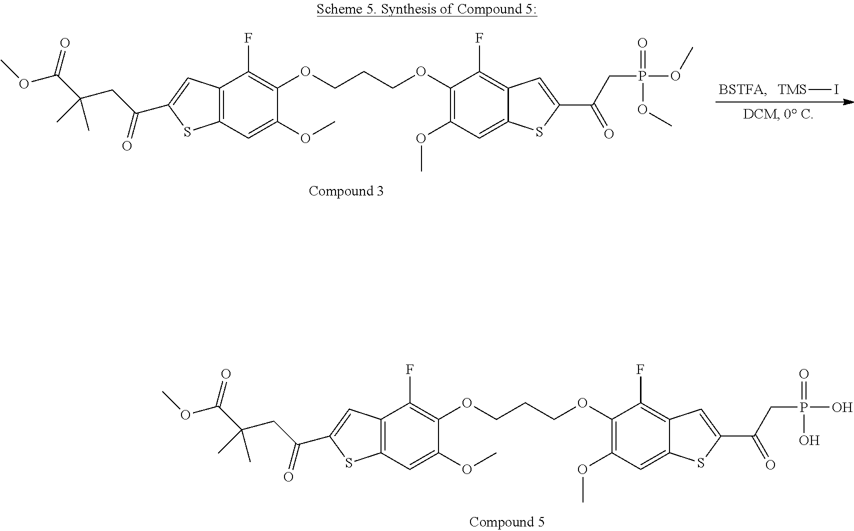

(2-(4-Fluoro-5-(3-((4-fluoro-6-methoxy-2-(4-methoxy-3,3-dimethyl-4-oxobutanoyl)benzo[b]thiophen-5-yl)oxy)propoxy)-6-methoxybenzo[b]thiophen-2-yl)-2-oxoethyl)phosphonic acid (Compound 5): To a solution of methyl 4-(5-(3-((2-(2-(dimethoxyphosphoryl)acetyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoate Compound 3 (3.0 g, 4.12 mmol) in DCM (120 mL) was added N,O-bis(trimethylsilyl) trifluoroacetamide (6.4 g, 24.70 mmol) and Iodotrimethylsilane (4.1 g, 20.58 mmol) at 0° C. then the mixture was stirred under nitrogen atmosphere for 1 h. The reaction was concentrated under reduced pressure and the residue was purified by Reverse phase column to afford the desired product Compound 5 (1.69 g, 58.6%) as a light orange solid. LCMS (ESI)=m/z 699.2 (M−H). ¹H NMR (400 MHz, DMSO-d₆) δ 8.26 (s, 2H), 7.54 (d, J=4.2 Hz, 2H), 4.27 (t, J=6.1 Hz, 4H), 3.87 (s, 6H), 3.62-3.52 (m, 5H), 2.10 (q, J=6.2 Hz, 2H), 1.24 (s, 6H).

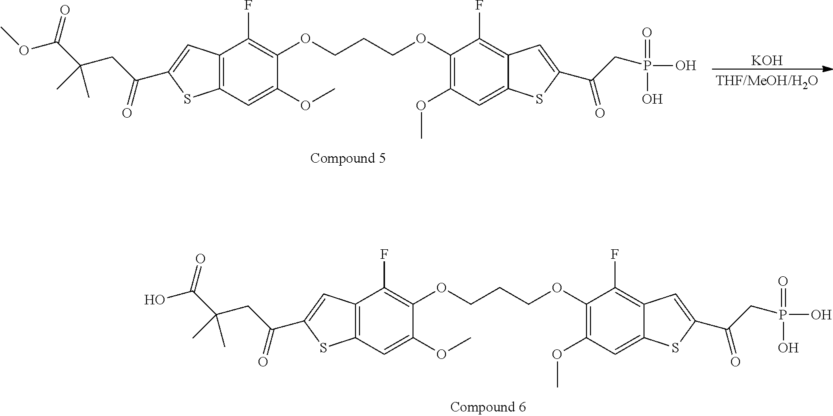 [SMILES: COC(=O)C(C)(C)CC(=O)c1cc2c(F)c(OCCCOc3c(OC)cc4sc(C(=O)CP(=O)(O)O)cc4c3F)c(OC)cc2s1 ; "KOH" ; COc1cc2sc(C(=O)CC(C)(C)C(=O)O)cc2c(F)c1OCCCOc1c(OC)cc2sc(C(=O)CP(=O)(O)O)cc2c1F]

4-(4-Fluoro-5-(3-((4-fluoro-6-methoxy-2-(2-phosphonoacetyl)benzo[b]thiophen-5-yl)oxy)propoxy)-6-methoxybenzo[b]thiophen-2-yl)-2,2-dimethyl-4-oxobutanoic acid: To a solution of (2-(4-fluoro-5-(3-((4-fluoro-6-methoxy-2-(4-methoxy-3,3-dimethyl-4-oxobutanoyl)benzo[b]thiophen-5-yl)oxy)propoxy)-6-methoxybenzo[b]thiophen-2-yl)-2-oxoethyl)phosphonic acid Compound 5 (21 mg, 0.03 mmol) in MeOH (0.7 mL), THF (0.7 mL), H₂O (0.7 mL) was added KOH (14 mg, 0.24 mmol) at RT, the reaction was stirred overnight at 40° C. The mixture was cooled to RT, concentrated under reduced pressure. The residue was purified by Preparative HPLC to afford the desired product Compound 6 (10 mg. 47%) as a white solid. ¹H NMR (400 MHz, DMSO-d₆) δ 8.22 (s, 1H), 8.01 (s, 1H), 7.45 (s, 1H), 7.36 (s, 1H), 4.28 (m, 4H), 3.86 (s, 3H), 3.80 (s, 3H), 3.29 (m, 2H), 3.17 (d, J=20.7 Hz, 2H), 2.05 (m, 2H), 1.24 (s, 6H).

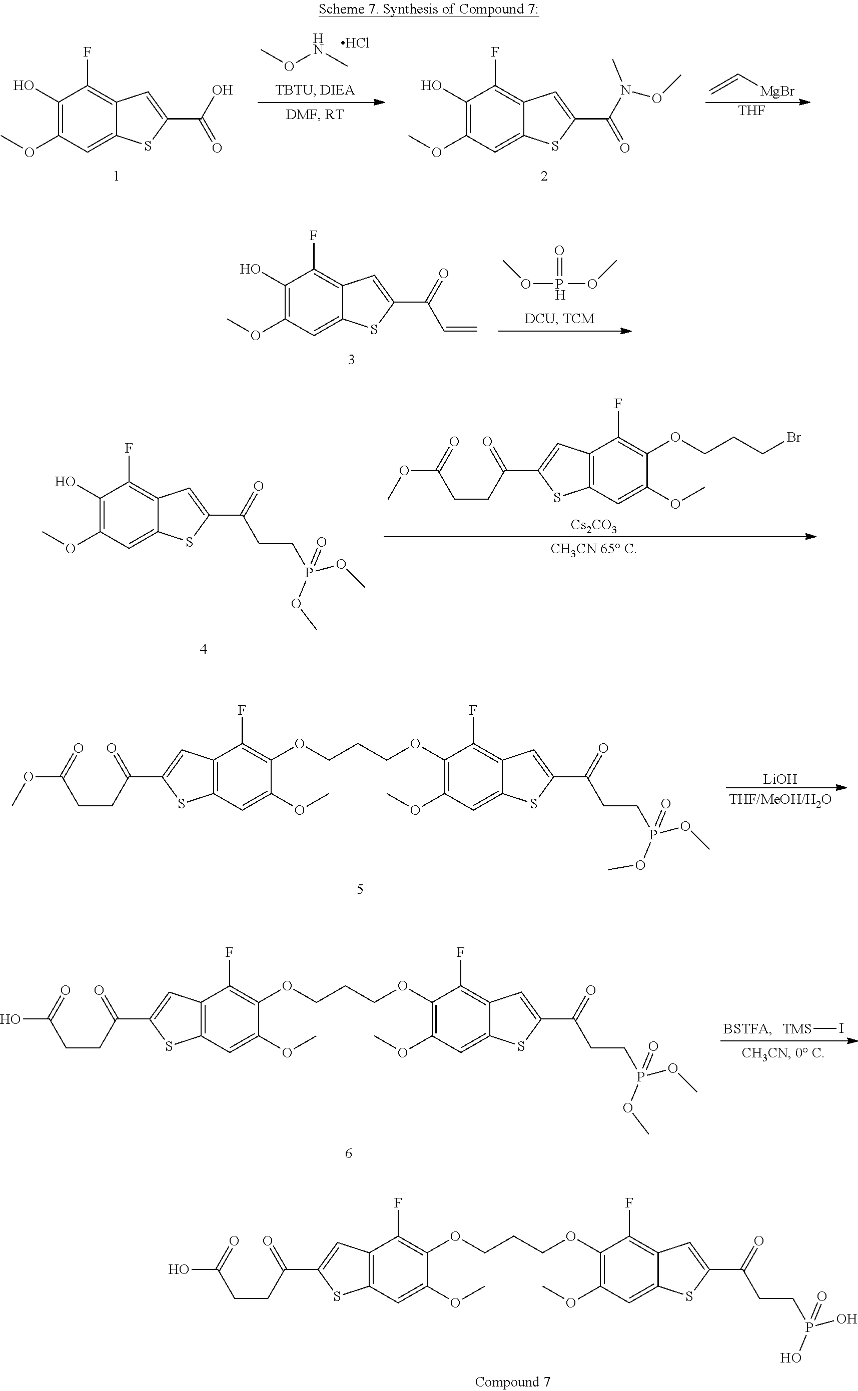

4-Fluoro-5-hydroxy-N,6-dimethoxy-N-methylbenzo[b]thiophene-2-carboxamide (2): To a solution of 4-fluoro-5-hydroxy-6-methoxybenzo[b]thiophene-2-carboxylic acid (3.46 g, 14.3 mmol) in DMF (35 mL) was added N,O-dimethylhydroxylamine (1.95 g, 20 mmol), TBTU (5.05 g, 15.7 mmol) and DIEA (4.6 g, 35.7 mmol). The mixture was stirred overnight at RT. After which, the reaction was diluted with water and extracted with EtOAc. The organic layers were dried over Na₂SO₄, concentrated under reduced pressure. The crude product was purified by silica gel chromatography to afford compound 2 (1.33 g) as a solid. ¹H NMR (400 MHz, DMSO-d₆) δ 7.96 (s, 1H), 7.43 (s, 1H), 3.90 (s, 3H), 3.80 (s, 3H), 3.32 (s, 3H).

1-(4-Fluoro-5-hydroxy-6-methoxybenzo[b]thiophen-2-yl)prop-2-en-1-one (3): To a solution of 4-fluoro-5-hydroxy-N,6-dimethoxy-N-methylbenzo[b]thiophene-2-carboxamide 2 (1.26 g, 4.42 mmol) in THF (25 mL) was added vinyl magnesium bromide (22.1 ml, 22 mmol) at 0° C., the reaction mixture was stirred at RT for 30 min. The reaction was added to ice cold 1M HCl solution at 0° C., extracted with EtOAc. The combined organic layers were dried over Na₂SO₄ and concentrated under reduced pressure. The crude product was purified by silica gel chromatography to afford compound 3 (564 mg) as a solid. ¹H NMR (400 MHz, DMSO-d₆) δ 9.55 (s, 1H), 8.40 (s, 1H). 7.57 (dd, J=16.9, 10.4 Hz, 1H), 7.49 (s, 1H), 6.38 (dd, J=16.9, 1.9 Hz, 1H), 5.95 (dd, J=10.3, 1.9 Hz, 1H), 3.92 (s, 3H).

Dimethyl (3-(4-fluoro-5-hydroxy-6-methoxybenzo[b]thiophen-2-yl)-3-oxopropyl)phosphonate (4): To a solution of 1-(4-fluoro-5-hydroxy-6-methoxybenzo[b]thiophen-2-yl)prop-2-en-1-one 3 (539 mg, 2.1 mmol) in DCM (5 mL) was added dimethyl phosphonate (940 mg, 8.54 mmol) and DBU (162.4 mg, 1.07 mmol) at RT, the mixture was stirred at 65° C. for 2 h. TLC showed SM disappeared. The mixture was diluted with water and extracted with DCM. The organic layers were dried over Na₂SO₄ and concentrated under reduced pressure. The crude product was purified by silica gel chromatography to afford compound 4 (343 mg) as a solid. ¹H NMR (400 MHz, DMSO-d₆) δ 8.24 (s, 1H), 7.44 (s, 1H), 3.88 (s, 3H), 3.63 (s, 3H), 3.61 (s, 3H), 3.23 (m, 2H), 2.14-2.04 (m, 2H).

Methyl 4-(5-(3-((2-(3-(dimethoxyphosphoryl)propanoyl)-4-fluoro-6-methoxybenzo[b]thiophen-5-yl)oxy)propoxy)-4-fluoro-6-methoxybenzo[b]thiophen-2-yl)-4-oxobutanoate (5): To a solution of dimethyl (3-(4-fluoro-5-hydroxy-6-methoxybenzo[b]thiophen-2-yl)-3-oxopropyl)phosphonate 4 (148 mg, 0.41 mmol) in CH₃CN (2 mL) was added methyl 4-(5-(3-bromopropoxy)-4-fluoro-6-methoxy benzo[b]thiophen-2-yl)-4-oxobutanoate A (177 mg, 0.41 mmol) and Cs₂CO₃ (399.3 mg, 1.23 mmol) at RT, the reaction mixture was stirred at 65° C. for 2 h. The reaction was diluted with water and extracted with DCM. The organic layers were dried over Na₂SO₄ and concentrated under reduced pressure. The crude product was purified by silica gel chromatography to afford compound 5. ¹H NMR (400 MHz, Chloroform-d) δ 7.93 (s, 2H), 7.05 (s, 2H), 4.39 (d, J=6.1 Hz, 4H), 3.91 (s, 6H), 3.78 (dd, J=10.8, 2.5 Hz, 6H), 3.72 (d, J=2.5 Hz, 3H), 3.32 (d, J=6.8 Hz, 2H), 2.80 (t, J=6.7 Hz, 2H), 2.31-2.17 (m, 4H), 0.87 (s, 2H).

4-(4-Fluoro-5-(3-((4-fluoro-2-(3-(hydroxy(methoxy)phosphoryl)propanoyl)-6-methoxybenzo[b]thiophen-5-yl)oxy)propoxy)-6-methoxybenzo[b]thiophen-2-yl)-4-oxobutanoic acid (6): To a solution of methyl 4-(5-(3-((2-(3-(dimethoxyphosphoryl)propanoyl)-4-fluoro-6-methoxybenzo[b]thiophen-5-yl)oxy)propoxy)-4-fluoro-6-methoxybenzo[b]thiophen-2-yl)-4-oxobutanoate 5 (109 mg, 0.15 mmol) in THF/MeOH/H₂O (0.5 mL/0.5 mL/0.5 mL) was added LiOH (14.6 mg, 0.61 mmol), the mixture was stirred overnight at RT. The reaction mixture was diluted with 1M HCl solution and extracted with EtOAc, The organic layers were dried over Na₂SO₄, and concentrated under reduced pressure. The crude product was purified by Preparative HPLC to afford the compound 6 (82 mg) as a white solid. LCMS (ESI)=m/z 685 (M−H)

4-(4-Fluoro-5-(3-((4-fluoro-6-methoxy-2-(3-phosphonopropanoyl)benzo[b]thiophen-5-yl)oxy)propoxy)-6-methoxybenzo[b]thiophen-2-yl)-4-oxobutanoic acid (Compound 7): To a solution of 4-(4-fluoro-5-(3-((4-fluoro-2-(3-(hydroxy(methoxy)phosphoryl)propanoyl)-6-methoxybenzo[b]thiophen-5-yl)oxy)propoxy)-6-methoxybenzo[b]thiophen-2-yl)-4-oxobutanoic acid 6 (50 mg, 0.07 mmol) in CH₃CN was added BSTFA (75 mg, 0.29 mmol) and TMSI (87.4 mg, 0.44 mmol) at 0° C. After 1 h, the reaction mixture was concentrated, and the crude product was purified by preparative HPLC to give the desired compound Compound 7 (18 mg) as a white solid. LCMS (ESI)=m/z 670.95 (M−H); ¹H NMR (400 MHz, DMSO-d₆) δ 8.23 (s, 1H), 8.14 (s, 1H), 7.53 (s, 2H), 4.27 (t, J=6.1 Hz, 4H), 3.87 (s, 6H), 3.30 (s, 2H), 3.24-3.20 (m, 2H), 2.59 (t, J=6.4 Hz, 2H), 2.08 (p, J=6.2 Hz, 2H), 1.89 (dt, J=16.3, 7.7 Hz, 2H).

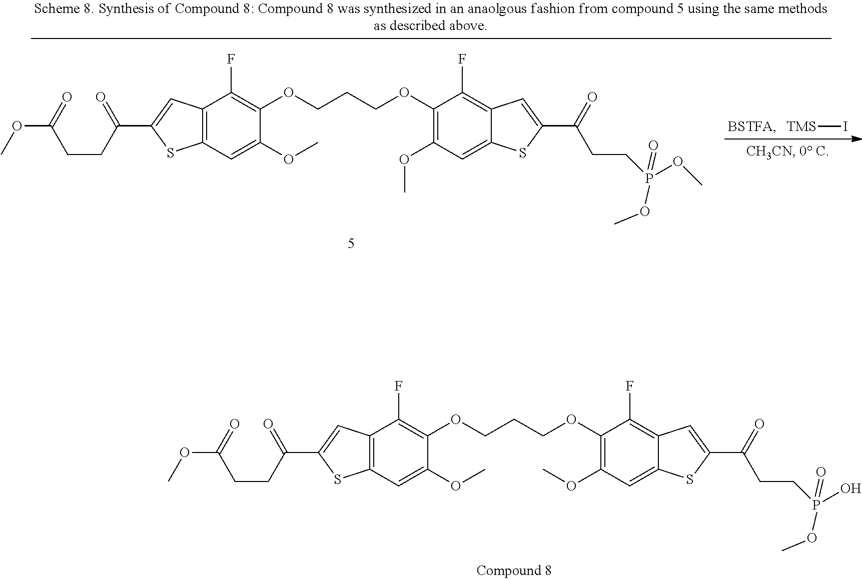

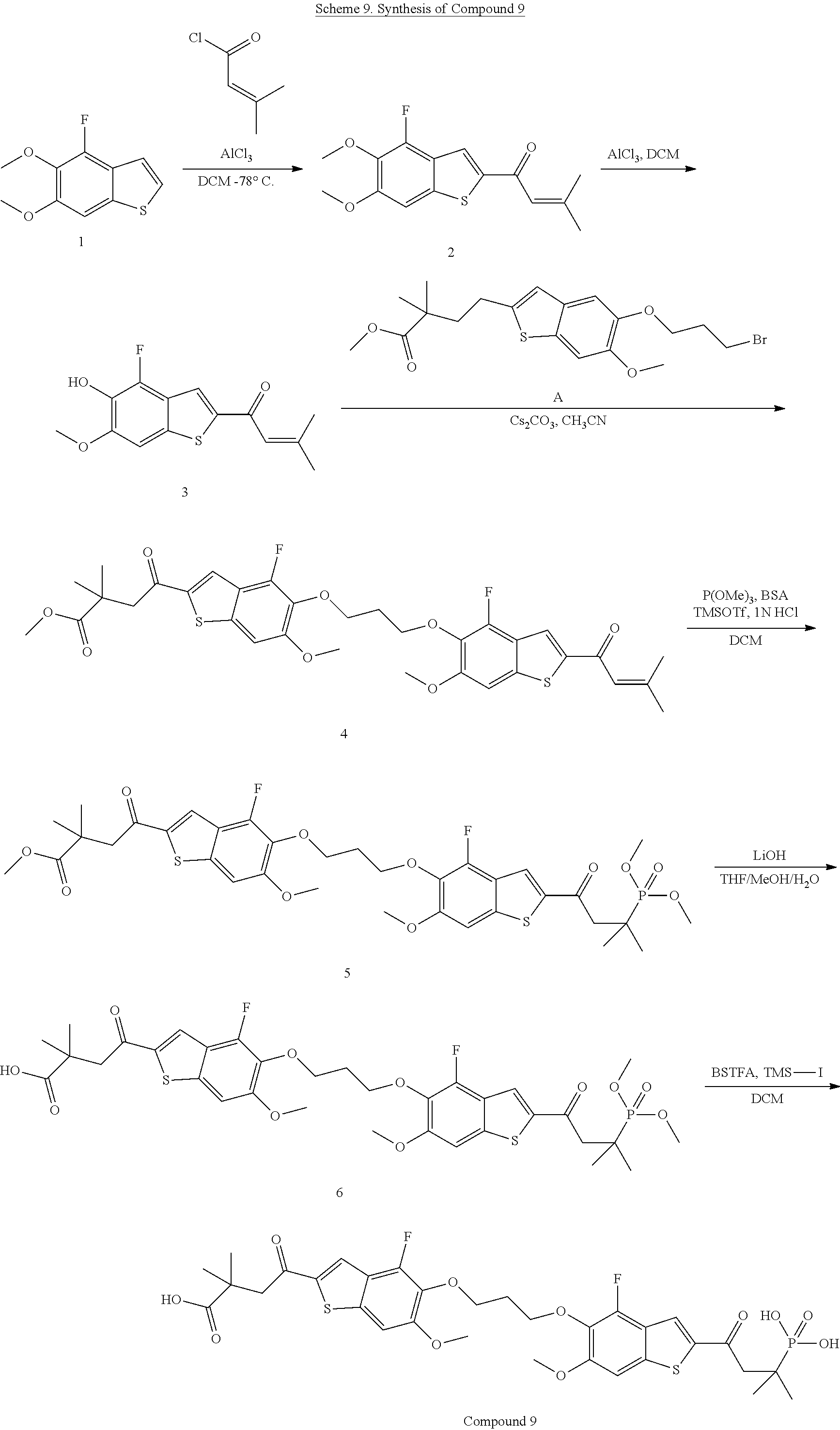

1-(4-Fluoro-5,6-dimethoxybenzo[b]thiophen-2-yl)-3-methylbut-2-en-1-one (2): To a solution of AlCl₃ (1.4 g, 10.41 mmol) in DCM (20 mL) was added 4-fluoro-5,6-dimethoxybenzo[b]thiophene (1.7 g, 8.01 mmol) 3-methylbut-2-enoyl chloride (1.43 g, 12.01 mmol) at −78° C. The reaction was stirred overnight at RT. After which, the reaction mixture was added to ice-water and extracted with EtOAc, The combined organic layers were dried over Na₂SO₄, and concentrated under reduced pressure. The crude product was purified by silica gel chromatography to afford compound 2 (2.18 g) as a solid. ¹H NMR (400 MHz, DMSO-d₆) δ 8.29 (s, 1H), 7.55 (s, 1H), 7.11 (s, 1H), 3.91 (s, 3H), 3.84 (s, 3H), 2.19 (d, J=1.2 Hz, 3H), 2.02 (d, J=1.3 Hz, 3H).

1-(4-Fluoro-5-hydroxy-6-methoxybenzo[b]thiophen-2-yl)-3-methylbut-2-en-1-one (3): To a solution of 1-(4-fluoro-5,6-dimethoxybenzo[b]thiophen-2-yl)-3-methylbut-2-en-1-one 2 (1.58 g, 5.37 mmol) in DCM (20 mL) was added AlCl₃ (6.4 g, 48.3 mmol) at 0° C., the reaction was stirred overnight at RT, then the mixture was slowly added to ice cold water, extracted with THF. The combined organic layers were dried over Na₂SO₄ and concentrated under reduced pressure. The crude product was purified by silica gel chromatography to afford compound 3 (1.45 g) as a yellow solid. ¹H NMR (400 MHz, Chloroform-d) δ 7.90 (d, J=1.3 Hz, 1H), 7.07 (s, 1H), 6.78 (d, J=2.0 Hz, 1H), 4.01 (d, J=1.4 Hz, 3H), 2.28 (d, J=1.5 Hz, 3H), 2.05 (d, J=1.5 Hz, 3H).

Methyl 4-(4-fluoro-5-(3-((4-fluoro-6-methoxy-2-(3-methylbut-2-enoyl)benzo[b]thiophen-5-yl)oxy)propoxy)-6-methoxybenzo[b]thiophen-2-yl)-2,2-dimethyl-4-oxobutanoate (4): To a solution of 1-(4-fluoro-5-hydroxy-6-methoxybenzo[b]thiophen-2-yl)-3-methylbut-2-en-1-one 3 (941 mg, 3.36 mmol) in DMF (15 mL) was added methyl 4-(5-(3-bromopropoxy)-6-methoxybenzo[b]thiophen-2-yl)-2,2-dimethylbutanoate A (1.55 g, 3.36 mmol), Cs₂CO₃ (5.5 g, 16.8 mmol) and the reaction mixture was stirred at 65° C. for 2 h. The reaction was cooled to RT, diluted with water and extracted with EtOAc, the combined organic layers were dried over Na₂SO₄, and concentrated under reduced pressure. The crude product was purified by preparative HPLC to afford compound 4 (375 mg) as a white solid. ¹H NMR (400 MHz, DMSO-d₆) δ 8.23 (s, 2H), 7.52 (d, J=9.2 Hz, 2H), 7.09 (s, 1H), 4.27 (t, J=6.1 Hz, 4H), 3.86 (s, 6H), 3.56 (s, 3H), 2.20 (s, 3H), 2.09 (q, J=6.3 Hz, 2H), 2.03 (s, 3H), 1.23 (s, 6H).

Methyl 4-(5-(3-((2-(3-(dimethoxyphosphoryl)-3-methylbutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoate (5): To a mixture of P(OMe)₃ (347.4 mg, 2.8 mmol) and N,O-bis(trimethylsilyl)acetamide) (569 mg, 2.8 mmol) in DCM (4 mL) was added TMSOTf (622.3 mg, 2.8 mmol) at 0° C. After 30 min, methyl 4-(4-fluoro-5-(3-((4-fluoro-6-methoxy-2-(3-methylbut-2-enoyl)benzo[b]thiophen-5-yl)oxy)propoxy)-6-methoxybenzo[b]thiophen-2-yl)-2,2-dimethyl-4-oxobutanoate 4 (370 mg, 0.55 mmol) was added and the reaction was stirred at 0° C. for 1 h. The enolsilane intermediate was hydrolyzed by stirring the reaction mixture with 3 mL of 1M HCl for 3 h. The organic layer was separated, dried over Na₂SO₄, and concentrated under reduced pressure. The crude product was purified by silica gel chromatography to give compound 5 (178 mg) as a white solid. ¹H NMR (400 MHz, DMSO-d₆) δ 8.24 (s, 1H), 8.12 (s, 1H), 7.53 (d, J=3.1 Hz, 2H), 4.27 (t, J=6.1 Hz, 4H), 3.86 (s, 6H), 3.65 (s, 3H), 3.62 (s, 3H), 3.57 (s, 3H), 2.08 (q, J=6.1 Hz, 2H), 1.23 (s, 12H).

4-(5-(3-((2-(3-(Dimethoxyphosphoryl)-3-methylbutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid (6): To a solution of methyl 4-(5-(3-((2-(3-(dimethoxyphosphoryl)-3-methylbutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoate 5 (68 mg, 0.08 mmol) in THF/MeOH/H₂O (0.5 mL/0.5 mL/0.5 mL) was added LiOH (8.5 mg, 0.35 mmol) and the mixture was stirred overnight at RT then the reaction was acidified by adding 1M HCl solution, extracted with EtOAc, the combined organic layers were dried over Na₂SO₄, and concentrated under reduced pressure. The crude product was purified by preparative HPLC to afford compound 6 (65 mg) as a white solid.

4-(4-Fluoro-5-(3-((4-fluoro-6-methoxy-2-(3-methyl-3-phosphonobutanoyl)benzo[b]thiophen-5-yl)oxy)propoxy)-6-methoxybenzo[b]thiophen-2-yl)-2,2-dimethyl-4-oxobutanoic acid (Compound 9): To a solution of 4-(5-(3-((2-(3-(dimethoxyphosphoryl)-3-methylbutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid 6 (63 mg, 0.08 mmol) in DCM was added BSTFA (85.7 mg, 0.33 mmol) and TMSI (99.9 mg, 0.50 mmol) sequentially at 0° C., the reaction was stirred for 1 h and then concentrated under reduced pressure. The crude product was purified by preparative HPLC to afford the desired compound Compound 9 (25 mg) as white solid. LCMS (ESI)=m/z 727.05 (M−H); ¹H NMR (400 MHz, DMSO-d₆) δ 8.21 (s, 1H), 8.00 (s, 1H), 7.50 (s, 2H), 4.26 (t, J=6.2 Hz, 4H), 3.86 (s, 6H), 3.34 (s, 2H), 3.09 (d, J=9.3 Hz, 2H), 2.07 (t, J=6.7 Hz, 2H), 1.22 (s, 12H).

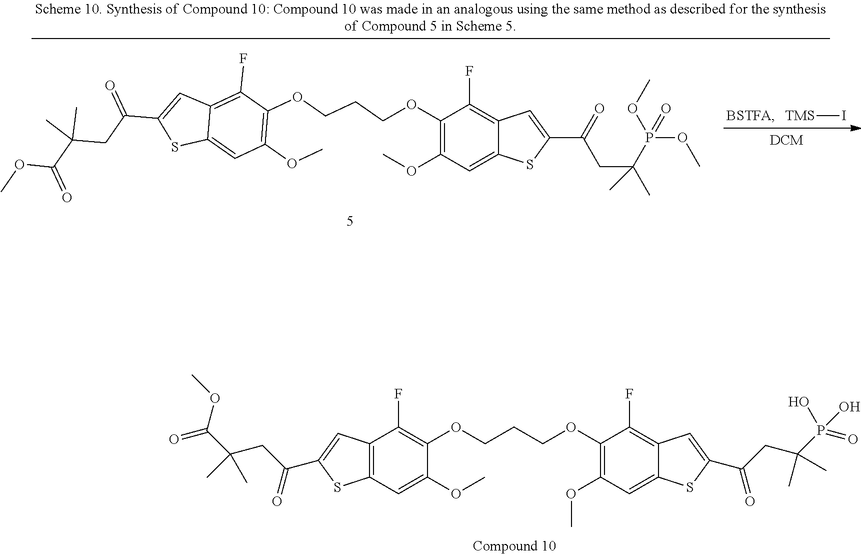

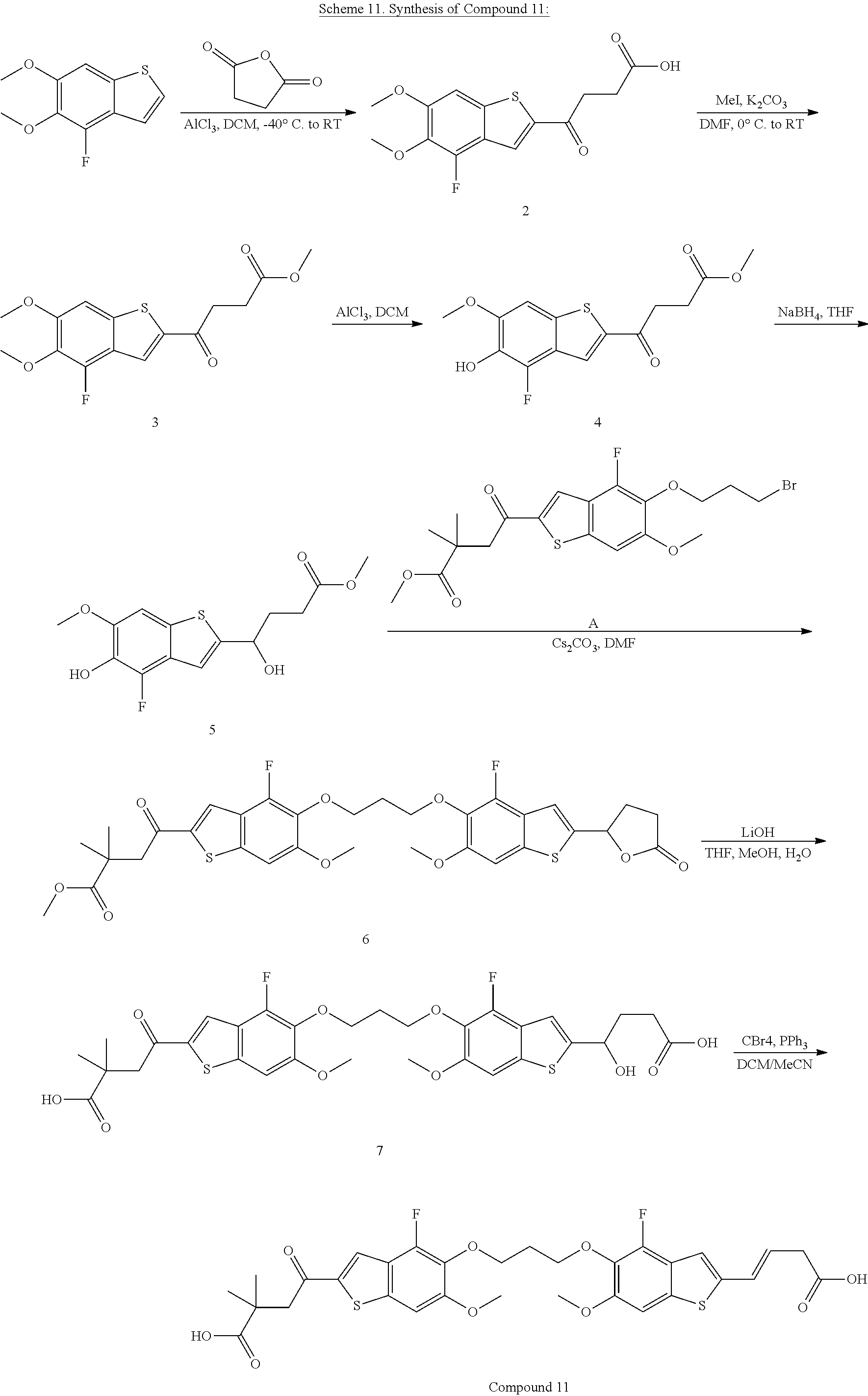

4-(4-Fluoro-5,6-dimethoxybenzo[b]thiophen-2-yl)-4-oxobutanoic acid (2): To a solution of 4-fluoro-5,6-dimethoxybenzo[b]thiophene (2 g, 9.43 mmol) and succinic anhydride (1.4 g, 14.13 mmol) in DCM (90 mL) was added AlCl₃ (2.5 g, 18.86 mmol) portion wise and the reaction was stirred overnight at RT. The reaction was slowly quenched with ice cold water (500 mL), extracted with EtOAc (400 mL×3). The combined organic layers were washed with brine, dried over Na₂SO₄ and concentrated under reduced pressure. The residue was purified by silica gel chromatography to afford the desired product 2 (1.8 g, 62%) as a yellow solid. ¹H NMR (400 MHz, DMSO-d₆) δ 12.17 (s, 1H), 8.31 (s, 1H), 7.57 (s, 1H), 3.92 (s, 3H), 3.86 (s, 3H), 3.30 (s, 2H), 2.59 (t, J=6.4 Hz, 2H).

Methyl 4-(4-fluoro-5,6-dimethoxybenzo[b]thiophen-2-yl)-4-oxobutanoate (3): The solution of 4-(4-fluoro-5,6-dimethoxybenzo[b]thiophen-2-yl)-4-oxobutanoic acid 2 (5.44 g, 17.4 mmol), K₂CO₃ (7.2 g, 52.3 mmol) and CH₃I (4.9 g, 34.87 mmol) in DMF (155 mL) was stirred at RT for 12 h. The reaction mixture was diluted into 800 mL of water and extracted with EtOAc (400 mL×3), the combined organic layers were concentrated, and the residue was purified by silica gel column chromatography to afford the desired product 3 (4.57 g, 81% yield) as a yellow solid. LCMS (ESI)=m/z 326.9 (M+H).

Methyl 4-(4-fluoro-5-hydroxy-6-methoxybenzo[b]thiophen-2-yl)-4-oxobutanoate (4): To a suspension of AlCl₃ (9.4 g, 70.5 mmol) in DCM (225 mL) was added 4-(4-fluoro-5,6-dimethoxybenzo[b]thiophen-2-yl)-4-oxobutanoic acid 3 (4.5 g, 14 mmol) at 0° C. The mixture was warmed to ambient temperature and stirred for 16 hours under nitrogen atmosphere. The mixture was quenched with water (500 mL) and acidified to pH˜2 by adding slowly 1 M HCl, extracted with EtOAc (300 mL×3). The combined organic layers were washed with brine, dried over Na₂SO₄ and concentrated under reduced pressure. The residue was purified by silica gel chromatography to afford the desired product 4 (3.9 g, 90%) as a light yellow solid. ¹H NMR (400 MHz, DMSO-d6) δ 9.51 (s, 1H), 8.24 (s, 1H), 7.46 (s, 1H), 3.91 (s, 3H), 3.60 (s, 3H), 3.37 (t, J=6.4 Hz, 2H), 2.67 (t, J=6.3 Hz, 2H).

Methyl 4-(4-fluoro-5-hydroxy-6-methoxybenzo[b]thiophen-2-yl)-4-hydroxybutanoate (5): To a solution of methyl 4-fluoro-5-hydroxy-6-methoxybenzo[b]thiophene-2-carboxylate 4 (400 mg, 1.33 mmol) in THF (5 mL) was added portion wise NaBH₄ (38 mg, 1.33 mmol) at 0° C. and the reaction was allowed to stir at RT for 1 h, after which the reaction was diluted with water (10 mL) and extracted with EtOAc (10 mL×3). The combined organic layer was washed with brine, dried over Na₂SO₄ and concentrated under reduced pressure. The residue was purified by silica gel chromatography to afford the desired product 5 (220 mg, 54%) as a light yellow solid. LCMS (ESI)=m/z 314.9 (M+H).

Methyl 4-(4-fluoro-5-(3-((4-fluoro-6-methoxy-2-(5-oxotetrahydrofuran-2-yl)benzo[b]thiophen-5-yl)oxy)propoxy)-6-methoxybenzo[b]thiophen-2-yl)-2,2-dimethyl-4-oxobutanoate (6): To a solution of methyl 4-(4-fluoro-5-hydroxy-6-methoxybenzo[b]thiophen-2-yl)-4-hydroxybutanoate 5 (200 mg, 0.64 mmol) and methyl 4-(5-(3-bromopropoxy)-4-fluoro-6-methoxybenzo[b]thiophen-2-yl)-2, 2-dimethyl-4-oxobutanoate A (310 mg, 0.67 mmol) in CH₃CN (10 mL) was added Cs₂CO₃ (1.01 g, 3.18 mmol), the mixture was stirred at 65° C. for 2 hours. The reaction was filtered and concentrated under reduced pressure. The residue was purified by silica gel chromatography to afford the desired product 6 (180 mg, 43%) as a light yellow solid.

4-(5-(3-((2-(3-Carboxy-1-hydroxypropyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid (7): To a solution of methyl 4-(4-fluoro-5-(3-((4-fluoro-6-methoxy-2-(5-oxotetrahydrofuran-2-yl)benzo[b]thiophen-5-yl)oxy)propoxy)-6-methoxybenzo[b]thiophen-2-yl)-2,2-dimethyl-4-oxobutanoate 6 (170 mg, 0.26 mmol) in THF/MeOH/H₂O (1 mL/5 ml/1 ml) was added LiOH (216 mg, 3.18 mmol), the reaction was stirred overnight at RT. After which, the mixture was acidified to pH=1 by adding 1M HCl solution, extracted with EtOAc (15 mL×3), the combined organic layer was dried over Na₂SO₄, and concentrated. The crude product was purified by silica gel chromatography to afford the desired product 7 (155 mg, 92%) as a light yellow solid. LCMS (ESI)=m/z 665.2 (M−H).

(E)-4-(5-(3-((2-(3-carboxy-3-methylbutanoyl)-4-fluoro-6-methoxybenzo[b]thiophen-5-yl)oxy)propoxy)-4-fluoro-6-methoxybenzo[b]thiophen-2-yl)but-3-enoic acid (Compound 11): To a solution of 4-(5-(3-((2-(3-carboxy-1-hydroxypropyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid 7 (90 mg, 0.14 mmol) in CH₃CN (0.3 mL) and DCM (1 mL) was added CBr₄ (93 mg, 0.28 mmol) and PPh₃ (74 mg, 0.28 mmol) at −25° C. under nitrogen atmosphere. The mixture was stirred overnight at RT. After which, the reaction mixture was concentrated under reduced pressure, purified by preparative TLC (DCM/MeOH=10/1, v/v) to give the desired product Compound 11 (18 mg, 20%) as a white solid. ¹H NMR (400 MHz, DMSO-d6) δ 8.24 (s, 1H), 7.53 (s, 1H), 7.41 (s, 1H), 7.21 (s, 1H), 6.76 (d, J=16.0 Hz, 1H), 6.06 (m, 1H), 4.24 (m, 4H), 3.86 (s, 3H), 3.82 (s, 3H), 3.36 (s, 2H), 3.23 (d, J=7.2 Hz, 2H), 2.07 (m, 2H), 1.23 (s, 6H).

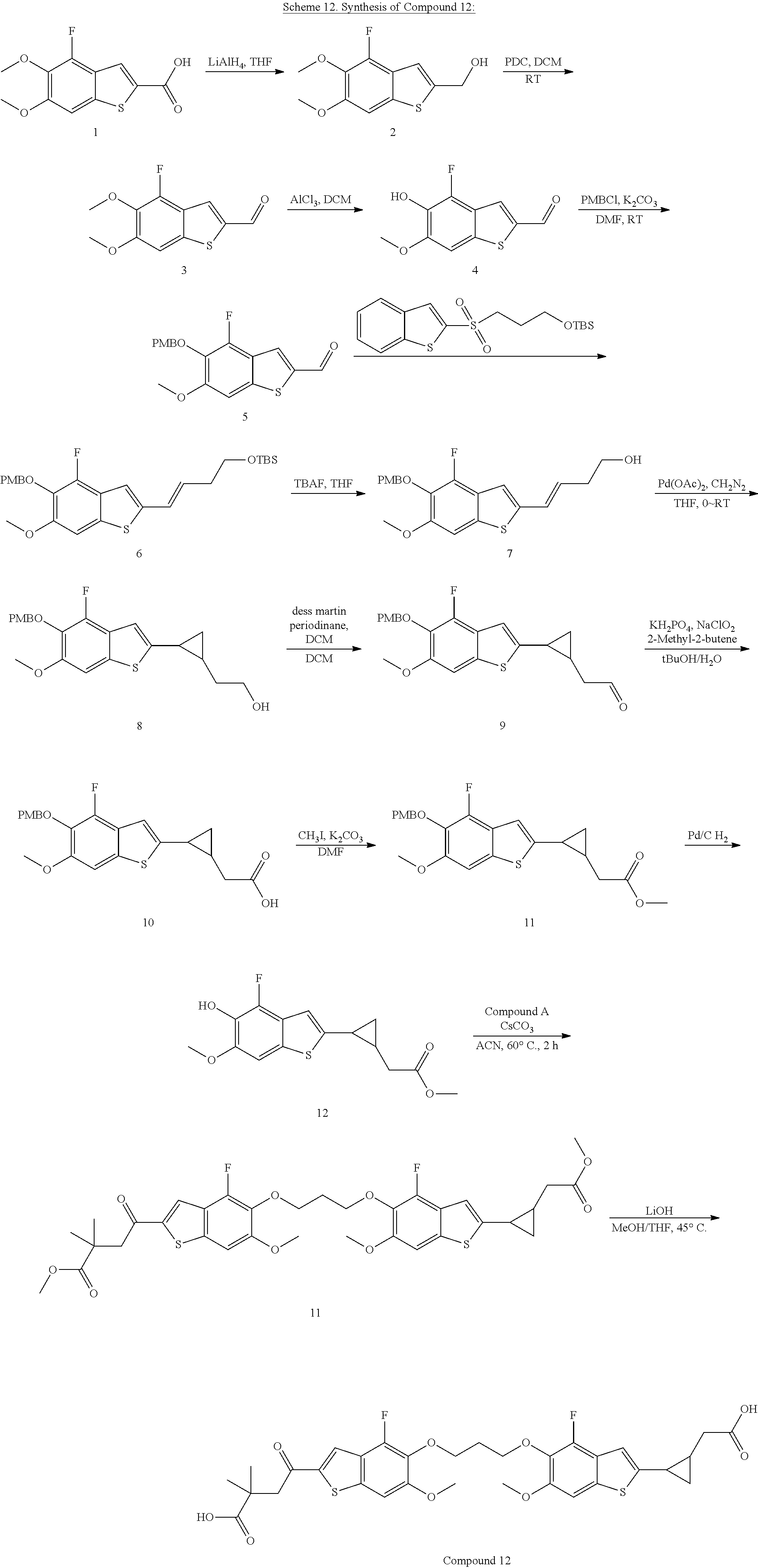

(4-Fluoro-5, 6-dimethoxybenzo[b]thiophen-2-yl) methanol (2): To a solution of 4-fluoro-5, 6-dimethoxybenzo[b]thiophene-2-carboxylic acid 1 (3.84 g, 14.98 mmol) in THF (45 mL) was added Lithium aluminohydride (1M, 44.9 mL, 45.0 mmol) at 0° C. The reaction mixture was stirred at 0° C. for 3 h. LCMS showed completion of the reaction. The reaction mixture was carefully quenched with ice cold water, acidified to pH˜3 by adding TN HCl, extracted with EtOAc (30×3 mL). The combined organic phases were concentrated and purified by silica gel column to give the desired product 2 (1.64 g, 45% yield) as a yellow solid. ¹H NMR (400 MHz, DMSO-d₆) δ 7.45 (s, 1H), 7.17 (s, 1H), 5.63 (t, J=5.8 Hz, 1H), 4.69 (dd, J=5.9, 1.1 Hz, 2H), 3.87 (s, 3H), 3.81 (s, 3H).

4-Fluoro-5, 6-dimethoxybenzo[b]thiophene-2-carbaldehyde (3): To a solution of (4-fluoro-5,6-dimethoxybenzo[b]thiophen-2-yl)methanol 2 (1.64 g, 6.76 mmol) in DCM (40 mL) was added PDC (5.09 g, 13.53 mmol), the mixture was stirred at RT for 16 h. after which, the mixture was concentrated and the crude material was purified by silica gel column chromatography to afford the compound 3 (1.43 g, 88% yield) as a yellow semi-solid. ¹H NMR (400 MHz, DMSO-d₆) δ 10.04 (s, 1H), 8.37 (s, 1H), 7.63 (s, 1H), 3.93 (s, 3H), 3.86 (s, 3H).

4-Fluoro-5-hydroxy-6-methoxybenzo[b]thiophene-2-carbaldehyde (4): To the solution of 4-fluoro-5, 6-dimethoxybenzo[b]thiophene-2-carbaldehyde 3 (1.43 g, 5.95 mmol) in DCM (60 mL) was added AlCl₃ (6.35 g, 47.62 mmol) at 0° C. The mixture was stirred overnight at RT, LCMS showed completion of the reaction. The mixture was slowly added to ice-water and extracted with DCM (40×3 mL). The organic layers were dried over Na₂SO₄ and concentrated to give the crude product which was purified by silica gel column chromatography to afford the compound 4 (1.12 g, 83%) as a yellow solid. ¹H NMR (400 MHz, DMSO-d₆) δ 10.02 (s, 1H), 9.61 (s, 1H), 8.30 (s, 1H), 7.52 (s, 1H), 3.93 (s, 3H).

4-Fluoro-6-methoxy-5-((4-methoxybenzyl)oxy)benzo[b]thiophene-2-carbaldehyde (5): To a solution of 4-fluoro-5-hydroxy-6-methoxybenzo[b]thiophene-2-carbaldehyde 4 (100 mg, 0.44 mmol) in DMF (3 mL) was added PMBCl (84 mg, 0.53 mmol) and K₂CO₃ (91.2 mg, 0.66 mmol), the reaction was stirred overnight at RT. LCMS showed completion of the reaction, the mixture was diluted with water and extracted with EtOAc, the organic layers were dried over Na₂SO₄, and concentrated to give the crude product which was purified by silica gel column chromatography to afford the compound 5 (140 mg, 90%). ¹H NMR (400 MHz, DMSO-d₆) δ 10.02 (s, 1H), 8.33 (s, 1H), 7.62 (s, 1H), 7.38-7.29 (m, 2H), 6.94-6.87 (m, 2H), 5.03 (s, 2H), 3.95 (s, 3H), 3.74 (s, 3H).

Tert-butyl((4-(4-fluoro-6-methoxy-5-((4-methoxybenzyl)oxy)benzo[b]thiophen-2-yl)but-3-en-1-yl)oxy)dimethylsilane (6): To the solution of 4-fluoro-5-hydroxy-6-methoxybenzo[b]thiophene-2-carbaldehyde 5 (580 mg, 1.67 mmol) and 2-((3-((tert-butyldimethylsilyl)oxy)propyl)sulfonyl) benzo[d]thiazole (531.2 mg, 2.51 mmol) in THF (15 mL) was added KHMDS (1M) (3.36 mL, 3.36 mmol) at RT and the reaction mixture was stirred for 1 h under N₂ atmosphere. LCMS showed completion of the reaction, the mixture was then diluted with water, extracted with EtOAc (15 mL×3), the organic layers were dried over Na₂SO₄ and concentrated under reduced pressure. The crude product was purified by silica gel column chromatography to afford the compound 6 (760 mg, 90%) as a white solid.

4-(4-Fluoro-6-methoxy-5-((4-methoxybenzyl)oxy)benzo[b]thiophen-2-yl)but-3-en-1-ol (7): To a solution of Tert-butyl((4-(4-fluoro-6-methoxy-5-((4-methoxybenzyl)oxy)benzo[b]thiophen-2-yl)but-3-en-1-yl)oxy)dimethylsilane 6 (670 mg, 1.33 mmol) in THF (13 mL) was added TBAF (1.4 g, 5.33 mmol) at RT and the reaction was stirred overnight. LCMS showed completion of the reaction, the mixture was diluted with water and extracted with EtOAc (15×3 mL). The organic layers were dried over Na₂SO₄ and concentrated under reduced pressure, the crude product was purified by silica gel column chromatography to afford the compound 7 (300 mg, 58%) as yellow oil.

2-(2-(4-Fluoro-6-methoxy-5-((4-methoxybenzyl)oxy)benzo[b]thiophen-2-yl)cyclopropyl)ethan-1-ol (8): To the solution of diazomethane (0.1 M in diethyl ether, 30 mL, 30.8 mmol) was added 4-(4-fluoro-6-methoxy-5-((4-methoxybenzyl)oxy)benzo[b]thiophen-2-yl)but-3-en-1-ol 7 (600 mg, 1.54 mmol) and Pd(OAc)₂ (69.4 mg, 0.31 mmol) in THF (3 mL) at 0° C., the mixture was stirred overnight. LCMS showed the desired product, the reaction mixture was concentrated and purified by reverse phase column to afford the compound 8 (250 mg, 40%).

2-(2-(4-Fluoro-6-methoxy-5-((4-methoxybenzyl)oxy)benzo[b]thiophen-2-yl)cyclopropyl)acetaldehyde (9): To the solution of 2-(2-(4-fluoro-6-methoxy-5-((4-methoxybenzyl)oxy)benzo[b]thiophen-2-yl)cyclopropyl)ethan-1-ol 8 (300 mg, 0.74 mmol) in DCM (12 mL) was added Dess martin periodinane (632 mg, 1.49 mmol) at 0° C. and the reaction was allowed stir at RT for 2 h. The mixture was diluted with water and extracted with EtOAc (15×3 mL), the organic layers were dried over Na₂SO₄ and concentrated under reduced pressure to give the compound 9 as a yellow solid which was used directly without further purification; LCMS (ESI)=m/z 401.2 (M+H).

2-(2-(4-Fluoro-6-methoxy-5-((4-methoxybenzyl)oxy)benzo[b]thiophen-2-yl)cyclopropyl)acetic acid (10): To the solution of 2-(2-(4-fluoro-6-methoxy-5-((4-methoxybenzyl)oxy)benzo[b]thiophen-2-yl)cyclopropyl)acetaldehyde 9 (628 mg, 1.57 mmol) in tBuOH/H₂O (3/1, v/v, 10 mL) was added KH₂PO₄ (258 mg, 1.90 mmol), 2-Methyl-2-butene (166 mg, 2.37 mmol), and NaClO₂ (171 mg, 1.90 mmol) at 0° C. The mixture was stirred overnight at RT. LCMS showed completion of the reaction, the reaction was diluted with water and extracted with EtOAc, organic layers were dried over Na₂SO₄ and concentrated under reduced pressure, the crude compound was purified by silica gel column chromatography to afford the compound 10 (400 mg, 80% purity).

Methyl 2-(2-(4-fluoro-6-methoxy-5-((4-methoxybenzyl)oxy)benzo[b]thiophen-2-yl)cyclopropyl)acetate (11): To the solution of 2-(2-(4-fluoro-6-methoxy-5-((4-methoxybenzyl)oxy)benzo[b]thiophen-2-yl)cyclopropyl)acetic acid 10 (670 mg, 1.61 mmol) in DMF (3 mL) was added CH₃I (342.9 mg, 2.42 mmol) and K₂CO₃ (667.8 mg, 4.83 mmol) and the reaction mixture was stirred at RT overnight. The mixture was diluted with water and extracted with EtOAc, The organic layers were dried over Na₂SO₄, concentrated under reduced pressure and the crude product was purified by silica gel column chromatography to afford the compound 11 (156 mg, 22% yield). ¹H NMR (400 MHz, DMSO-d₆) δ 7.38 (s, 1H), 7.35-7.29 (m, 2H), 6.97 (s, 1H), 6.93-6.86 (m, 2H), 3.86 (s, 3H), 3.74 (s, 4H), 3.62 (s, 3H), 2.04 (dt, J=9.1, 4.9 Hz, 1H), 1.40-1.31 (m, 1H), 1.06 (dt, J=8.9, 5.1 Hz, 1H), 0.97 (dt, J=8.4, 5.3 Hz, 1H).

Methyl 2-(2-(4-fluoro-5-hydroxy-6-methoxybenzo[b]thiophen-2-yl)cyclopropyl)acetate (12): To a solution of methyl 2-(2-(4-fluoro-6-methoxy-5-((4-methoxybenzyl)oxy)benzo[b]thiophen-2-yl)cyclopropyl)acetate 11 (156 mg, 0.36 mmol) in THF (5 mL) was added HCl (1 drop) and Pd/C (20 mg, 10%). The mixture was stirred at 60° C. under H₂ atmosphere overnight. The reaction mixture was cooled and carefully filtered through Celite pad and the filtrate was concentrated under reduced pressure, the crude material was purified by preparative TLC to afford the compound 12 (62 mg, 55% yield) as a yellow solid.

Methyl 4-(4-fluoro-5-(3-((4-fluoro-6-methoxy-2-(2-(2-methoxy-2-oxoethyl)cyclopropyl)benzo[b]thiophen-5-yl)oxy)propoxy)-6-methoxybenzo[b]thiophen-2-yl)-2,2-dimethyl-4-oxobutanoate (13): To a solution of methyl 2-(2-(4-fluoro-5-hydroxy-6-methoxybenzo[b]thiophen-2-yl)cyclopropyl)acetate 12 (52.6 mg, 0.169 mmol) in CH₃CN (2.5 mL) was added methyl 4-(5-(3-bromopropoxy)-4-fluoro-6-methoxybenzo[b]thiophen-2-yl)-2,2-dimethyl-4-oxobutanoate A (93.83 mg, 0.203 mmol) and Cs₂CO₃ (276.12 mg, 0.847 mmol) at 65° C. for 4 h. The reaction mixture was concentrated under reduced pressure and the residue was purified by silica gel column chromatography to afford the desired product 13 (100 mg, 85% yield) as a yellow solid.

4-(5-(3-((2-(2-(carboxymethyl)cyclopropyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid (Compound 12): To a solution of methyl 4-(4-fluoro-5-(3-((4-fluoro-6-methoxy-2-(2-(2-methoxy-2-oxoethyl)cyclopropyl)benzo[b]thiophen-5-yl)oxy)propoxy)-6-methoxybenzo[b]thiophen-2-yl)-2,2-dimethyl-4-oxobutanoate 13 (130 mg, 0.188 mmol) and LiOH (41.96 mg, 1.5 mmol) in THF/MeOH/H₂O (2/2/1, v/v/v, 5 mL) was stirred at 40° C. for 14 h. The reaction was concentrated and purified by silica gel column chromatography to afford the desired product Compound 12 (80 mg, 64% yield) as a yellow solid. ¹H NMR (400 MHz, DMSO-d₆) δ 8.23 (s, 1H), 7.52 (s, 1H), 7.35 (s, 1H), 6.96 (s, 1H), 4.25 (t, J=6.2 Hz, 2H), 4.20 (t, J=6.1 Hz, 2H), 3.86 (s, 3H), 3.79 (s, 3H), 3.45-3.40 (m, 2H), 2.37-2.0 (m, 2H), 2.05 (dp, J=12.0, 5.7 Hz, 3H), 1.35 (s, 1H), 1.23 (s, 6H), 1.08-0.92 (m, 2H). ¹⁹F NMR (376 MHz, DMSO-d₆) δ −134.15, −137.06.

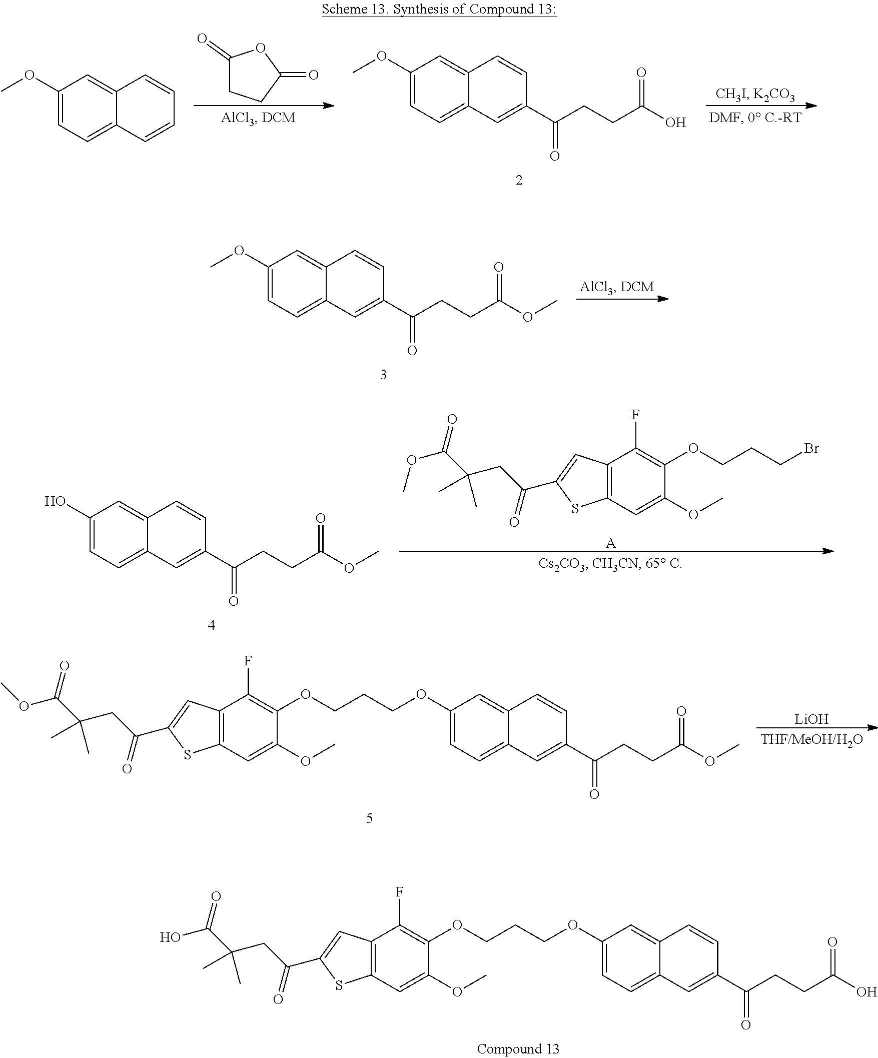

4-(6-Methoxynaphthalen-2-yl)-4-oxobutanoic acid (2): To a solution of 2-methoxynaphthalene (10.0 g. 63.21 mmol) and dihydrofuran-2,5-dione (12.65 g, 126.42 mmol) in DCM (200 mL) was added portion wise AlCl₃ (16.84 g, 126.40 mmol) at 0° C. The mixture was stirred at room temperature overnight. After which, the reaction mixture was carefully quenched with ice cold water (500 mL) and extracted with EtOAc (400 mL×3). The combined organic layer was washed with brine, dried over Na₂SO₄ and concentrated under reduced pressure. The residue was purified by silica gel chromatography to afford the desired product 2 (1.6 g, 10%) as a yellow solid. ¹H NMR (400 MHz, DMSO-d₆) δ 8.65 (s, 1H), 8.07 (d, J=9.0 Hz, 1H), 8.01-7.90 (m, 2H), 7.44 (d, J=2.2 Hz, 1H), 7.29 (dd, J=8.9, 2.4 Hz, 1H), 3.94 (s, 3H), 3.44 (t, J=6.3 Hz, 2H), 2.73 (d, J=12.6 Hz, 2H).

Methyl 4-(6-Methoxynaphthalen-2-yl)-4-oxobutanoate (3): To a solution of 4-fluoro-5, 6-dimethoxybenzo[b]thiophene-2-carboxylic acid 2 (1.6 g, 6.12 mmol) in DMF (30 mL) was added K₂CO₃ (2.11 g, 15.3 mmol) and CH₃I (8.3 g, 58.5 mmol) at ° C. The mixture was stirred at RT for 3 hours. After which, the reaction mixture was diluted with water (300 mL) and extracted with EtOAc (150 mL×3). The combined organic layers were washed with brine, dried over Na₂SO₄ and concentrated under reduced pressure. The residue was purified by silica gel chromatography to afford the desired product 3 (1.54 g, 95%) as a yellow solid. LCMS (ESI)=m/z 273.2 (M+H).

Methyl 4-(6-Hydroxynaphthalen-2-yl)-4-oxobutanoate (4): To a solution of methyl 4-(6-methoxynaphthalen-2-yl)-4-oxobutanoate 3 (125 mg, 0.46 mmol) in DCM (4 mL) was added AlCl₃ (368 mg, 2.74 mmol) at 0° C. The mixture was stirred at 45° C. overnight, TLC showed the starting material was disappeared. The reaction mixture was diluted with water (10 mL) and extracted with EtOAc (20 mL×3). The combined organic layer was washed with brine, dried over Na₂SO₄ and concentrated under reduced pressure. The residue was purified by silica gel chromatography to afford the desired product 4 (25 mg, 21%) as a light yellow solid. ¹H NMR (400 MHz, DMSO-d6) δ 10.20 (s, 1H), 8.57 (s, 1H), 7.99 (d, J=9.6 Hz, 1H), 7.87 (d, J=8.7 Hz, 1H), 7.76 (d, J=8.7 Hz, 1H), 7.17 (dd, J=6.9, 2.3 Hz, 2H), 3.61 (s, 3H), 3.40 (t, J=6.3 Hz, 2H), 2.69 (t, J=6.3 Hz, 2H).

4-(5-(3-((6-(3-Carboxypropanoyl)naphthalen-2-yl)oxy)propoxy)-4-fluoro-6-methoxybenzo[b]thiophen-2-yl)-2,2-dimethyl-4-oxobutanoic acid (5): To a solution of methyl 4-(6-hydroxynaphthalen-2-yl)-4-oxobutanoate 4 (20 mg, 0.07 mmol) and methyl 4-(5-(3-bromopropoxy)-4-fluoro-6-methoxybenzo[b]thiophen-2-yl)-2, 2-dimethyl-4-oxobutanoate A (38 mg, 0.08 mmol) in CH₃CN (40 mL) was added Cs₂CO₃ (126 mg, 0.38 mmol). The mixture was stirred at 65° C. for 2 hours. The reaction was cooled to RT, filtered and concentrated under reduced pressure. The residue was purified by silica gel chromatography to afford the desired product 5 (36 mg, 73%) as a light yellow solid. LCMS (ESI)=m/z 639.1 (M+H).

4-(5-(3-((6-(3-Carboxypropanoyl)naphthalen-2-yl)oxy)propoxy)-4-fluoro-6-methoxybenzo[b]thiophen-2-yl)-2,2-dimethyl-4-oxobutanoic acid (Compound 13): To a solution of methyl 4-(4-fluoro-6-methoxy-5-(3-((6-(4-methoxy-4-oxobutanoyl)naphthalen-2-yl)oxy)propoxy)benzo[b]thiophen-2-yl)-2,2-dimethyl-4-oxobutanoate 5 (30 mg, 0.046 mmol) in THF/MeOH/H₂O (0.5 mL/0.5 ml/0.5 ml) was added LiOH (18 mg, 0.46 mmol) at RT. The reaction mixture was stirred overnight at RT, LCMS showed completion of the reaction. The reaction mixture was adjusted pH to 1 by adding 1M HCl and extracted with EtOAc (5 mL×3). The combined organic layer was dried over Na₂SO₄, and concentrated under reduced pressure. The residue was purified by preparative TLC to afford the desired product Compound 13 (23 mg, 80%) as a white solid. ¹H NMR (400 MHz, DMSO-d6) δ 12.10 (s, 2H), 8.63 (d, J=1.6 Hz, 1H), 8.27 (s, 1H), 8.05 (d, J=9.2 Hz, 1H), 7.96 (d, J=10.0 Hz, 1H), 7.91 (d, J=8.8 Hz, 1H), 7.55 (s, 1H), 7.45 (s, 1H), 7.27 (dd, J=8.8, 2.4 Hz, 1H), 4.37 (t, J=6.0 Hz, 2H), 4.26 (t, J=6.0 Hz, 2H), 3.83 (s, 3H), 3.39-3.34 (m, 4H), 2.63 (t, J=6.2 Hz, 2H), 2.22 (m, 2H), 1.22 (s, 6H). LCMS (ESI)=m/z 609.2 (M+H).

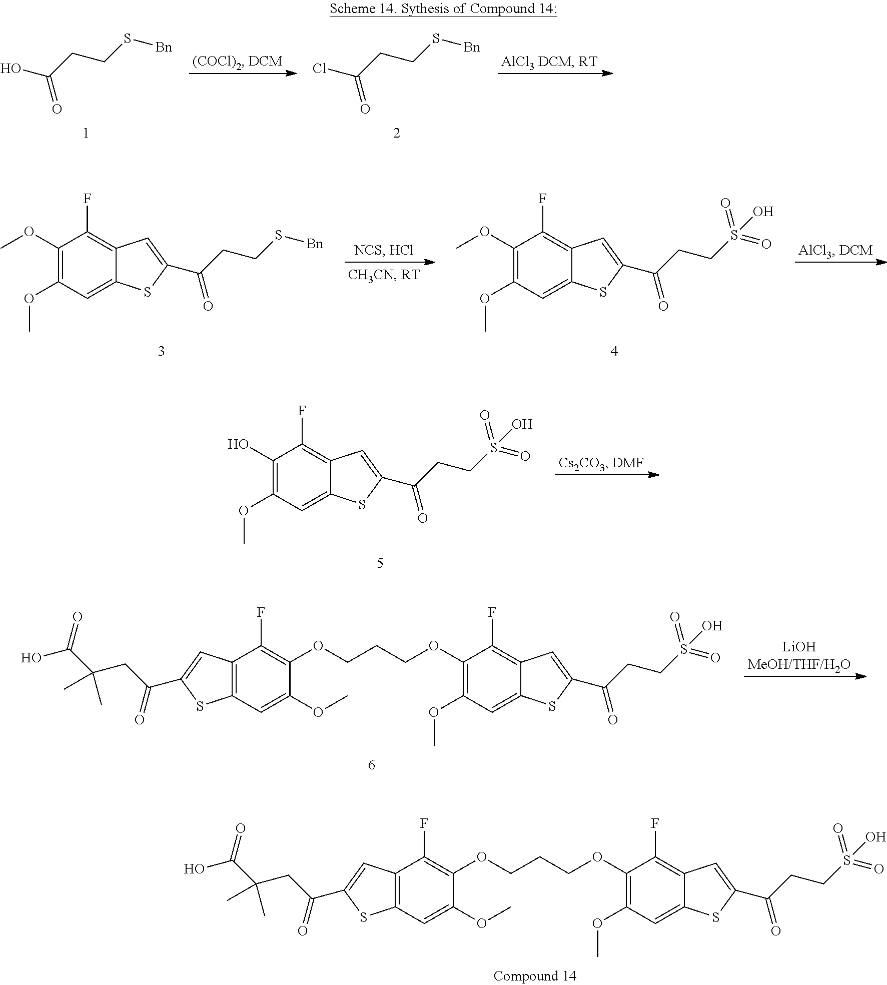

3-(Benzylthio)propanoyl chloride (2): To a solution of 3-(benzylthio)propanoic acid 1 (820 mg, 4.18 mmol) in DCM (35 mL) was added oxalyl chloride (1.6 g, 12.53 mmol) at 0° C. followed by one drop of DMF was added to the reaction mixture. The reaction was stirred at RT for 3 h under nitrogen atmosphere. After which, the mixture concentrated under reduced pressure to afford the desired product 2 as yellow oil which was carried over to the next step.

3-(Benzylthio)-1-(4-fluoro-5,6-dimethoxybenzo[b]thiophen-2-yl)propan-1-one (3): To a solution of 4-fluoro-5,6-dimethoxybenzo[b]thiophene (446 mg, 2.1 mmol) in DCM (12 mL) was added 3-(benzylthio)propanoyl chloride 2 (900 mg, 4.2 mmol) and AlCl₃ (336 mg, 2.52 mmol) at 0° C. under nitrogen atmosphere. The reaction was stirred overnight at RT. The reaction mixture was diluted with water (20 mL) and extracted with DCM (10 mL×3), the combined organic layers were dried over Na₂SO₄ and concentrated under reduced pressure. The residue was purified by silica gel chromatography to afford the desired product 3 (510 mg, 62%) as a yellow solid. ¹H NMR (400 MHz, CDCl3) δ 7.84 (s, 1H), 7.35-7.03 (m, 5H), 7.06 (s, 1H), 3.99 (s, 3H), 3.97 (s, 2H), 3.96 (s, 3H), 3.14 (t, J=7.2 Hz, 2H), 2.85 (t, J=7.2 Hz, 2H).

3-(4-Fluoro-5,6-dimethoxybenzo[b]thiophen-2-yl)-3-oxopropane-1-sulfonic acid (4): To a solution of NCS (684 mg, 5.12 mmol) in CH₃CN (5 mL) was added 1M HCl (2 mL, 2 mmol,), the reaction mixture was stirred at 0° C. for 10 min. Then a solution of 3-(benzylthio)-1-(4-fluoro-5,6-dimethoxybenzo[b]thiophen-2-yl)propan-1-one 3 (800 mg, 2.04 mmol) in 3 mL CH₃CN was added to the reaction and stirred at RT for 1 h. After which, the mixture was directly concentrated under reduced pressure, the crude product was purified by reverse phase column to afford the desired product 4 (445 mg, 62%) as a solid. ¹H NMR (400 MHz, CDCl3) δ 7.96 (s, 1H), 7.08 (s, 1H), 3.99 (s, 3H), 3.96 (s, 3H), 3.93 (t, J=7.2 Hz, 2H), 3.45 (t, J=7.2 Hz, 2H).

3-(4-Fluoro-5-hydroxy-6-methoxybenzo[b]thiophen-2-yl)-3-oxopropane-1-sulfonic acid (5): To a solution of 3-(4-fluoro-5,6-dimethoxybenzo[b]thiophen-2-yl)-3-oxopropane-1-sulfonic acid 4 (90 mg, 0.26 mmol) in DCM (4 mL) was added AlCl₃ (242 mg, 1.8 mmol) at 0° C. Then the mixture was stirred overnight at RT. After which, the reaction mixture was carefully diluted with water (10 mL) and extracted with EtOAc (10 mL×3). The combined organic layers were dried over Na₂SO₄ and concentrated under reduced pressure. The residue was purified by reverse phase column to afford the desired product 5 (47 mg, 89%) as a white solid. LCMS (ESI)=m/z 333.1 (M+H).

3-(4-Fluoro-5-(3-((4-fluoro-6-methoxy-2-(4-methoxy-3,3-dimethyl-4-oxobutanoyl)benzo[b]thiophen-5-yl)oxy)propoxy)-6-methoxybenzo[b]thiophen-2-yl)-3-oxopropane-1-sulfonic acid (6): To a solution of 3-(4-fluoro-5-hydroxy-6-methoxybenzo[b]thiophen-2-yl)-3-oxopropane-1-sulfonic acid 5 (47 mg, 0.14 mmol) in DMF (1 mL) was added Cs₂CO₃ (184 mg, 0.56 mmol) and methyl 4-(5-(3-bromopropoxy)-4-fluoro-6-methoxybenzo[b]thiophen-2-yl)-2,2-dimethyl-4-oxobutanoate A (68 mg, 0.14 mmol) at RT. The mixture was heated to 65° C. and the reaction was stirred for 2 h. The reaction mixture was concentrated under reduced pressure. The residue was purified by preparative TLC (DCM/MeOH=10/1, v/v) to afford the desired product 6 (52 mg, 52%) as a yellow solid. ¹H NMR (400 MHz, DMSO-d₆) δ 8.25 (s, 1H), 8.14 (s, 1H), 7.54 (d, J=7.7 Hz, 2H), 4.27 (t, J=6.1 Hz, 4H), 3.87 (d, J=5.5 Hz, 6H), 3.29 (s, 2H), 2.77 (t, J=7.5 Hz, 2H), 2.08 (q, J=6.2 Hz, 2H), 1.23 (s, 6H).

4-(4-fluoro-5-(3-((4-fluoro-6-methoxy-2-(3-sulfopropanoyl)benzo[b]thiophen-5-yl)oxy)propoxy)-6-methoxybenzo[b]thiophen-2-yl)-2,2-dimethyl-4-oxobutanoic acid (Compound 14): To a solution of 3-(4-fluoro-5-(3-((4-fluoro-6-methoxy-2-(4-methoxy-3,3-dimethyl-4-oxobutanoyl)benzo[b]thiophen-5-yl)oxy)propoxy)-6-methoxybenzo[b]thiophen-2-yl)-3-oxopropane-1-sulfonic acid 6 (45 mg, 0.062 mmol) in THF/MeOH/H₂O (1.5 mL) was added LiOH (4.5 mg, 0.189 mmol) at RT and the reaction was stirred overnight, TLC showed SM disappeared. The mixture was concentrated under reduced pressure. The residue was purified by preparative HPLC to afford the desired product Compound 14 (7 mg, 92%) as a yellow solid. ¹H NMR (400 MHz, DMSO-d₆) δ 8.23 (s, 1H), 8.14 (s, 1H), 7.53 (d, J=2.7 Hz, 2H), 4.27 (t, J=6.1 Hz, 4H), 3.87 (d, J=4.4 Hz, 6H), 3.35 (s, 2H), 3.29 (t, J=7.5 Hz, 2H), 2.77 (t, J=7.5 Hz, 2H), 2.09 (t, J=6.1 Hz, 2H), 1.23 (s, 6H). LCMS (ESI)=m/z 699 (M−H).

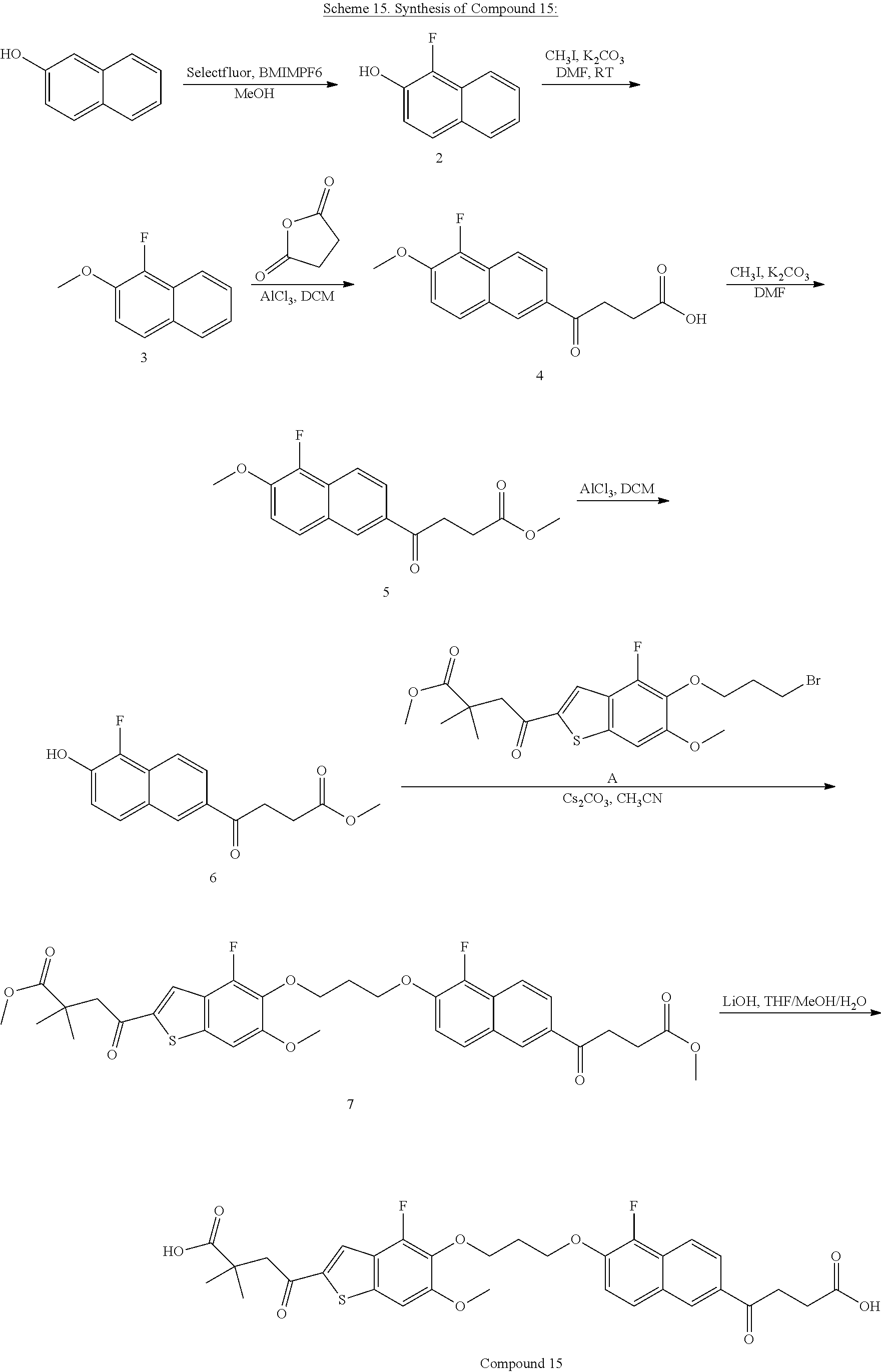

1-Fluoronaphthalen-2-ol (2): To a solution of naphthalen-2-ol (2.0 g, 14 mmol) in MeOH (40 mL) was added Selectfluor (2.7 g, 8 mmol) and BMIMPF6 (13.79 g, 48 mmol) at RT. Then the mixture was stirred at room temperature overnight. After which, the reaction mixture was quenched with ice water (100 mL) and extracted with EtOAc (40 mL×3), the combined organic layer was washed with brine, dried over Na₂SO₄ and concentrated under reduced pressure. The crude compound was purified by silica gel chromatography to afford the desired product 2 (480 mg, 21%) as oil. ¹H NMR (400 MHz, Chloroform-d) δ 7.96 (d, J=8.4 Hz, 1H), 7.78 (d, J=8.4 Hz, 1H), 7.59-7.48 (m, 2H), 7.38 (t, J=7.6 Hz, 1H), 7.23 (t, J=8.6 Hz, 1H).

1-Fluoro-2-methoxynaphthalene (3): To a solution of 1-fluoronaphthalen-2-ol 2 (480 mg, 2.96 mmol) in DMF (8 mL) was added K₂CO₃ (1.02 g, 7.40 mmol) and CH₃I (1.05 g, 7.40 mmol) at 0° C. Then the reaction mixture was stirred at RT under nitrogen atmosphere for 3 hours. The mixture was diluted with water (60 mL) and extracted with EtOAc (30 mL×3). The combined organic layers were washed with brine, dried over Na₂SO₄ and concentrated under reduced pressure. The residue was purified by silica gel chromatography to afford the desired product 3 (390 mg, 74%) as oil. LCMS (ESI)=m/z 177 (M+H).

4-(5-Fluoro-6-methoxynaphthalen-2-yl)-4-oxobutanoic acid (4): To a solution of 1-fluoro-2-methoxynaphthalene 3 (380 mg, 2.16 mmol) and dihydrofuran-2,5-dione (431 mg, 4.31 mmol) in DCM (8 mL) was added AlCl₃ (431 mg, 3.24 mmol) at 0° C. slowly and the mixture was stirred overnight at room temperature. The mixture was quenched with ice cold water (20 mL) and extracted with EtOAc (20 mL×3). The combined organic layer was washed with brine, dried over Na₂SO₄ and concentrated under reduced pressure. The residue was purified by silica gel chromatography to afford the desired product 4 (330 mg, 55%) as a yellow solid. ¹H NMR (400 MHz, Methanol-d₄) δ 8.56 (d, J=1.6 Hz, 1H), 8.06-7.98 (m, 2H), 7.85 (d, J=9.2 Hz, 1H), 7.51 (1, J=8.8 Hz, 1H), 4.03 (s, 3H), 3.42 (t, J=6.4 Hz, 2H), 2.75 (t, J=6.4 Hz, 2H).

Methyl 4-(5-fluoro-6-methoxynaphthalen-2-yl)-4-oxobutanoate (5): To a solution of 4-(5-fluoro-6-methoxynaphthalen-2-yl)-4-oxobutanoic acid 4 (300 mg, 1.09 mmol) in DMF (3 mL) was added K₂CO₃ (450 mg, 3.26 mmol) and CH₃I (231 mg, 1.63 mmol) at 0° C. Then the mixture was stirred at room temperature under nitrogen atmosphere for 3 hours. The mixture was diluted with water (30 mL) and extracted with EtOAc (15 mL×3). The combined organic layer was washed with brine, dried over Na₂SO₄ and concentrated under reduced pressure. The residue was purified by silica gel chromatography to afford the desired product (300 mg, 95%) as oil. LCMS (ESI)=m/z 291 (M+H).

Methyl 4-(5-fluoro-6-hydroxynaphthalen-2-yl)-4-oxobutanoate (6): To a suspension of AlCl₃ (326 mg, 2.5 mmol) in DCM (1 mL) was added methyl 4-(5-fluoro-6-methoxynaphthalen-2-yl)-4-oxobutanoate 5 (71 mg, 0.25 mmol) at 0° C. The reaction mixture was warmed to ambient temperature and stirred for 16 hours under nitrogen atmosphere. After which, the mixture was quenched with water (10 mL), adjusted pH to 2 by adding 1M HCl, and then extracted with EtOAc (6 mL×3). The combined organic layer was washed with brine, dried over Na₂SO₄ and concentrated under reduced pressure. The residue was purified by silica gel chromatography to afford the desired product 6 (51 mg, 72%) as a light yellow solid. LCMS (ESI)=m/z 275.1 (M+H).

Methyl 4-(4-fluoro-5-(3-((1-fluoro-6-(4-methoxy-4-oxobutanoyl)naphthalen-2-yl)oxy)propoxy)-6-methoxybenzo[b]thiophen-2-yl)-2,2-dimethyl-4-oxobutanoate (7): To a solution of methyl 4-(5-fluoro-6-hydroxynaphthalen-2-yl)-4-oxobutanoate 6 (35 mg, 0.11 mmol) and methyl 4-(5-(3-bromopropoxy)-4-fluoro-6-methoxybenzo[b]thiophen-2-yl)-2, 2-dimethyl-4-oxobutanoate A (51 mg, 0.12 mmol) in CH₃CN (40 mL) was added Cs₂CO₃ (92 mg, 0.38 mmol), the mixture was stirred at 65° C. under nitrogen atmosphere for 2 hours. The mixture was cooled to RT, filtered and concentrated under reduced pressure. The residue was purified by silica gel chromatography to afford the desired product 7 (52 mg, 62%) as a light yellow solid. LCMS (ESI)=m/z 657.2 (M+H).

4-(5-(3-((6-(3-Carboxypropanoyl)-1-fluoronaphthalen-2-yl)oxy)propoxy)-4-fluoro-6-methoxybenzo[b]thiophen-2-yl)-2,2-dimethyl-4-oxobutanoic acid (Compound 15): To a solution of methyl 4-(4-fluoro-5-(3-((1-fluoro-6-(4-methoxy-4-oxobutanoyl)naphthalen-2-yl)oxy)propoxy)-6-methoxybenzo[b]thiophen-2-yl)-2,2-dimethyl-4-oxobutanoate 7 (25 mg, 0.038 mmol) in THF/MeOH/H₂O (0.5 mL/0.5 ml/0.5 ml) was added LiOH (10 mg, 0.38 mmol). The mixture was stirred overnight at RT. The mixture was adjusted pH to 1 by adding 1M HCl and extracted with EtOAc (5 mL×3). The combined organic layer was dried over Na₂SO₄, and concentrated under reduced pressure, the crude product was purified by preparative TLC to afford the desired product Compound 15 (18 mg, 75%) as a white solid. LCMS (ESI)=m/z 627.2 (M+H); ¹H NMR (400 MHz, DMSO-d₆) δ 12.11 (s, 2H), 8.73 (s, 1H), 8.25 (s, 1H), 8.07-7.97 (m, 3H), 7.68 (d, J=9.2 Hz, 1H), 7.53 (s, 1H), 4.49 (t, J=6.0 Hz, 2H), 4.26 (t, J=6.0 Hz, 2H), 3.83 (s, 3H), 3.38 (m, 4H), 2.67-2.60 (m, 2H), 2.25-2.17 (m, 2H), 1.23 (s, 6H).

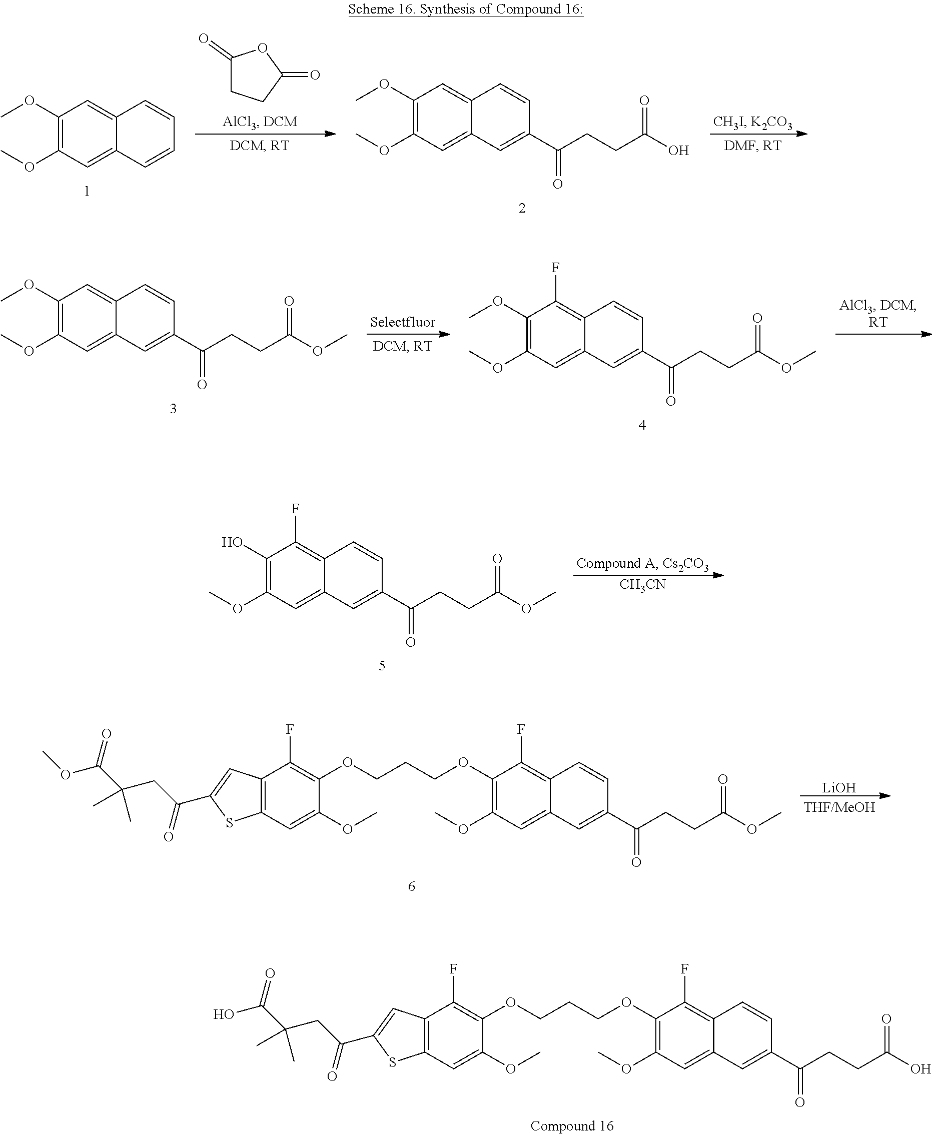

4-(6,7-Dimethoxynaphthalen-2-yl)-4-oxobutanoic acid (2): To a solution of dihydrofuran-2,5-dione (3.2 g, 31.8 mmol, 2.0 eq.) in DCM (40 mL) was added AlCl₃ (6.4 g, 47.8 mmol) and stirred at 0° C. for 10 min, then 2,3-dimethoxynaphthalene (3.0 g, 15.9 mmol) in DCM (15 mL) was added into the suspension slowly. The reaction mixture was stirred at RT for 14 h. The reaction mixture was quenched with water (60 mL), and extracted with CHCl₃/IPA (3/1, v/v, 40×3 mL). The combined organic phase was concentrated, and the residue was purified by silica gel column chromatography to afford the desired product 2 (2.8 g, 60% yield) as a deep yellow solid. ¹H NMR (400 MHz, Chloroform-d) 68.34 (d, J=1.8 Hz, 1H), 7.87 (dd, J=8.6, 1.8 Hz, 1H), 7.69 (d, J=8.5 Hz, 1H), 7.20 (s, 1H), 7.11 (s, 1H), 3.99 (s, 3H), 3.99 (s, 3H), 3.39 (t, J=6.7 Hz, 2H), 2.78 (t, J=6.7 Hz, 2H).

Methyl 4-(6, 7-dimethoxynaphthalen-2-yl)-4-oxobutanoate (3): To a solution of 4-(6, 7-dimethoxynaphthalen-2-yl)-4-oxobutanoic acid 2 (2.8 g, 10.40 mmol) in DMF (40 mL) was added K₂CO₃ (3.5 g, 26 mmol) and CH₃I (2.2 g, 15.6 mmol) at 0° C. The mixture was stirred at RT for 12 h. LCMS showed completion of the reaction. The mixture was extracted with EtOAc (40×3 mL), and the organic layer was dried over Na₂SO₄, and concentrated under reduced pressure and the crude product was purified by silica gel column chromatography to afford the compound 3 (2.7 g, 93%) as a yellow solid.

Methyl 4-(5-fluoro-6, 7-dimethoxynaphthalen-2-yl)-4-oxobutanoate (4): To a solution of methyl 4-(6, 7-dimethoxynaphthalen-2-yl)-4-oxobutanoate 3 (1 g, 3.3 mmol) in DMF (8 mL) was added Selectfluor (1.7 g, 4.96 mmol) at RT. The mixture was stirred at 65° C. for 2 h. The reaction mixture was cooled to RT and quenched with water (80 mL), extracted with EtOAc (20×3 mL), and the combined organic layer was dried over Na₂SO₄, concentrated under reduced pressure and the crude material was purified by silica gel column chromatography to afford the compound 4 (116 mg, 11%) as a yellow solid. ¹H NMR (400 MHz, Chloroform-d) 68.37 (t, J=1.8 Hz, 1H), 8.01 (d, J=8.7 Hz, 1H), 7.94 (dd, J=8.8, 1.6 Hz, 1H), 7.08 (d, J=1.6 Hz, 1H), 4.12 (d, J=2.0 Hz, 3H), 4.01 (s, 3H), 3.73 (s, 4H), 3.43 (t, J=6.7 Hz, 2H), 2.82 (t, J=6.7 Hz, 3H); ¹⁹F NMR (376 MHz, Chloroform-d) δ −141.86.

Methyl 4-(5-fluoro-6-hydroxy-7-methoxynaphthalen-2-yl)-4-oxobutanoate (5): The mixture of methyl 4-(5-fluoro-6, 7-dimethoxynaphthalen-2-yl)-4-oxobutanoate 4 (110 mg, 0.34 mmol) and AlCl₃ (155.9 g, 1.03 mmol) in DCM (5 mL) was stirred at 25° C. for 12 h. LCMS showed completion of the reaction, the reaction mixture was quenched with ice water (20 mL) and was extracted with CHCl₃/IPA (3/1, v/v, 15 mL×3). The combined organic phase was concentrated and purified by silica gel column chromatography to afford the desired product 5 (88 mg, 83% yield) as a white solid. ¹H NMR (400 MHz, Chloroform-d) 68.38 (s, 1H), 8.02-7.93 (m, 2H), 7.08 (s, 1H), 5.89 (s, 1H), 4.07 (s, 3H), 3.73 (s, 3H), 3.43 (t, J=6.7 Hz, 2H), 2.82 (t, J=6.7 Hz, 2H).

Methyl 4-(4-fluoro-5-(3-((1-fluoro-3-methoxy-6-(4-methoxy-4-oxobutanoyl)naphthalen-2-yl)oxy)propoxy)-6-methoxybenzo[b]thiophen-2-yl)-2,2-dimethyl-4-oxobutanoate (6): The mixture of methyl 4-(5-fluoro-6-hydroxy-7-methoxynaphthalen-2-yl)-4-oxobutanoate 5 (35 mg, 0.081 mmol, 1.0 eq.), methyl 4-(5-(3-bromopropoxy)-4-fluoro-6-methoxybenzo[b]thiophen-2-yl)-2,2-dimethyl-4-oxobutanoate A (41.4 mg, 0.089 mmol) and Cs₂CO₃ (85.7 mg, 0.24 mmol) in CH₃CN (6 mL) was stirred at 65° C. for 4 h. LCMS showed completion of the reaction, the solvent was removed under reduced pressure and the residue was purified by silica gel column chromatography to afford the desired product 6 (25 mg, 44% yield) as a white solid. ¹H NMR (400 MHz, Chloroform-d) δ 8.36 (s, 11H), 8.00-7.92 (m, 2H), 7.88 (s, 1H), 7.05 (s, 1H), 7.01 (s, 1H), 4.52 (t, J=6.1 Hz, 2H), 4.40 (t, J=6.1 Hz, 2H), 3.94 (s, 3H), 3.86 (s, 3H), 3.73 (s, 3H), 3.70 (s, 3H), 3.43 (t, J=6.7 Hz, 2H), 3.28 (s, 2H), 2.83 (t, J=6.6 Hz, 2H), 2.28 (t, J=6.1 Hz, 2H), 1.35 (s, 6H).

4-(5-(3-((6-(3-Carboxypropanoyl)-1-fluoro-3-methoxynaphthalen-2-yl)oxy)propoxy)-4-fluoro-6-methoxybenzo[b]thiophen-2-yl)-2,2-dimethyl-4-oxobutanoic acid (Compound 16): To a solution of compound methyl 4-(4-fluoro-5-(3-((1-fluoro-3-methoxy-6-(4-methoxy-4-oxobutanoyl)naphthalen-2-yl)oxy)propoxy)-6-methoxybenzo[b]thiophen-2-yl)-2,2-dimethyl-4-oxobutanoate 6 (35 mg, 0.051 mmol) and LiOH (12.2 mg, 0.305 mmol) in THF/MeOH/H₂O (2/2/1, v/v/v, 5 mL) was stirred at 40° C. for 14 h. LCMS showed completion of the reaction. The reaction mixture was acidified by adding 1N HCl to pH˜3, extracted with EtOAc (15×3 mL). The combined organic phase was concentrated under reduced pressure to afford the desired product Compound 16 (28 mg, 82% yield) as a yellow solid. ¹H NMR (400 MHz, DMSO-d6) δ 12.13 (s, 2H), 8.61 (s, 1H), 8.23 (s, 1H), 8.02-7.85 (m, 2H), 7.51 (s, 2H), 4.41 (t, J=6.1 Hz, 2H), 4.29 (t, J=6.1 Hz, 2H), 3.91 (s, 3H), 3.84 (s, 3H), 3.37 (s, 2H), 2.64 (t, J=6.3 Hz, 2H), 2.13 (d, J=6.1 Hz, 2H), 1.22 (s, 6H).

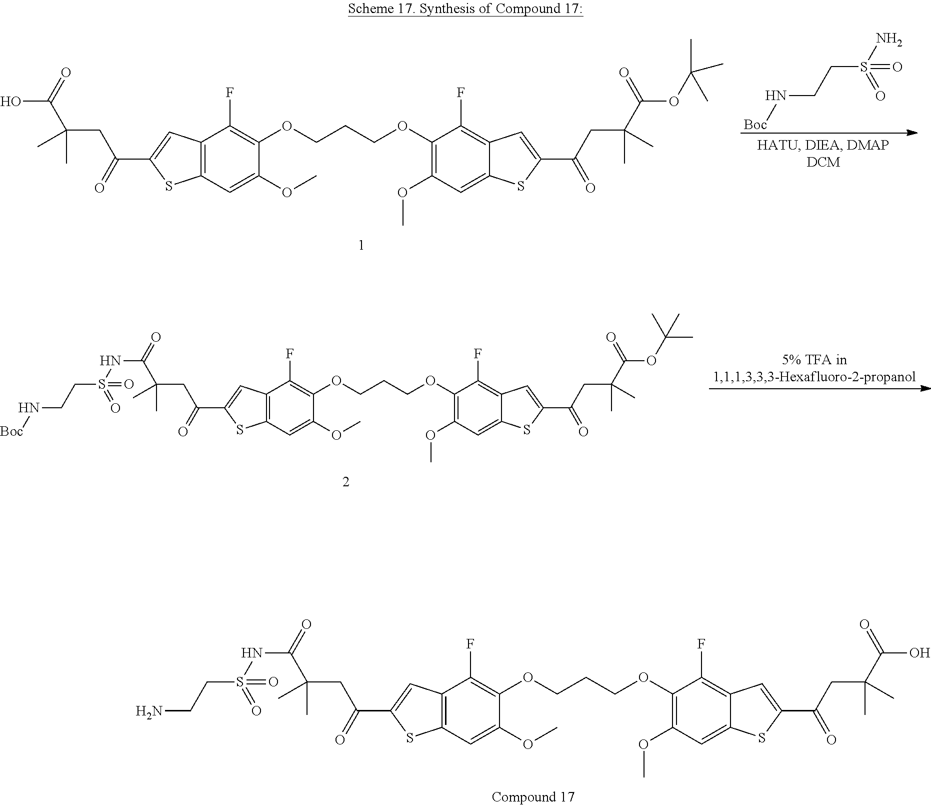 [SMILES: COc1cc2sc(C(=O)CC(C)(C)C(=O)O)cc2c(F)c1OCCCOc1c(OC)cc2sc(C(=O)CC(C)(C)C(=O)OC(C)(C)C)cc2c1F ; CC(C)(C)OC(=O)NCCS(N)(=O)=O ; COc1cc2sc(C(=O)CC(C)(C)C(=O)NS(=O)(=O)CCNC(=O)OC(C)(C)C)cc2c(F)c1OCCCOc1c(OC)cc2sc(C(=O)CC(C)(C)C(=O)OC(C)(C)C)cc2c1F ; COc1cc2sc(C(=O)CC(C)(C)C(=O)O)cc2c(F)c1OCCCOc1c(OC)cc2sc(C(=O)CC(C)(C)C(=O)NS(=O)(=O)CCN)cc2c1F]

Tert-butyl 4-(5-(3-((2-(4-((2-((tert-butoxycarbonyl)amino)ethyl)sulfonamido)-3,3-dimethyl-4-oxobutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoate: To a mixture of 4-(5-(3-((2-(4-(tert-butoxy)-3,3-dimethyl-4-oxobutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid 1 (77 mg, 0.1 mmol), tert-butyl N-(2-sulfamoylethyl)carbamate (46 mg, 0.2 mmol), DIEA (66 mg, 0.51 mmol, 0.09 mL) in dichloromethane (5 mL) was added sequentially HATU (58.6 mg, 0.15 mmol) and DMAP (12.6 mg, 0.10 mmol). After 2 h, DBU (23.5 mg, 0.15 mmol, 0.02 mL) was added. The reaction mixture was stirred at room temperature for 21 h. LCMS showed completion of the reaction, the reaction diluted with water, and the organic layer was extracted DCM and the organics were concentrated under reduced pressure. The residue was purified by silica chromatography to give the compound 2 (50 mg, 51%). LCMS (ESI)=m/z 955.2 (M+H), 977.2 (M+Na).

4-(5-(3-((2-(4-((2-Aminoethyl)sulfonamido)-3,3-dimethyl-4-oxobutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid (Compound 17): A mixture of compound 2 (37 mg, 0.04 mmol) in 5% TFA in 1,1,1,3,3,3-Hexafluoro-2-propanol (3 mL) was stirred at RT for 0.5 h, and mixture was concentrated. LCMS showed completion of the reaction, to the residue was added MTBE and the suspension was re-concentrated. Repeated this process twice. The residue was purified by reverse phase HPLC; C-18 column, 10 to 80% acetonitrile in water with 0.1% formic acid over 30 min. The fractions were combined and freeze dried to afford 4-(5-(3-((2-(4-((2-aminoethyl)sulfonamido)-3,3-dimethyl-4-oxobutanoyl)-4-fluoro-6-methoxybenzo[b]thiophen-5-yl)oxy)propoxy)-4-fluoro-6-methoxybenzo[b]thiophen-2-yl)-2,2-dimethyl-4-oxobutanoic acid Compound 17 as a white powder (20.2 mg, 33%); ¹H NMR (400 MHz, DMSO-d₆) δ 8.23 (s, 1H), 8.14 (s, 1H), 7.52 (d, J=15.7 Hz, 2H), 6.70 (s, 1H), 4.31-4.23 (m, 4H), 3.85 (d, J=6.9 Hz, 6H), 3.41 (d, J=72.6 Hz, 2H), 3.21-3.06 (m, 4H), 3.00 (t, J=6.6 Hz, 2H), 2.08 (p, J=6.0 Hz, 2H), 1.21 (s, 6H), 1.15 (s, 6H). LCMS (ESI)=m/z=799.2 (M+H).

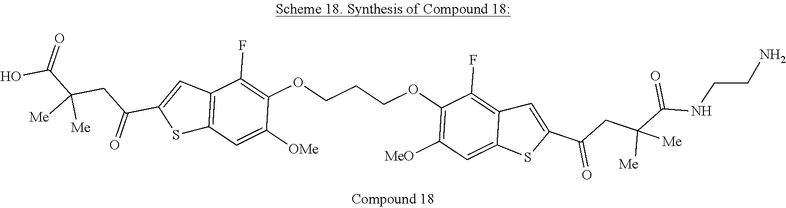 [SMILES: COc1cc2sc(C(=O)CC(C)(C)C(=O)O)cc2c(F)c1OCCCOc1c(OC)cc2sc(C(=O)CC(C)(C)C(=O)NCCN)cc2c1F]

Compound 18 was synthesized in an analogous fashion using the same methods as described above.

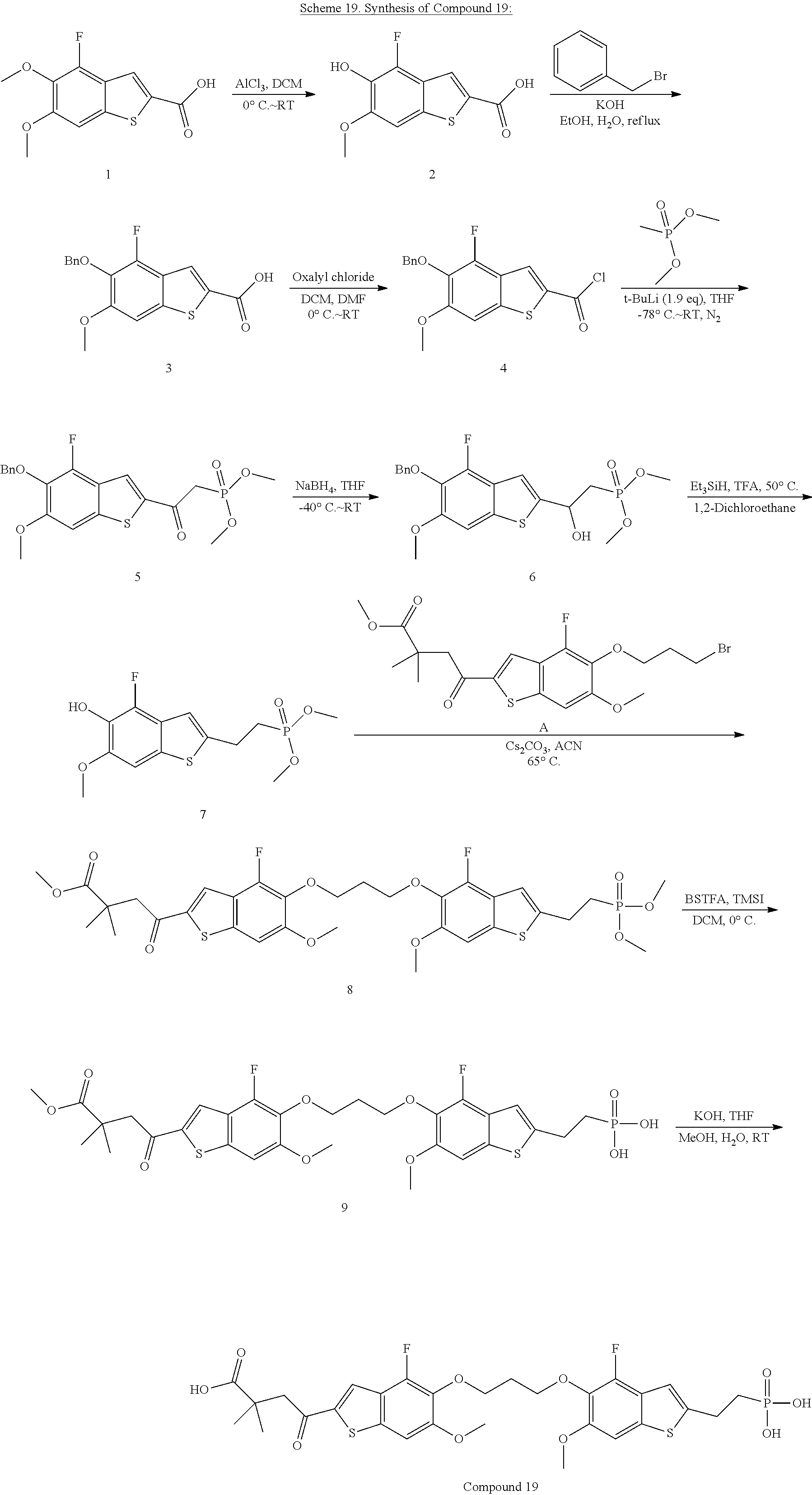

Compound 19 was synthesized using the same methods as described above.

Compound 20, Compound 21, Compound 22, Compound 24, Compound 25, Compound 26, Compound 27, Compound 28, Compound 29, Compound 30, and Compound 31 were synthesized using similar methods as above.

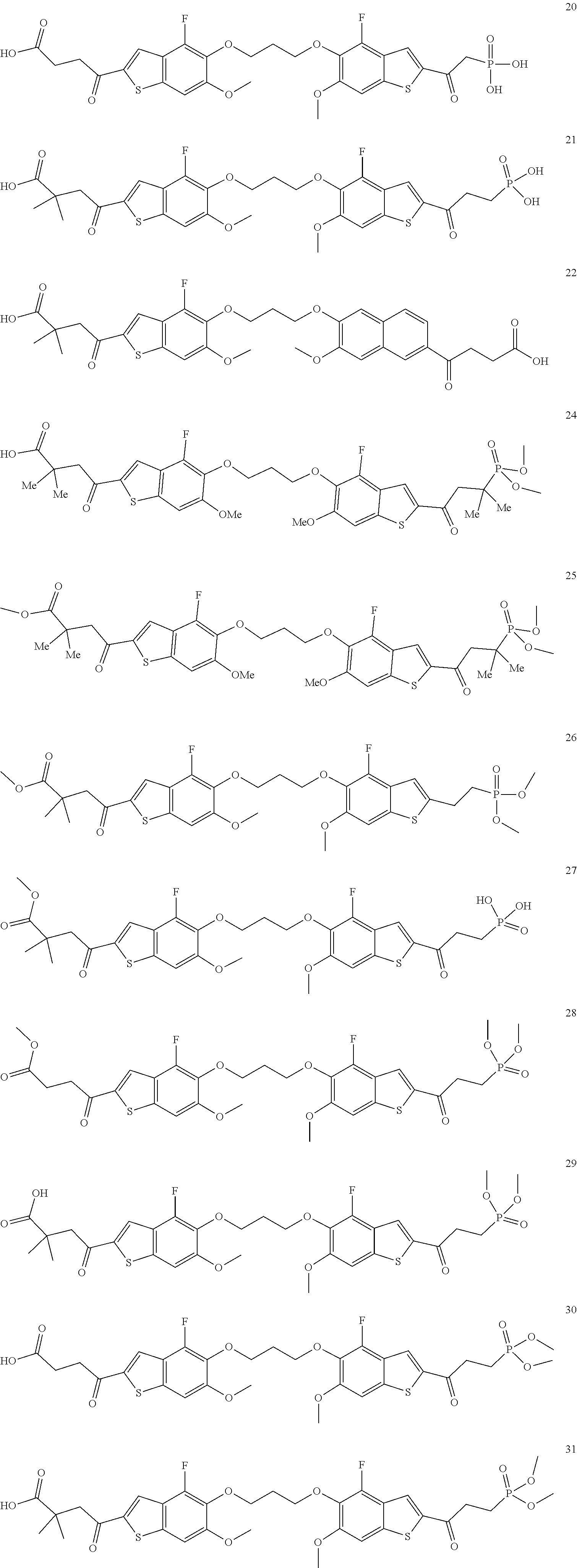

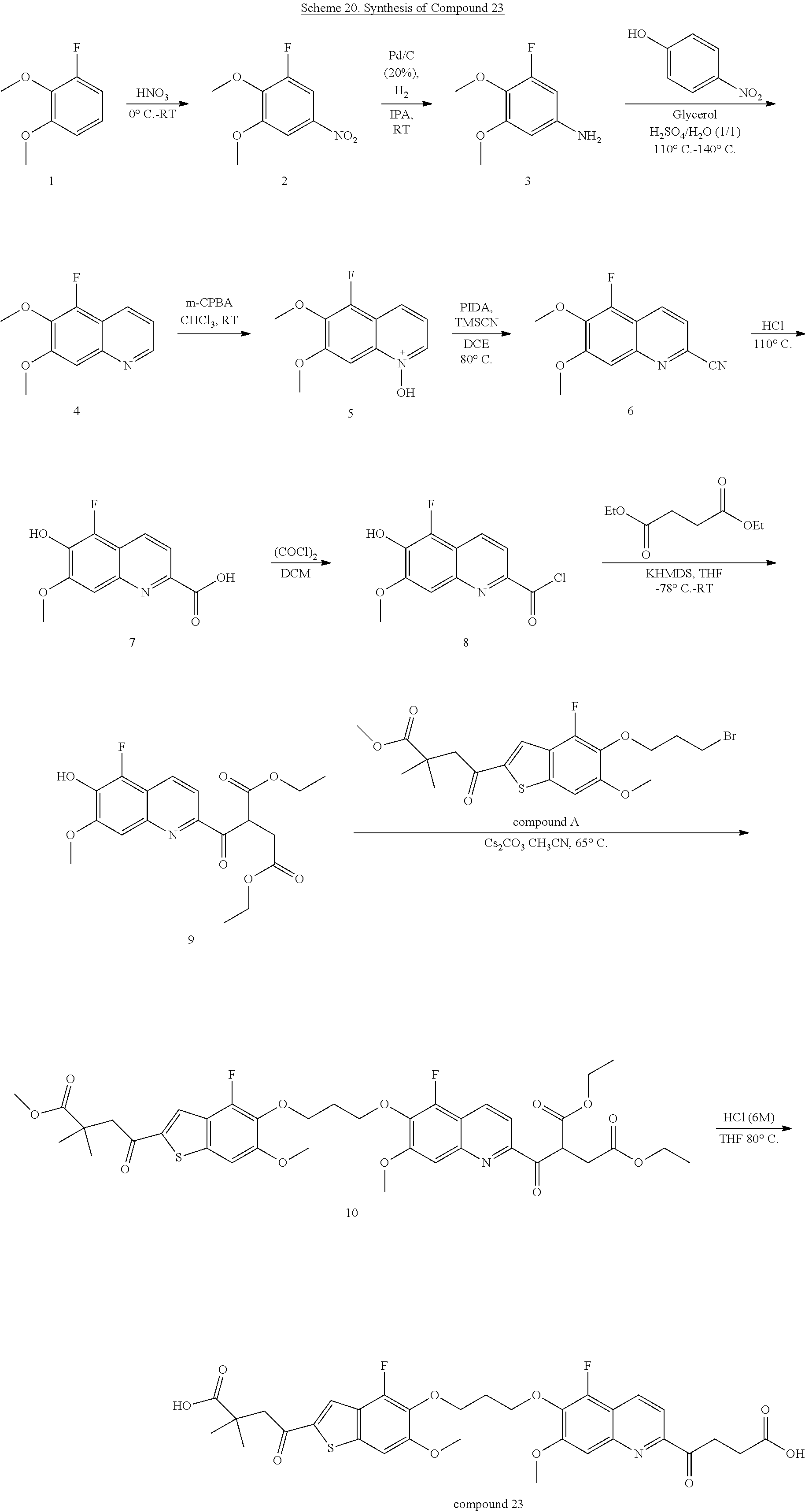

1-Fluoro-2,3-dimethoxy-5-nitrobenzene (2): The solution of 1-fluoro-2, 3-dimethoxybenzene (12 g, 76.84 mmol, 1.0 eq.) dissolved in 120 mL HNO₃ at 0° C. was stirred at RT for 30 mins. TLC showed the starting material was consumed. The reaction mixture was poured into water (300 mL), extracted with EtOAc (150 mL×3). Then the combined organic phases were washed with aqNaHCO₃, dried over Na₂SO₄ and concentrated and the crude product which was purified by silica gel chromatography to afford the desired product 2 (11 g, 55%) as a white solid. ¹H NMR (400 MHz, DMSO-d₆) δ 7.87-7.84 (m, 1H), 7.73-7.12 (m, 1H), 3.95 (s, 6H).

3-Fluoro-4,5-dimethoxyaniline (3): To a solution of 1-fluoro-2,3-dimethoxy-5-nitrobenzene 2 (11 g, 54.68 mmol, 1.0 eq.) in IPA (400 mL) was added Pd/C (20%, lg). Then the mixture was stirred at RT under hydrogen atmosphere overnight. TLC showed starting material was consumed. The mixture was filtered, and the filtrate was concentrated under reduced pressure to afford the desired product 3 (9.3 g, 98%) as a white solid. ¹H NMR (400 MHz, DMSO-d₆) δ 6.05 (s, 1H), 5.95 (dd, J=12.8, 2.4 Hz, 1H), 5.11 (s, 2H), 3.70 (s, 3H), 3.60 (s, 3H).

5-Fluoro-6,7-dimethoxyquinoline (4): To a solution of 3-fluoro-4,5-dimethoxyaniline 3 (7.3 g, 42.59 mmol, 1.0 eq.) dissolved in H₂O (20 mL) and concentrated H₂SO₄ (18 mL), to which was added a mixture of 4-nitrophenol (6.5 g, 46.80 mmol) and glycerol (23.5 g, 255.6 mmol). Then the reaction mixture was stirred at 140° C. for 4 hours. The mixture was cooled to room temperature, poured into ice cold water. The mixture was adjusted to pH 8 with aqNaHCO₃, extracted with EtOAc (100 mL×3). The combined organic phase was dried over Na₂SO₄ and concentrated and the crude product which was purified by silica gel chromatography to afford the desired product 4 (6.76 g, 76%) as a white solid. ¹H NMR (400 MHz, Chloroform-d) δ 8.80 (dd, J=4.4, 1.6 Hz, 1H), 8.28 (dd, J=8.4, 1.6 Hz, 1H), 7.32 (dd, J=8.4, 4.4 Hz, 1H), 7.28 (d, J=1.7 Hz, 1H), 4.09 (s, 3H), 4.02 (s, 3H).

5-Fluoro-1-hydroxy-6,7-dimethoxyquinolin-1-ium (5): To a solution of 5-fluoro-6,7-dimethoxyquinoline 4 (8.4 g, 40 mmol, 1.0 eq.) dissolved in DCM (130 mL) was added a solution of MCPBA (14 g, 80 mmol) at 0° C. Then the mixture was stirred at RT overnight. The reaction mixture was poured into water, extracted with EtOAc (300 mL×3). The combined organic phase was dried over Na₂SO₄ and concentrated under reduced pressure to give the crude product 5 (9 g, 96%) as a white solid; LCMS [M+1]+=224.1

5-Fluoro-6,7-dimethoxyquinoline-2-carbonitrile (6): To a solution of 5-fluoro-1-hydroxy-6,7-dimethoxyquinolin-1-ium 5 (11.4 g, 51.07 mmol, 1.0 eq.) dissolved in DCE (300 mL) was added a mixture of TMSCN (66 g, 204 mmol) and (Diacetoxyiodo)benzene (20 g, 204 mmol) at RT. Then the mixture was stirred at RT overnight. The reaction mixture was concentrated under reduced pressure, the crude product was purified by silica gel chromatography to afford the desired product 6 (5.2 g, 44%) as a white solid. ¹H NMR (400 MHz, DMSO-d₆) δ 8.56 (d, J=8.4 Hz, 1H), 7.95 (d, J=8.4 Hz, 1H), 7.48 (s, 1H), 4.02 (s, 6H).

5-Fluoro-6-hydroxy-7-methoxyquinoline-2-carboxylic acid (7): To a solution of 5-fluoro-6,7-dimethoxyquinoline-2-carbonitrile 6 (1 g, 4.31 mmol, 1.0 eq.) dissolved in concentrated HCl (10 mL) and the mixture was stirred overnight at 110° C. The reaction mixture was concentrated under reduced pressure, the crude product was purified by silica gel chromatography to afford the desired product 7 (820 mg, 80%) as a white solid. ¹H NMR (400 MHz, DMSO-d₆) δ 8.41 (d, J=8.8 Hz, 1H), 8.01 (d, J=8.8 Hz, 1H), 7.45 (s, 1H), 4.02 (s, 3H).

5-Fluoro-6-hydroxy-7-methoxyquinoline-2-carbonyl chloride (8): To a solution of 5-fluoro-6-hydroxy-7-methoxyquinoline-2-carboxylic acid 7 (800 mg, 3.37 mmol, 1.0 eq.) dissolved in anhydrous DCM was added a solution of oxalyl chloride (1.29 g, 10 mmol) at 0° C. Then one drop of DMF was added and the reaction was stirred at RT for 3 hrs. The reaction mixture was concentrated under vacuum to give the crude product 8 which was used directly. LCMS [M+1]⁺=252.0

Diethyl 2-(5-fluoro-6-hydroxy-7-methoxyquinoline-2-carbonyl)succinate (9): To a solution of diethyl succinate (554 mg, 6.36 mmol, 1.0 eq.) dissolved in anhydrous THF (15 mL) was added a solution of KHMDS (5.8 ml, 5.72 mmol) dropwise at −78° C. and stirred at that temperature for 30 mins. Then a solution of 5-fluoro-6-hydroxy-7-methoxyquinoline-2-carbonyl chloride 8 (814 mg, 3.37 mmol, 1.0 eq.) in THF (5 mL) was added. The mixture was warmed to RT and stirred overnight. The reaction was quenched with aq NH₄Cl (20 mL) at 0° C. and extracted with EA (300 mL×3). The combined organic phase was dried over Na₂SO₄ and concentrated, the crude product was purified by silica gel chromatography to afford the desired product 9 (720 mg, 54%) as a white solid. ¹H NMR (400 MHz, Chloroform-d) δ 8.39 (d, J=8.8 Hz, 1H), 8.05 (d, J=8.8 Hz, 1H), 7.33 (s, 1H), 5.53 (t, J=7.2 Hz, 1H), 4.06 (s, 3H), 3.70 (s, 3H), 3.66 (s, 3H), 3.11-3.03 (m, 2H).

Diethyl 2-(5-fluoro-6-(3-((4-fluoro-6-methoxy-2-(4-methoxy-3,3-dimethyl-4-oxobutanoyl)benzo[b]thiophen-5-yl)oxy)propoxy)-7-methoxyquinoline-2-carbonyl)succinate (10): To a solution of diethyl 2-(5-fluoro-6-hydroxy-7-methoxyquinoline-2-carbonyl)succinate 9 (300 mg, 0.76 mmol, 1.0 eq.) and methyl 4-(5-(3-bromopropoxy)-4-fluoro-6-methoxybenzo[b]thiophen-2-yl)-2, 2-dimethyl-4-oxobutanoate Compound A (352 mg, 0.76 mmol) in CH₃CN (10 mL) was added Cs₂CO₃ (994 mg, 3.05 mmol). The mixture was stirred at 65° C. under nitrogen atmosphere for 2 hours. LCMS showed completion of the reaction. The mixture was filtered and concentrated under reduced pressure. The residue was purified by silica gel chromatography (DCM/MeOH=80/1, v/v) to afford the desired product 10 (420 mg, 71%) as a light-yellow solid; LCMS [M+1]⁺=774.1

4-(5-(3-((2-(3-Carboxypropanoyl)-5-fluoro-7-methoxyquinolin-6-yl)oxy)propoxy)-4-fluoro-6-methoxybenzo[b]thiophen-2-yl)-2,2-dimethyl-4-oxobutanoic acid (Compound 23): To a solution of diethyl 2-(5-fluoro-6-(3-((4-fluoro-6-methoxy-2-(4-methoxy-3,3-dimethyl-4-oxobutanoyl)benzo[b]thiophen-5-yl)oxy)propoxy)-7-methoxyquinoline-2-carbonyl)succinate 10 (80 mg, 0.1 mmol, 1.0 eq.) in THF (1 mL) was added 6M HCl (1 mL) at rt. Then the mixture was stirred overnight at 80° C. LCMS showed completion of the reaction. The mixture was concentrated, the crude product was purified by silica gel chromatography (DCM/MeOH=10/1) to afford the desired product Compound 23 (20 mg, 30%) as a light-yellow solid; ¹H NMR (400 MHz, Chloroform-d) δ 8.27 (d, J=8.8 Hz, 1H), 7.92 (d, J=8.8 Hz, 1H), 7.82 (s, 1H), 7.26 (s, 1H), 6.97 (s, 1H) 4.47 (t, J=6.0 Hz, 2H), 4.31 (t, J=6.0 Hz, 2H), 3.92 (s, 3H), 3.79 (s, 3H), 3.59 (s, 2H), 3.21 (s, 2H), 2.72 (t, J=6.7 Hz, 2H), 2.19-2.16 (m, 2H), 1.27 (s, 6H).

The following compounds are synthesized according to the methods described above.

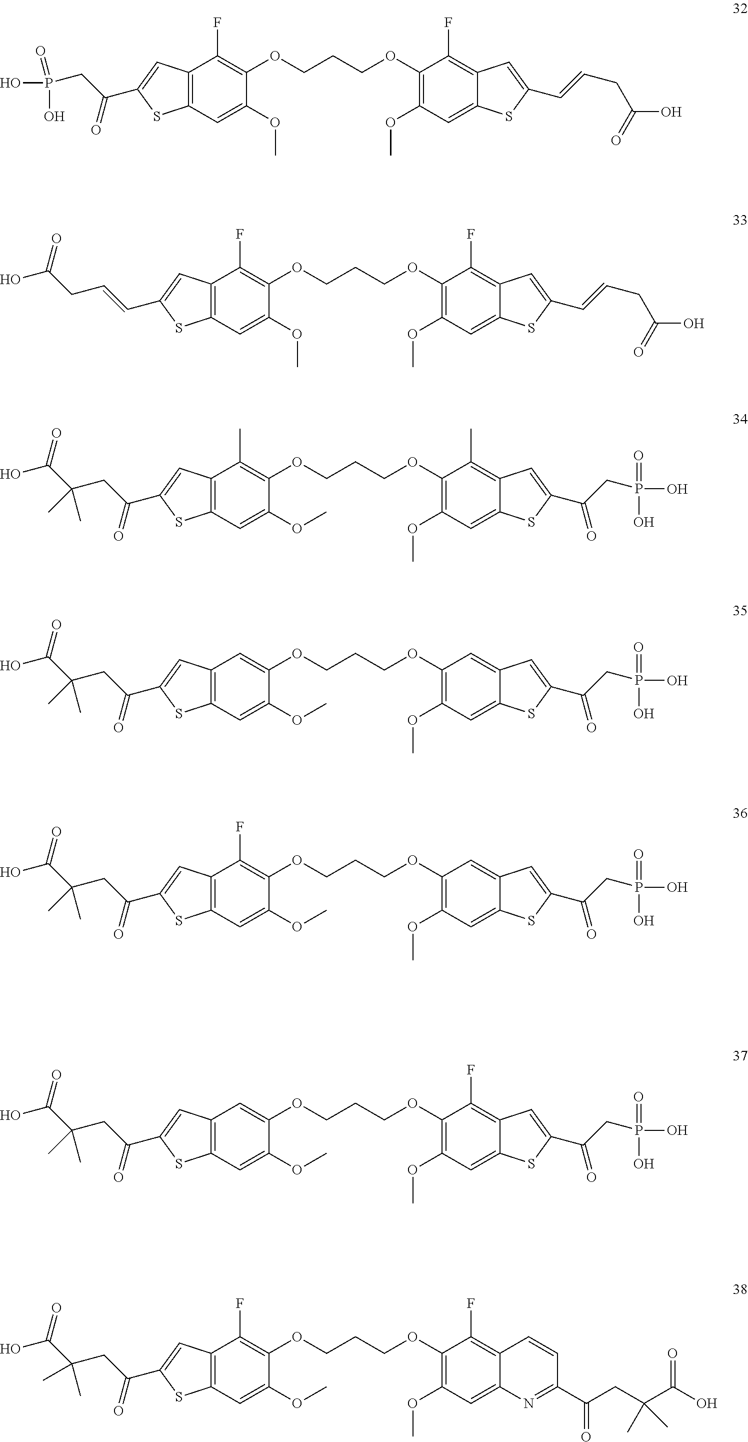

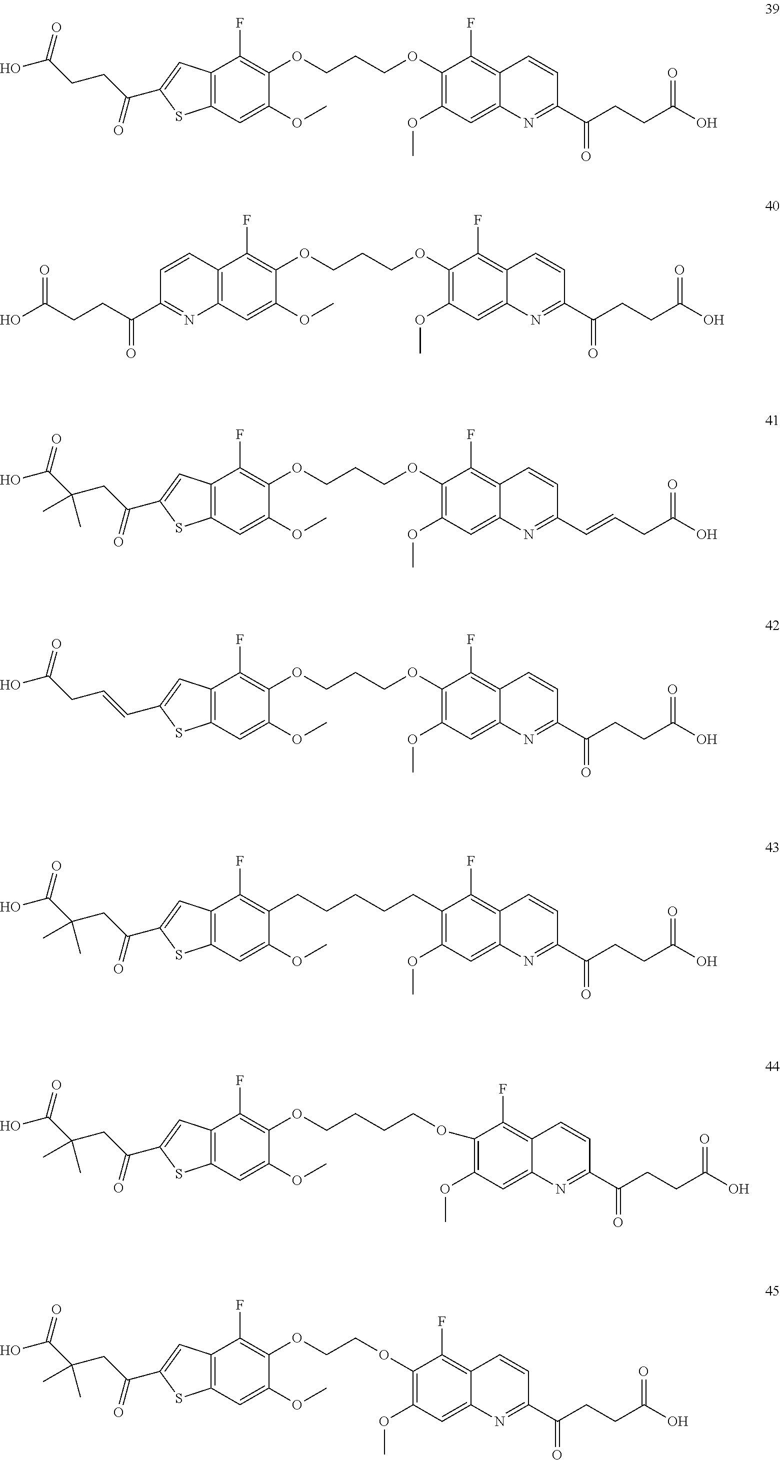

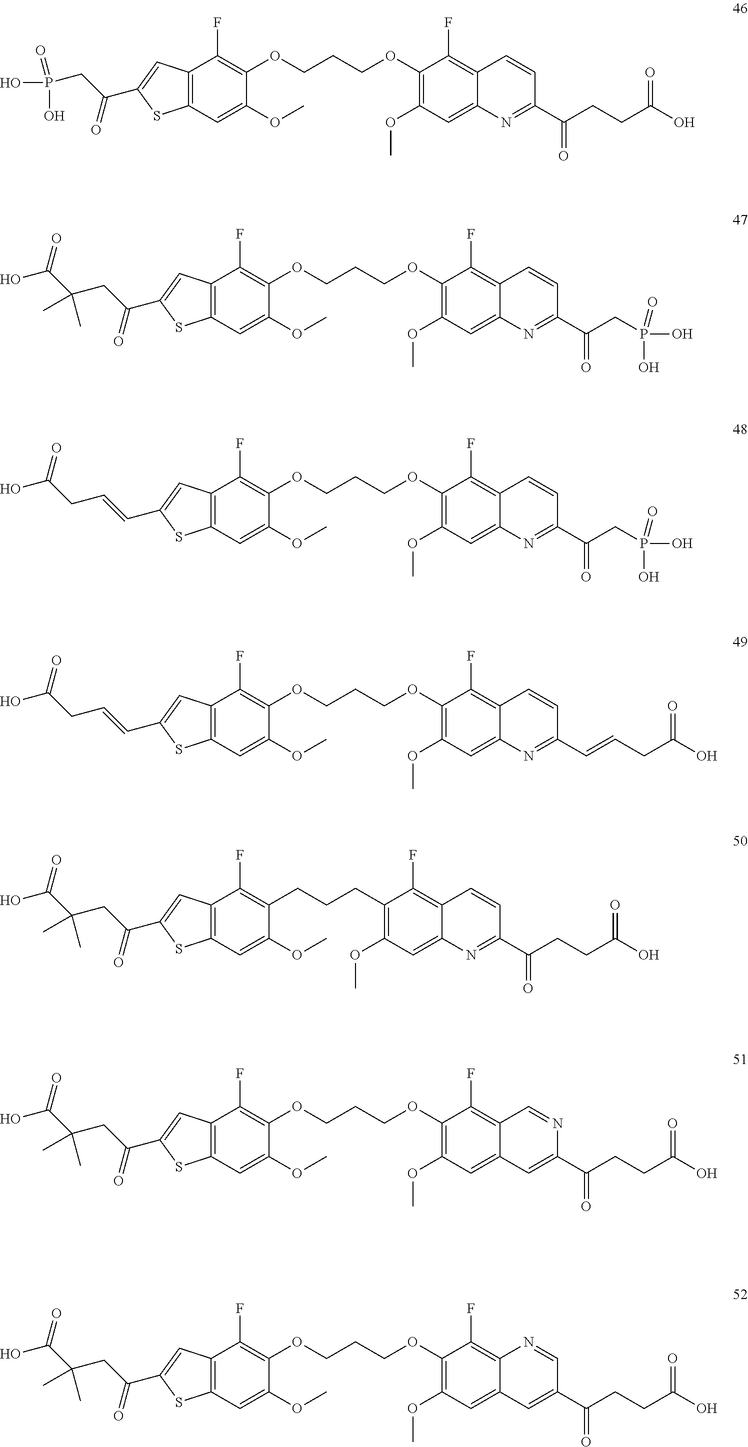

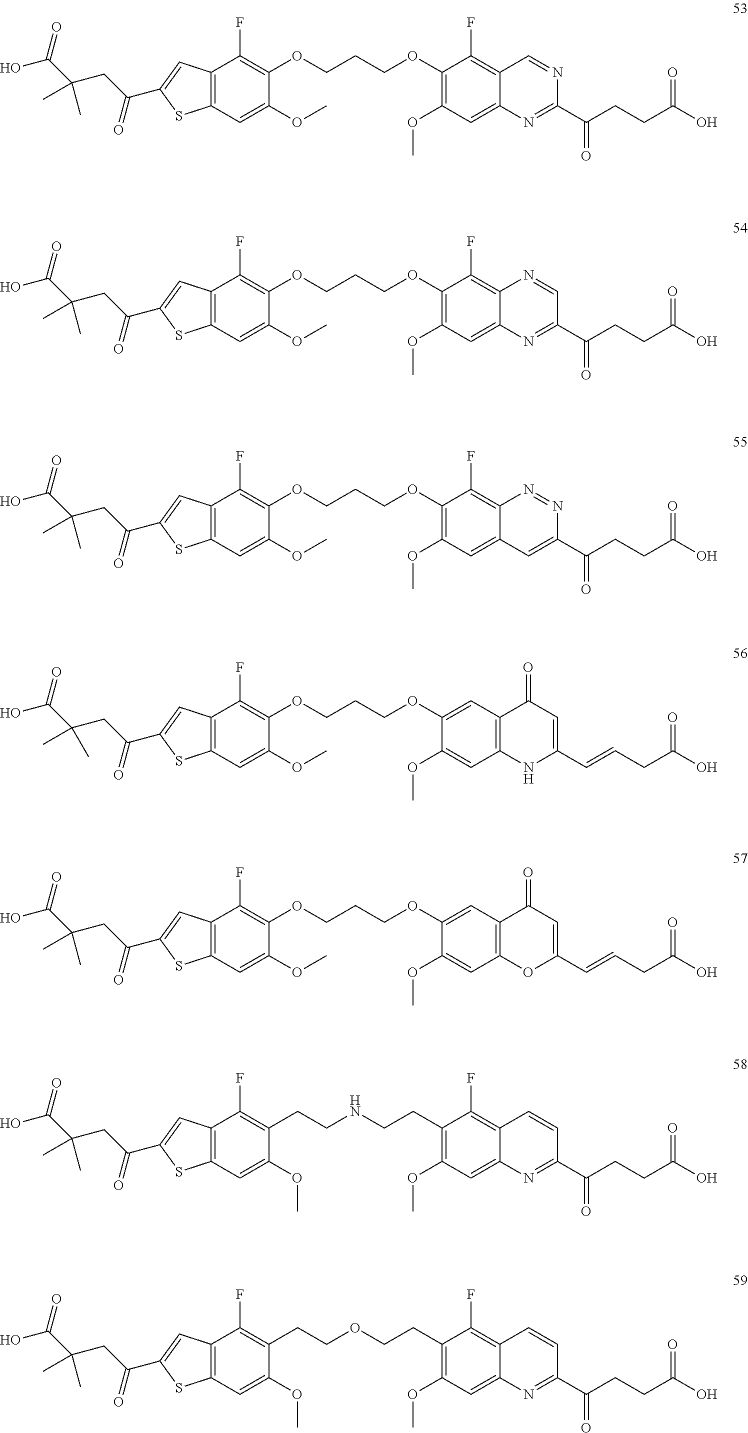

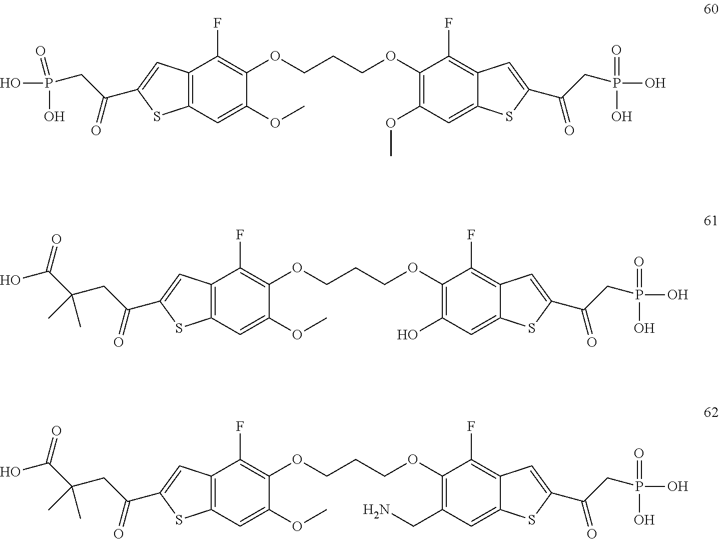 [SMILES: COc1cc2sc(C(=O)CP(=O)(O)O)cc2c(F)c1OCCCOc1c(OC)cc2sc(C(=O)CP(=O)(O)O)cc2c1F ; COc1cc2sc(C(=O)CC(C)(C)C(=O)O)cc2c(F)c1OCCCOc1c(O)cc2sc(C(=O)CP(=O)(O)O)cc2c1F ; COc1cc2sc(C(=O)CC(C)(C)C(=O)O)cc2c(F)c1OCCCOc1c(CN)cc2sc(C(=O)CP(=O)(O)O)cc2c1F]

### Example 2: In Vitro Cell Based Activity of Select STING Agonists

Certain STING compounds of the disclosure were evaluated in an in vitro cell based assay.

293-Dual hSTING-R232 and mSTING cells (Invivogen) were seeded at 25,000 cells/well (50 ul) and co-cultured with STING agonists (2× concentration, 50 ul) in clear, half-area, 96-well polystyrene plates. Cells and test samples were prepared in DMEM supplemented with 10% FBS, Glutamax, and PenStrep. After overnight incubation at 37 C in a CO2 incubator, 20 ul of supernatant was added to QUANTI-Blue detection reagent (Invivogen). After one hour incubation at 37 C, plates were read at 640/650 nm on EnVision plate reader (PerkinElmer). OD values were plotted by log(agonist) vs. response, variable slope, four parameter curve fit (Log transform) using GraphPad Prism software to determine EC₅₀ values.

Table 1 provides the results for ISG reporter induction 293 human/mouse reporter cells. (“A” means EC₅₀≤50 nM; “B” means >50 nM and <150 nM; “C” means >150 nM).

TABLE 1
Activity of select compounds in SG reporter
induction 293 human and mouse reporter cells

| | ISG reporter | ISG reporter |
|---|---|---|
| | induction 293 | induction 293 |
| | human reporter cells | mouse reporter |
| Compound No. | (EC₅₀) nM | cells (EC₅₀) nM |
| 1 | C | B |
| 2 | C | B |
| 3 | C | C |
| 4 | C | B |
| 5 | B | B |
| 6 | A | A |
| 7 | A | A |
| 8 | C | B |
| 9 | C | C |
| 10 | C | C |
| 11 | A | A |
| 12 | C | B |
| 13 | B | B |
| 14 | C | A |
| 15 | C | C |
| 16 | C | C |
| 17 | C | C |
| 18 | C | C |
| 19 | C | C |
| 20 | A | A |
| 21 | C | A |
| 22 | C | C |
| 23 | A | A |
| 32 | B | C |
| 33 | C | C |
| 34 | C | C |

### Example 3: Thermal Shift and Surface Plasmon Resonance of Select STING Agonists

Thermal shift assay experimental methods were followed based on the published methods described in Ramanjulu et al. Nature, 2018, 564, 439. Results are provided in Table 2.

TABLE 2
Thermal shift of select STING agonists

| | hSTING WT DSF |
|---|---|
| | (delta TmD @ 3.12 |
| Compound No. | uM) |
| 1 | 20 |
| 5 | −0.1 |
| 6 | 20 |
| 7 | 20 |

Surface plasmon resonance assay experimental methods were followed based on the published methods described in Pan et al., Science, 2020 369, eaba6098. (“A” means EC₅₀≤20 nM). Results are provided in Table 3.

TABLE 3
Surface plasmon resonance of select STING agonists

| | hSTING WT SPR |
|---|---|
| Compound No. | (Kd) nM |
| 1 | A |
| 6 | A |
| 7 | A |
| 8 | A |

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. A compound of Formula I: 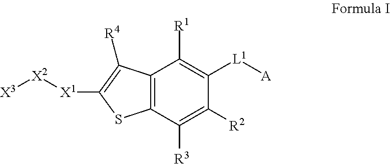
or a pharmaceutically acceptable salt, solvate, or stereoisomer thereof;
wherein:
L¹ is selected from C₁-C₆alkylene, 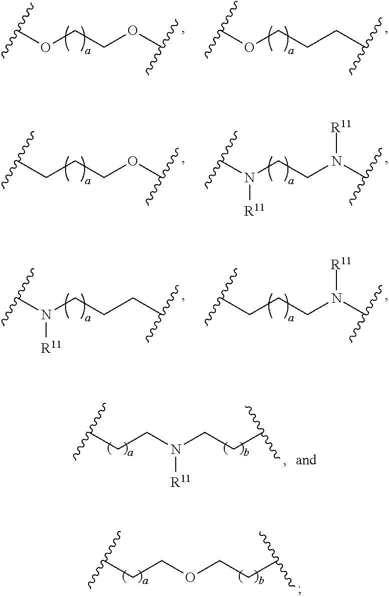
wherein any substitutable carbon in L¹ is optionally substituted with one or more substituents selected from halogen, alkoxy, C₁₋₆alkyl, C₁₋₆alkylamino, C₃₋₁₀cycloalkyl, hydroxy, cyano, —NR^{9a}R^{9b}, and —COOR¹⁰;
A is selected from 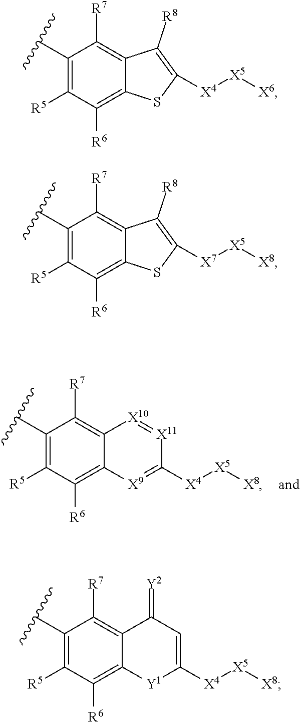
R¹, R², R³, R⁴, R⁵, R⁶, R⁷ and R⁸ are independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, amino-C₁₋₆alkyl, C₁₋₆alkylamino-C₁₋₆alkyl, di-C₁₋₆alkylamino-C₁₋₆alkyl, and —COOR¹⁰;
X¹ and X⁴ are independently selected from —C(O)—, —C(R^{12a}R^{12b})—, —CH═CH—, and —C(R^{12a}R^{12b})C(R^{13a}R^{13b})—;
X² and X⁵ are independently selected from —(C(R^{12a}R^{12b}))₁₋₆— and —(C(R^{12a}R^{12b})C(R^{13a}R^{13b}))₁₋₃—;
X³ is selected from —COOR¹⁰, —CONR^{9a}R^{9b}, —C(O)NR^{9a}S(O)₂R⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁶ is selected from —C(O)NR^{9a}S(O)₂R¹⁷, —C(O)NR^{9a}R¹⁸, —S(O)₂OH, —S(O)OH, —P(O)(OR¹⁵)₂, —OP(O)(OR¹⁵)₂, and —P(O)(NR^{16a}R^{16b})(OR¹⁵);
X⁷ is —CH═CH—, C₂alkynyl, or —C(R^{12a}R^{12c})C(R^{13a}R^{13c})—;
X⁸ is selected from —COOR¹⁰, —CONR^{9a}R^{9b}, —C(O)NR^{9a}S(O)₂R⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁹, X¹⁰, and X¹¹ are independently selected from CR¹⁹ and N;
Y¹ is independently selected from CH₂, O, NH, and S;
Y² is independently selected from O, NH, and S;
R^{9a} and R^{9b} are independently selected from hydrogen, C₁₋₆alkyl, C₃₋₁₀cycloalkyl, aryl, aryl-C₁₋₆alkyl, amino-C₁₋₆alkyl, C₁₋₆alkylamino-C₁₋₆alkyl, di-C₁₋₆alkylamino-C₁₋₆alkyl, and heteroaryl;
R¹⁰ is independently selected from hydrogen, C₁₋₆alkyl, C₃₋₁₀cycloalkyl, aryl, aryl-C₁₋₆alkyl, and heteroaryl;
R¹¹ is independently selected from hydrogen and C₁₋₆alkyl;
R^{12a}, R^{12b}, R^{13a}, and R^{13b} are independently selected from hydrogen and C₁₋₆alkyl;
or R^{12b} and R^{13b} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R^{12c} and R^{13c} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R¹⁴ is selected from C₁₋₆alkyl, C₃₋₁₀cycloalkyl, halo-C₁₋₆alkyl, amino-C₁₋₆alkyl, C₁₋₆alkyl-amino-C₁₋₆alkyl, di-C₁₋₆alkylamino-C₁₋₆alkyl, aryl, aryl-C₁₋₆alkyl, and heteroaryl;
R¹⁵, R^{16a}, and R^{16b} are independently selected from hydrogen, C₁₋₆alkyl, and C₃₋₁₀cycloalkyl;
R¹⁷ is amino-C₁₋₆alkyl, C₁₋₆alkyl-amino-C₁₋₆alkyl, di-C₁₋₆alkylamino-C₁₋₆alkyl, C₃₋₁₀cycloalkyl-amino-C₁₋₆alkyl, aryl, aryl-C₁₋₆alkyl, or heteroaryl;
R¹⁸ is amino-C₁₋₆alkyl, C₁₋₆alkylamino-C₁₋₆alkyl, di-C₁₋₆alkylamino-C₁₋₆alkyl, C₃₋₁₀cycloalkyl-amino-C₁₋₆alkyl, C₁₋₆alkyl, C₃₋₁₀cycloalkyl, aryl, aryl-C₁₋₆alkyl, or heteroaryl; and
R¹⁹ is independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, and —COOR¹⁰; and
each of a and b is an integer independently selected from 0, 1, 2, 3, 4, and 5.

2. A compound of Formula II: 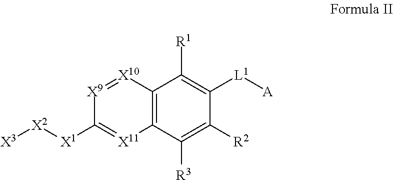
or a pharmaceutically acceptable salt, solvate, or stereoisomer thereof;
wherein:
L¹ is selected from C₁-C₆alkylene, 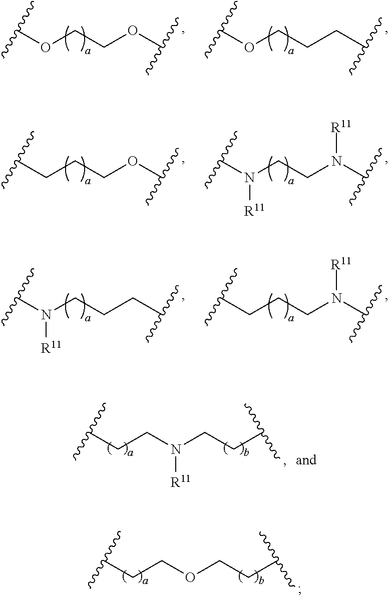
wherein any substitutable carbon in L is optionally substituted with one or more substituents selected from halogen, alkoxy, C₁₋₆alkyl, C₁₋₆alkylamino, C₃₋₁₀cycloalkyl, hydroxy, cyano, —NR^{9a}R^{9b}, and —COOR¹⁰;
A is selected from 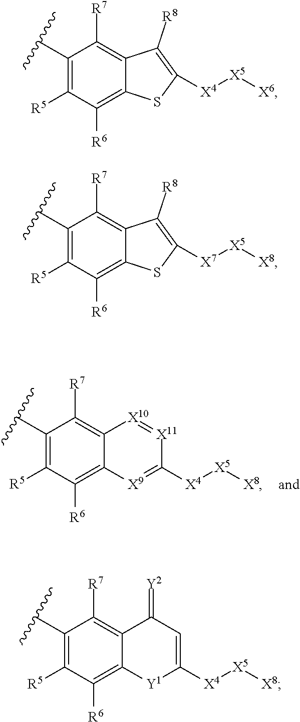
R¹, R², R³, R⁴, R⁵, R⁶, R⁷ and R⁸ are independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, amino-C₁₋₆alkyl, C₁₋₆alkylamino-C₁₋₆alkyl, di-C₁₋₆alkylamino-C₁₋₆alkyl, and —COOR¹⁰;
X¹ and X⁴ are independently selected from —C(O)—, —C(R^{12a}R^{12b})—, —CH═CH—, and —C(R^{12a}R^{12b})C(R^{13a}R^{13b})—;
X² and X⁵ are independently selected from —(C(R^{12a}R^{12b}))₁₋₆ and —(C(R^{12a}R^{12b})C(R^{13a}R^{13b}))₁₋₃—;
X³ is selected from —COOR¹⁰, —CONR^{9a}R^{9b}, —C(O)NR^{9a}S(O)₂R⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁶ is selected from —C(O)NR^{9a}S(O)₂R¹⁷, —C(O)NR^{9a}R¹⁸, —S(O)₂OH, —S(O)OH, —P(O)(OR¹⁵)₂, —OP(O)(OR¹⁵)₂, and —P(O)(NR^{16a}R^{16b})(OR¹⁵);
X⁷ is —CH═CH—, C₂alkynyl, or —C(R^{12a}R^{12c})C(R^{13a}R^{13c})—;
X⁸ is selected from —COOR¹⁰, —CONR^{9a}R^{9b}, —C(O)NR^{9a}S(O)₂R¹⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁹, X¹⁰, and X¹¹ are independently selected from CR¹⁹ and N;
Y¹ is independently selected from CH₂, O, NH, and S;
Y² is independently selected from O, NH, and S;
R^{9a} and R^{9b} are independently selected from hydrogen, C₁₋₆alkyl, C₃₋₁₀cycloalkyl, aryl, aryl-C₁₋₆alkyl, amino-C₁₋₆alkyl, C₁₋₆alkylamino-C₁₋₆alkyl, di-C₁₋₆alkylamino-C₁₋₆alkyl, and heteroaryl;
R¹⁰ is independently selected from hydrogen, C₁₋₆alkyl, C₃₋₁₀cycloalkyl, aryl, aryl-C₁₋₆alkyl, and heteroaryl;
R¹¹ is independently selected from hydrogen and C₁₋₆alkyl;
R^{12a}, R^{12b}, R^{13a}, and R^{13b} are independently selected from hydrogen and C₁₋₆alkyl;
or R^{12b} and R^{13b} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R^{12c} and R^{13c} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R¹⁴ is selected from C₁₋₆alkyl, C₃₋₁₀cycloalkyl, halo-C₁₋₆alkyl, amino-C₁₋₆alkyl, C₁₋₆alkyl-amino-C₁₋₆alkyl, di-C₁₋₆alkylamino-C₁₋₆alkyl, aryl, aryl-C₁₋₆alkyl, and heteroaryl;
R¹⁵, R^{16a}, and R^{16b} are independently selected from hydrogen, C₁₋₆alkyl, and C₃₋₁₀cycloalkyl;
R¹⁷ is amino-C₁₋₆alkyl, C₁₋₆alkyl-amino-C₁₋₆alkyl, di-C₁₋₆alkylamino-C₁₋₆alkyl, C₃₋₁₀cycloalkyl-amino-C₁₋₆alkyl, aryl, aryl-C₁₋₆alkyl, or heteroaryl;
R¹⁸ is amino-C₁₋₆alkyl, C₁₋₆alkylamino-C₁₋₆alkyl, di-C₁₋₆alkylamino-C₁₋₆alkyl, C₃₋₁₀cycloalkyl-amino-C₁₋₆alkyl, C₁₋₆alkyl, C₃₋₁₀cycloalkyl, aryl, aryl-C₁₋₆alkyl, or heteroaryl; and
R¹⁹ is independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, and —COOR¹⁰; and
each of a and b is an integer independently selected from 0, 1, 2, 3, 4, and 5.

3. A compound of Formula iii: 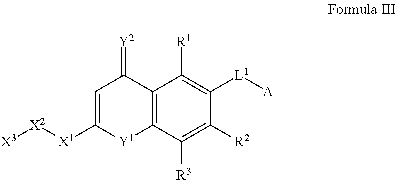
or a pharmaceutically acceptable salt, solvate, or stereoisomer thereof;
wherein:
L¹ is selected from C₁-C₆alkylene, 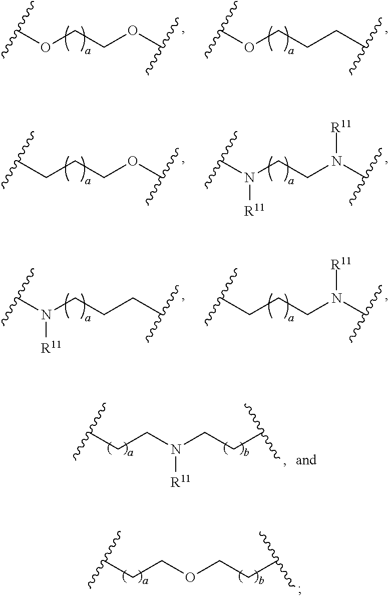
wherein any substitutable carbon in L¹ is optionally substituted with one or more substituents selected from halogen, alkoxy, C₁₋₆alkyl, C₁₋₆alkylamino, C₃₋₁₀cycloalkyl, hydroxy, cyano, —NR^{9a}R^{9b}, and —COOR¹⁰;
A is selected from 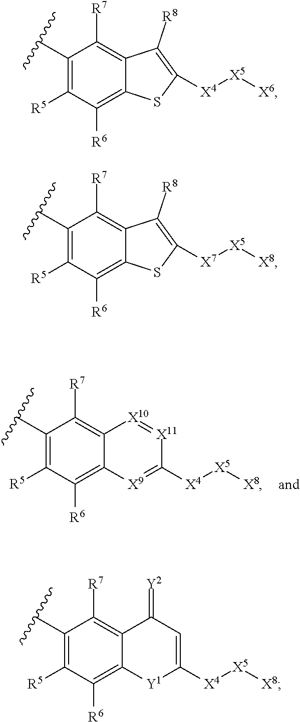
R¹, R², R³, R⁴, R⁵, R⁶, R⁷ and R⁸ are independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, amino-C₁₋₆alkyl, C₁₋₆alkylamino-C₁₋₆alkyl, di-C₁₋₆alkylamino-C₁₋₆alkyl, and —COOR¹⁰;
X¹ and X⁴ are independently selected from —C(O)—, —C(R^{12a}R^{12b})—, —CH═CH—, and —C(R^{12a}R^{12b})C(R^{13a}R^{13b})—;
X² and X⁵ are independently selected from —(C(R^{12a}R^{12b}))₁₋₆— and —(C(R^{12a}R^{12b})C(R^{13a}R^{13b}))₁₋₃—;
X³ is selected from —COOR¹⁰, —CONR^{9a}R^{9b}, —C(O)NR^{9a}S(O)₂R¹⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁶ is selected from —C(O)NR^{9a}S(O)₂R¹⁷, —C(O)NR^{9a}R¹⁸, —S(O)₂OH, —S(O)OH, —P(O)(OR¹⁵)₂, —OP(O)(OR¹⁵)₂, and —P(O)(NR^{16a}R^{16b})(OR¹⁵);
X⁷ is —CH═CH—, C₂alkynyl, or —C(R^{12a}R^{12c})C(R^{13a}R^{13c})—;
X⁸ is selected from —COOR¹⁰, —CONR^{9a}R^{9b}, —C(O)NR^{9a}S(O)₂R¹⁴, —S(O)₂OR¹⁰, —S(O)OR¹⁰, —P(O)(OR¹⁵)₂, —OP(O)(OR¹⁵)₂, and —P(O)(NR^{16a}R^{16b})(OR¹⁵);
X⁹, X¹⁰, and X¹¹ are independently selected from CR¹⁹ and N;
Y¹ is independently selected from CH₂, O, NH, and S;
Y² is independently selected from O, NH, and S;
R^{9a} and R^{9b} are independently selected from hydrogen, C₁₋₆alkyl, C₃₋₁₀cycloalkyl, aryl, aryl-C₁₋₆alkyl, amino-C₁₋₆alkyl, C₁₋₆alkylamino-C₁₋₆alkyl, di-C₁₋₆alkylamino-C₁₋₆alkyl, and heteroaryl;
R¹⁰ is independently selected from hydrogen, C₁₋₆alkyl, C₃₋₁₀cycloalkyl, aryl, aryl-C₁₋₆alkyl, and heteroaryl;
R¹¹ is independently selected from hydrogen and C₁₋₆alkyl;
R^{12a}, R^{12b}, R^{13a}, and R^{13b} are independently selected from hydrogen and C₁₋₆alkyl;
or R^{12b} and R^{13b} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R^{12c} and R^{13c} are joined together to form a C₃-C₁₀cycloalkyl optionally substituted with a substituent selected from halogen, alkoxy, C₁₋₆alkyl, C₃₋₁₀cycloalkyl, hydroxy, cyano, —NR^{9a}R^{9b}, and —COOR¹⁰;
R¹⁴ is selected from C₁₋₆alkyl, C₃₋₁₀cycloalkyl, halo-C₁₋₆alkyl, amino-C₁₋₆alkyl, C₁₋₆alkyl-amino-C₁₋₆alkyl, di-C₁₋₆alkylamino-C₁₋₆alkyl, aryl, aryl-C₁₋₆alkyl, and heteroaryl;
R¹⁵, R^{16a}, and R^{16b} are independently selected from hydrogen, C₁₋₆alkyl, and C₃₋₁₀cycloalkyl;
R¹⁷ is amino-C₁₋₆alkyl, C₁₋₆alkyl-amino-C₁₋₆alkyl, di-C₁₋₆alkylamino-C₁₋₆alkyl, C₃₋₁₀cycloalkyl-amino-C₁₋₆alkyl, aryl, aryl-C₁₋₆alkyl, or heteroaryl;
R¹⁸ is amino-C₁₋₆alkyl, C₁₋₆alkylamino-C₁₋₆alkyl, di-C₁₋₆alkylamino-C₁₋₆alkyl, C₃₋₁₀cycloalkyl-amino-C₁₋₆alkyl, C₁₋₆alkyl, C₃₋₁₀cycloalkyl, aryl, aryl-C₁₋₆alkyl, or heteroaryl; and
R¹⁹ is independently selected from hydrogen, halogen, alkoxy, C₁₋₆alkyl, C₃₋₁₀cycloalkyl, halo-C₁₋₆alkyl, hydroxyl-C₁₋₆alkyl, hydroxy, —NR^{9a}R^{9b}, and —COOR¹⁰; and
each of a and b is an integer independently selected from 0, 1, 2, 3, 4, and 5.

4. The compound of claim 1 of Formula Ia: 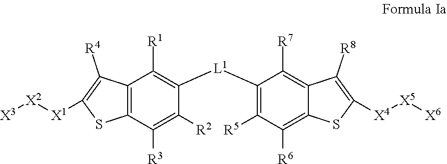
or a pharmaceutically acceptable salt, solvate, or stereoisomer thereof.

5. The compound of claim 1 of Formula Ib: 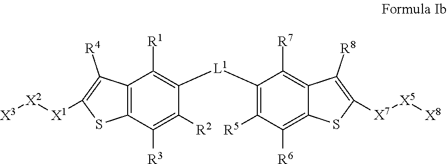
or a pharmaceutically acceptable salt, solvate, or stereoisomer thereof.

6. The compound of claim 1 of Formula Ic: 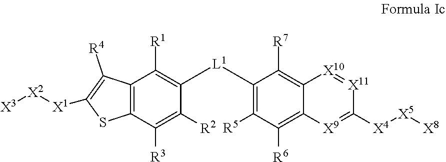
or a pharmaceutically acceptable salt, solvate, or stereoisomer thereof.

7. The compound of claim 1 of Formula Id: 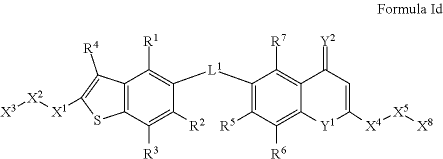
or a pharmaceutically acceptable salt, solvate, or stereoisomer thereof.

8. The compound of claim 2 of Formula IIa: 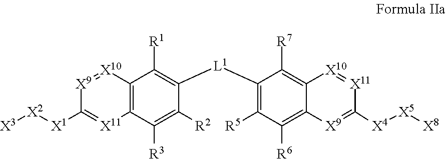
or a pharmaceutically acceptable salt, solvate, or stereoisomer thereof.

9. The compound of claim 2 of Formula IIb: 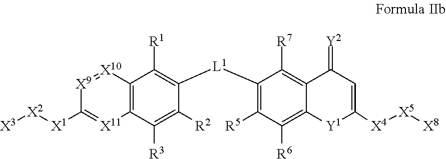
or a pharmaceutically acceptable salt, solvate, or stereoisomer thereof.

10. The compound of claim 3 of Formula IIIa: 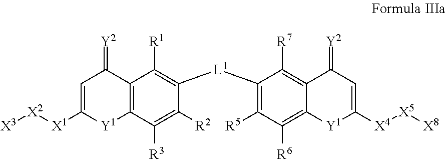
or a pharmaceutically acceptable salt, solvate, or stereoisomer thereof.

11. The compound of any one of claims 1-10, wherein L¹ is selected from C₁-C₆alkylene,

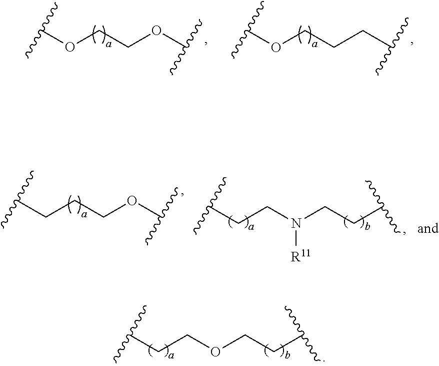

12. The compound of any one of claims 1-10, wherein L¹ is

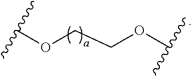

13. The compound of any one of claims 1-10, wherein L¹ is selected from

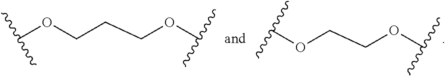 [SMILES: *OCCCO* ; *OCCO*]

14. The compound of claim 4, wherein the compound of Formula Ia is a compound of Formula (Iai) (Iaii), (Iaiii), or (Iaiv): 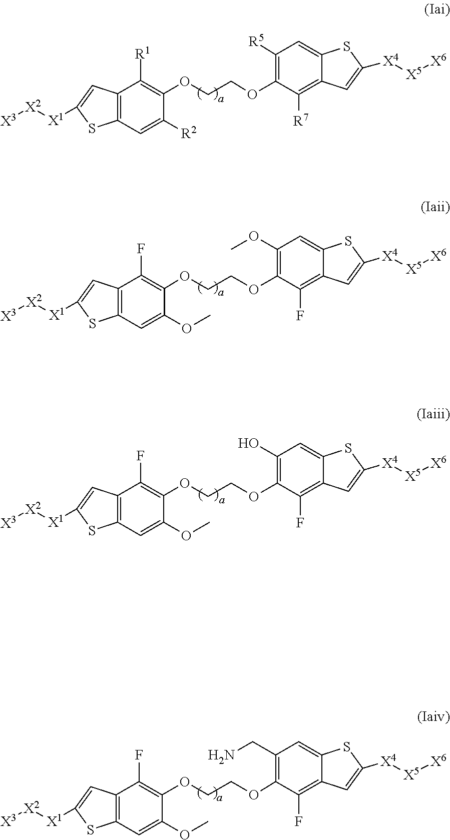
or a pharmaceutically acceptable salt, solvate, or stereoisomer thereof.

15. The compound of claim 5, wherein the compound of Formula Ib is a compound of Formula (Ibi) or (Ibii): 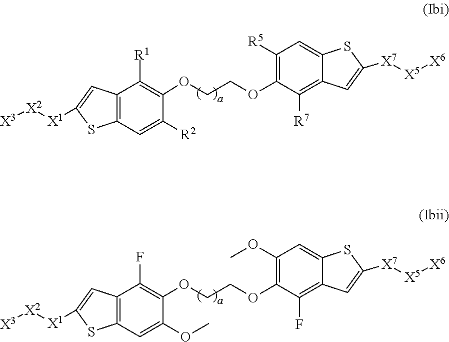
or a pharmaceutically acceptable salt, solvate, or stereoisomer thereof.

16. The compound of claim 6, wherein the compound of Formula Ic is a compound of Formula (Ici), (Icii), (Iciii), (Iciv): 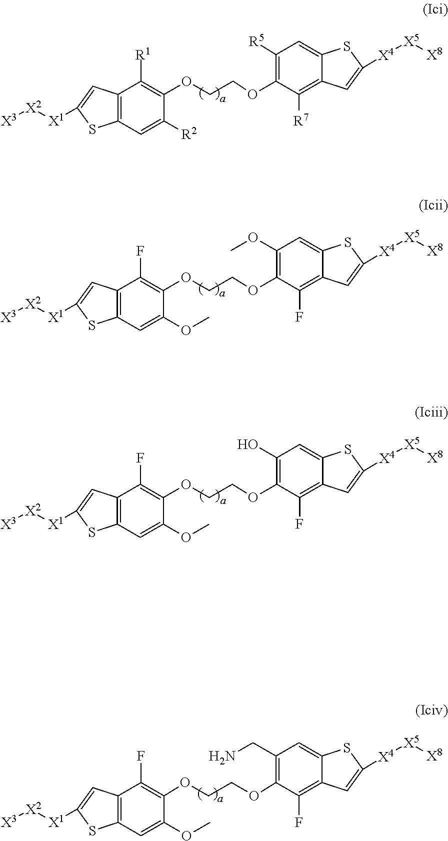
or a pharmaceutically acceptable salt, solvate, or stereoisomer thereof.

17. The compound of claim 7, wherein the compound of Formula Id is a compound of Formula (Idi): 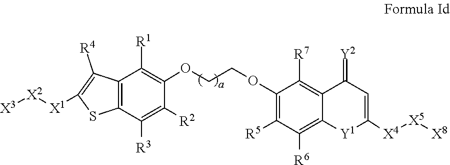
or a pharmaceutically acceptable salt, solvate, or stereoisomer thereof.

18. The compound of claim 8, wherein the compound of Formula IIa is a compound of Formula (IIai): 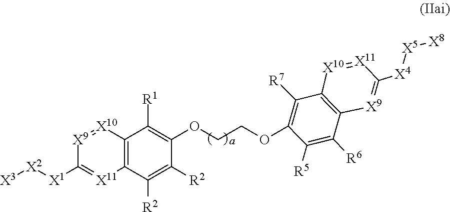
or a pharmaceutically acceptable salt, solvate, or stereoisomer thereof.

19. The compound of any one of claims 1-18, wherein R¹ and R⁷ are independently selected from hydrogen and halogen; R² is selected from hydrogen and alkoxy; and, R⁵ is selected from alkoxy, hydroxy, and amino-C₁₋₆alkyl.

20. The compound of any one of claims 1-18, wherein R¹ and R⁷ are each halogen and R² and R⁵ are each alkoxy.

21. The compound of claim 19, wherein R¹ is halogen.

22. The compound of claim 19, wherein R⁷ is halogen.

23. The compound of claim 21 or 22, wherein halogen is fluorine.

24. The compound of any one of claims 1-23, wherein R² is alkoxy.

25. The compound of any one of claims 1-24, wherein R⁵ is selected from alkoxy and hydrogen.

26. The compound of any one of claims 1-25, wherein alkoxy is methoxy.

27. The compound of any one of claims 1-26, wherein a is 2 or 3.

28. The compound of claim 27, wherein a is 2.

29. The compound of any one of claims 1-28, wherein 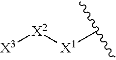
is selected from 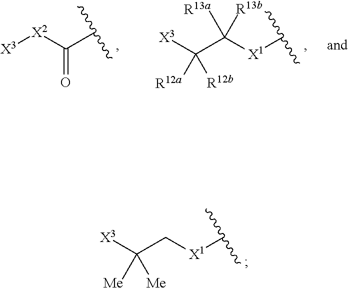
wherein 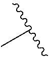
indicates a point of attachment to the rest of the compound.

30. The compound of claim 29, wherein 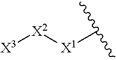
is selected from 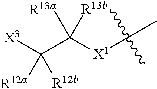
and 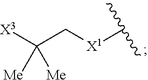
wherein 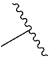
indicates a point of attachment to the rest of the compound.

31. The compound of claim 29, wherein 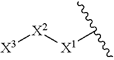
is selected from 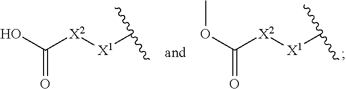
wherein 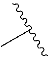
indicates a point of attachment to the rest of the compound.

32. The compound of claim 29, wherein 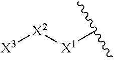
is selected from 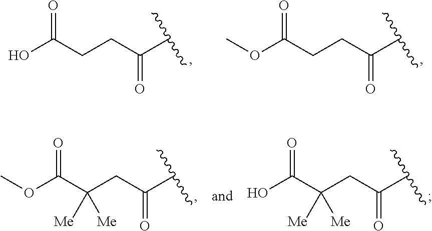
wherein 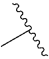
indicates a point of attachment to the rest of the compound.

33. The compound of any one of claims 1-4, 6-14, and 16-32, wherein X⁴ is —C(O)— and X⁵ is selected from —CH₂—, —CH₂CH₂—, and —CH₂C(CH₃)₂—.

34. The compound of any one of claims 1-4, 6-14, and 16-32, wherein 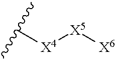
is selected from 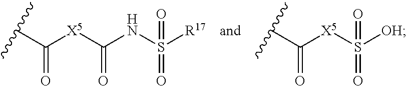
wherein 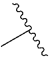
indicates a point of attachment to the rest of the compound.

35. The compound of any one of claims 1-4, 6-14, and 16-32, wherein 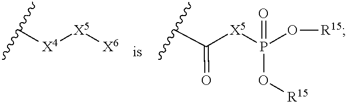
wherein 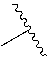
indicates a point of attachment to the rest of the compound.

36. The compound of any one of claims 1-4, 6-14, and 16-32, wherein 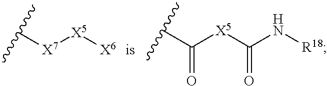
wherein 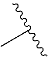
indicates a point of attachment to the rest of the compound.

37. The compound of claim 34, wherein R¹⁷ is amino-C₁₋₆alkyl, C₁₋₆alkyl-amino-C₁₋₆ alkyl, or di-C₁₋₆alkylamino-C₁₋₆alkyl.

38. The compound of claim 37, wherein R¹⁷ is amino-C₁₋₆alkyl.

39. The compound of claim 36, wherein R¹⁸ is amino-C₁₋₆alkyl, C₁₋₆alkyl-amino-C₁₋₆ alkyl, di-C₁₋₆alkylamino-C₁₋₆alkyl, or C₁₋₆alkyl.

40. The compound of claim 39, wherein R¹⁸ is amino-C₁₋₆alkyl.

41. The compound of claim 35, wherein R¹⁵ is independently selected from hydrogen, C₁₋₆alkyl, and C₃₋₁₀cycloalkyl.

42. The compound of claim 41, wherein R¹⁵ is hydrogen.

43. The compound of claim 41, wherein R¹⁵ is C₁₋₆alkyl.

44. The compound of any one of claims 1-3, 5, 11-13, 15, and 19-32, wherein 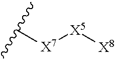
is selected from 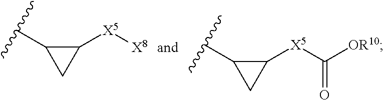
wherein 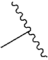
indicates a point of attachment to the rest of the compound.

45. The compound of any one of claims 1-3, 5, 11-13, 15, and 19-32, wherein 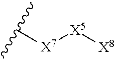
is selected from 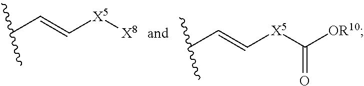
wherein 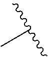
indicates a point of attachment to the rest of the compound.

46. The compound of any one of claims 1-3, 5, 11-13, 15, and 19-32, wherein 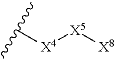
is selected from 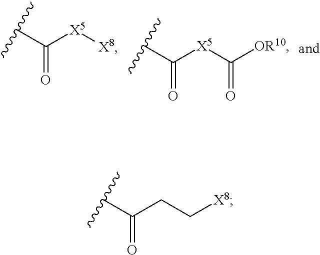
wherein 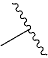
indicates a point of attachment to the rest of the compound.

47. The compound of any one of claims 1-46, wherein R¹⁰ is hydrogen, C₁₋₆alkyl, or C₃₋₁₀cycloalkyl.

48. The compound of claim 47, wherein R¹⁰ is hydrogen.

49. The compound of any one of claims 1-48, wherein X⁵ is selected from —CH₂—, —CH₂CH₂—, and —CH₂C(CH₃)₂—.

50. The compound of any one of claims 1-3, 6, 8, 11-13, and 18-43, wherein X⁹, X¹⁰, and X¹¹ are independently selected from CR¹⁹ and N.

51. The compound of claim 50, wherein X⁹ is N and X¹⁰ and X¹¹ are CR¹⁹ and N.

52. The compound of claim 50, wherein X¹⁰ is N and X⁹ and X¹¹ are CR¹⁹ and N.

53. The compound of claim 50, wherein X¹¹ is N and X⁹ and X¹⁰ are CR¹⁹ and N.

54. The compound of any one of claims 50-53, wherein R¹⁹ is independently selected from hydrogen, halogen, C₁₋₆alkyl, and alkoxy.

55. The compound of any one of claims 50-53, wherein R¹⁹ is hydrogen.

56. The compound of any one of claims 1-3, 7, 9-13, 17, and 19-43, wherein Y¹ is O or N and Y² is O.

57. The compound of claim 4, of the formula: 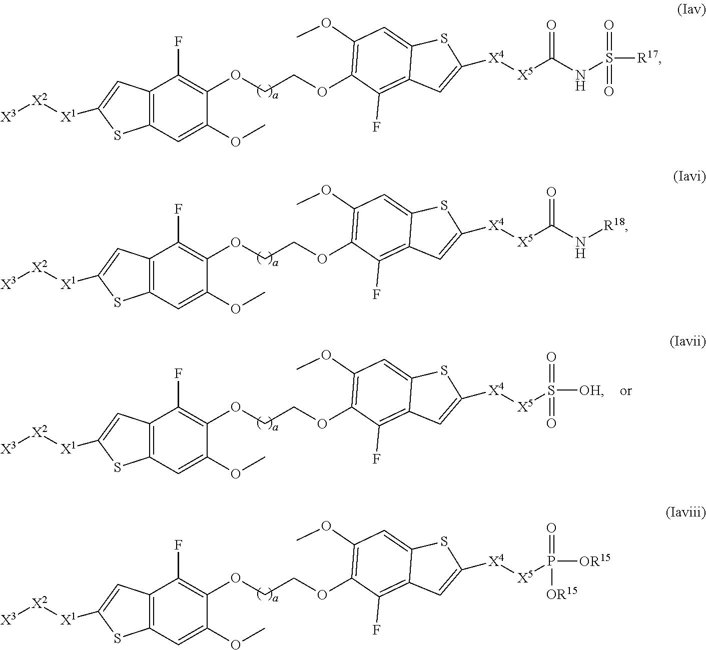
or a pharmaceutically acceptable salt, solvate, or stereoisomer thereof.

58. The compound of claim 57, of the formula: 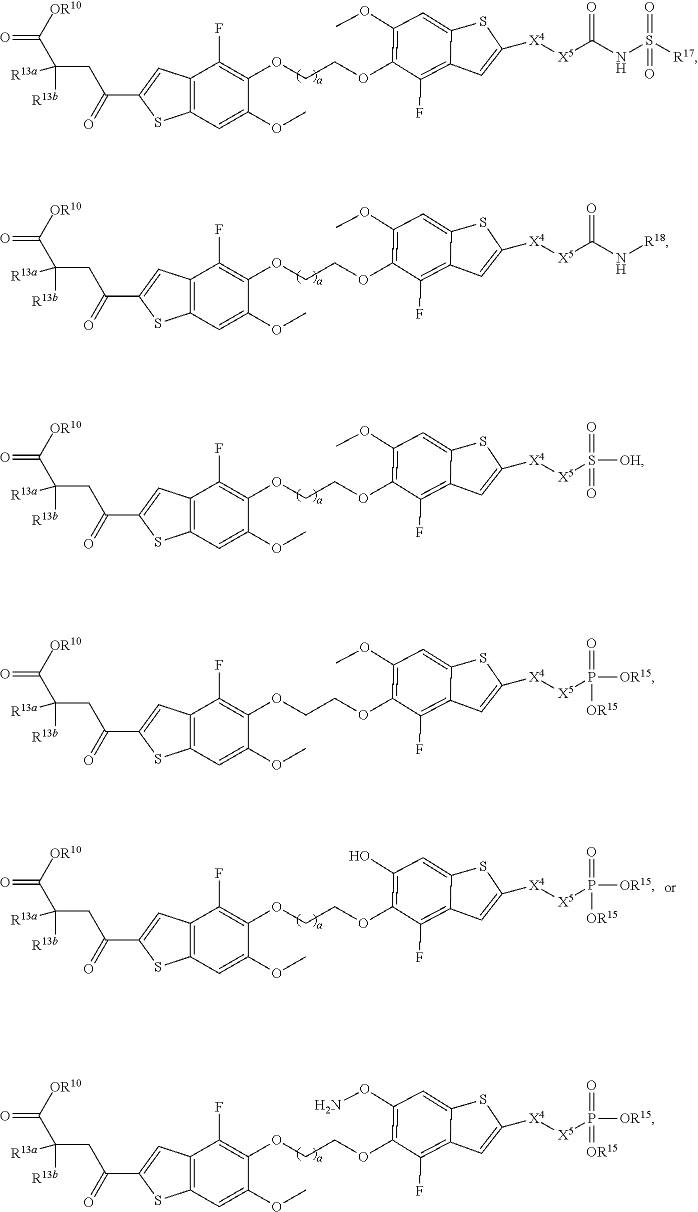
or a pharmaceutically acceptable salt, solvate, or stereoisomer thereof.

59. The compound of claim 5, of the formula: 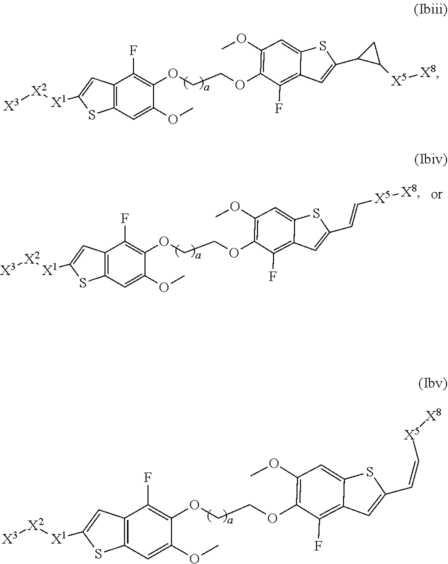
or a pharmaceutically acceptable salt, solvate, or stereoisomer thereof.

60. The compound of claim 59, of the formula: 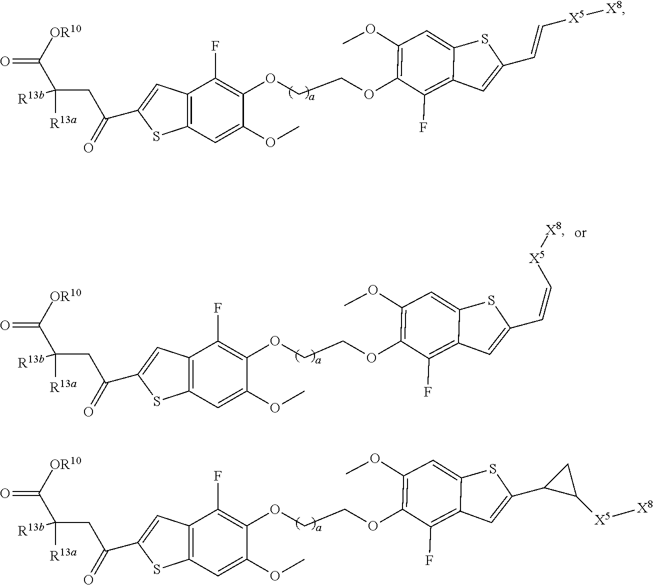
or a pharmaceutically acceptable salt, solvate, or stereoisomer thereof.

61. The compound of claim 1, wherein the compound is selected from: 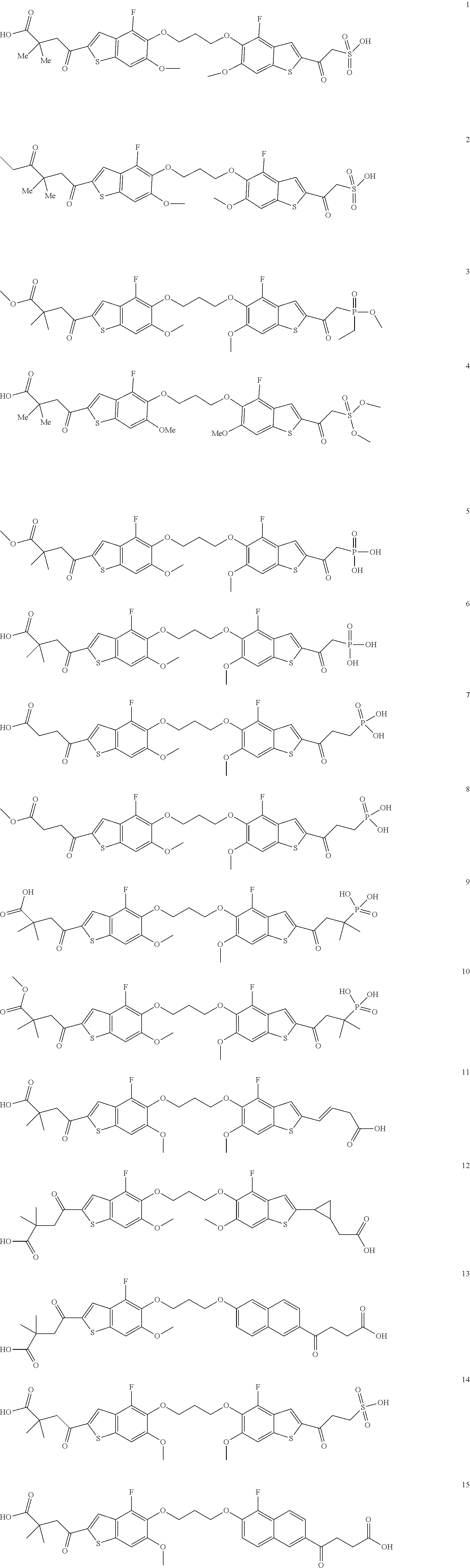 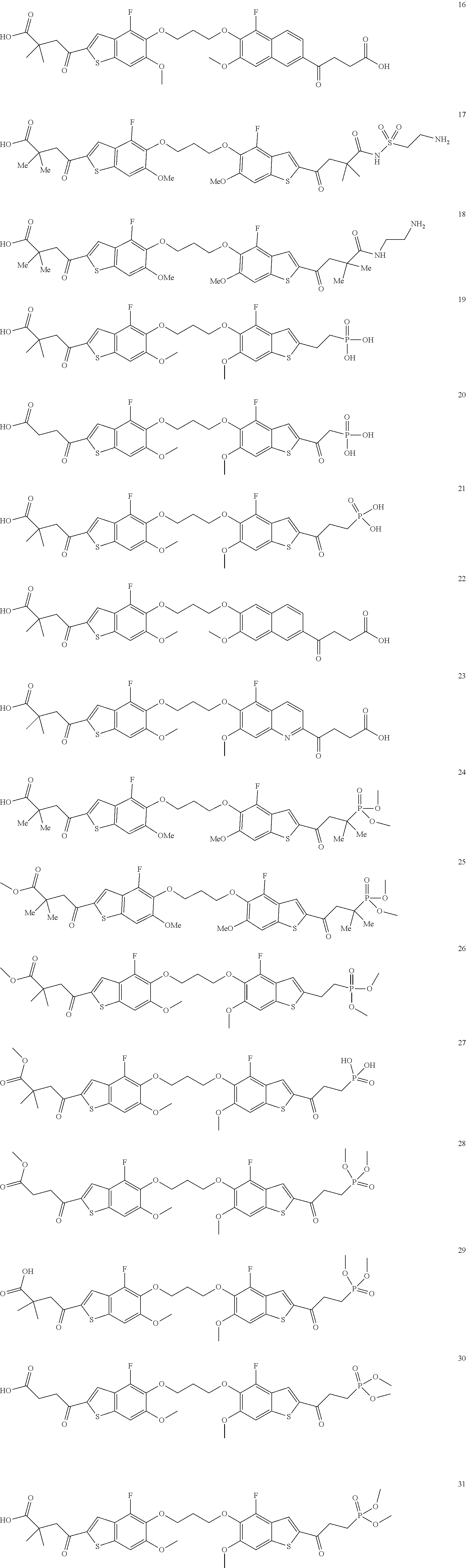
or a pharmaceutically acceptable salt, solvate, or stereoisomer thereof.

62. The compound of claim 1, wherein the compound is selected from: 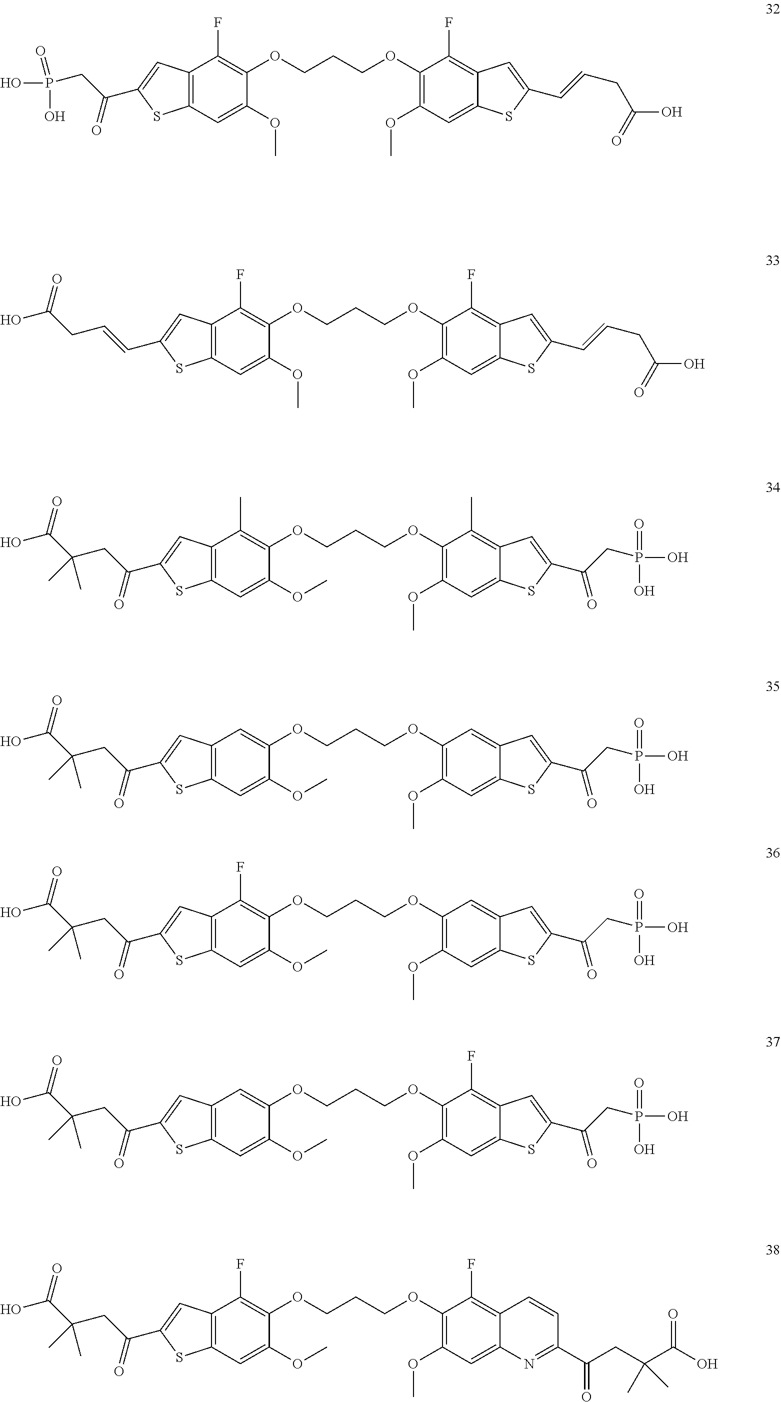 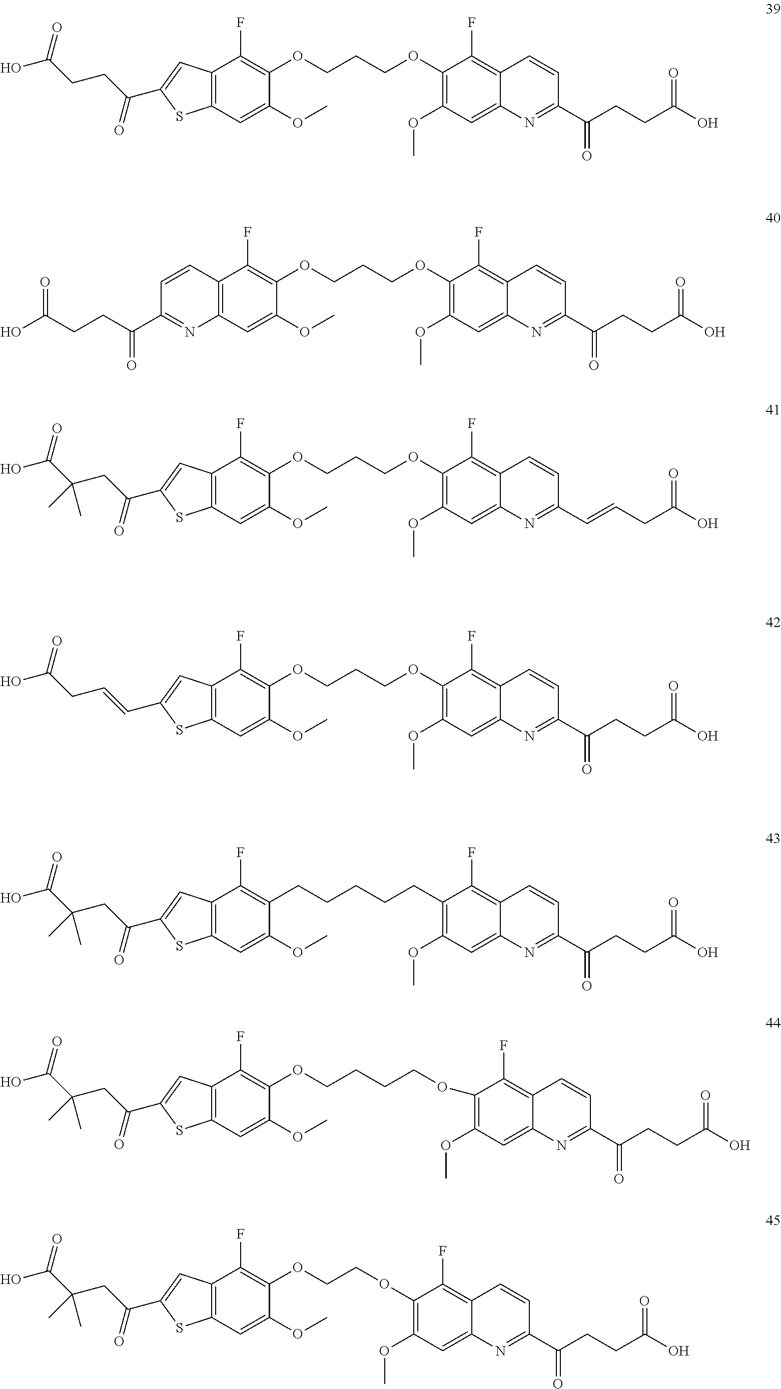 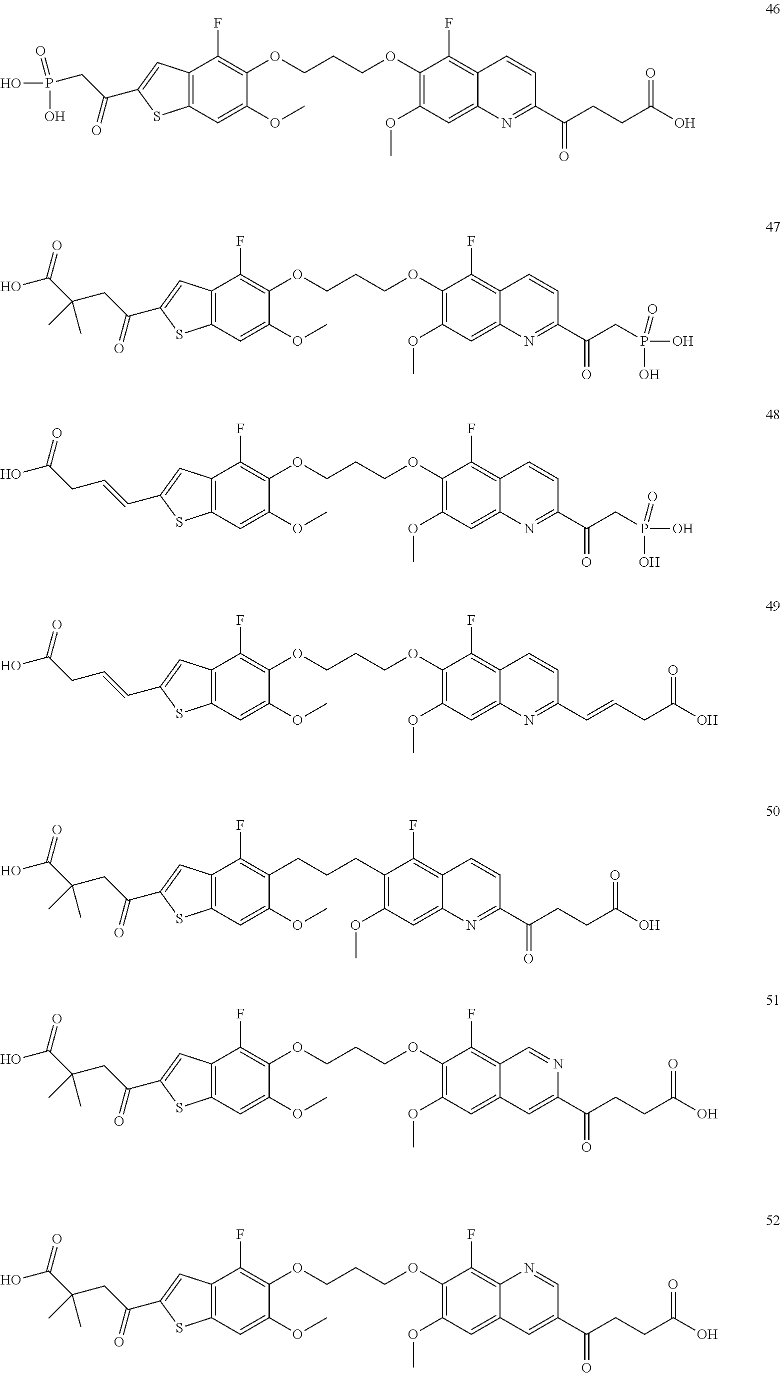 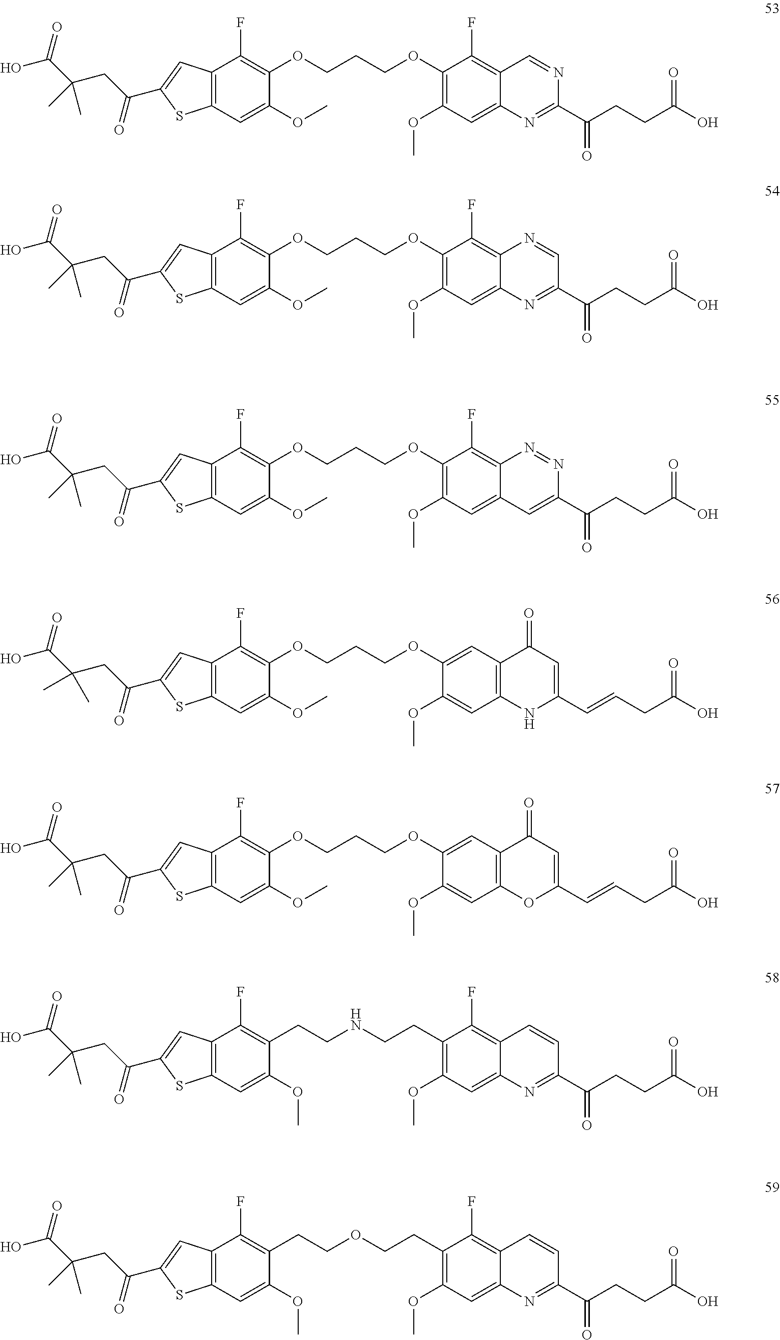 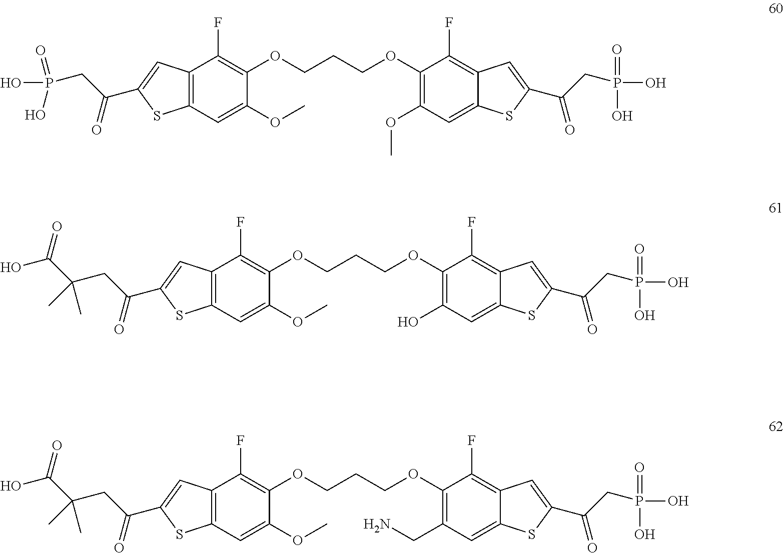 [SMILES: COc1cc2sc(C(=O)CP(=O)(O)O)cc2c(F)c1OCCCOc1c(OC)cc2sc(C(=O)CP(=O)(O)O)cc2c1F ; COc1cc2sc(C(=O)CC(C)(C)C(=O)O)cc2c(F)c1OCCCOc1c(O)cc2sc(C(=O)CP(=O)(O)O)cc2c1F ; COc1cc2sc(C(=O)CC(C)(C)C(=O)O)cc2c(F)c1OCCCOc1c(CN)cc2sc(C(=O)CP(=O)(O)O)cc2c1F]
or a pharmaceutically acceptable salt, solvate, or stereoisomer thereof.

63. The compound of claim 1, wherein the compound is selected from: 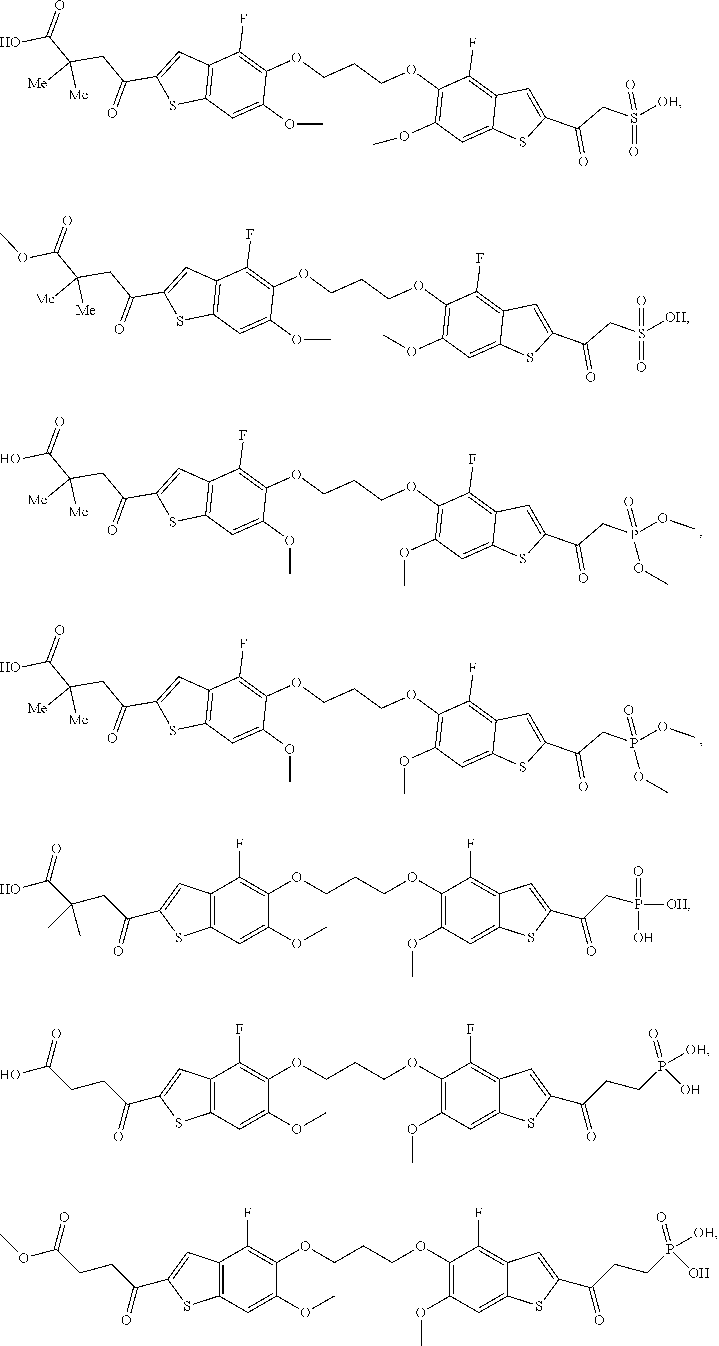 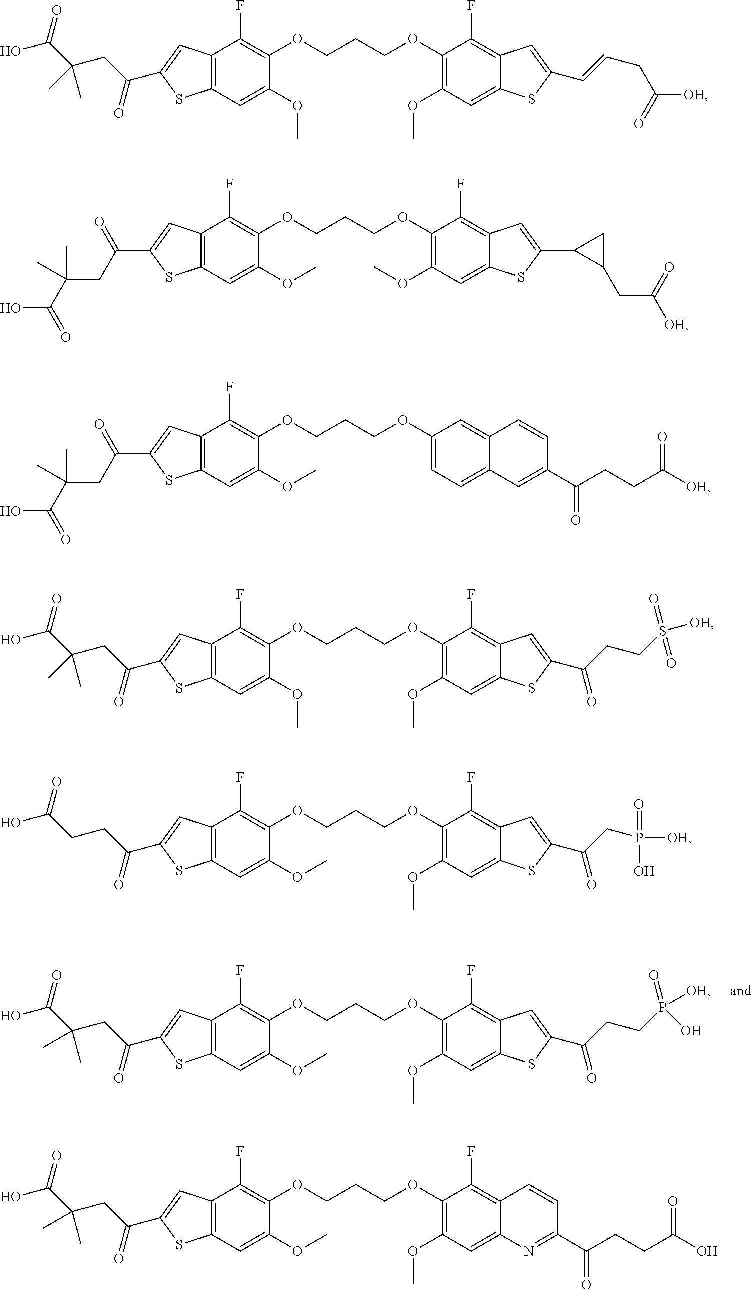
or a pharmaceutically acceptable salt, solvate, or stereoisomer thereof.

64. The compound of claim 1, wherein the compound is selected from: 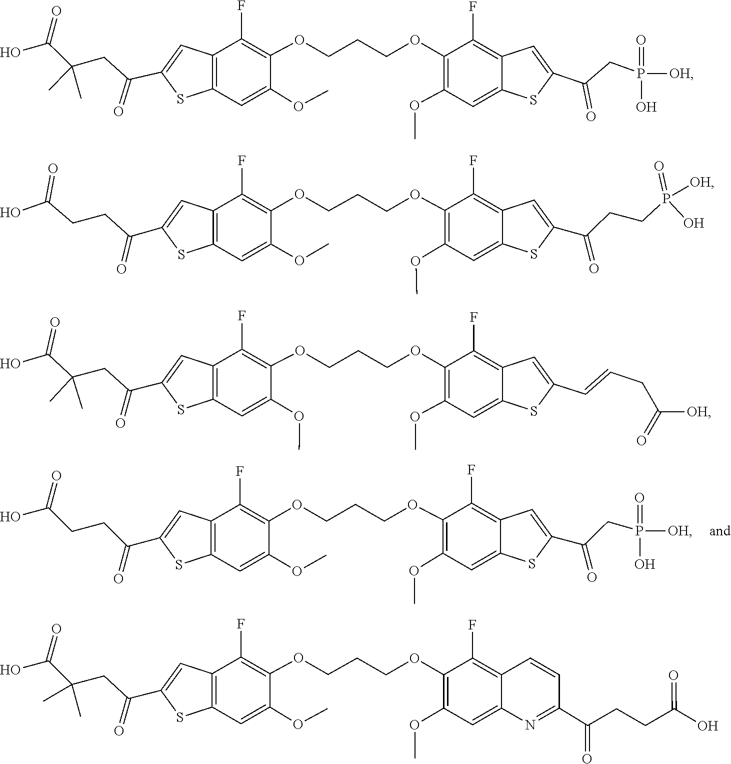 [SMILES: COc1cc2sc(C(=O)CC(C)(C)C(=O)O)cc2c(F)c1OCCCOc1c(OC)cc2sc(C(=O)CP(=O)(O)O)cc2c1F ; COc1cc2sc(C(=O)CCC(=O)O)cc2c(F)c1OCCCOc1c(OC)cc2sc(C(=O)CCP(=O)(O)O)cc2c1F ; COc1cc2sc(/C=C/CC(=O)O)cc2c(F)c1OCCCOc1c(OC)cc2sc(C(=O)CC(C)(C)C(=O)O)cc2c1F ; COc1cc2sc(C(=O)CCC(=O)O)cc2c(F)c1OCCCOc1c(OC)cc2sc(C(=O)CP(=O)(O)O)cc2c1F ; COc1cc2nc(C(=O)CCC(=O)O)ccc2c(F)c1OCCCOc1c(OC)cc2sc(C(=O)CC(C)(C)C(=O)O)cc2c1F]
or a pharmaceutically acceptable salt, solvate, or stereoisomer thereof.

65. A pharmaceutical composition comprising a compound or a pharmaceutically acceptable salt, solvate, or stereoisomer thereof of any one of claims 1-64 and a pharmaceutically acceptable excipient.

66. A method of treating a disease or disorder mediated by STING in a subject in need thereof comprising administering a compound or a pharmaceutically acceptable salt, solvate, or stereoisomer thereof of any one of claims 1-64 or the pharmaceutical composition of claim 65.

67. A method of inducing an immune response in a subject in need thereof comprising administering a compound or a pharmaceutically acceptable salt, solvate, or stereoisomer thereof of any one of claims 1-64 or the pharmaceutical composition of claim 65.

68. The method of claim 67, wherein the immune response induces STING-dependent type I interferon product in a subject in need thereof.

69. The method of claim 67 or 68, wherein the immune response induces STING-dependent cytokine production in a subject in need thereof.

70. A method of treating abnormal cellular proliferation in a subject in need thereof comprising administering a compound or a pharmaceutically acceptable salt, solvate, or stereoisomer thereof of any one of claims 1-64 or the pharmaceutical composition of claim 65.

71. The method of claim 70, wherein the abnormal cellular proliferation is cancer.

72. The method of claim 71, wherein the cancer is a solid tumor.

73. The method of claim 71, wherein the cancer is selected from lymphoma, head and neck squamous cell carcinoma, urothelial cancer, lung cancer, prostate cancer, SCLC, bladder cancer, and melanoma.

74. Use of a compound or a pharmaceutically acceptable salt, solvate, or stereoisomer thereof of any one of claims 1-64 or the pharmaceutical composition of claim 65 for the treatment of a disease or disorder mediated by STING in a subject in need thereof.

75. Use of a compound or a pharmaceutically acceptable salt, solvate, or stereoisomer thereof of any one of claims 1-64 or the pharmaceutical composition of claim 65 for inducing an immune response in a subject in need thereof.

76. The use of claim 75, wherein the immune response induces STING-dependent type I interferon product in a subject in need thereof.

77. The use of claim 75 or 76, wherein the immune response induces STING-dependent cytokine production in a subject in need thereof.

78. Use of a compound or a pharmaceutically acceptable salt, solvate, or stereoisomer thereof of any one of claims 1-64 or the pharmaceutical composition of claim 65 for the treatment of abnormal cellular proliferation in a subject in need thereof.

79. The use of claim 78, wherein the abnormal cellular proliferation is cancer.

80. The use of claim 79, wherein the cancer is a solid tumor.

81. The use of claim 79, wherein the cancer is selected from lymphoma, head and neck squamous cell carcinoma, urothelial cancer, lung cancer, prostate cancer, SCLC, bladder cancer, and melanoma.

82. Use of a compound or a pharmaceutically acceptable salt, solvate, or stereoisomer thereof of any one of claims 1-64 or the pharmaceutical composition of claim 65 for the preparation of a medicament for the treatment of a disease or disorder mediated by STING in a subject in need thereof.

83. Use of a compound or a pharmaceutically acceptable salt, solvate, or stereoisomer thereof of any one of claims 1-64 or the pharmaceutical composition of claim 65 for the preparation of a medicament for inducing an immune response in a subject in need thereof.

84. Use of a compound or a pharmaceutically acceptable salt, solvate, or stereoisomer thereof of any one of claims 1-64 or the pharmaceutical composition of claim 65 for the preparation of a medicament for the treatment of abnormal cellular proliferation in a subject in need thereof.

85. A kit comprising a compound or a pharmaceutically acceptable salt, solvate, or stereoisomer thereof of any one of claims 1-64 and instructions for use of the compound.

86. A kit comprising the pharmaceutical composition of claim 65 and instructions for use of the compound.
